SEQUENCE LISTING (43 sequences)

SEQ ID NO: 1 - DNA, 1398 bp, Homo sapiens
  source 1..1398: mol_type = genomic DNA; note = human PARK2 ENSG00000185345.24
atgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtag

SEQ ID NO: 2 - DNA, 1746 bp, Homo sapiens
  source 1..1746: mol_type = genomic DNA; note = human PINK1 ENSG00000158828.8
atggcggtgcgacaggcgctgggccgcggcctgcagctgggtcgagcgctgctgctgcgcttcacgggcaagcccggccgggcctacggcttggggcggccgggcccggcggcgggctgtgtccgcggggagcgtccaggctgggccgcaggaccgggcgcggagcctcgcagggtcgggctcgggctccctaaccgtctccgcttcttccgccagtcggtggccgggctggcggcgcggttgcagcggcagttcgtggtgcgggcctggggctgcgcgggcccttgcggccgggcagtctttctggccttcgggctagggctgggcctcatcgaggaaaaacaggcggagagccggcgggcggtctcggcctgtcaggagatccaggcaatttttacccagaaaagcaagccggggcctgacccgttggacacgagacgcttgcagggctttcggctggaggagtatctgatagggcagtccattggtaagggctgcagtgctgctgtgtatgaagccaccatgcctacattgccccagaacctggaggtgacaaagagcaccgggttgcttccagggagaggcccaggtaccagtgcaccaggagaagggcaggagcgagctccgggggcccctgccttccccttggccatcaagatgatgtggaacatctcggcaggttcctccagcgaagccatcttgaacacaatgagccaggagctggtcccagcgagccgagtggccttggctggggagtatggagcagtcacttacagaaaatccaagagaggtcccaagcaactagcccctcaccccaacatcatccgggttctccgcgccttcacctcttccgtgccgctgctgccaggggccctggtcgactaccctgatgtgctgccctcacgcctccaccctgaaggcctgggccatggccggacgctgttcctcgttatgaagaactatccctgtaccctgcgccagtacctttgtgtgaacacacccagcccccgcctcgccgccatgatgctgctgcagctgctggaaggcgtggaccatctggttcaacagggcatcgcgcacagagacctgaaatccgacaacatccttgtggagctggacccagacggctgcccctggctggtgatcgcagattttggctgctgcctggctgatgagagcatcggcctgcagttgcccttcagcagctggtacgtggatcggggcggaaacggctgtctgatggccccagaggtgtccacggcccgtcctggccccagggcagtgattgactacagcaaggctgatgcctgggcagtgggagccatcgcctatgaaatcttcgggcttgtcaatcccttctacggccagggcaaggcccaccttgaaagccgcagctaccaagaggctcagctacctgcactgcccgagtcagtgcctccagacgtgagacagttggtgagggcactgctccagcgagaggccagcaagagaccatctgcccgagtagccgcaaatgtgcttcatctaagcctctggggtgaacatattctagccctgaagaatctgaagttagacaagatggttggctggctcctccaacaatcggccgccactttgttggccaacaggctcacagagaagtgttgtgtggaaacaaaaatgaagatgctctttctggctaacctggagtgtgaaacgctctgccaggcagccctcctcctctgctcatggagggcagccctgtga

SEQ ID NO: 3 - DNA, 7584 bp, Homo sapiens
  source 1..7584: mol_type = genomic DNA; note = human LRRK2 ENSG00000188906.17
atggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcgcgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatattggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgttaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagattttgacagtgaaagtggaaggttgtccaaaacaccctaagggcattatttcgcgtagagatgtggaaaaatttctttcaaaaaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctaggaagccttaaaaatgtcatgctggtattgggctacaaccggaaaaatactgaaggtacacaaaagcagaaagagatacaatcttgcttgaccgtttgggacatcaatcttccacatgaagtgcaaaatttagaaaaacacattgaagtgagaaaagaattagctgaaaaaatgagacgaacatctgttgagtaa

SEQ ID NO: 4 - DNA, 1764 bp, Homo sapiens
  source 1..1764: mol_type = genomic DNA; note = human c-Rel ENSG00000162924.16
atggcctccggtgcgtataacccgtatatagagataattgaacaacccaggcagaggggaatgcgttttagatacaaatgtgaagggcgatcagcaggcagcattccaggggagcacagcacagacaacaaccgaacatacccttctatccagattatgaactattatggaaaaggaaaagtgagaattacattagtaacaaagaatgacccatataaacctcatcctcatgatttagttggaaaagactgcagagacggctactatgaagcagaatttggacaagaacgcagacctttgtttttccaaaatttgggtattcgatgtgtgaagaaaaaagaagtaaaagaagctattattacaagaataaaggcaggaatcaatccattcaatgtccctgaaaaacagctgaatgatattgaagattgtgacctcaatgtggtgagactgtgttttcaagtttttctccctgatgaacatggtaatttgacgactgctcttcctcctgttgtctcgaacccaatttatgacaaccgtgctccaaatactgcagaattaaggatttgtcgtgtaaacaagaattgtggaagtgtcagaggaggagatgaaatatttctactttgtgacaaagttcagaaagatgacatagaagttcgttttgtgttgaacgattgggaagcaaaaggcatcttttcacaagctgatgtacaccgtcaagtagccattgttttcaaaactccaccatattgcaaagctatcacagaacccgtaacagtaaaaatgcagttgcggagaccttctgaccaggaagttagtgaatctatggattttagatatctgccagatgaaaaagatacttacggcaataaagcaaagaaacaaaagacaactctgcttttccagaaactgtgccaggatcacgttaattttcctgagagaccaagacctggtctcctcggttcaattggagaaggaagatacttcaaaaaagaaccaaacttgttttctcatgatgcagttgtgagagaaatgcctacaggggtttcaagtcaagcagaatcctactatccctcacctgggcccatctcaagtggattgtcacatcatgcctcaatggcacctctgccttcttcaagctggtcatcagtggcccaccccaccccacgctcaggcaatacaaacccactgagtagtttttcaacaaggacacttccttctaattcgcaaggtatcccaccattcctgagaatacctgttgggaatgatttaaatgcttctaatgcttgcatttacaacaatgccgatgacatagtcggaatggaagcgtcatccatgccatcagcagatttatatggtatttctgatcccaacatgctgtctaattgttctgtgaatatgatgacaaccagcagtgacagcatgggagagactgataatccaagacttctgagcatgaatcttgaaaacccctcatgtaattcagtgttagacccaagagacttgagacagctccatcagatgtcctcttccagtatgtcagcaggcgccaattccaatactactgtttttgtttcacaatcagatgcatttgagggatctgacttcagttgtgcagataacagcatgataaatgagtcgggaccatcaaacagtactaatccaaacagtcatggttttgttcaagatagtcagtattcaggtattggcagtatgcaaaatgagcaattgagtgactcctttccatatgaattttttcaagtataa

SEQ ID NO: 5 - DNA, 2112 bp, Homo sapiens
  source 1..2112: mol_type = genomic DNA; note = human ATG7 ENSG00000197548.13
atggcggcagctacgggggatcctggactctctaaactgcagtttgccccttttagtagtgccttggatgttgggttttggcatgagttgacccagaagaagctgaacgagtatcggctggatgaagctcccaaggacattaagggttattactacaatggtgactctgctgggctgccagctcgcttaacattggagttcagtgcttttgacatgagtgctcccaccccagcccgttgctgcccagctattggaacactgtataacaccaacacactcgagtctttcaagactgcagataagaagctccttttggaacaagcagcaaatgagatatgggaatccataaaatcaggcactgctcttgaaaaccctgtactcctcaacaagttcctcctcttgacatttgcagatctaaagaagtaccacttctactattggttttgctatcctgccctctgtcttccagagagtttacctctcattcaggggccagtgggtttggatcaaaggttttcactaaaacagattgaagcactagagtgtgcatatgataatctttgtcaaacagaaggagtcacagctcttccttacttcttaatcaagtatgatgagaacatggtgctggtttccttgcttaaacactacagtgatttcttccaaggtcaaaggacgaagataacaattggtgtatatgatccctgtaacttagcccagtaccctggatggcctttgaggaattttttggtcctagcagcccacagatggagtagcagtttccagtctgttgaagttgtttgcttccgtgaccgtaccatgcagggggcgagagacgttgcccacagcatcatcttcgaagtgaagcttccagaaatggcatttagcccagattgtcctaaagcagttggatgggaaaagaaccagaaaggaggcatgggaccaaggatggtgaacctcagtgaatgtatggaccctaaaaggttagctgagtcatcagtggatctaaatctcaaactgatgtgttggagattggttcctactttagacttggacaaggttgtgtctgtcaaatgtctgctgcttggagccggcaccttgggttgcaatgtagctaggacgttgatgggttggggcgtgagacacatcacatttgtggacaatgccaagatctcctactccaatcctgtgaggcagcctctctatgagtttgaagattgcctagggggtggtaagcccaaggctctggcagcagcggaccggctccagaaaatattccccggtgtgaatgccagaggattcaacatgagcatacctatgcctgggcatccagtgaacttctccagtgtcactctggagcaagcccgcagagatgtggagcaactggagcagctcatcgaaagccatgatgtcgtcttcctattgatggacaccagggagagccggtggcttcctgccgtcattgctgcaagcaagagaaagctggtcatcaatgctgctttgggatttgacacatttgttgtcatgagacatggtctgaagaaaccaaagcagcaaggagctggggacttgtgtccaaaccaccctgtggcatctgctgacctcctgggctcatcgctttttgccaacatccctggttacaagcttggctgctacttctgcaatgatgtggtggccccaggagattcaaccagagaccggaccttggaccagcagtgcactgtgagtcgtccaggactggccgtgattgcaggagccctggccgtggaattgatggtatctgttttgcagcatccagaagggggctatgccattgccagcagcagtgacgatcggatgaatgagcctccaacctctcttgggcttgtgcctcaccagatccggggatttctttcacggtttgataatgtccttcccgtcagcctggcatttgacaaatgtacagcttgttcttccaaagttcttgatcaatatgaacgagaaggatttaacttcctagccaaggtgtttaattcttcacattccttcttagaagacttgactggtcttacattgctgcatcaagaaacccaagctgctgagatctgggacatgagcgatgatgagaccatctga

SEQ ID NO: 6 - DNA, 1545 bp, Homo sapiens
  source 1..1545: mol_type = genomic DNA; note = human VMAT2 ENSG00000165646.14
atggccctgagcgagctggcgctggtccgctggctgcaggagagccgccgctcgcggaagctcatcctgttcatcgtgttcctggcgctgctgctggacaacatgctgctcactgtcgtggtccccatcatcccaagttatctgtacagcattaagcatgagaagaatgctacagaaatccagacggccaggccagtgcacactgcctccatctcagacagcttccagagcatcttctcctattatgataactcgactatggtcaccgggaatgctaccagagacctgacacttcatcagaccgccacacagcacatggtgaccaacgcgtccgctgttccttccgactgtcccagtgaagacaaagacctcctgaatgaaaacgtgcaagttggtctgttgtttgcctcgaaagccaccgtccagctcatcaccaaccctttcataggactactgaccaacagaattggctatccaattcccatatttgcgggattctgcatcatgtttgtctcaacaattatgtttgccttctccagcagctatgccttcctgctgattgccaggtcgctgcagggcatcggctcgtcctgctcctctgtggctgggatgggcatgcttgccagtgtctacacagatgatgaagagagaggcaacgtcatgggaatcgccttgggaggcctggccatgggggtcttagtgggcccccccttcgggagtgtgctctatgagtttgtggggaagacggctccgttcctggtgctggccgccctggtactcttggatggagctattcagctctttgtgctccagccgtcccgggtgcagccagagagtcagaaggggacacccctaaccacgctgctgaaggacccgtacatcctcattgctgcaggctccatctgctttgcaaacatgggcatcgccatgctggagccagccctgcccatctggatgatggagaccatgtgttcccgaaagtggcagctgggcgttgccttcttgccagctagtatctcttatctcattggaaccaatatttttgggatacttgcacacaaaatggggaggtggctttgtgctcttctgggaatgataattgttggagtcagcattttatgtattccatttgcaaaaaacatttatggactcatagctccgaactttggagttggttttgcaattggaatggtggattcgtcaatgatgcctatcatgggctacctcgtagacctgcggcacgtgtccgtctatgggagtgtgtacgccattgcggatgtggcattttgtatggggtatgctataggtccttctgctggtggtgctattgcaaaggcaattggatttccatggctcatgacaattattgggataattgatattctttttgcccctctctgcttttttcttcgaagtccacctgccaaagaagaaaaaatggctattctcatggatcacaactgccctattaaaacaaaaatgtacactcagaataatatccagtcatatccgataggtgaagatgaagaatctgaaagtgactga

SEQ ID NO: 7 - DNA, 1611 bp, Homo sapiens
  source 1..1611: mol_type = genomic DNA; note = human GBA ENSG00000177628.16
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 8 - DNA, 423 bp, Homo sapiens
  source 1..423: mol_type = genomic DNA; note = human SNCA ENSG00000145335.17
atggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaa

SEQ ID NO: 9 - DNA, 2502 bp, Homo sapiens
  source 1..2502: mol_type = genomic DNA; note = human MAPT ENSG00000186868.18
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtga

SEQ ID NO: 10 - DNA, 636 bp, Homo sapiens
  source 1..636: mol_type = genomic DNA; note = human GDNF ENSG00000168621.15
atgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctga

SEQ ID NO: 11 - protein, 465 aa, Homo sapiens
  source 1..465: note = human PARK2 ENSG00000185345.24
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 12 - protein, 581 aa, Homo sapiens
  source 1..581: note = human PINK1 ENSG00000158828.8
MAVRQALGRGLQLGRALLLRFTGKPGRAYGLGRPGPAAGCVRGERPGWAAGPGAEPRRVGLGLPNRLRFFRQSVAGLAARLQRQFVVRAWGCAGPCGRAVFLAFGLGLGLIEEKQAESRRAVSACQEIQAIFTQKSKPGPDPLDTRRLQGFRLEEYLIGQSIGKGCSAAVYEATMPTLPQNLEVTKSTGLLPGRGPGTSAPGEGQERAPGAPAFPLAIKMMWNISAGSSSEAILNTMSQELVPASRVALAGEYGAVTYRKSKRGPKQLAPHPNIIRVLRAFTSSVPLLPGALVDYPDVLPSRLHPEGLGHGRTLFLVMKNYPCTLRQYLCVNTPSPRLAAMMLLQLLEGVDHLVQQGIAHRDLKSDNILVELDPDGCPWLVIADFGCCLADESIGLQLPFSSWYVDRGGNGCLMAPEVSTARPGPRAVIDYSKADAWAVGAIAYEIFGLVNPFYGQGKAHLESRSYQEAQLPALPESVPPDVRQLVRALLQREASKRPSARVAANVLHLSLWGEHILALKNLKLDKMVGWLLQQSAATLLANRLTEKCCVETKMKMLFLANLECETLCQAALLLCSWRAAL

SEQ ID NO: 13 - protein, 2527 aa, Homo sapiens
  source 1..2527: note = human PARK2 ENSG00000185345.24
MASGSCQGCEEDEETLKKLIVRLNNVQEGKQIETLVQILEDLLVFTYSERASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 14 - protein, 587 aa, Homo sapiens
  source 1..587: note = human c-Rel ENSG00000162924.16
MASGAYNPYIEIIEQPRQRGMRFRYKCEGRSAGSIPGEHSTDNNRTYPSIQIMNYYGKGKVRITLVTKNDPYKPHPHDLVGKDCRDGYYEAEFGQERRPLFFQNLGIRCVKKKEVKEAIITRIKAGINPFNVPEKQLNDIEDCDLNVVRLCFQVFLPDEHGNLTTALPPVVSNPIYDNRAPNTAELRICRVNKNCGSVRGGDEIFLLCDKVQKDDIEVRFVLNDWEAKGIFSQADVHRQVAIVFKTPPYCKAITEPVTVKMQLRRPSDQEVSESMDFRYLPDEKDTYGNKAKKQKTTLLFQKLCQDHVNFPERPRPGLLGSIGEGRYFKKEPNLFSHDAVVREMPTGVSSQAESYYPSPGPISSGLSHHASMAPLPSSSWSSVAHPTPRSGNTNPLSSFSTRTLPSNSQGIPPFLRIPVGNDLNASNACIYNNADDIVGMEASSMPSADLYGISDPNMLSNCSVNMMTTSSDSMGETDNPRLLSMNLENPSCNSVLDPRDLRQLHQMSSSSMSAGANSNTTVFVSQSDAFEGSDFSCADNSMINESGPSNSTNPNSHGFVQDSQYSGIGSMQNEQLSDSFPYEFFQV

SEQ ID NO: 15 - protein, 703 aa, Homo sapiens
  source 1..703: note = human ATG7 ENSG00000197548.13
MAAATGDPGLSKLQFAPFSSALDVGFWHELTQKKLNEYRLDEAPKDIKGYYYNGDSAGLPARLTLEFSAFDMSAPTPARCCPAIGTLYNTNTLESFKTADKKLLLEQAANEIWESIKSGTALENPVLLNKFLLLTFADLKKYHFYYWFCYPALCLPESLPLIQGPVGLDQRFSLKQIEALECAYDNLCQTEGVTALPYFLIKYDENMVLVSLLKHYSDFFQGQRTKITIGVYDPCNLAQYPGWPLRNFLVLAAHRWSSSFQSVEVVCFRDRTMQGARDVAHSIIFEVKLPEMAFSPDCPKAVGWEKNQKGGMGPRMVNLSECMDPKRLAESSVDLNLKLMCWRLVPTLDLDKVVSVKCLLLGAGTLGCNVARTLMGWGVRHITFVDNAKISYSNPVRQPLYEFEDCLGGGKPKALAAADRLQKIFPGVNARGFNMSIPMPGHPVNFSSVTLEQARRDVEQLEQLIESHDVVFLLMDTRESRWLPAVIAASKRKLVINAALGFDTFVVMRHGLKKPKQQGAGDLCPNHPVASADLLGSSLFANIPGYKLGCYFCNDVVAPGDSTRDRTLDQQCTVSRPGLAVIAGALAVELMVSVLQHPEGGYAIASSSDDRMNEPPTSLGLVPHQIRGFLSRFDNVLPVSLAFDKCTACSSKVLDQYEREGFNFLAKVFNSSHSFLEDLTGLTLLHQETQAAEIWDMSDDETI

SEQ ID NO: 16 - protein, 514 aa, Homo sapiens
  source 1..514: note = human VMAT2 ENSG00000165646.14
MALSELALVRWLQESRRSRKLILFIVFLALLLDNMLLTVVVPIIPSYLYSIKHEKNATEIQTARPVHTASISDSFQSIFSYYDNSTMVTGNATRDLTLHQTATQHMVTNASAVPSDCPSEDKDLLNENVQVGLLFASKATVQLITNPFIGLLTNRIGYPIPIFAGFCIMFVSTIMFAFSSSYAFLLIARSLQGIGSSCSSVAGMGMLASVYTDDEERGNVMGIALGGLAMGVLVGPPFGSVLYEFVGKTAPFLVLAALVLLDGAIQLFVLQPSRVQPESQKGTPLTTLLKDPYILIAAGSICFANMGIAMLEPALPIWMMETMCSRKWQLGVAFLPASISYLIGTNIFGILAHKMGRWLCALLGMIIVGVSILCIPFAKNIYGLIAPNFGVGFAIGMVDSSMMPIMGYLVDLRHVSVYGSVYAIADVAFCMGYAIGPSAGGAIAKAIGFPWLMTIIGIIDILFAPLCFFLRSPPAKEEKMAILMDHNCPIKTKMYTQNNIQSYPIGEDEESESD

SEQ ID NO: 17 - protein, 536 aa, Homo sapiens
  source 1..536: note = human GBA ENSG00000177628.16
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 18 - protein, 140 aa, Homo sapiens
  source 1..140: note = human SNCA ENSG00000145335.17
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 19 - protein, 833 aa, Homo sapiens
  source 1..833: note = human MAPT ENSG00000186868.18
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQEELRVPGRQRKAPERPLANEISAHVQPGPCGEASGVSGPCLGEKEPEAPVPLTASLPQHRPVCPAPPPTGGPQEPSLEWGQKGGDWAEKGPAFPKPATTAYLHTEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 20 - protein, 211 aa, Homo sapiens
  source 1..211: note = human GDNF ENSG00000168621.15
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 21 - DNA, 1151 bp, synthetic construct
  source 1..1151: mol_type = other DNA; note = Monoamine Oxidase B MAOB
acagaagcacttcacctgtttatttcttgaaaagccattctatgttgctgcttcccacttttagttctcagaggtggctttcctactgtccagaagacataaacttctgttttccctggagtcaccttagcttaaactcctataatcttgttgaatcatccctgtgtttggctaaaccaggctcctcatacccacgttccagggtcacacaggtaacttggcttgctgcttctgggggctctgaatcccacaatgccgatccaccacctcttccctgctgccactagtctcctggggtctagcctcaacagcttccaccaaggacacactgcattgcttaagagtgtatttaaggtgcggcatttgtgatgctagggaggagccttcctgacttaatcacgtagggggagtggggaaagaagtgggtatttggaaggagggtgaggtgggctgggaatgccaagaaatggtagtactgtgtggtgaatcaaagtgggtcttggaatcttcctagcttttcttttcttttcttttctttttttttctttctgactttttatcttatgttgtcactcaccatgttttgttttcctcatctgcaaaatggattgttgtaagcttgaacaggaatcatgcataaagtactgagcacagtgcctggtaccttgaaatcactcaatggaagtttcttattatttgccctacacccaaggagtaattggggccctgaaggaagacaccccaatttgccgtcctcccagccgccaagcctgatagtgcactagggaggctgatgggaaaataccgtgcagaaggctgaggagtggggattctggggcgggactctttgaagtcctaggtgacctctccgcccaggcacccgccctccccgggcctgggctgcagagctgcgggcgggggcggtggccctgcgctgcacccggcctgcctgcgcgtaggcgggcgggcggggctgcgcgtccgggctcccggggctggtaatatagcggctcgccgaggcgctggtgcacgggggcagcgcgcagcaggccggcgggcaggcgggcgggctggctggcaggcaggactgggatcgaggcccagaaaacggagcagcgggcaccagggaggcctggaacggggcgagcgcc

SEQ ID NO: 22 - DNA, 1161 bp, synthetic construct
  source 1..1161: mol_type = other DNA; note = GTP cyclohydrolase 1 GCH1
cattatgagataatccctcaagtgaggaaaaaggtccatttattaatctcaaagaaaacagttacagcagatgtcactggttaagagttcagttggtgaatagcatttcacaatttgtaccaacatctggggaaagacgctttgcatggaactgtaaaacaattgagcaccaaatctgcacaactgcgtttctagaaaatgcgatgggttttatagagatgaggtcttgctatgttttccaggctggtctcgaactcttggcctcaagcgatcctcccgcctcggtctccccaagcgccgggagtacaggcgtgagccaccgacggaaatggattttaagtgaaagtcctatcttcgtttgcaaatcaatttttccaggatcaaagttctagcaaccataaatcctatccttctagacatagggacccacataagggccacttgatgtgacacttgccccgacagcggtgccacctggcccctaggtggcaggtgcgtttttaactaaacccaagagtctgaagtcacattattctcttgccttgaaagcttaagagttggttcctaatcggtggcttaactcggtctctctccaggcaaggccacctgtttgctgatctttcacagggcgaggggaccggagcccttcaatgcagcgccctcttggcctgaagagggggcgactgggcgcccagaccgcgtctttctcactgagatcccagctcctggacgactgcctcttttcgggttcggctcattccgcaataagtggaggggtcccgcctgatcctaggggccggcgcccttttccttccctccctgcgcttgcgaacccctccgggcgtctccggagccgcgacccctgctggggaccccgggggcgggaggccgaggggcgcggcgagggccgtgacgcgaggcggggccggccaatgggagcgctccgcggcgcaggctgagccgccgggccataaaaaggaggcgcggccgggctttccagcctgtggccgctcccggctcggagtgtgatctaagcaggttgcgtaccttcctcaggtgactccggccacagcccattgtccgcggccaccggcggagtttagccgcagacctcgaagcgccccggggtccttcccgaacggcagcggctgcggcgggtcc

SEQ ID NO: 23 - DNA, 2006 bp, synthetic construct
  source 1..2006: mol_type = other DNA; note = Nuclear Receptor Subfamily 4 Group A Member 2 NR4A2
ccacttcctggttttattttttttctcgcgcccgctagggcatcgccgagcagcggcggcggcggcagcagcagcggcagccgcaacatctgggggaaacttaaggtggtcacgtaggtttccccgaggctgcggcgcaaaagagcccgcggcttcctggggcagccgcagggcatctgatggcaacgcctccccagccgccggcgagctgggggcagcagggagtaggcaaaggcggggctgcagattagggttgagcagcccgggatcccgaatagttccacggagccatcggccggtactgagaacacgagtaggggggcaagttgcaaaagggtcccggctgcctggaaacgagccacaccgagaaaggaggggcggctgggacgctctgcgcgagtagaaaagcgggggcggctccgaactctccctcccaaggcccagccgctggaagccccgcgggagaagccctaggaagccctcccagctccggccgggagacggggagggggcttgccctccggagctgcggcgtttcctttgctagaaagctccctgcgctctcggcgtggctaggggaaatgacttgcaaagcagaagcggcggcgacttggacggccgcgggggtgggacacgctcccagagccagctcgggcccctctcctggctgggctgtaggctcacaccttacgctttgcggagacgctgcccccaatccccacccgcttcgcttttcttgtccagtctttctgggctctaagattgaggctgtgatagaccttgtgtttcaaacgaagatcgatttattcccttaaaactcgtggaaggggtacgagttggtgggcacagaggagtatcgaaagtattagagtttgcagaaggtgggaatcagagtacctttctgcagaaggcgcacctgttgcctgctgctcggctgcattgtctgcgaaaggaaaaggggcgacgcttcactctgacttttggggtttctaaagagctgggtcacggtcactgtctaccctctccccttcctctcccccccccccgcaagcaccacgtcctccatcgaacgtgggcactgcatggaaataaggaaacatagaaaaataagccctacccccactcccattccctttcagatgggagtgtggggggtgggggtggggtagagagagtgagagagagagataattagaatgaatatatgccagaagaggaggaggcctgggacaggaaaagggagtaaaaggggatgaaccgggtagggaaatcccacccgaactgcgtgagccctctgagcgtctcgtgtcatgggacatctgtacgctcttccgctaagggggtgacgaaggtgggtgggagaggttagggcgctgcaaggcacatcccattccgtgcggctccaggtgcgtactacctggccacgccggccttgccttccgccggtgcttttgttgcaccctccccacatgtgagcggccgagccgctgcgcgggcgcaggggcttcggggaaagtgaagtgtcgcgacgctgcgggctgcgcagacctgggagaggtcacacctctttcggaaaaaaaaaaagaaaagaaaaaaaacaccaaaaaccacccaagctggctaccaaggtgaacgcagagcggttcccaccttaaaatcggccctgctcgtgacgtcaggtcggaaatataccaaagcgagcgcgggccaggagtccagggagcgcggcagcgcggcgattgggcggcgggccgctgacgcgcgctgacgcgcggagactttaggtgcatgttggcagcggcagcgcaagccacataaacaaaggcacattggcggccagggccagtccgcccggcggctcgcgcacggctccgcggtcccttttgcctgtccagccggccgcctgtccctgctccctccctccgtgaggtgtccgggttcccttcgcccagctctcccacccctacccgaccccggcgcccgggctcccagagggaactgcactt

SEQ ID NO: 24 - DNA, 3015 bp, synthetic construct
  source 1..3015: mol_type = other DNA; note = Mouse Tyrosine Hydroxylase TH
ctgctaggggctgcttcccagctactcctcttggctccgtggcttgccttccagcctgtgtgctgtctggagagcctttaaagcctcacttccaccaactagaagtctctccccaaccctgccctgacctcaagtgcacctcttcaaagtcaggtttagcagctgcagctgggggccctgaatcccacccctgctgtcttccttgaagacagaagtgttgggagctgaggatctgggctagagactggctgtatgatccagagaagtagtgtgcttctgggcctcagatttcccttctgtagaacaggtttgtctgaaatggagaggttggtgctcctctgcagggcctagtgggagtcaccatgagtggttaaaagatccagcttgtcttttggtgagctttgagaggaggtaacagggctgagttctggaagcctgaccaagggcagacttaaggggcctcttggagttgttctcatcaaatggggatgggacacagctaaagtgcccagggcttctctgtgcccacagatgctttagatcttggcacagtgtggtctaccagctgtctctctctgtgtatatatatgtatttcatagacagtgtacagtggcctggtttgtgctatcaggctggatatggacagaggcaagagtttgtggcagcagttatctcccaagagagtccaaagacatcatgttttcaagtttaggccaggtgctacttgagagagctcagacacagacaaaggtctggagagcacatgtcctccacccccacctagcttctgttgcaagcacctccagccgagacaagagaacgaattaaaaagcaatatttgtgtcagtgtaagacatttgccgaaaggttaaatccacattcgtgttgctgcagagcagccccctatgcaggatttgttagatacagctccgtcctaccctgtgccagctgagcaaacgccaggctgggtggggtggaacccagcctgggtttgcctcaccctgcaatccccccagcaccctctaaaggaggaccctgtggtgggcatgcagacctagggactgggcatagataacctttgggtttgggcaacagcccccactcctcaggattgaaggctaaggtgcagccagctctgccttcatggtgggaatgtctccacgtgacccctttctgggctgtggagaacactcagagaagagtcctgggatgccaggcaggccagggatgtgctgggcatgttgagacaggagtgggctaagccagcagagttgctgacccaggaagagttcagaaaggggcatggaacatggggaggggtccatagtgagagagagcaggcagtgcagagtaaatagtccctgagctgggggttatgggatttgcaggagcttgctcagagaaggcagaggagagatgctgcgccaagctgggtatcacagagcctcagactcctggaacaggaactgtgggggtcaggtcagcaggggaggttagggagtgttccctttgtactgacttagcatttatcctgcttctaggggggaaggggggccagtgggggatgcacagcaaggcagtgatgtggcaggcagcctgcgggagctcctggttcctggtgtgaaaaagctgggaaggaagagggctgggtctggtaagtacagcaggcagttggctcctgagagtccaagccctgtctagagggtggagtgagatttcagagggagagctaaacggggtgggggctggggagtccaggcttctggctcctgctaatactcagtgtgctgggtcctcagaacctcagggtggccattttcagggtgagagctctgtcctttggcacttctgcagactccagtatccagaggaataaagatggtactcttcctcagttcccttagtgagaggacacctttctctgaagggcttgggcagttgtcctgaaccattgcctgaaggaaggacttgactccagggacatagaatgggctcagcataagtcccctgtagtagagaaaggtcccctctctggtctccttagagatcctgtttccttggctgaggaagctagggtggatctttgtgtaagtgggtgtggatgctcactggaaatcaaaaggccccttggtgttagaccttggggtgccatgggagagttgatcactgagtgcgcccttacatgggggccagctgagaatggggctgcctctagctcgagaccatgatgcagggagtgagtgggggagttcaggatactcttaactaaagcagaggtctgtccccccagggaggggaggtcagaagaccctagggagatgccaaaggctagggttggcaccatgttgcaggctgtgtcttcaaggagatgataatcagaggaatcgaacctgcaaaagtgggccagtcttagatacactatagaggaataatcttctgaaacattctgtgtctcataggacctgcctgaggacccagccccagtgccagcacatacactggggcagtgagtagatagtatactttgttacatgggctggggggacatggcctgtgccctggaggggacttgaagacatccaaaaagctagtgagagggctcctagatttatttgtctccaagggctatatatagccttcctaacatgaacccttgggtaatccagcatgggcgctcccatatgccctggtttgattagagagctctagatgtctcctgtcccagaacaccagccagcccctgtcttcatgtcgtgtctagggcggagggtgattcagaggcaggtgcctgcgacagtggatgcaattagatctaatgggacggaggcctctctcgtccgtcgccctcgctctgtgcccacccccgcctccctcaggcacagcaggcgtggagaggatgcgcaggaggtaggaggtgggggacccagaggggctttgacgtcagcctggcctttaagaggccgcctgcctggcaagggccgtggagacagaactcgggaccaccagcttgcact

SEQ ID NO: 25 - DNA, 5856 bp, synthetic construct
  source 1..5856: mol_type = other DNA; note = Solute carrier family 6 member 3 SLC6A3
ttgaggaattctgtgcttcttccctcttggtcttcagccaatatgaaaaataaacacacaacaggaacgcaacaattcagcagccagggctagggacatacctggtgcggagggcagagccccgaggcattcacgggcattcctctgggaagggatgggtgcgttctcagcgcctgagatggtacagctggggcacgggagctgggaagtgccagccctcaccacagggctggggcactggcccatggaggcctcaagacagacactctggtttgctcctccttcccctgcacccctccccactcctatcaaagtgcaaggctgtgtgagctctagcgtggctttctactaacaatgcatggatatcacttccaccttcccccacacttccagagagcaattttacaatcttctaagagcattcaataatgtcttcatgactgttcatctgtattcttttttcttttttttttcttttttaacatgaggtctcgctctgtcacccaggatggaatacagttgtgattacaactcactgcctcaacctcctaggttcaagtgatccttgcacctcagcctcctgagcagctgagactacaggtgtgcaccacagcaccggctaagttatttttgtgtgtgtttttttgttgttgttgttgtttttttgtagagacagggtctgactattgttgccaaggctggttttgaactcctggtctcaagcgatcctcctgcctcagcctcccaaagtgctgagatgacaggcatgagccaccaagcctgggcttgtattgtttatactcagtaataaagagcaaaatttatataagcaaaatcagccactgtaaagagagtttagacataaccatcacaactgcttcctcaaaaagtgtctgtacaagccacatccacctagggcaggtggcaccaccccgcgtgagagagctgggcggaggatggacagggcttcatcgcgggaactagctcctgggccagcagggtaacccgggaaatcctgtccaaacggctacataaacccccctgcaacgctgagcaggagagcaggagcgtctacaccgccccagccagggcgacttctgtcatttggatccagcagcagcgtgtttcagtcgagcgcacgcacacacagacacacagacacacacagactcacaaacacacatacacactcacacacacacaggcgagcacatacacttacacacatacacactcacactcacacaggtgcgcacacacacacaacgactgaagcaggtcgcaggtggaggctctaacaggcaactttccctgcatccaagtttgcacaaaacctcacccatgagctatcacgagctatgccacgcacccggtgacaacctcaaccggcacgacccctccggtgggtaaaacacccgacgaaggggttccgcggcgcgagcaggaggccgcgttgggagagggcgtcgggtgcggagctcgcgagtctccggcaagccgccgccgccctggggccacctgaccccgctcctgaacgcggggcctcagaggcgagttttgggtctacacagcaaaatgtggcccgttagaaagcgtccttcctcaccgcgcccagaacaagaagcggcgctgcctggcgagtcctcgcccggcctccccgccctgcccagagcaccagcgcgcagcccgcgcccgccaccacccccaacacagacaaagcccccgcgagatggagcggcggtgtacaaaacccactccgatcggatctgggatgcgccgcacccctagtagggttaagttccacgagaagaaaccaggacccccgagtcacagccatagacacccacgcgtcccctaagccggtgactgcaccaggtgcccacccggttctgagcctggagcggggccaggggcagccctgcaccctcctcgcgcctcggacggcctcagggtcccatgtggcgctatcggggggcgctcggcacccgcgactgttggcgactttggagacggcgcggtctgcgggacgcggggaccccagactgtgcccggtccccggcccccgcccctgcgccccacttcggggtctgcggaaggagcggggcgccccgatgccgcgagcgacgctggcctcacctggccgcccgggcctgggctttgcacgcgagtcctggcgccgagagcgttccgcgaagcctcccctcccgctccgcagcgctgggcggtctcagcctcggcctcgggctcttatccagtagacagggtccgccccgccccgccctcccgccgacggggtcgccctcgcgtcctggctcctcctcctcacctccccgggccgcgcccccgcccccgcctcccggacgcagatggctgggagcaagcggcggccacggctccactgtcctgcgcgtcccggagcctcggacacggggtgcgggggcctgggggtccgagacgtcgcggatctagacgggcgccttccacgcgggaactgtctgggccgccctgcgccctcgcccgcacccctgggggctgccccgggcacccgctatcttcggacgcccagggctggcggctcccagctggacggggagggagccgagcggctgcgcgcggggaactgctgggaccccgtcgggccgacccttccctccctccccatcaggggcccctacggtggccccgcgctctggggaccacgtccctcccaccccgccgctccacgctctgaccagcgggctcgggaccccgccgaggactctgcacgcttcccggcgtgcgggctgcggagacgcgggtccttccgtggccttggggtctccgcgacctcgaggcgaccgaggtcccctaggcctagtccccgccttgccggcatctagtgcgcctgcatcggaaccgaggggcccgcctcgcactcgcctaagaaaaccatttcccgggacgggcctggaaagccctggcggttggtatccggctccagggccgaatggactccgaggctttccttccgagatgcccgggcgggatttcttccattcagcgcgcggaggaatggagcccccaggccgccaaggcccaggatgtccaggtccttagaaggcaccgaggtgagtggccgcgcccagctctgtgcgtcgcccctagggtcgcccgaaccccggcgttcctccccgtgtaccctcgatgctcccgtgagcaccctgccctgggcgtccaggcgagccctggggctctccagccattccagcctgcagcccgcgctcagcagtgagggaggcagggacccttggtgcctgactccgcagtacccgcccgcagcgcggtgccccaccccctgcgcccgccccgcactccccgcacaccaccactcccctccccgccccccgctcccccgctccccggctccggggctggcacccaggcagaaggcactcagggctgtgacttcacgctgagttggaatcaagtgggagcctagtgaccccctggggctgccatgacaaaaaactccacagcctgggggctcacacagaagccttcagttctaacggttccggtgtcccgaggccaaggcctggccgactctgtgtctggtgagggccgcttcggggttgcagacagttctctccctgtatctccccacgctaggggctggggagagctctccggggtctgttttatgagggcactggtcccattcctcagggtggagccctcctggcctgaccacctgaaggcctcctgacaccacctcccatcaggggtgggacttcatcgccagggaggtgaggggacttcaggagagggggatgtgccacctcgaagctgtagtgtgtcagtggctgccacagaagggatctctctacccctattatctccatggcacaggtttcaataaaaaggagtgagccagggcttggccccagagcagcagggttgaggaaacagtctcccaaggaaaagactttgcaggtgactaagggaggctgttaaataaaacacacagagagaatggatgttcaggtatattaaaaatacacaggccaggagtgagggctcacgcttgtaatcccagcactctgggaggccgaggaaggaggatctcttgaggccaggagtttgagaccagcctgggccacatggcaaaaccctgactttgaaaaaaaaaattagccaggcatggtggcccacacctgtagtcccagctactggggaggcagaggtgggagaatagcttgaagctgggaggtggaggttgtagtgagctgagattgcgccatgggccacaggatgagaccctgtctcgaaacaaaacaacacacaggcaaacagcgtttgggaagggattgcaagcgctgcaggagcgcagaattgtgacccaaagctaacagggtacaggcaggaaggtgcagggactagaggtcacacagatccaggcagccacatccttactgaccagtagcacacagagaatgcacctgccacactcaggaggggccactgagtgatcacacgcatgtaacctgcactctgccaagacaaagagcattcctccacccctgcctcaaccccgacttccaaccacatgaattaggataaaggagctttgggtgctctaagaacaaactaactgcgggagttgggctcatggatgacacagagaaagcgcagctgccagacccatgggtctccatcctcccacaacaggaatgctccgtgaactgtgcaggtagaagcagtgtggttatgagctgatcagacctgatcaaacggccgacctttgcatgcttggaactttgccctaaatgccaagtcggctttattatcattgtggttgctgctatggctgctgtgggctcagggcgttgggtgcaggcaggtggagcccccctgcctctgcacttcctgcccatgctcggcacctctccccactggctgggaccgcagctgcagagcccacaaggagcccctggccactgactgctgtctccaaagcaaccttgccccacgggtccccccctccttgggtgctgcctcctgggaggccctcagtgctggatgttgcgccgtgtctcgcctgatttggaagacagcgtggatgctccccccatgagtgctgtatctgagaggggacaaggacagtggcagtgagatgacagtcatttacctgcctgcgtatttactatattgagggtgtcctgtgtgctttcagaggaagtcccagggagcccagctcagactttaaagaccctgacctctcactggtgctgggaaggccctgggggtagggggcttggggagggcaagggtgaggggaccagtaggcaggagtggcttttgtccaggggtgtgggaggtcagatcccagaccttgtctgtccaggttctgagaatgatggaaggcgctggctccaggcaggaggccctgctcactgctgcacccctgtgccctccccgggggccacctcaggaggaagatagtttcctctcgcatggcagggacagcagtacacttccaatgtcttctctcagagctgtatcattttccggctcactgctggttttagtcacagacatcttgatggtcttgccactgcacagcatgtcacagtggtctgccacactgatgacatcaggctgaatcaacctggggagcaaaaaatgaaaaatgccaagatgccttggtaagacacgtgcttgctaaaaattgaaagagaaaataacacattttcaaaacctgccaccttggtgaactttctgtgggtccattggtctgattggtcaaaatatatatccaaaatgaaagaaaacacgctgtctctggcaagcaagactaccactaagaaagaagtatca

SEQ ID NO: 26 - DNA, 469 bp, synthetic construct
  source 1..469: mol_type = other DNA; note = Synapsin 1 SYN1
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 27 - DNA, 1337 bp, synthetic construct
  source 1..1337: mol_type = other DNA; note = Mouse alpha-calcium-calmodulin dependent kinase II Camk2a
cccttaattaacattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagctctagagcgggggatccactagttctagagcggccgc

SEQ ID NO: 28 - DNA, 2710 bp, synthetic construct
  source 1..2710: mol_type = other DNA; note = Neurofilament medium chain NEFM minipromoter
cttggtatgttcttttggactgaaaactgatggagttggaagtgacttttgtagtgaaaatgaggctggagtaattaatattgttccagtgttctggttatgcgcaggagtgaatcatgatacccaaattacaatgactcatctcttctgagagaataaattgtgattctatgaaagttttgcttcactaggaattttttgaagggttctgtttgttctagtttagaaacctacacaggcaatatgggctttgcaaaggtataaaattttcttaaataaaactaaacaaagatctattttgattttcagcgttggattttttttttttcttgcctttcctttgcttctgggagaaagtatcttcagacaggaagctttgagctactcataaattctccctaacactgcagaaacatatttcactatgaaatggtggagatgacagtaagagcctcttgtttgaggggcaaagctgagacatcctgaatgctatggaagtttaatcttccatgctgacgcaatcacgggaatattgtgctcctggggttttctttattgccagctactgtctaatgccaggacaaaaggaataatttgattacagccgaggaaagcacagtttgttcctgttacttttaagatgtgcacgcatattaaggttaacaagatttgtggttgtgaagtataattctaaagcctcacaagcatgaagggtgtgtggacgttttcctgagtcatcctagggaacaaaagggagtgggcagagctagttaccagagttggaaagggatgcatcagtcattacattcaggggctgttagttggttccttttgcaaatgggctgtgaactgcagatgtccctgggagagaaaatagaaaattactagcaaattcctaagagaacctggcctctttaaagaagagaagacacattggggctggggatgacatcccaaaactcccagcgccggggagctcaggctcgggcggtgcctggctcccctccccggttcccgccgcaggaggctgctgagtcactcccgcctgagccacggccgcccccagccagccacgcgattggcgggttagcccgggcgcagcgccctcttcccagctcccgcacccccagggcttcccgcaggcagagggtgggagccccggcgagcgaccagttggggcttggtaggaaccccctaaaaatgcgccccacttgtgcattcgtgcatgggcgcattttggttcagaattaattgagagttgttcactctactatttattaactagatgatccgatagttgctgcaaagtaacttgtggttaatgcaggtcagtagagagctgatctcactgcaggccggaggcaacgcccaattcccacgtcttcccctcccccacccaacaagggctgggcagctttaatgcggaacgccggtgcagcagcacggagcacactcccagactacagtggcgcccgcactcacgcccttcaagtgccccacccctcccacacagtggccaaggagccaagccctatccctctgcgatctgtccattgtgccaaccctgcattgcagggaagtcttagcctgtgattccagcttcgctcctgcaaagatagtcactacaggcaggtttaaattgtatttactcggagagaagagggggaagggaaccccctgccccctgccaacctgacaggccaggctgggtgacaagccccccgggtggctgcacctcccactcgccgacctgggcgctgtccttggtgctgccggcgccatagcgagcacaagcgccttaccttctgcccctgcggaagctgcggacgtcccccaaacgttaaaaacgccatatcccactttttatacaaataactaccccggcctgttctctttgtccccttcccgaagccttcaggcagaccgttccgcaaagtctttttttttgttttgttttgtcttctagacccagctcctcggcgctcatctccagctgctttaagacaaggggtgggggaaggggagggaggcaagaaaagatgagggtgggggaggggaaaagagggaatgcaaggggaaggagggaggagacggggagaaggaaagattggaagaaaaggatctccgaggaaggggctgagagaagggcagggtgaactggactaaaggccagagtaggaaggagaagaggggccaaaaaagaaggggatgaaattaagcacagaagatgggtaaagaaaaaagtatcagggaaagggcaaaataagagaaagccttgaggataagagggtagaaggctaaagaacaaggggaccactgggtcggggaagcgctgcctgaacggcgggacagtgacaaagaaagggcgctggcgatattcgcaccaagggtgcgaaacgcaatcgggaggtgagaaatggaaagaaggcgaatgcccggctacaagtagcctgggactgaaaggggacctgggggaggggctgggcccagggcagaaaagtccaggttcccatgcggcctgggcccacgtggagcgggcgctgaatcaccgttcagccgcccccctcccctcctccccgaccggtgcccgcagtccccgcctcctcggccgccgcctccacggggcggggccctggcccgggaccagcgccgcggctataaatgggctgcggcgaggccggcagaacgctgtgacagccacacgccccaa

SEQ ID NO: 29 - DNA, 2693 bp, synthetic construct
  source 1..2693: mol_type = other DNA; note = Neurofilament light chain NEFL minipromoter
cttggtatgttcttttggactgaaaactgatggagttggaagtgacttttgtagtgaaaatgaggctggagtaattaatattgttccagtgttctggttatgcgcaggagtgaatcatgatacccaaattacaatgactcatctcttctgagagaataaattgtgattctatgaaagttttgcttcactaggaattttttgaagggttctgtttgttctagtttagaaacctacacaggcaatatgggctttgcaaaggtataaaattttcttaaataaaactaaacaaagatctattttgattttcagcgttggattttttttttttcttgcctttcctttgcttctgggagaaagtatcttcagacaggaagctttgagctactcataaattctccctaacactgcagaaacatatttcactatgaaatggtggagatgacagtaagagcctcttgtttgaggggcaaagctgagacatcctgaatgctatggaagtttaatcttccatgctgacgcaatcacgggaatattgtgctcctggggttttctttattgccagctactgtctaatgccaggacaaaaggaataatttgattacagccgaggaaagcacagtttgttcctgttacttttaagatgtgcacgcatattaaggttaacaagatttgtggttgtgaagtataattctaaagcctcacaagcatgaagggtgtgtggacgttttcctgagtcatcctagggaacaaaagggagtgggcagagctagttaccagagttggaaagggatgcatcagtcattacattcaggggctgttagttggttccttttgcaaatgggctgtgaactgcagatgtccctgggagagaaaatagaaaattactagcaaattcctaagagaacctggcctctttaaagaagagaagacacattggggctggggatgacatcccaaaactcccagcgccggggagctcaggctcgggcggtgcctggctcccctccccggttcccgccgcaggaggctgctgagtcactcccgcctgagccacggccgcccccagccagccacgcgattggcgggttagcccgggcgcagcgccctcttcccagctcccgcacccccagggcttcccgcaggcagagggtgggagccccggcgagcgaccagttggggcttggtaggaaccccctaaaaatgcgccccacttgtgcattcgtgcatgggcgcattttggttcagaattaattgagagttgttcactctactatttattaactagatgatccgatagttgctgcaaagtaacttgtggttaatgcaggtcagtagagagctgatctcactgcaggccggaggcaacgcccaattcccacgtcttcccctcccccacccaacaagggctgggcagctttaatgcggaacgccggtgcagcagcacggagcacactcccagactacagtggcgcccgcactcacgcccttcaagtgccccacccctcccacacagtggccaaggagccaagccctatcccttcagggctggccgcagcggcttccctgaaaatcagccaactgcaaggcttatcgaaatcatcaggtcgtgtgctacacatgtgtaaaaagaggaaagcggactttaaatgtgctgcggttggtggtagcaagcaggaatttagcttggtgaggatccaggcagcttgaagctcccggctgcggagcgcgcggctgcctcagcaagtcagtctctgtgtttccaactctttctctgccctccaccacccacacactgcaacacgttaaagccatctgcggcttcattctcagttaagaaatgtgaaaatctagaaacactaacaggcggattaactgctgtaaaggtttaaaaatgctaaaccaatacctgcagtagtgccgcagtttcacgagtgtgtgtgtgtgtgtgtgtgtgtgtgcgcgcgcgcgcgcgcactcgcgcgcacattccctatgtgttaagcagctcattaaagaaaaagaaaaataatcaggagaaaggaagatgaattgcagaaagtgccagaaagctagaaagaaattaaaactcttctccatacatactgcatacacataacctagcctatttatttgtatctaaaattccctagccgcaccatcaccgtaaacaccaagggaaaaaattaaggaggttcctggtgggaaaagggcgagttggggggacagggtgtctgcgaggtgacgggatacagaaaactagggtgtcaaaagggagcaagaacctgttttgggggcaacttaaggatccaagtgtcacggggtctgggcaatgcaggacgggaggggctgcgtgagtgagtacagaagggaaatgagtgagggggcatgggatctcagagaaaatcagggccctctgagcaaagtggaaaggacgaccgccgcagctcctcgggccgtagctcgaccccgccttcccttttgcgcagaatcctcgccttggctgcagcagcgcgctgcccccactggccggcgtgccgtgatcgatcgcaggctgcgtcaggagcctcccggcgtataaataggggtggcagaacggcgccgagccgcacacagccatccatcctcccccttccctctctcccctgtcctctctctccgggctcccaccgccgccgcgggccggggagccaccg

SEQ ID NO: 30 - DNA, 3045 bp, synthetic construct
  source 1..3045: mol_type = other DNA; note = Neurofilament heavy chain NEFH promoter
ttgggaaactcagcacactctacttgcctctgggagattcacaatgctcactgccaagtccaggctttgggaagataactccaagaactgcgggtggatggatggatggatggatagatagatgaatatatcgttctactcacttccaaatggtaagaatcacagggaggaaagtgaaagcgattcgagccccaaggactcagggcagaaccattcacagcccctccagaccacaagtttggctggaaaacagacggaacttattcattcattgattcatttacacaccacagagtggtaagagcagcattagagattagcactgggaaccatgggaaccaccaggaggggacaattaactaaatgtgggggtgaaggcggccagggatggctttctggaggtgagcctgaagggtcctttgggggaactgacctcagggctccagccctcatgccattttctccagccacaagagtcatgcttggggcttcttggactacataggcagcttcaatctgatggctgtggccccttggcctcaacagaatacatcttggagccccctttttaccccaaacccccattcctccttgctgtcagctgcttgtgagccttctcacatccagagaatgtatcagcattgtgcagactgaaaagacccagaggaacaaggctccaatggcaaaattccaagtagaatgacaaataaatggggagccatctgagagcaagggagtcctgcccaacacccgccccatgcctttctcagggacctcagaccagccactcacctccatcctcccagcaccacctgcaaccagccccttgccctctgcaaactggagcacgactggatctttagatgggggaaaaatgcttcatcatgttctgctgcttcatgcaaaaccagaaactccctccccctcttccctcctcccagcgcactctccttccagtaaaaagtggttaaagggacagcgccatcaatttcccagctctgagggtctgcttagaactagggggctggaaggagacagagggcaaagagaaaggaactggcagaggtctttcctgggggatatgtctgttctgtcctggggatcctggagcaggaaaacccgcgtaaagtaggggtgtagtgggtgttgagataactgcctgggggaggttcagagtggaagtacgagtctacaaactctcaagggcgtctcagggctcccagcatccccaggggtcctttcgcaggggtccctaagcaggaggggaacagcccagaaaacacggaactggacccccgacaggaagtccagggaggggtccctggctcactatgtgaccctgctggatcacttgcctcccctctcgggtcccctcagcacagtgtccctcccttccttcccctaaagtaaaagcagagggttaatctctttccccgccccacgcccaacaaagagcaggccctgtccccggtgctgaagcgccagccgcagcaccacccccactcccacagcataaaacatgagccaaaaccaataaagagccaaatgtcacagccgttgcagggccccctaaatcctggggaccccttcttctacctgacatcctattggggtgagggactttggtactcagaaagcatctcatcacttccctgtaagagagaagggatgccgactcaggcgcctgcttgtctgttacaggagtgggggaagagaggacaagttgaggctgagaagatggggagggggagggagaaaagaggacttcctagtgttgacagaacggcaagatgtgggttccccatccccagttcagccagagacccctcaaagtggaacttcctggggcagtcgggggtcaggagttggagcttgtctctggggcaagaccccttcgttgtacagatggaaaaacaagggtgggaggacacagcttgtccaaggtcattcgaccagcaaactgcctagctgaccccagtgtgcagaagctggctcgggtgacacccatcatttccccccaccccacacaggggccagctctctcaacttcatgcccaagccctcctacggtacccccactgtaggttctctgcccctcaaactcagcccagctttctcctgcctgttcaggggaccttctgcccgcttcgctgagggtccgtcccctttactggggctggcagcagggtctcccatctcctctctcgggggccactgcagactttttagagaacgccttgcctccccccaaccccacccatccggggttccctctctccatcctctgcagtgtctcccatacccccattcagggtagccttgctattctccccaactccaggtcccccttcatctattccggggctggccgcggagtttcctgagcgctctccaagtgggtcctctagatgttaggagaacactgtacctcccccggtcaggggtctcctgtctccgttctatggagcgtccatgctcccattcaggactgccttgctccctcctctgttccggggctggctgcacagtctctgcaccccctatcctgaaagcctctcttaactatttggaaagcctcgtgtcctgtctcatacagggatcccctcatcctaatgactgcaatcttccattgctccatcccgagggcatcctgcccctattcccatcaggtttctccttgtcctctccctgtttcaagtcccctttcttattccgaacacactcgcaggctcttccgacgcgcacccgggggtcctcactggcccactccgggagtcctctgcccgcttccccgacctcgagggtctcctctgacgcagcgtcgattccccttccctcctcggtcccctgccccgcccctctcactgcggcggagccggtcggccggggggccgcaggggaggaggcggagagggcggggccctcctccccaccctctcactgccaaggggttggacccggccgcggcggctataaaagggccggcgccctggtgctgccgcagtgcctcccgccccgtcccggcctcgcgcacctgctcaggcc

SEQ ID NO: 31 - DNA, 2888 bp, synthetic construct
  source 1..2888: mol_type = other DNA; note = Mouse Thy-1 cell surface antigen mTHY1
ggttatccaggcttctgaaggttcaagcaaagaaagggttacaaccttaaaaggagagcgtcccggggtatgggtagaagactgctccaccccgacccccagggtccctaaccgtcttttccctgggcgagtcagcccaatcacaggactgagagtgcctctttagtagcagcaagccacttcggacacccaaatggaacacctccagtcagccctcgccgaccaccccaccccctccatccttttccctcagcctccgattggctgaatctagagtccctccctgctcccccctctctccccacccctggtgaaaactgcgggcttcagcgctgggtgcagcaactggaggcgttggcgcaccaggaggaggctgcagctaggggagtccaggtgagagcaggccgacgggagggacccgcacatgcaaggaccgccgcagggcgaggatgcaagccttccccagctacagttttgggaaaggataccagggcgctcctatatgggggcgcgggaactggggaaagaaggtgctcccaggtcgaggtgggagaggaaggcagtgcggggtcacgggctttctccctgctaacggacgctttcgaagagtgggtgccggaggagaaccatgaggaaggacatcaaggacagcctttggtccccaagctcaaatcgctttagtggtgcgaatagagggaggaggtgggtggcaaactggagggagtccccagcgggtgacctcgtggctggctgggtgcggggcaccgcaggtaagaaaaccgcaatgttgcgggaggggactgggtggcaggcgcgggggaggggaaagctagaaaggatgcgagggagcggaggggggagggagcgggagaatctcaactggtagaggaagattaaaatgaggaaatagcatcagggtggggttagccaagccgggcctcagggaaagggcgcaaagtttgtctgggtgtgggcttaggtgggctgggtatgagattcggggcgccgaaaacactgctgcgcctctgccaaatcacgctacccctgtatctagttctgccaggcttctccagccccagccccaattcttttctctagtgttcccccttccctcccctgaatctcaagcccacactccctcctccataacccactgttatcaaatctaagtcatttgccacccaacaaccatcaggaggcggaagcagacgggaggagtttgagatcaacttgggctacatcacgagttccaggctcaccaaggcttcttaaggagaccttgtctctaaaattaattaattaattaattaatagtcccctttctctgccacagaaccttgggatctggctcctggtcgcagctccccccaccccaggctgacattcactgccatagcccatccggaaatcctagtctatttccccatggatcttgaactgcagagagaatggcagagtggcccgccctgtgcaaaggatgttcctagcctaggtggagctcgcgaactcgcagactgtgcctctcttgggcaaggacaggctagacagcctgccggtgtgttgagctagggcactgtggggaaggcagagaacctgtgcagggcagcaatgaacacaggaccagaaaactgcagccctaggaacactcaagagctggccatttgcaagcatctctggcctccgtgcttctcactcatgtcccatgtcttatacaggcctctgtggcacctcgcttgcctgatctcatccctagccgttaagctttctgcatgacttatcacttggggcataatgctggatacctaccattttcttagaccccatcaaaatcctatttgagtgtacggttcggagaacctcatttatccggtaaatgtcttttactctgctctcagggagctgaggcaggacatcctgagatacattgggagaggagatacagtttcaataaaataataggttgggtggaggtacatgcctataatgccaccactcaggaaatggtggcagcttcgtgagtttgaggccaacccaagaaacatagtgaaaccctgtcagtaaataagtaagcaagtatttgagtatctactatatgctagggctgacctggacattaggggtcatcttctgaacaaactagtgcttgagggaggtatttggggtttttgtttgtttaatggatctgaatgagttccagagactggctacacagcgatatgactgagcttaacacccctaaagcatacagtcagaccaattagacaataaaaggtatgtatagcttaccaaataaaaaaattgtattttcaagagagtgtctgtctgtgtagccctggctgttcttgaactcactctgtagaccaggctggcctggaaatccatctgcctgcctctgcctctctgcctctctgcctctctgcctctctctctgcctctctctgcctctctctgcccctctctgcccctctctgcccctctctgccgccctctgccttttgccctctgccctctgttctctggcctctgccctctgccctctggcctctggcctctgcctctgcctcttgagtgctggaatcaaaggtgtgagctctgtaggtcttaagttccagaagaaagtaatgaagtcacccagcagggaggtgctcagggacagcacagacacacacccaggacataggctcccacttccttggctttctctgagtggcaaaggaccttaggcagtgtcactccctaagagaaggggataaagagaggggctgaggtattcatcatgtgctccgtggatctcaagccctcaaggtaaatggggacccacctgtcctaccagctggctgacctgtagctttccccaccacagaatccaagtcggaactcttggcacc

SEQ ID NO: 32 - DNA, 1807 bp, synthetic construct
  source 1..1807: mol_type = other DNA; note = Rat Neuron specific enolase rNSE promoter
gagctcctcctctgctcgcccaatccttccaaccccctatggtggtatggctgacacagaaaatgtctgctcctgtatgggacatttgcccctcttctccaaatataagacaggatgaggcctagcttttgctgctccaaagttttaaaagaacacattgcacggcatttagggactctaaagggtggaggaggaatgagggaattgcatcatgccaaggctggtcctcatccatcactgcttccagggcccagagtggcttccaggaggtattcttacaaaggaagcccgatctgtagctaacactcagagcccattttcctgcgttaacccctcccgacctcatatacaggagtaacatgatcagtgacctgggggagctggccaaactgcgggacctgcccaagctgagggccttggtgctgctggacaacccctgtgccgatgagactgactaccgccaggaggccctggtgcagatggcacacctagagcgcctagacaaagagtactatgaggacgaggaccgggcagaagctgaggagatccgacagaggctgaaggaggaacaggagcaagaactcgacccggaccaagacatggaaccgtacctcccgccaacttagtggctcctctagcctgcagggacagtaaaggtgatggcaggaaggcagcccccggaggtcaaaggctgggcacgcgggaggagaggccagagtcagaggctgcgggtatctcagatatgaaggaaagatgagagaggctcaggaagaggtaagaaaagacacaagagaccagagaagggagaagaattagagagggaggcagaggaccgctgtctctacagacatagctggtagagactgggaggaagggatgaaccctgagcgcatgaagggaaggaggtggctggtggtatatggaggatgtagctgggccagggaaaagatcctgcactaaaaatctgaagctaaaaataacaggacacggggtggagaggcgaaaggagggcagagtgaggcagagggactgagaggcctggggatgtgggcattccggtagggcacacagttcacttgtcttctctttttccaggaggccaaagatgctgacgtcaagaactcataataccccagtggggaccaccgcattcatagccctgttacaagaagtgggagatgttcctttttgtcccagactggaaatccattacatcccgaggctcaggttctgtggtggtcatctctgtgtggcttgttctgtgggcctacctaaagtcctaagcacagctctcaagcagatccgaggcgactaagatgctagtaggggttgtctggagagaagagccgaggaggtgggctgtgatggatcagttcagctttcaaataaaaaggcgtttttatattctgtgtcgagttcgtgaacccctgtggtgggcttctccatctgtctgggttagtacctgccactatactggaataaggggacgcctgcttccctcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgag

SEQ ID NO: 33 - DNA, 561 bp, synthetic construct
  source 1..561: mol_type = other DNA; note = Dopa Carboxylase DDC promoter
ccatggtgggaggagagactgagcccaaaccatgttttagatgctgataggcttaagggtaacagcacaggagtttgagatgcatgcggctcaacacctaatctacatctcacttcactttctcatctggggaagtgggcttgggaccctgagcctcccgggtatcacagggtcctaatagtccctcacagaaggagcagacccagagtgagcactccccaaatgccacgccgtcccttcctcacccttggagtggagcctggtcgttctcaaagttgctgggagagtcccaggagccctggccccaaatctgcatcctacacagtgcctgggaacacagggcccattttttccttggcctctcgccagtcccagcaggccctgatgctcctctccatcctgctaggatggctctctccccctgggggcagagtgggggcaggaggtggtgggagggtggagggatgctgctcagtaaataatgcagagccggcagctctgattggcttcggggaggcagacactctgtctacataaatggcaatcacatcttctgtgcct

SEQ ID NO: 34 - DNA, 1151 bp, synthetic construct; identical to SEQ ID NO: 21
  source 1..1151: mol_type = other DNA; note = catechol-O-methyltransferase COMT promoter

SEQ ID NO: 35 - DNA, 499 bp, synthetic construct
  source 1..499: mol_type = other DNA; note = Glucosylceramidase beta 1 Promoter 1 GBA P1
atagaatgttaaggtcccaagatcagaaacttgctcattgcagtgggtctaacactcagtaggtcctcaacaaacattcgttaagatactaaagtggcagggtggggccctgtaaacagcttcaggaccctgtgcttgtaggggcaacgtggtgccctccaaggaagacagggaggtgggaggagcactgcccagagatggcgtcaggctgcaagacttcttgaataattcagcatcataacaacccagcctcaggaagggatagggcacggccaggacgaaacattaggaggcgatggacaatgggattcccacggggcagcttctgcgcactggacgttccctaacctgaggctctctaaagaggaaggttaggaatcctctgagcttcggtgggctggactcactgtgggaattcaatcgccccatccaccaacagtgtgctggcgggaaaacgccgacacgcatgcgtagttctcgcgccggctcctctctctct

SEQ ID NO: 36 - DNA, 500 bp, synthetic construct
  source 1..500: mol_type = other DNA; note = Glucosylceramidase beta 1 Promoter 2 GBA P2
cctccctccagtcctgtcatcccactgccagtgtcccaccctcttgtgccctgcacttcactggctaataacccccctcactttttcctctgtgccgccatcctggataattccccacccacgaatggtccctcctcatctcagagagctctccatgcacacctgttaccgtttctgtctttatctgtaaatatctgtgtgtctgacttccatgcctcacacacctctatagggcaaagactgtcttaaacatcttggtagtgtcaagtattttgcacagtgaagtttttttttttaaattatatcagctttatttgtacctttttgacatttctatcaaaaaagaagtgtgcctgctgtggttcccatcctctgggatttaggagcctctaccccattctccatgcaaatctgtgttctaggctcttcctaagttgtcacccatacatgccctccagagttttatagggcatataatctgtaacagatcagaggaagcc

SEQ ID NO: 37 - DNA, 232 bp, synthetic construct
  source 1..232: mol_type = other DNA; note = Human eukaryotic translation elongation factor 1 alpha 1 short form EFS promoter
ggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagg

SEQ ID NO: 38 - DNA, 1179 bp, synthetic construct
  source 1..1179: mol_type = other DNA; note = Human eukaryotic translation elongation factor 1 alpha 1 EEF1A1 promoter
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 39 - DNA, 850 bp, synthetic construct
  source 1..850: mol_type = other DNA; note = Chicken beta actin CBA promoter
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgct

SEQ ID NO: 40 - DNA, 589 bp, synthetic construct
  source 1..589: mol_type = other DNA; note = Cytomegalovirus CMV promoter
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 41 - protein, 6 aa, synthetic construct
  source 1..6: note = Protospacer Adjacent Motif PAM
NNGRRT

SEQ ID NO: 42 - protein, 6 aa, synthetic construct
  source 1..6: note = Protospacer Adjacent Motif PAM
NNGRRV

SEQ ID NO: 43 - DNA, 505 bp, synthetic construct
  source 1..505: mol_type = other DNA; note = Human phosphoglycerate kinase 1 hPGK promoter
gggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagg

(19) United States
(12) Patent Application Publication
(10) Pub. No.: US 2026/0209695 A1
(43) Pub. Date: Jul. 23, 2026

(54) METHODS AND COMPOSITIONS FOR ENGINEERED DA NEURONAL CELLS

(71) Applicant: Kenai Therapeutics, Inc., Cambridge, MA (US)

(72) Inventor: Howard Federoff, Cambridge, MA (US)

(73) Assignee: Kenai Therapeutics, Inc., Cambridge, MA (US)

(21) Appl. No.: 19/551,539

(22) Filed: Feb. 26, 2026

Related U.S. Application Data
(63) Continuation of application No. PCT/US2024/044518, filed on Aug. 29, 2024.
(60) Provisional application No. 63/579,861, filed on Aug. 31, 2023.

(51) Int. Cl. C12N 5/0793; A61K 35/30; C07K 14/475; C12N 9/24

(52) U.S. Cl. CPC C12N 5/0619; A61K 35/30; C07K 14/475; C12N 9/2402; C12Y 302/01045; C12N 2506/45; C12N 2510/00

(57) ABSTRACT

Disclosed are novel strategies for the treatment of patients with Parkinson's disease and other secondary Parkinsonian disorders. Disclosed are DA neuronal cells that have been modified in vitro with a genetic insertion of GDNF. The GDNF coding sequence is inserted under the transcriptional control of a promotor such that secreted proteins are made and taken up by the endogenous cells after the administered engineered cells have matured to a Neuronal Mature Cell Type to promote survival of endogenous neurons. Also disclosed are DA neuronal cells that have been modified with a genetic insertion of GBA in vitro and are hemizygous null for SNCA. The GBA coding sequence is inserted under the transcriptional control of a ubiquitous promotor and the secreted proteins are made and taken up by the graft and endogenous cells immediately upon transplantation to promote long-term graft integrity.

### CROSS-REFERENCE TO RELATED APPLICATIONS

This application is a continuation application of PCT/US2024/044518 filed on Aug. 29, 2024 and entitled “METHODS AND COMPOSITIONS FOR ENGINEERED DA NEURONAL CELLS,” which claims the benefit of U.S. Provisional Application No. 63/579,861, filed on Aug. 31, 2023, now expired, and entitled “METHODS AND COMPOSITIONS FOR ENGINEERED DA NEURONAL CELLS,” the entire contents of each of which are incorporated by reference herein.

### SEQUENCE LISTING

The instant application contains a Sequence Listing XML which has been submitted electronically in XML format and is hereby incorporated by reference in its entirety. The attached sequence listing is 108,847 bytes in size and has the file name “87874_00116.xml” and has a creation date of Aug. 13, 2024.

### FIELD OF THE INVENTION

The present disclosure relates generally to gene therapy and/or gene editing for treatment of disorders associated with central nervous system degeneration, such as Parkinson's Disease.

### BACKGROUND

### Gene Editing Technologies

Since their inception, gene editing technologies have proved a useful tool in the development of in vitro disease models. Replacement of endogenous genomic sequences with exogenous donor DNA/RNA via homologous recombination (HR) and correct insertion of exogenous DNA/RNA at defined mammalian chromosomal locations was first developed in the 1980s (Smithies et al., 1984), and subsequently applied to the genomic modification of mouse embryonic stem cells (PSCs) (Hasty et al., 1991). The discovery of I-SceI yeast meganuclease (Jacquier and Dujon, 1985), which promotes HR endogenous cellular mechanisms to repair DNA/RNA double-strand breaks (DSBs) in the presence of donor DNA/RNA, lead to the establishment of genome-editing strategies in murine cells (Choulika et al., 1995) and PSCs (Cohen-Tannoudji et al., 1998) based on proteins derived from unicellular organisms.

The advent of zinc-finger nuclease (ZFN) technology improved efficiency in genome-editing of mammalian cells (Bibikova et al., 2001), leading to the generation of the first knockout rat (Geurts et al., 2009). Following use in animals and cellular models (Petersen and Niemann, 2015), ZFNs-based genome editing was exploited for the correction of genetic mutations in patient-derived iPSCs (Soldner et al., 2011; Reinhardt et al., 2013; Kiskinis et al., 2018; Wang et al., 2018; Korecka et al., 2019), or for insertion of known disease-relevant mutations in iPSCs derived from healthy individuals (Verheyen et al., 2018), allowing direct investigation of specific genomic alterations and disease phenotypes. In addition, ZFNs were applied for the generation of engineered lines to study cell fate determination and improve iPSCs differentiation protocols (Hockemeyer et al., 2009), as well as to produce cell type-specific reporter systems for the investigation of disease pathogenesis (Zhang et al., 2016).

Genome editing technology further advanced with the advent of transcription activator-like effector nucleases (TALENs), which proved to be an efficient technology for the generation of animal models (Tesson et al., 2011). TALENs were further employed in the study of neurological disorders through the introduction of disease-causing mutations in control iPSCs (Wen et al., 2014; Lenzi et al., 2015; Akiyama et al., 2019) and/or correction of genetic mutations in patient-derived iPSCs (Maetzel et al., 2014; Wen et al., 2014; Li H. L. et al., 2015; Tanaka et al., 2018; Akiyama et al., 2019) leading to greater confidence in disease-underlying mechanisms and development of therapeutic approaches. Moreover, TALENs technology was used to develop reporter lines for stem cell-based research (Cerbini et al., 2015; Pei et al., 2015).

Rapidly following the development of TALENs technology, clustered regularly interspaced short palindromic repeats (CRISPR) with the CRISPR-associated protein (Cas13) system (Gasiunas et al., 2012; Jinek et al., 2012) demonstrated revolutionary potential to engineer the genome of mammalian cells in culture (Cong et al., 2013; Mali et al., 2013) and animal models (Wang H. et al., 2013). As for ZFNs or TALENs, CRISPR-Cas13 uses distinct RNA cleavage and binding modules. However, CRISPR-Cas13 system uses its own natural endonuclease and relies on a CRISPR RNA (crRNA) and a trans-activating RNA (transRNA) to specifically bind target RNA sequences and activating Cas13. Therefore, the long and complex process of engineered nuclease production was rapidly overcome by the plasticity and simplicity of generating different CRISPR-based approaches, which require only the design of a specific target-matching RNA. The extraordinary efficacy of CRISPR-Cas13, together with its great versatility for the generation of a broad range of substitutions, duplications, deletions, inversions, and many other complex alterations up to chromosomal rearrangements, have transformed the genome-editing field. There were, however, several limitations that required further improvements. Increased efficiency and reduction of off-target effects have been achieved through the engineering of Cas13 protein (Kleinstiver et al., 2015, 2016; Anders et al., 2016; Slaymaker et al., 2016; Chen et al., 2017; Casini et al., 2018; Hu et al., 2018; Lee et al., 2018; Nishimasu et al., 2018) and amendments to the design and structure of the guide RNA (Jinek et al., 2012; Hsu et al., 2013; Cui et al., 2018; Filippova et al., 2019; Moon et al., 2019), as well as the discovery and application of Cas proteins with different and specific gene-editing properties (Zetsche et al., 2015; Abudayyeh et al., 2016; Burstein et al., 2017). CRISPR-based technology has further developed to allow transcriptional inhibition (CRISPR interference, CRISPRi) or activation (CRISPR activation, CRISPRa). This CRISPR-based transcriptional modulation is achieved by repressor or activator transcription domains fused to a catalytically inactive Cas13 (dCas13) and guide RNAs directed to the promoter or regulatory regions of specific genes (Gilbert et al., 2013).

CRISPR-based engineering technologies have enabled researchers to dissect the function of specific genetic elements or correct disease-causing mutations. In parallel, CRISPR tools are now being implemented for active control and modulation of desired messenger RNAs (mRNAs). This allows the interrogation of transcriptome dynamics and the establishment of causal links between observed transcriptional changes and cellular phenotypes. Previously, RNA interference (RNAi) technology enabled inhibition of desired transcripts using micro RNAs (miRNAs), but this carried significant off-target effects due to cross-reaction with targets of limited sequence similarity and mis-targeting effects linked to endogenous miRNAs (Flynt and Lai, 2008). Investigation of Cas proteins able to target RNA led to the development of an engineered RNA-guided and RNA-targeting enzyme (CasRx) (Konermann et al., 2018), which showed improved efficiency in knocking down endogenous mRNA levels compared to RNAi technology, allowing ready manipulation of alternative splicing in human cells. Moreover, Konermann and colleagues successfully applied Cas-Rx editing in a patient-derived cortical neuronal model of FTD to modulate the balance of tau isoforms. Some forms of FTD with parkinsonism linked to chromosome 17 (FTDP-17) and other tauopathies are caused by mutations in the intron following exon 10 of MAPT (Boeve and Hutton, 2008). These variants disrupt an intronic splicing site and increase expression of the 4R tau isoform that contains more microtubule-binding domains (Kar et al., 2005), inducing pathological changes and driving the progression of neurodegeneration (Schoch et al., 2016). CasRx-mediated exon exclusion reduced 4R tau expression to a level similar to unaffected control neurons, suggesting that this technology can be exploited for transcriptional modulation in in vitro models. Interestingly, the small size of CasRx was amenable to packaging in adeno-associated virus (AAV) for delivery into post-mitotic neurons, encouraging future clinical applications in treatment of neurological disorders, and could be paired with an array encoding multiple guide RNAs for multiplexing. Therefore, CasRx technology paves the way for transcriptome engineering and RNA-targeting therapeutic applications.

### Differentiation to Lineage Specific Cell Populations

There is need for methods of producing DA neuronal cells from pluripotent cells, since such cells could be used both therapeutically and in disease models, e.g., to identify new therapeutics for treatments for Parkinson's disease and other and secondary Parkinsonian disorders, including but not limited to idiopathic Parkinson's, vascular parkinsonism, drug-induced parkinsonism and non-ideopathic Parkinson's disease disorders including but not limited to Parkin and other familial and genetic disease.

Various efforts have been made to generate midbrain DA neurons from pluripotent cells. For example, methodologies for generating midbrain DA neurons from pluripotent cells typically require use of both LDN-193189, an inhibitor of BMP signaling (inhibits ALK 1/2/3/6, blocks SMAD 1/5/8), and SB-431542, an inhibitor of TGF-beta signaling (inhibits ALK 4/5/7, blocks SMAD 2/3), as described, e.g., U.S. Pat. No. 10,280,398, which is herein incorporated by reference in its entirety. Since these methods utilize the combination of two inhibitors of Small Mothers Against Decapetaplegic (SMAD) signaling, these methods are typically referred to as “dual SMAD inhibition”, or “dual SMADi.”

One method to make DA neurons using dual SMAD inhibition comprises differentiating pluripotent stem cells, comprising exposing a plurality of pluripotent stem cells to at least one inhibitor of TGFβ/Activin-Nodal signaling, at least one inhibitor of bone morphogenetic protein (BMP) signaling, at least two activators of Sonic hedgehog (SHH) signaling, such as purmorphamine and SHH C25II, and at least one inhibitor of glycogen synthase kinase 3β (GSK3B) signaling that activates wingless (Wnt) signaling, wherein the exposure of the cells to the at least one inhibitor of TGFβ/Activin-Nodal signaling and at least one inhibitor of BMP signaling begins on day 0, wherein the cells are exposed to the at least one inhibitor of GSK3B signaling on the third (3rd) day through the eleventh (11th) day from the initial exposure of the cells to the at least one inhibitor of TGFβ/Activin-Nodal signaling and the at least one inhibitor of BMP signaling in amounts effective to produce a cell population comprising at least about 10% differentiated cells expressing both forkhead box protein A2 (FOXA2) and LIM homeobox transcription factor 1 alpha (LMX1A). U.S. Pat. Nos. 10,858,625, and 10,273,452 which is herein incorporated by reference in its entirety, discloses another method to make DA neurons using dual SMAD techniques. Methods to obtain enrich populations of midbrain dopaminergic (DA) neurons are described in U.S. Pat. No. 10,828,335, which is herein incorporated by reference in its entirety. Methods to prepare pluripotent stem cells for neural differentiation are described in U.S. Pat. No. 9,487,752, which is herein incorporated by reference in its entirety.

Others have generating midbrain DA neurons from pluripotent cells using mono-SMAD inhibition (mono-SMADi). See e.g., U.S. Pat. No. 10,590,383 which is herein incorporated by reference in its entirety. Generally, the method comprises culturing human pluripotent cells in the presence of the following signaling modulators: (a) a single inhibitor of Small Mothers Against Decapentaplegic (SMAD) signaling, (b) at least one activator of Sonic hedgehog (SHH) signaling, and (c) at least one activator of wingless (Wnt) signaling; and culturing the cells in the presence of the modulators for a period of time sufficient to provide a cell composition comprising FOXA2+/LMX1+ cells; wherein the culturing does not comprise culturing the human pluripotent cells in the presence of a second inhibitor of Small Mothers Against Decapentaplegic (SMAD) signaling.

### GBA Deficiency Promotes SNCA/α-Synuclein Accumulation

Loss-of-function mutations in the gene encoding GBA (glucocerebrosidase, B, acid), the enzyme deficient in the lysosomal storage disorder Gaucher disease, elevate the risk of Parkinson disease (PD), which is characterized by the misprocessing of SNCA/α-synuclein. It has been discovered that loss of GBA function resulted in increased levels of SNCA via inhibition of the autophagic pathway in SK-N-SH neuroblastoma cells, primary rat cortical neurons, or the rat striatum. Du T T, Wang L, Duan C L, Lu L L, Zhang J L, Gao G, Qiu X B, Wang X M, Yang H. GBA deficiency promotes SNCA/α-synuclein accumulation through autophagic inhibition by in expressed PPP2A. Autophagy. 2015; 11 (10): 1803-20. doi: 10.1080/15548627.2015.1086055. PMID: 26378614; PMCID: PMC4824589. These findings demonstrate that loss of GBA function may contribute to SNCA accumulation through inhibition of autophagy via PPP2A inactivation, thereby providing a mechanistic basis for the increased PD risk associated with GBA deficiency.

There is a great need in the art for new compositions and methods of treating and preventing central nervous system degeneration.

### SUMMARY

DA Neuronal Cells with Delayed Expression of Exogenous GDNF

In cell transplantation therapies, one major challenge is to avoid cells that have low viability, engraftment, proliferation, migration, innervation, differentiation, or function. This is more likely to happen when the engineered genes are partially or not fully expressed, expressed at the wrong time, or are misregulated during expression.

To avoid these pitfalls, it is desirable to include transcriptional regulation such that the genes are not expressed until after the graft has been established, i.e., the graft has incorporated into the host tissue. In some embodiments, the exogenous gene under control of an endogenous promotor are not expressed until the transplanted cells (DA neuronal cells) have differentiated into a more a mature cell type in vivo such as immature neurons, mature neurons or neurons. Delaying expression of knock-in genes, such as GDNF, is expected to increase the transplanted engineered DA neuronal cells' therapeutic potential by improving the function of the Neuronal Mature Cell Type that the transplanted engineered DA neuronal cells differentiate into in vivo. Delaying expression of knock-in genes, such as GDNF, is expected to increase the transplanted engineered DA neuronal cells' therapeutic potential by improving the function of Neuronal Mature Cell Types that are in the intracranial, intra-putamen environment found near the transplanted engineered DA neuronal cells, i.e, endogenous Neuronal Mature Cell Types. The exogenous genetic sequence(s) to be expressed or co-expressed once the transplanted cell has differentiated to a Neuronal Mature Cell Type in vivo are introduced by gene targeted insertion using sequence-specific endonuclease reagents, so that their coding sequences are transcribed under the control of endogenous promoters present at a selected loci expressed in Neuronal Mature Cell Types. Alternatively, loci that are not expressed during neural cell differentiation can be used as “safe-harbor loci” for the integration of expression cassettes without any adverse consequences on the differentiation of engineered pluripotent cells to engineered DA neuronal cells in vitro or differentiation of the transplanted DA neuronal cells to a Neuronal Mature Cell Type in vivo. In some embodiments, the selected site for exogenous gene insertion is a safe harbor locus, highly expressive locus, temporally expressed locus, or a gene locus for interruption. In some embodiments, the safe harbor locus is AAVS1, CCR5, hROSA26, collagen, HTRP, H11, beta-2 microglobulin, GAPDH, TCR or RUNX1, or loci meeting the criteria of a genome safe harbor as defined herein. In some embodiments, a gene locus for interruption comprising GDNF, GBA PARK2, PINK1, DJ-1, LRRK2, c-Rel, ATG7, VMAT2 or SNCA. In some embodiments, the safe harbor locus is selected from the list in table 1.

These cell engineering strategies, tend to reinforce the therapeutic potential of transplanted engineered DA neuronal cells in general, in particular by increasing their viability, engraftment, proliferation, migration, innervation and/or differentiation or by increasing the function of the Neuronal Mature Cell Type that the transplanted engineered DA neuronal cells differentiate into in vivo and/or survival of endogenous neurons. The strategies may be carried out on cells originating from patients as part of autologous treatment strategies, as well as from donors, as part of allogeneic treatment strategies.

DA Neuronal Cells which Immediately Express Exogenous GBA and are Hemizygous Null for SNCA

Expressing knock-in genes such as GBA as soon as such modified DA neuronal cells are transplanted is expected to increase the transplanted engineered DA neuronal cells' therapeutic potential by improving long-term graft integrity. The exogenous genetic sequence(s) is expressed or co-expressed upon transplantation. In some embodiments, the exogenous genetic sequence(s) are transcribed under the control of endogenous ubiquitous promoter(s). Alternatively, loci pre-identified as absent of endogenously-expressing genes during DA neuronal cell transplantation can be used as “safe-harbor loci” for the integration of expression cassettes under control of ubiquitous promoters.

### Engineered Pluripotent Cell Populations

Disclosed is a population of engineered pluripotent cells, wherein the engineered pluripotent cells comprise an exogenous knock-in polynucleotide sequence encoding a wild-type Parkinson protein 2, E3 ubiquitin protein ligase (PARK2), a PTEN-induced putative kinase 1 (PINK1), a protein deglycase DJ-1 (DJ-1), a Leucine Rich Repeat Kinase 2 (LRRK2), an alpha-synuclein (SNCA), a Proto-oncogene c-Rel (c-Rel), a Ubiquitin-like modifier-activating enzyme (ATG7), Synaptic vesicular amine transporter (VMAT2), glucocerebrosidase (GBA), and/or Glial cell line-derived neurotrophic factor (GDNF) gene. In some embodiments, the knock-in polynucleotide sequence is under the control of a GDNF promotor (SEQ ID NOS: 21-30). In some embodiments, the knock-in polynucleotide sequence is under the control of a GBA promotor (SEQ ID NOS: 31-40 or 43).

### Engineered DA Neuronal Cell Populations

Disclosed is a population of engineered DA neuronal cells, wherein the engineered DA neuronal cells comprise an exogenous knock-in PARK2, PINK1, DJ-1, LRRK2, c-Rel, ATG7, VMAT2, GBA, SNCA+/− and/or GDNF gene. In some embodiments, the knock-in polynucleotide sequence is under the control of a GDNF promotor (SEQ ID NOS: 21-30). In some embodiments, the knock-in polynucleotide sequence is under the control of a GBA promotor (SEQ ID NOS: 31-40 or 43).

### Gene Editing Systems

The engineered pluripotent cells and/or engineered DA neuronal cells discuses above can be made by any known gene editing system known to those of skill in the art. As such, the population of engineered pluripotent cells and/or engineered DA neuronal cells discuses above can comprise a nuclease (Cas protein, TALE nucleases, or zinc-finger nuclease), a repair template comprising a PARK2, PINK1, DJ-1, LRRK2, c-Rel, ATG7, VMAT2, GBA, hemizygous null SNCA, and/or GDNF gene or functional fragment or variant thereof.

The engineered pluripotent cells and/or engineered DA neuronal cells discuses above can be made by any known gene editing system known to those of skill in the art. As such, the population of engineered pluripotent cells and/or engineered DA neuronal cells discuses above can comprise a Cas protein, guide RNA, a repair template comprising a PARK2, PINK1, DJ-1, LRRK2, c-Rel, ATG7, VMAT2, GBA, hemizygous null SNCA, and/or GDNF gene or functional fragment or variant thereof. As such, the population of engineered pluripotent cells and/or engineered DA neuronal cells discuses above can comprise a nuclease (Cas protein, TALE nucleases, or zinc-finger nuclease), a repair template comprising a PARK2, PINK1, DJ-1, LRRK2, c-Rel, ATG7, VMAT2, GBA, SNCA+/− and/or GDNF gene or functional fragment or variant thereof in combination with an edit to (or disruption of) the SNCA and/or MAPT gene to render the cell population hemizygous null for SNCA and/or MAPT. In some embodiments, the repair template does not comprise SNCA.

### Recombinant Gene Vector

Disclosed are various embodiments of, and methods related to, a recombinant gene vector comprising a PARK2, PINK1, DJ-1, LRRK2, c-Rel, ATG7, VMAT2, GBA, SNCA or GDNF gene or functional fragment or variant thereof. Disclosed are various embodiments of, and methods related to, a recombinant gene vector comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 or a complement or RNA equivalent thereof.

### Methods

Contacting Pluripotent Cells In Vitro with a Gene Editing System

In some embodiments, disclosed is a method of making engineered DA neuronal cells, comprising contacting a pluripotent cell with a gene editing system comprising a nuclease (Cas protein, TALE nucleases, or zinc-finger nuclease), and a repair template comprising a functional PARK2, PINK1, DJ-1, LRRK2, c-Rel, ATG7, VMAT2, GBA, hemizygous SNCA, hemizygous MAPT and/or GDNF gene or a functional variant or fragment thereof. In some embodiments, the repair template is SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 or a complement or RNA equivalent thereof.

In some embodiments, disclosed is a method of making engineered DA neuronal cells, comprising contacting a pluripotent cell with a gene editing system comprising Cas protein or a polynucleotide encoding a Cas protein; a guide-RNA (gRNA); and a repair template comprising a functional PARK2, PINK1, DJ-1, LRRK2, c-Rel, ATG7, VMAT2, GBA, hemizygous SNCA, hemizygous MAPT and/or GDNF gene or a functional variant or fragment thereof. In some embodiments, the repair template is a knock-in repair template selected from the group comprising or consisting of Seq ID Nos: 1-10. In some embodiments, the gene editing system is capable of enhancing the viability, engraftment, proliferation, migration, innervation, differentiation, long-term graft integrity, survival of endogenous neurons, or function of the administered engineered DA neuronal cells compared to wildtype neuronal cells. In some embodiments, the administered engineered DA neuronal cells differentiate in vivo to a Neuronal Mature Cell Type with enhanced function because they are derived from the administered engineered DA neuronal cells. In some embodiments, the administered engineered DA neuronal cells administered in vivo and have an effect on endogenous Neuronal Mature Cell Types.

Contacting Neural Cells Ex Vivo or In Vivo with a Gene Editing System

In some embodiments, disclosed is a method of contacting a neuron with a gene editing system comprising a nuclease (Cas protein, TALE nucleases, or zinc-finger nuclease), and a repair template comprising a functional PARK2, PINK1, DJ-1, LRRK2, c-Rel, ATG7, VMAT2, GBA, SNCA+/− and/or GDNF gene or a functional variant or fragment thereof. In some embodiments, the repair template is a knock-in repair template selected from the group comprising or consisting of Seq ID Nos: 1-10.

In some embodiments, disclosed is a method of contacting a neuron with a gene editing system comprising Cas protein or a polynucleotide encoding a Cas protein; a guide-RNA (gRNA); and a repair template comprising a functional PARK2, PINK1, DJ-1, LRRK2, c-Rel, ATG7, VMAT2, GBA, SNCA+/− and/or GDNF gene or a functional variant or fragment thereof. In some embodiments, the repair template is a knock-in repair template selected from the group comprising or consisting of Seq ID Nos: 1-10. In some embodiments, the gene editing system is capable of enhancing the viability, engraftment, proliferation, migration, innervation, differentiation, long-term graft integrity, survival of endogenous neurons, or function of the administered engineered DA neuronal cells compared to wildtype neuronal cells. In some embodiments, the neuron is in the patient's body when it is contacted with the engineered DA neuronal cells. In some embodiments, the neuron is contacted with the gene editing system ex vivo, and then transplanted back into the patient's body in vivo following the contacting step.

Contacting Endogenous Wildtype Neural Cells In Vivo with Engineered DA Neuronal Cells

In some embodiments, disclosed is a method of contacting a neuron with engineered DA neuronal cells, comprising contacting a pluripotent cell with a gene editing system comprising a nuclease (Cas protein, TALE nucleases, or zinc-finger nuclease), and a repair template comprising a functional PARK2, PINK1, DJ-1, LRRK2, c-Rel, ATG7, VMAT2, GBA, hemizygous SNCA, hemizygous MAPT and/or GDNF gene or a functional variant or fragment thereof. In some embodiments, the repair template is a knock-in repair template selected from the group comprising or consisting of Seq ID Nos: 1-10.

In some embodiments, disclosed is a method of contacting a neuron with engineered DA neuronal cells, comprising contacting a pluripotent cell with a gene editing system comprising Cas protein or a polynucleotide encoding a Cas protein; a guide-RNA (gRNA); and a repair template comprising a functional PARK2, PINK1, DJ-1, LRRK2, c-Rel, ATG7, VMAT2, GBA, hemizygous SNCA, hemizygous MAPT and/or GDNF gene or a functional variant or fragment thereof. In some embodiments, the repair template is a knock-in repair template selected from the group comprising or consisting of Seq ID Nos: 1-10. In some embodiments, the engineered DA neuronal cell is capable of enhancing the viability, engraftment, proliferation, migration, innervation, differentiation, long-term graft integrity, survival of endogenous wildtype neurons, or function of the endogenous wildtype neuronal cells compared to non-contacted, wildtype neuronal cells. In some embodiments, the endogenous wildtype neuron is in the patient's body when it is contacted with the engineered DA neuronal cells. In some embodiments, the endogenous wildtype neuron contacted with the engineered DA neuronal cells ex vivo, and then transplanted back into the patient's body in vivo following the contacting step.

### Transplantation

The engineered DA neuronal cells, recombinant gene vector or gene editing system can be administered in various ways. In some embodiments, the administering step comprises systemic, parenteral, intravenous, cerebral, cerebrospinal, intrathecal, intracisternal, intraputaminal, intrahippocampal, intra-striatal, or intra-cerebroventricular administration. In some embodiments, the administering step comprises intravenous, cerebral, cerebrospinal, intrathecal, intracisternal, intraputaminal, intrahippocampal, intra-striatal, or intra-cerebroventricular injection. In some embodiments, the administering step comprises intrathecal injection with Threndelenburg tilting. In some embodiments, the administering step comprises direct injection into the pars compacta of the substantia nigra of the brain. In some embodiments, the administering step comprises introducing the engineered DA neuronal cells, recombinant gene vector or gene editing system into the subject's brain or cerebrospinal fluid (CSF).

In some embodiments, 1×10⁹-1×10¹⁴ recombinant gene vector genomes per kilogram body mass of the subject (vg/kg) of the gene therapy vector are administered to the subject. In some embodiments, 1×10⁹-1×10¹⁴ recombinant gene vector genomes per kilogram body mass of the subject (vg/kg) of the gene therapy vector are administered to the subject's brain. In some embodiments, 1×10⁹-1×10¹⁴ recombinant gene vector genomes per kilogram body mass of the subject (vg/kg) of the gene therapy vector are administered to the subject's CSF. In some embodiments, 1×10⁷-1×10⁹ recombinant gene vector genomes per kilogram body mass of the subject (vg/kg) of the gene therapy vector are administered to the subject.

### Treatment Outcomes

The methods of the disclosure (administering engineered DA neuronal cells, recombinant gene vector or gene editing system) may have various effects. In some embodiments, the administered engineered DA neuronal cells have increased viability, engraftment, proliferation, migration, innervation, differentiation, long-term graft integrity, survival of endogenous neurons, or function of the administered engineered DA neuronal cells. In some embodiments, the administered engineered DA neuronal cells do not have a changed effect. In some embodiments, the administered engineered DA neuronal cells do not have increased viability, engraftment, proliferation, migration, innervation, differentiation, long-term graft integrity, survival of endogenous neurons, or function of the administered engineered DA neuronal cells. In some embodiments, it is only the cells the administered engineered DA neuronal cells differentiate into (e.g., Neuronal Mature Cell Types) that have a changed effect. In some embodiments, the administered engineered DA neuronal cells cause endogenous Neuronal Mature Cell Types to have increased viability, engraftment, proliferation, migration, innervation, differentiation, long-term graft integrity, survival of endogenous neurons, or function of the administered engineered DA neuronal cells.

In some embodiments, the administered engineered DA neuronal cells having an engineered GBA gene have increased cell viability, engraftment, proliferation, migration, innervation, differentiation, long-term graft integrity, survival of endogenous neurons, or function of the administered engineered DA neuronal cells. In some embodiments, the administered engineered DA neuronal cells having an engineered GBA gene and disrupted SNCA gene have increased cell viability, engraftment, proliferation, migration, innervation, differentiation, long-term graft integrity, survival of endogenous neurons, or function of the administered engineered DA neuronal cells. In some embodiments, the administered engineered DA neuronal cells having an engineered GDNF gene have increased cell viability, engraftment, proliferation, migration, innervation, differentiation, long-term graft integrity, survival of endogenous neurons, or function of the administered engineered DA neuronal cells. In some embodiments, the administration of the engineered DA neuronal cells treats or inhibits onset of the Parkinson's Disease and secondary Parkinsonian disorders in the subject. In some embodiments, the administration of the engineered DA neuronal cells treats or inhibits onset of bradykinesia. In some embodiments, the administration of the engineered DA neuronal cells treats or inhibits onset of bradyphrenia. In some embodiments, the administration of the engineered DA neuronal cells treats or inhibits onset of neurochemical related disorders or decline relating to impairment of dopamine, acetylcholine, serotonin, and/or norepinephrine signaling.

### Sequences

In some embodiments, the PARK2, PINK1, LRRK2, c-Rel, ATG7, VMAT2, GBA, SNCA, MAPT or GDNF gene (or vector) comprises the nucleic acid sequence set forth in SEQ ID NOs: 1-10, respectively.

In some embodiments, the PARK2, PINK1, LRRK2, c-Rel, ATG7, VMAT2, GBA, SNCA, MAPT or GDNF protein comprises the amino acid sequence set forth in SEQ ID NOs: 11-20, respectively.

In some embodiments, the gene is the GDNF gene, and the wild-type GDNF protein comprises the amino acid sequence set forth in any of SEQ ID NOs: 10.

In some embodiments, the gene is the GBA gene, and the wild-type GBA protein comprises the amino acid sequence set forth in any of SEQ ID NOs: 7.

In some embodiments, the polynucleotide comprises a sequence having at least 70%, 75%, 80%, 85%, 95%, or 99% identity to a PARK2, PINK1, LRRK2, c-Rel, ATG7, VMAT2, GBA, or GDNF polynucleotide sequence set forth in SEQ ID NOs: 1-7 and 10, respectively. In some embodiments, the polynucleotide is codon-optimized. In some embodiments, the polynucleotide comprises less than 40, less than 30, less than 20, or 10 or fewer CpG islands. In some embodiments, the polynucleotide comprises at least 1, at least 2, at least 3, at least 4, at least 5, at least 6, or at least 10 CpG islands. In some embodiments, it comprises between 5 and 20 CpG islands.

Sequence IDs

SEQ ID No 1: >human PARK2 ENSG00000185345.24
ATGATAGTGTTTGTCAGGTTCAACTCCAGCCATGGTTTCCCAGTGGAGGTCGATTCTGACA
CCAGCATCTTCCAGCTCAAGGAGGTGGTTGCTAAGCGACAGGGGGTTCCGGCTGACCAGT
TGCGTGTGATTTTCGCAGGGAAGGAGCTGAGGAATGACTGGACTGTGCAGAATTGTGACC
TGGATCAGCAGAGCATTGTTCACATTGTGCAGAGACCGTGGAGAAAAGGTCAAGAAATG
AATGCAACTGGAGGCGACGACCCCAGAAACGCGGCGGGAGGCTGTGAGCGGGAGCCCCA
GAGCTTGACTCGGGTGGACCTCAGCAGCTCAGTCCTCCCAGGAGACTCTGTGGGGCTGGC
TGTCATTCTGCACACTGACAGCAGGAAGGACTCACCACCAGCTGGAAGTCCAGCAGGTAG
ATCAATCTACAACAGCTTTTATGTGTATTGCAAAGGCCCCTGTCAAAGAGTGCAGCCGGG
AAAACTCAGGGTACAGTGCAGCACCTGCAGGCAGGCAACGCTCACCTTGACCCAGGGTCC
ATCTTGCTGGGATGATGTTTTAATTCCAAACCGGATGAGTGGTGAATGCCAATCCCCACAC
TGCCCTGGGACTAGTGCAGAATTTTTCTTTAAATGTGGAGCACACCCCACCTCTGACAAGG
AAACATCAGTAGCTTTGCACCTGATCGCAACAAATAGTCGGAACATCACTTGCATTACGT
GCACAGACGTCAGGAGCCCCGTCCTGGTTTTCCAGTGCAACTCCCGCCACGTGATTTGCTT
AGACTGTTTCCACTTATACTGTGTGACAAGACTCAATGATCGGCAGTTTGTTCACGACCCT
CAACTTGGCTACTCCCTGCCTTGTGTGGCTGGCTGTCCCAACTCCTTGATTAAAGAGCTCC
ATCACTTCAGGATTCTGGGAGAAGAGCAGTACAACCGGTACCAGCAGTATGGTGCAGAGG
AGTGTGTCCTGCAGATGGGGGGCGTGTTATGCCCCCGCCCTGGCTGTGGAGCGGGGCTGC
TGCCGGAGCCTGACCAGAGGAAAGTCACCTGCGAAGGGGGCAATGGCCTGGGCTGTGGG
TTTGCCTTCTGCCGGGAATGTAAAGAAGCGTACCATGAAGGGGAGTGCAGTGCCGTATTT
GAAGCCTCAGGAACAACTACTCAGGCCTACAGAGTCGATGAAAGAGCCGCCGAGCAGGC
TCGTTGGGAAGCAGCCTCCAAAGAAACCATCAAGAAAACCACCAAGCCCTGTCCCCGCTG
CCATGTACCAGTGGAAAAAAATGGAGGCTGCATGCACATGAAGTGTCCGCAGCCCCAGTG
CAGGCTCGAGTGGTGCTGGAACTGTGGCTGCGAGTGGAACCGCGTCTGCATGGGGGACCA
CTGGTTCGACGTGTAG
SEQ ID No 2: >human PINK1 ENSG00000158828.8
ATGGCGGTGCGACAGGCGCTGGGCCGCGGCCTGCAGCTGGGTCGAGCGCTGCTGCTGCGC
TTCACGGGCAAGCCCGGCCGGGCCTACGGCTTGGGGCGGCCGGGCCCGGCGGCGGGCTGT
GTCCGCGGGGAGCGTCCAGGCTGGGCCGCAGGACCGGGCGCGGAGCCTCGCAGGGTCGG
GCTCGGGCTCCCTAACCGTCTCCGCTTCTTCCGCCAGTCGGTGGCCGGGCTGGCGGCGCGG
TTGCAGCGGCAGTTCGTGGTGCGGGCCTGGGGCTGCGCGGGCCCTTGCGGCCGGGCAGTC
TTTCTGGCCTTCGGGCTAGGGCTGGGCCTCATCGAGGAAAAACAGGCGGAGAGCCGGCGG
GCGGTCTCGGCCTGTCAGGAGATCCAGGCAATTTTTACCCAGAAAAGCAAGCCGGGGCCT
GACCCGTTGGACACGAGACGCTTGCAGGGCTTTCGGCTGGAGGAGTATCTGATAGGGCAG
TCCATTGGTAAGGGCTGCAGTGCTGCTGTGTATGAAGCCACCATGCCTACATTGCCCCAGA
ACCTGGAGGTGACAAAGAGCACCGGGTTGCTTCCAGGGAGAGGCCCAGGTACCAGTGCA
CCAGGAGAAGGGCAGGAGCGAGCTCCGGGGGCCCCTGCCTTCCCCTTGGCCATCAAGATG
ATGTGGAACATCTCGGCAGGTTCCTCCAGCGAAGCCATCTTGAACACAATGAGCCAGGAG
CTGGTCCCAGCGAGCCGAGTGGCCTTGGCTGGGGAGTATGGAGCAGTCACTTACAGAAAA
TCCAAGAGAGGTCCCAAGCAACTAGCCCCTCACCCCAACATCATCCGGGTTCTCCGCGCC
TTCACCTCTTCCGTGCCGCTGCTGCCAGGGGCCCTGGTCGACTACCCTGATGTGCTGCCCT
CACGCCTCCACCCTGAAGGCCTGGGCCATGGCCGGACGCTGTTCCTCGTTATGAAGAACT
ATCCCTGTACCCTGCGCCAGTACCTTTGTGTGAACACACCCAGCCCCCGCCTCGCCGCCAT
GATGCTGCTGCAGCTGCTGGAAGGCGTGGACCATCTGGTTCAACAGGGCATCGCGCACAG
AGACCTGAAATCCGACAACATCCTTGTGGAGCTGGACCCAGACGGCTGCCCCTGGCTGGT
GATCGCAGATTTTGGCTGCTGCCTGGCTGATGAGAGCATCGGCCTGCAGTTGCCCTTCAGC
AGCTGGTACGTGGATCGGGGCGGAAACGGCTGTCTGATGGCCCCAGAGGTGTCCACGGCC
CGTCCTGGCCCCAGGGCAGTGATTGACTACAGCAAGGCTGATGCCTGGGCAGTGGGAGCC
ATCGCCTATGAAATCTTCGGGCTTGTCAATCCCTTCTACGGCCAGGGCAAGGCCCACCTTG
AAAGCCGCAGCTACCAAGAGGCTCAGCTACCTGCACTGCCCGAGTCAGTGCCTCCAGACG
TGAGACAGTTGGTGAGGGCACTGCTCCAGCGAGAGGCCAGCAAGAGACCATCTGCCCGA
GTAGCCGCAAATGTGCTTCATCTAAGCCTCTGGGGTGAACATATTCTAGCCCTGAAGAATC
TGAAGTTAGACAAGATGGTTGGCTGGCTCCTCCAACAATCGGCCGCCACTTTGTTGGCCA
ACAGGCTCACAGAGAAGTGTTGTGTGGAAACAAAAATGAAGATGCTCTTTCTGGCTAACC
TGGAGTGTGAAACGCTCTGCCAGGCAGCCCTCCTCCTCTGCTCATGGAGGGCAGCCCTGT
GA
SEQ ID No 3: >human LRRK2 ENSG00000188906.17
ATGGCTAGTGGCAGCTGTCAGGGGTGCGAAGAGGACGAGGAAACTCTGAAGAAGTTGAT
AGTCAGGCTGAACAATGTCCAGGAAGGAAAACAGATAGAAACGCTGGTCCAAATCCTGG
AGGATCTGCTGGTGTTCACGTACTCCGAGCGCGCCTCCAAGTTATTTCAAGGCAAAAATAT
CCATGTGCCTCTGTTGATCGTCTTGGACTCCTATATGAGAGTCGCGAGTGTGCAGCAGGTG
GGTTGGTCACTTCTGTGCAAATTAATAGAAGTCTGTCCAGGTACAATGCAAAGCTTAATG
GGACCCCAGGATGTTGGAAATGATTGGGAAGTCCTTGGTGTTCACCAATTGATTCTTAAA
ATGCTAACAGTTCATAATGCCAGTGTAAACTTGTCAGTGATTGGACTGAAGACCTTAGATC
TCCTCCTAACTTCAGGTAAAATCACCTTGCTGATATTGGATGAAGAAAGTGATATTTTCAT
GTTAATTTTTGATGCCATGCACTCATTTCCAGCCAATGATGAAGTCCAGAAACTTGGATGC
AAAGCTTTACATGTGCTGTTTGAGAGAGTCTCAGAGGAGCAACTGACTGAATTTGTTGAG
AACAAAGATTATATGATATTGTTAAGTGCGTTAACAAATTTTAAAGATGAAGAGGAAATT
GTGCTTCATGTGCTGCATTGTTTACATTCCCTAGCGATTCCTTGCAATAATGTGGAAGTCCT
CATGAGTGGCAATGTCAGGTGTTATAATATTGTGGTGGAAGCTATGAAAGCATTCCCTAT
GAGTGAAAGAATTCAAGAAGTGAGTTGCTGTTTGCTCCATAGGCTTACATTAGGTAATTTT
TTCAATATCCTGGTATTAAACGAAGTCCATGAGTTTGTGGTGAAAGCTGTGCAGCAGTACC
CAGAGAATGCAGCATTGCAGATCTCAGCGCTCAGCTGTTTGGCCCTCCTCACTGAGACTAT
TTTCTTAAATCAAGATTTAGAGGAAAAGAATGAGAATCAAGAGAATGATGATGAGGGGG
AAGAAGATAAATTGTTTTGGCTGGAAGCCTGTTACAAAGCATTAACGTGGCATAGAAAGA
ACAAGCACGTGCAGGAGGCCGCATGCTGGGCACTAAATAATCTCCTTATGTACCAAAACA
GTTTACATGAGAAGATTGGAGATGAAGATGGCCATTTCCCAGCTCATAGGGAAGTGATGC
TCTCCATGCTGATGCATTCTTCATCAAAGGAAGTTTTCCAGGCATCTGCGAATGCATTGTC
AACTCTCTTAGAACAAAATGTTAATTTCAGAAAAATACTGTTATCAAAAGGAATACACCT
GAATGTTTTGGAGTTAATGCAGAAGCATATACATTCTCCTGAAGTGGCTGAAAGTGGCTG
TAAAATGCTAAATCATCTTTTTGAAGGAAGCAACACTTCCCTGGATATAATGGCAGCAGT
GGTCCCCAAAATACTAACAGTTATGAAACGTCATGAGACATCATTACCAGTGCAGCTGGA
GGCGCTTCGAGCTATTTTACATTTTATAGTGCCTGGCATGCCAGAAGAATCCAGGGAGGA
TACAGAATTTCATCATAAGCTAAATATGGTTAAAAAACAGTGTTTCAAGAATGATATTCA
CAAACTGGTCCTAGCAGCTTTGAACAGGTTCATTGGAAATCCTGGGATTCAGAAATGTGG
ATTAAAAGTAATTTCTTCTATTGTACATTTTCCTGATGCATTAGAGATGTTATCCCTGGAA
GGTGCTATGGATTCAGTGCTTCACACACTGCAGATGTATCCAGATGACCAAGAAATTCAG
TGTCTGGGTTTAAGTCTTATAGGATACTTGATTACAAAGAAGAATGTGTTCATAGGAACTG
GACATCTGCTGGCAAAAATTCTGGTTTCCAGCTTATACCGATTTAAGGATGTTGCTGAAAT
ACAGACTAAAGGATTTCAGACAATCTTAGCAATCCTCAAATTGTCAGCATCTTTTTCTAAG
CTGCTGGTGCATCATTCATTTGACTTAGTAATATTCCATCAAATGTCTTCCAATATCATGG
AACAAAAGGATCAACAGTTTCTAAACCTCTGTTGCAAGTGTTTTGCAAAAGTAGCTATGG
ATGATTACTTAAAAAATGTGATGCTAGAGAGAGCGTGTGATCAGAATAACAGCATCATGG
TTGAATGCTTGCTTCTATTGGGAGCAGATGCCAATCAAGCAAAGGAGGGATCTTCTTTAAT
TTGTCAGGTATGTGAGAAAGAGAGCAGTCCCAAATTGGTGGAACTCTTACTGAATAGTGG
ATCTCGTGAACAAGATGTACGAAAAGCGTTGACGATAAGCATTGGGAAAGGTGACAGCC
AGATCATCAGCTTGCTCTTAAGGAGGCTGGCCCTGGATGTGGCCAACAATAGCATTTGCCT
TGGAGGATTTTGTATAGGAAAAGTTGAACCTTCTTGGCTTGGTCCTTTATTTCCAGATAAG
ACTTCTAATTTAAGGAAACAAACAAATATAGCATCTACACTAGCAAGAATGGTGATCAGA
TATCAGATGAAAAGTGCTGTGGAAGAAGGAACAGCCTCAGGCAGCGATGGAAATTTTTCT
GAAGATGTGCTGTCTAAATTTGATGAATGGACCTTTATTCCTGACTCTTCTATGGACAGTG
TGTTTGCTCAAAGTGATGACCTGGATAGTGAAGGAAGTGAAGGCTCATTTCTTGTGAAAA
AGAAATCTAATTCAATTAGTGTAGGAGAATTTTACCGAGATGCCGTATTACAGCGTTGCTC
ACCAAATTTGCAAAGACATTCCAATTCCTTGGGGCCCATTTTTGATCATGAAGATTTACTG
AAGCGAAAAAGAAAAATATTATCTTCAGATGATTCACTCAGGTCATCAAAACTTCAATCC
CATATGAGGCATTCAGACAGCATTTCTTCTCTGGCTTCTGAGAGAGAATATATTACATCAC
TAGACCTTTCAGCAAATGAACTAAGAGATATTGATGCCCTAAGCCAGAAATGCTGTATAA
GTGTTCATTTGGAGCATCTTGAAAAGCTGGAGCTTCACCAGAATGCACTCACGAGCTTTCC
ACAACAGCTATGTGAAACTCTGAAGAGTTTGACACATTTGGACTTGCACAGTAATAAATT
TACATCATTTCCTTCTTATTTGTTGAAAATGAGTTGTATTGCTAATCTTGATGTCTCTCGAA
ATGACATTGGACCCTCAGTGGTTTTAGATCCTACAGTGAAATGTCCAACTCTGAAACAGTT
TAACCTGTCATATAACCAGCTGTCTTTTGTACCTGAGAACCTCACTGATGTGGTAGAGAAA
CTGGAGCAGCTCATTTTAGAAGGAAATAAAATATCAGGGATATGCTCCCCCTTGAGACTG
AAGGAACTGAAGATTTTAAACCTTAGTAAGAACCACATTTCATCCCTATCAGAGAACTTTC
TTGAGGCTTGTCCTAAAGTGGAGAGTTTCAGTGCCAGAATGAATTTTCTTGCTGCTATGCC
TTTCTTGCCTCCTTCTATGACAATCCTAAAATTATCTCAGAACAAATTTTCCTGTATTCCAG
AAGCAATTTTAAATCTTCCACACTTGCGGTCTTTAGATATGAGCAGCAATGATATTCAGTA
CCTACCAGGTCCCGCACACTGGAAATCTTTGAACTTAAGGGAACTCTTATTTAGCCATAAT
CAGATCAGCATCTTGGACTTGAGTGAAAAAGCATATTTATGGTCTAGAGTAGAGAAACTG
CATCTTTCTCACAATAAACTGAAAGAGATTCCTCCTGAGATTGGCTGTCTTGAAAATCTGA
CATCTCTGGATGTCAGTTACAACTTGGAACTAAGATCCTTTCCCAATGAAATGGGGAAATT
AAGCAAAATATGGGATCTTCCTTTGGATGAACTGCATCTTAACTTTGATTTTAAACATATA
GGATGTAAAGCCAAAGACATCATAAGGTTTCTTCAACAGCGATTAAAAAAGGCTGTGCCT
TATAACCGAATGAAACTTATGATTGTGGGAAATACTGGGAGTGGTAAAACCACCTTATTG
CAGCAATTAATGAAAACCAAGAAATCAGATCTTGGAATGCAAAGTGCCACAGTTGGCATA
GATGTGAAAGACTGGCCTATCCAAATAAGAGACAAAAGAAAGAGAGATCTCGTCCTAAA
TGTGTGGGATTTTGCAGGTCGTGAGGAATTCTATAGTACTCATCCCCATTTTATGACGCAG
CGAGCATTGTACCTTGCTGTCTATGACCTCAGCAAGGGACAGGCTGAAGTTGATGCCATG
AAGCCTTGGCTCTTCAATATAAAGGCTCGCGCTTCTTCTTCCCCTGTGATTCTCGTTGGCAC
ACATTTGGATGTTTCTGATGAGAAGCAACGCAAAGCCTGCATGAGTAAAATCACCAAGGA
ACTCCTGAATAAGCGAGGGTTCCCTGCCATACGAGATTACCACTTTGTGAATGCCACCGA
GGAATCTGATGCTTTGGCAAAACTTCGGAAAACCATCATAAACGAGAGCCTTAATTTCAA
GATCCGAGATCAGCTTGTTGTTGGACAGCTGATTCCAGACTGCTATGTAGAACTTGAAAA
AATCATTTTATCGGAGCGTAAAAATGTGCCAATTGAATTTCCCGTAATTGACCGGAAACG
ATTATTACAACTAGTGAGAGAAAATCAGCTGCAGTTAGATGAAAATGAGCTTCCTCACGC
AGTTCACTTTCTAAATGAATCAGGAGTCCTTCTTCATTTTCAAGACCCAGCACTGCAGTTA
AGTGACTTGTACTTTGTGGAACCCAAGTGGCTTTGTAAAATCATGGCACAGATTTTGACAG
TGAAAGTGGAAGGTTGTCCAAAACACCCTAAGGGCATTATTTCGCGTAGAGATGTGGAAA
AATTTCTTTCAAAAAAAAGGAAATTTCCAAAGAACTACATGTCACAGTATTTTAAGCTCCT
AGAAAAATTCCAGATTGCTTTGCCAATAGGAGAAGAATATTTGCTGGTTCCAAGCAGTTT
GTCTGACCACAGGCCTGTGATAGAGCTTCCCCATTGTGAGAACTCTGAAATTATCATCCGA
CTATATGAAATGCCTTATTTTCCAATGGGATTTTGGTCAAGATTAATCAATCGATTACTTG
AGATTTCACCTTACATGCTTTCAGGGAGAGAACGAGCACTTCGCCCAAACAGAATGTATT
GGCGACAAGGCATTTACTTAAATTGGTCTCCTGAAGCTTATTGTCTGGTAGGATCTGAAGT
CTTAGACAATCATCCAGAGAGTTTCTTAAAAATTACAGTTCCTTCTTGTAGAAAAGGCTGT
ATTCTTTTGGGCCAAGTTGTGGACCACATTGATTCTCTCATGGAAGAATGGTTTCCTGGGT
TGCTGGAGATTGATATTTGTGGTGAAGGAGAAACTCTGTTGAAGAAATGGGCATTATATA
GTTTTAATGATGGTGAAGAACATCAAAAAATCTTACTTGATGACTTGATGAAGAAAGCAG
AGGAAGGAGATCTCTTAGTAAATCCAGATCAACCAAGGCTCACCATTCCAATATCTCAGA
TTGCCCCTGACTTGATTTTGGCTGACCTGCCTAGAAATATTATGTTGAATAATGATGAGTT
GGAATTTGAACAAGCTCCAGAGTTTCTCCTAGGTGATGGCAGTTTTGGATCAGTTTACCGA
GCAGCCTATGAAGGAGAAGAAGTGGCTGTGAAGATTTTTAATAAACATACATCACTCAGG
CTGTTAAGACAAGAGCTTGTGGTGCTTTGCCACCTCCACCACCCCAGTTTGATATCTTTGC
TGGCAGCTGGGATTCGTCCCCGGATGTTGGTGATGGAGTTAGCCTCCAAGGGTTCCTTGGA
TCGCCTGCTTCAGCAGGACAAAGCCAGCCTCACTAGAACCCTACAGCACAGGATTGCACT
CCACGTAGCTGATGGTTTGAGATACCTCCACTCAGCCATGATTATATACCGAGACCTGAA
ACCCCACAATGTGCTGCTTTTCACACTGTATCCCAATGCTGCCATCATTGCAAAGATTGCT
GACTACGGCATTGCTCAGTACTGCTGTAGAATGGGGATAAAAACATCAGAGGGCACACCA
GGGTTTCGTGCACCTGAAGTTGCCAGAGGAAATGTCATTTATAACCAACAGGCTGATGTTT
ATTCATTTGGTTTACTACTCTATGACATTTTGACAACTGGAGGTAGAATAGTAGAGGGTTT
GAAGTTTCCAAATGAGTTTGATGAATTAGAAATACAAGGAAAATTACCTGATCCAGTTAA
AGAATATGGTTGTGCCCCATGGCCTATGGTTGAGAAATTAATTAAACAGTGTTTGAAAGA
AAATCCTCAAGAAAGGCCTACTTCTGCCCAGGTCTTTGACATTTTGAATTCAGCTGAATTA
GTCTGTCTGACGAGACGCATTTTATTACCTAAAAACGTAATTGTTGAATGCATGGTTGCTA
CACATCACAACAGCAGGAATGCAAGCATTTGGCTGGGCTGTGGGCACACCGACAGAGGA
CAGCTCTCATTTCTTGACTTAAATACTGAAGGATACACTTCTGAGGAAGTTGCTGATAGTA
GAATATTGTGCTTAGCCTTGGTGCATCTTCCTGTTGAAAAGGAAAGCTGGATTGTGTCTGG
GACACAGTCTGGTACTCTCCTGGTCATCAATACCGAAGATGGGAAAAAGAGACATACCCT
AGAAAAGATGACTGATTCTGTCACTTGTTTGTATTGCAATTCCTTTTCCAAGCAAAGCAAA
CAAAAAAATTTTCTTTTGGTTGGAACCGCTGATGGCAAGTTAGCAATTTTTGAAGATAAGA
CTGTTAAGCTTAAAGGAGCTGCTCCTTTGAAGATACTAAATATAGGAAATGTCAGTACTCC
ATTGATGTGTTTGAGTGAATCCACAAATTCAACGGAAAGAAATGTAATGTGGGGAGGATG
TGGCACAAAGATTTTCTCCTTTTCTAATGATTTCACCATTCAGAAACTCATTGAGACAAGA
ACAAGCCAACTGTTTTCTTATGCAGCTTTCAGTGATTCCAACATCATAACAGTGGTGGTAG
ACACTGCTCTCTATATTGCTAAGCAAAATAGCCCTGTTGTGGAAGTGTGGGATAAGAAAA
CTGAAAAACTCTGTGGACTAATAGACTGCGTGCACTTTTTAAGGGAGGTAATGGTAAAAG
AAAACAAGGAATCAAAACACAAAATGTCTTATTCTGGGAGAGTGAAAACCCTCTGCCTTC
AGAAGAACACTGCTCTTTGGATAGGAACTGGAGGAGGCCATATTTTACTCCTGGATCTTTC
AACTCGTCGACTTATACGTGTAATTTACAACTTTTGTAATTCGGTCAGAGTCATGATGACA
GCACAGCTAGGAAGCCTTAAAAATGTCATGCTGGTATTGGGCTACAACCGGAAAAATACT
GAAGGTACACAAAAGCAGAAAGAGATACAATCTTGCTTGACCGTTTGGGACATCAATCTT
CCACATGAAGTGCAAAATTTAGAAAAACACATTGAAGTGAGAAAAGAATTAGCTGAAAA
AATGAGACGAACATCTGTTGAGTAA
SEQ ID No 4: >human c-Rel ENSG00000162924.16
ATGGCCTCCGGTGCGTATAACCCGTATATAGAGATAATTGAACAACCCAGGCAGAGGGGA
ATGCGTTTTAGATACAAATGTGAAGGGCGATCAGCAGGCAGCATTCCAGGGGAGCACAGC
ACAGACAACAACCGAACATACCCTTCTATCCAGATTATGAACTATTATGGAAAAGGAAAA
GTGAGAATTACATTAGTAACAAAGAATGACCCATATAAACCTCATCCTCATGATTTAGTTG
GAAAAGACTGCAGAGACGGCTACTATGAAGCAGAATTTGGACAAGAACGCAGACCTTTG
TTTTTCCAAAATTTGGGTATTCGATGTGTGAAGAAAAAAGAAGTAAAAGAAGCTATTATT
ACAAGAATAAAGGCAGGAATCAATCCATTCAATGTCCCTGAAAAACAGCTGAATGATATT
GAAGATTGTGACCTCAATGTGGTGAGACTGTGTTTTCAAGTTTTTCTCCCTGATGAACATG
GTAATTTGACGACTGCTCTTCCTCCTGTTGTCTCGAACCCAATTTATGACAACCGTGCTCC
AAATACTGCAGAATTAAGGATTTGTCGTGTAAACAAGAATTGTGGAAGTGTCAGAGGAGG
AGATGAAATATTTCTACTTTGTGACAAAGTTCAGAAAGATGACATAGAAGTTCGTTTTGTG
TTGAACGATTGGGAAGCAAAAGGCATCTTTTCACAAGCTGATGTACACCGTCAAGTAGCC
ATTGTTTTCAAAACTCCACCATATTGCAAAGCTATCACAGAACCCGTAACAGTAAAAATG
CAGTTGCGGAGACCTTCTGACCAGGAAGTTAGTGAATCTATGGATTTTAGATATCTGCCAG
ATGAAAAAGATACTTACGGCAATAAAGCAAAGAAACAAAAGACAACTCTGCTTTTCCAG
AAACTGTGCCAGGATCACGTTAATTTTCCTGAGAGACCAAGACCTGGTCTCCTCGGTTCAA
TTGGAGAAGGAAGATACTTCAAAAAAGAACCAAACTTGTTTTCTCATGATGCAGTTGTGA
GAGAAATGCCTACAGGGGTTTCAAGTCAAGCAGAATCCTACTATCCCTCACCTGGGCCCA
TCTCAAGTGGATTGTCACATCATGCCTCAATGGCACCTCTGCCTTCTTCAAGCTGGTCATC
AGTGGCCCACCCCACCCCACGCTCAGGCAATACAAACCCACTGAGTAGTTTTTCAACAAG
GACACTTCCTTCTAATTCGCAAGGTATCCCACCATTCCTGAGAATACCTGTTGGGAATGAT
TTAAATGCTTCTAATGCTTGCATTTACAACAATGCCGATGACATAGTCGGAATGGAAGCGT
CATCCATGCCATCAGCAGATTTATATGGTATTTCTGATCCCAACATGCTGTCTAATTGTTCT
GTGAATATGATGACAACCAGCAGTGACAGCATGGGAGAGACTGATAATCCAAGACTTCTG
AGCATGAATCTTGAAAACCCCTCATGTAATTCAGTGTTAGACCCAAGAGACTTGAGACAG
CTCCATCAGATGTCCTCTTCCAGTATGTCAGCAGGCGCCAATTCCAATACTACTGTTTTTGT
TTCACAATCAGATGCATTTGAGGGATCTGACTTCAGTTGTGCAGATAACAGCATGATAAA
TGAGTCGGGACCATCAAACAGTACTAATCCAAACAGTCATGGTTTTGTTCAAGATAGTCA
GTATTCAGGTATTGGCAGTATGCAAAATGAGCAATTGAGTGACTCCTTTCCATATGAATTT
TTTCAAGTATAA
SEQ ID No 5: >human ATG7 ENSG00000197548.13
ATGGCGGCAGCTACGGGGGATCCTGGACTCTCTAAACTGCAGTTTGCCCCTTTTAGTAGTG
CCTTGGATGTTGGGTTTTGGCATGAGTTGACCCAGAAGAAGCTGAACGAGTATCGGCTGG
ATGAAGCTCCCAAGGACATTAAGGGTTATTACTACAATGGTGACTCTGCTGGGCTGCCAG
CTCGCTTAACATTGGAGTTCAGTGCTTTTGACATGAGTGCTCCCACCCCAGCCCGTTGCTG
CCCAGCTATTGGAACACTGTATAACACCAACACACTCGAGTCTTTCAAGACTGCAGATAA
GAAGCTCCTTTTGGAACAAGCAGCAAATGAGATATGGGAATCCATAAAATCAGGCACTGC
TCTTGAAAACCCTGTACTCCTCAACAAGTTCCTCCTCTTGACATTTGCAGATCTAAAGAAG
TACCACTTCTACTATTGGTTTTGCTATCCTGCCCTCTGTCTTCCAGAGAGTTTACCTCTCAT
TCAGGGGCCAGTGGGTTTGGATCAAAGGTTTTCACTAAAACAGATTGAAGCACTAGAGTG
TGCATATGATAATCTTTGTCAAACAGAAGGAGTCACAGCTCTTCCTTACTTCTTAATCAAG
TATGATGAGAACATGGTGCTGGTTTCCTTGCTTAAACACTACAGTGATTTCTTCCAAGGTC
AAAGGACGAAGATAACAATTGGTGTATATGATCCCTGTAACTTAGCCCAGTACCCTGGAT
GGCCTTTGAGGAATTTTTTGGTCCTAGCAGCCCACAGATGGAGTAGCAGTTTCCAGTCTGT
TGAAGTTGTTTGCTTCCGTGACCGTACCATGCAGGGGGCGAGAGACGTTGCCCACAGCAT
CATCTTCGAAGTGAAGCTTCCAGAAATGGCATTTAGCCCAGATTGTCCTAAAGCAGTTGG
ATGGGAAAAGAACCAGAAAGGAGGCATGGGACCAAGGATGGTGAACCTCAGTGAATGTA
TGGACCCTAAAAGGTTAGCTGAGTCATCAGTGGATCTAAATCTCAAACTGATGTGTTGGA
GATTGGTTCCTACTTTAGACTTGGACAAGGTTGTGTCTGTCAAATGTCTGCTGCTTGGAGC
CGGCACCTTGGGTTGCAATGTAGCTAGGACGTTGATGGGTTGGGGCGTGAGACACATCAC
ATTTGTGGACAATGCCAAGATCTCCTACTCCAATCCTGTGAGGCAGCCTCTCTATGAGTTT
GAAGATTGCCTAGGGGGTGGTAAGCCCAAGGCTCTGGCAGCAGCGGACCGGCTCCAGAA
AATATTCCCCGGTGTGAATGCCAGAGGATTCAACATGAGCATACCTATGCCTGGGCATCC
AGTGAACTTCTCCAGTGTCACTCTGGAGCAAGCCCGCAGAGATGTGGAGCAACTGGAGCA
GCTCATCGAAAGCCATGATGTCGTCTTCCTATTGATGGACACCAGGGAGAGCCGGTGGCT
TCCTGCCGTCATTGCTGCAAGCAAGAGAAAGCTGGTCATCAATGCTGCTTTGGGATTTGAC
ACATTTGTTGTCATGAGACATGGTCTGAAGAAACCAAAGCAGCAAGGAGCTGGGGACTTG
TGTCCAAACCACCCTGTGGCATCTGCTGACCTCCTGGGCTCATCGCTTTTTGCCAACATCC
CTGGTTACAAGCTTGGCTGCTACTTCTGCAATGATGTGGTGGCCCCAGGAGATTCAACCAG
AGACCGGACCTTGGACCAGCAGTGCACTGTGAGTCGTCCAGGACTGGCCGTGATTGCAGG
AGCCCTGGCCGTGGAATTGATGGTATCTGTTTTGCAGCATCCAGAAGGGGGCTATGCCATT
GCCAGCAGCAGTGACGATCGGATGAATGAGCCTCCAACCTCTCTTGGGCTTGTGCCTCAC
CAGATCCGGGGATTTCTTTCACGGTTTGATAATGTCCTTCCCGTCAGCCTGGCATTTGACA
AATGTACAGCTTGTTCTTCCAAAGTTCTTGATCAATATGAACGAGAAGGATTTAACTTCCT
AGCCAAGGTGTTTAATTCTTCACATTCCTTCTTAGAAGACTTGACTGGTCTTACATTGCTGC
ATCAAGAAACCCAAGCTGCTGAGATCTGGGACATGAGCGATGATGAGACCATCTGA
SEQ ID No 6: >human VMAT2 ENSG00000165646.14
ATGGCCCTGAGCGAGCTGGCGCTGGTCCGCTGGCTGCAGGAGAGCCGCCGCTCGCGGAAG
CTCATCCTGTTCATCGTGTTCCTGGCGCTGCTGCTGGACAACATGCTGCTCACTGTCGTGG
TCCCCATCATCCCAAGTTATCTGTACAGCATTAAGCATGAGAAGAATGCTACAGAAATCC
AGACGGCCAGGCCAGTGCACACTGCCTCCATCTCAGACAGCTTCCAGAGCATCTTCTCCTA
TTATGATAACTCGACTATGGTCACCGGGAATGCTACCAGAGACCTGACACTTCATCAGAC
CGCCACACAGCACATGGTGACCAACGCGTCCGCTGTTCCTTCCGACTGTCCCAGTGAAGA
CAAAGACCTCCTGAATGAAAACGTGCAAGTTGGTCTGTTGTTTGCCTCGAAAGCCACCGT
CCAGCTCATCACCAACCCTTTCATAGGACTACTGACCAACAGAATTGGCTATCCAATTCCC
ATATTTGCGGGATTCTGCATCATGTTTGTCTCAACAATTATGTTTGCCTTCTCCAGCAGCTA
TGCCTTCCTGCTGATTGCCAGGTCGCTGCAGGGCATCGGCTCGTCCTGCTCCTCTGTGGCT
GGGATGGGCATGCTTGCCAGTGTCTACACAGATGATGAAGAGAGAGGCAACGTCATGGG
AATCGCCTTGGGAGGCCTGGCCATGGGGGTCTTAGTGGGCCCCCCCTTCGGGAGTGTGCT
CTATGAGTTTGTGGGGAAGACGGCTCCGTTCCTGGTGCTGGCCGCCCTGGTACTCTTGGAT
GGAGCTATTCAGCTCTTTGTGCTCCAGCCGTCCCGGGTGCAGCCAGAGAGTCAGAAGGGG
ACACCCCTAACCACGCTGCTGAAGGACCCGTACATCCTCATTGCTGCAGGCTCCATCTGCT
TTGCAAACATGGGCATCGCCATGCTGGAGCCAGCCCTGCCCATCTGGATGATGGAGACCA
TGTGTTCCCGAAAGTGGCAGCTGGGCGTTGCCTTCTTGCCAGCTAGTATCTCTTATCTCATT
GGAACCAATATTTTTGGGATACTTGCACACAAAATGGGGAGGTGGCTTTGTGCTCTTCTGG
GAATGATAATTGTTGGAGTCAGCATTTTATGTATTCCATTTGCAAAAAACATTTATGGACT
CATAGCTCCGAACTTTGGAGTTGGTTTTGCAATTGGAATGGTGGATTCGTCAATGATGCCT
ATCATGGGCTACCTCGTAGACCTGCGGCACGTGTCCGTCTATGGGAGTGTGTACGCCATTG
CGGATGTGGCATTTTGTATGGGGTATGCTATAGGTCCTTCTGCTGGTGGTGCTATTGCAAA
GGCAATTGGATTTCCATGGCTCATGACAATTATTGGGATAATTGATATTCTTTTTGCCCCTC
TCTGCTTTTTTCTTCGAAGTCCACCTGCCAAAGAAGAAAAAATGGCTATTCTCATGGATCA
CAACTGCCCTATTAAAACAAAAATGTACACTCAGAATAATATCCAGTCATATCCGATAGG
TGAAGATGAAGAATCTGAAAGTGACTGA
SEQ ID No 7: >human GBA ENSG00000177628.16
ATGGAGTTTTCAAGTCCTTCCAGAGAGGAATGTCCCAAGCCTTTGAGTAGGGTAAGCATC
ATGGCTGGCAGCCTCACAGGATTGCTTCTACTTCAGGCAGTGTCGTGGGCATCAGGTGCCC
GCCCCTGCATCCCTAAAAGCTTCGGCTACAGCTCGGTGGTGTGTGTCTGCAATGCCACATA
CTGTGACTCCTTTGACCCCCCGACCTTTCCTGCCCTTGGTACCTTCAGCCGCTATGAGAGT
ACACGCAGTGGGCGACGGATGGAGCTGAGTATGGGGCCCATCCAGGCTAATCACACGGG
CACAGGCCTGCTACTGACCCTGCAGCCAGAACAGAAGTTCCAGAAAGTGAAGGGATTTGG
AGGGGCCATGACAGATGCTGCTGCTCTCAACATCCTTGCCCTGTCACCCCCTGCCCAAAAT
TTGCTACTTAAATCGTACTTCTCTGAAGAAGGAATCGGATATAACATCATCCGGGTACCCA
TGGCCAGCTGTGACTTCTCCATCCGCACCTACACCTATGCAGACACCCCTGATGATTTCCA
GTTGCACAACTTCAGCCTCCCAGAGGAAGATACCAAGCTCAAGATACCCCTGATTCACCG
AGCCCTGCAGTTGGCCCAGCGTCCCGTTTCACTCCTTGCCAGCCCCTGGACATCACCCACT
TGGCTCAAGACCAATGGAGCGGTGAATGGGAAGGGGTCACTCAAGGGACAGCCCGGAGA
CATCTACCACCAGACCTGGGCCAGATACTTTGTGAAGTTCCTGGATGCCTATGCTGAGCAC
AAGTTACAGTTCTGGGCAGTGACAGCTGAAAATGAGCCTTCTGCTGGGCTGTTGAGTGGA
TACCCCTTCCAGTGCCTGGGCTTCACCCCTGAACATCAGCGAGACTTCATTGCCCGTGACC
TAGGTCCTACCCTCGCCAACAGTACTCACCACAATGTCCGCCTACTCATGCTGGATGACCA
ACGCTTGCTGCTGCCCCACTGGGCAAAGGTGGTACTGACAGACCCAGAAGCAGCTAAATA
TGTTCATGGCATTGCTGTACATTGGTACCTGGACTTTCTGGCTCCAGCCAAAGCCACCCTA
GGGGAGACACACCGCCTGTTCCCCAACACCATGCTCTTTGCCTCAGAGGCCTGTGTGGGCT
CCAAGTTCTGGGAGCAGAGTGTGCGGCTAGGCTCCTGGGATCGAGGGATGCAGTACAGCC
ACAGCATCATCACGAACCTCCTGTACCATGTGGTCGGCTGGACCGACTGGAACCTTGCCCT
GAACCCCGAAGGAGGACCCAATTGGGTGCGTAACTTTGTCGACAGTCCCATCATTGTAGA
CATCACCAAGGACACGTTTTACAAACAGCCCATGTTCTACCACCTTGGCCACTTCAGCAAG
TTCATTCCTGAGGGCTCCCAGAGAGTGGGGCTGGTTGCCAGTCAGAAGAACGACCTGGAC
GCAGTGGCACTGATGCATCCCGATGGCTCTGCTGTTGTGGTCGTGCTAAACCGCTCCTCTA
AGGATGTGCCTCTTACCATCAAGGATCCTGCTGTGGGCTTCCTGGAGACAATCTCACCTGG
CTACTCCATTCACACCTACCTGTGGCGTCGCCAGTGA
SEQ ID No 8: >human SNCA ENSG00000145335.17
ATGGATGTATTCATGAAAGGACTTTCAAAGGCCAAGGAGGGAGTIGTGGCTGCTGCTGAG
AAAACCAAACAGGGTGTGGCAGAAGCAGCAGGAAAGACAAAAGAGGGTGTTCTCTATGT
AGGCTCCAAAACCAAGGAGGGAGTGGTGCATGGTGTGGCAACAGTGGCTGAGAAGACCA
AAGAGCAAGTGACAAATGTTGGAGGAGCAGTGGTGACGGGTGTGACAGCAGTAGCCCAG
AAGACAGTGGAGGGAGCAGGGAGCATTGCAGCAGCCACTGGCTTTGTCAAAAAGGACCA
GTTGGGCAAGAATGAAGAAGGAGCCCCACAGGAAGGAATTCTGGAAGATATGCCTGTGG
ATCCTGACAATGAGGCTTATGAAATGCCTTCTGAGGAAGGGTATCAAGACTACGAACCTG
AAGCCTAA
SEQ ID No 9: >human MAPT ENSG00000186868.18
ATGGCTGAGCCCCGCCAGGAGTTCGAAGTGATGGAAGATCACGCTGGGACGTACGGGTTG
GGGGACAGGAAAGATCAGGGGGGCTACACCATGCACCAAGACCAAGAGGGTGACACGGA
CGCTGGCCTGAAAGAATCTCCCCTGCAGACCCCCACTGAGGACGGATCTGAGGAACCGGG
CTCTGAAACCTCTGATGCTAAGAGCACTCCAACAGCGGAAGCTGAAGAAGCAGGCATTGG
AGACACCCCCAGCCTGGAAGACGAAGCTGCTGGTCACGTGACCCAAGAGGAGTTGAGAG
TTCCGGGCCGGCAGAGGAAGGCGCCTGAAAGGCCCCTGGCCAATGAGATTAGCGCCCAC
GTCCAGCCTGGACCCTGCGGAGAGGCCTCTGGGGTCTCTGGGCCGTGCCTCGGGGAGAAA
GAGCCAGAAGCTCCCGTCCCGCTGACCGCGAGCCTTCCTCAGCACCGTCCCGTTTGCCCAG
CGCCTCCTCCAACAGGAGGCCCTCAGGAGCCCTCCCTGGAGTGGGGACAAAAAGGCGGG
GACTGGGCCGAGAAGGGTCCGGCCTTTCCGAAGCCCGCCACCACTGCGTATCTCCACACA
GAGCCTGAAAGTGGTAAGGTGGTCCAGGAAGGCTTCCTCCGAGAGCCAGGCCCCCCAGGT
CTGAGCCACCAGCTCATGTCCGGCATGCCTGGGGCTCCCCTCCTGCCTGAGGGCCCCAGA
GAGGCCACACGCCAACCTTCGGGGACAGGACCTGAGGACACAGAGGGCGGCCGCCACGC
CCCTGAGCTGCTCAAGCACCAGCTTCTAGGAGACCTGCACCAGGAGGGGCCGCCGCTGAA
GGGGGCAGGGGGCAAAGAGAGGCCGGGGAGCAAGGAGGAGGTGGATGAAGACCGCGAC
GTCGATGAGTCCTCCCCCCAAGACTCCCCTCCCTCCAAGGCCTCCCCAGCCCAAGATGGGC
GGCCTCCCCAGACAGCCGCCAGAGAAGCCACCAGCATCCCAGGCTTCCCAGCGGAGGGTG
CCATCCCCCTCCCTGTGGATTTCCTCTCCAAAGTTTCCACAGAGATCCCAGCCTCAGAGCC
CGACGGGCCCAGTGTAGGGCGGGCCAAAGGGCAGGATGCCCCCCTGGAGTTCACGTTTCA
CGTGGAAATCACACCCAACGTGCAGAAGGAGCAGGCGCACTCGGAGGAGCATTTGGGAA
GGGCTGCATTTCCAGGGGCCCCTGGAGAGGGGCCAGAGGCCCGGGGCCCCTCTTTGGGAG
AGGACACAAAAGAGGCTGACCTTCCAGAGCCCTCTGAAAAGCAGCCTGCTGCTGCTCCGC
GGGGGAAGCCCGTCAGCCGGGTCCCTCAACTCAAAGCTCGCATGGTCAGTAAAAGCAAA
GACGGGACTGGAAGCGATGACAAAAAAGCCAAGACATCCACACGTTCCTCTGCTAAAAC
CTTGAAAAATAGGCCTTGCCTTAGCCCCAAACACCCCACTCCTGGTAGCTCAGACCCTCTG
ATCCAACCCTCCAGCCCTGCTGTGTGCCCAGAGCCACCTTCCTCTCCTAAATACGTCTCTT
CTGTCACTTCCCGAACTGGCAGTTCTGGAGCAAAGGAGATGAAACTCAAGGGGGCTGATG
GTAAAACGAAGATCGCCACACCGCGGGGAGCAGCCCCTCCAGGCCAGAAGGGCCAGGCC
AACGCCACCAGGATTCCAGCAAAAACCCCGCCCGCTCCAAAGACACCACCCAGCTCTGGT
GAACCTCCAAAATCAGGGGATCGCAGCGGCTACAGCAGCCCCGGCTCCCCAGGCACTCCC
GGCAGCCGCTCCCGCACCCCGTCCCTTCCAACCCCACCCACCCGGGAGCCCAAGAAGGTG
GCAGTGGTCCGTACTCCACCCAAGTCGCCGTCTTCCGCCAAGAGCCGCCTGCAGACAGCC
CCCGTGCCCATGCCAGACCTGAAGAATGTCAAGTCCAAGATCGGCTCCACTGAGAACCTG
AAGCACCAGCCGGGAGGCGGGAAGGTGCAGATAATTAATAAGAAGCTGGATCTTAGCAA
CGTCCAGTCCAAGTGTGGCTCAAAGGATAATATCAAACACGTCCCGGGAGGCGGCAGTGT
GCAAATAGTCTACAAACCAGTTGACCTGAGCAAGGTGACCTCCAAGTGTGGCTCATTAGG
CAACATCCATCATAAACCAGGAGGTGGCCAGGTGGAAGTAAAATCTGAGAAGCTTGACTT
CAAGGACAGAGTCCAGTCGAAGATTGGGTCCCTGGACAATATCACCCACGTCCCTGGCGG
AGGAAATAAAAAGATTGAAACCCACAAGCTGACCTTCCGCGAGAACGCCAAAGCCAAGA
CAGACCACGGGGCGGAGATCGTGTACAAGTCGCCAGTGGTGTCTGGGGACACGTCTCCAC
GGCATCTCAGCAATGTCTCCTCCACCGGCAGCATCGACATGGTAGACTCGCCCCAGCTCG
CCACGCTAGCTGACGAGGTGTCTGCCTCCCTGGCCAAGCAGGGTTTGTGA
SEQ ID No 10: >human GDNF ENSG00000168621.15
ATGAAGTTATGGGATGTCGTGGCTGTCTGCCTGGTGCTGCTCCACACCGCGTCCGCCTTCC
CGCTGCCCGCCGGTAAGAGGCCTCCCGAGGCGCCCGCCGAAGACCGCTCCCTCGGCCGCC
GCCGCGCGCCCTTCGCGCTGAGCAGTGACTCAAATATGCCAGAGGATTATCCTGATCAGT
TCGATGATGTCATGGATTTTATTCAAGCCACCATTAAAAGACTGAAAAGGTCACCAGATA
AACAAATGGCAGTGCTTCCTAGAAGAGAGCGGAATCGGCAGGCTGCAGCTGCCAACCCA
GAGAATTCCAGAGGAAAAGGTCGGAGAGGCCAGAGGGGCAAAAACCGGGGTTGTGTCTT
AACTGCAATACATTTAAATGTCACTGACTTGGGTCTGGGCTATGAAACCAAGGAGGAACT
GATTTTTAGGTACTGCAGCGGCTCTTGCGATGCAGCTGAGACAACGTACGACAAAATATT
GAAAAACTTATCCAGAAATAGAAGGCTGGTGAGTGACAAAGTAGGGCAGGCATGTTGCA
GACCCATCGCCTTTGATGATGACCTGTCGTTTTTAGATGATAACCTGGTTTACCATATTCTA
AGAAAGCATTCCGCTAAAAGGTGTGGATGTATCTGA
SEQ ID No 11: >human PARK2 ENSG00000185345.24
MIVFVRFNSSHGFPVEVDSDTSIFQLKEVVAKRQGVPADQLRVIFAGKELRNDWTVQNCDLD
QQSIVHIVQRPWRKGQEMNATGGDDPRNAAGGCEREPQSLTRVDLSSSVLPGDSVGLAVILH
TDSRKDSPPAGSPAGRSIYNSFYVYCKGPCQRVQPGKLRVQCSTCRQATLTLTQGPSCWDDVL
IPNRMSGECQSPHCPGTSAEFFFKCGAHPTSDKETSVALHLIATNSRNITCITCTDVRSPVLVFQ
CNSRHVICLDCFHLYCVTRLNDRQFVHDPQLGYSLPCVAGCPNSLIKELHHFRILGEEQYNRY
QQYGAEECVLQMGGVLCPRPGCGAGLLPEPDQRKVTCEGGNGLGCGFAFCRECKEAYHEGE
CSAVFEASGTTTQAYRVDERAAEQARWEAASKETIKKTTKPCPRCHVPVEKNGGCMHMKCP
QPQCRLEWCWNCGCEWNRVCMGDHWFDV
SEQ ID No 12: >human PINK1 ENSG00000158828.8
MAVRQALGRGLQLGRALLLRFTGKPGRAYGLGRPGPAAGCVRGERPGWAAGPGAEPRRVGL
GLPNRLRFFRQSVAGLAARLQRQFVVRAWGCAGPCGRAVFLAFGLGLGLIEEKQAESRRAVS
ACQEIQAIFTQKSKPGPDPLDTRRLQGFRLEEYLIGQSIGKGCSAAVYEATMPTLPQNLEVTKS
TGLLPGRGPGTSAPGEGQERAPGAPAFPLAIKMMWNISAGSSSEAILNTMSQELVPASRVALA
GEYGAVTYRKSKRGPKQLAPHPNIIRVLRAFTSSVPLLPGALVDYPDVLPSRLHPEGLGHGRTL
FLVMKNYPCTLRQYLCVNTPSPRLAAMMLLQLLEGVDHLVQQGIAHRDLKSDNILVELDPDG
CPWLVIADFGCCLADESIGLQLPFSSWYVDRGGNGCLMAPEVSTARPGPRAVIDYSKADAWA
VGAIAYEIFGLVNPFYGQGKAHLESRSYQEAQLPALPESVPPDVRQLVRALLQREASKRPSAR
VAANVLHLSLWGEHILALKNLKLDKMVGWLLQQSAATLLANRLTEKCCVETKMKMLFLAN
LECETLCQAALLLCSWRAAL
SEQ ID No 13: >human LRRK2 ENSG00000188906.17
MASGSCQGCEEDEETLKKLIVRLNNVQEGKQIETLVQILEDLLVFTYSERASKLFQGKNIHVPL
LIVLDSYMRVASVQQVGWSLLCKLIEVCPGTMQSLMGPQDVGNDWEVLGVHQLILKMLTVH
NASVNLSVIGLKTLDLLLTSGKITLLILDEESDIFMLIFDAMHSFPANDEVQKLGCKALHVLFER
VSEEQLTEFVENKDYMILLSALTNFKDEEEIVLHVLHCLHSLAIPCNNVEVLMSGNVRCYNIV
VEAMKAFPMSERIQEVSCCLLHRLTLGNFFNILVLNEVHEFVVKAVQQYPENAALQISALSCL
ALLTETIFLNQDLEEKNENQENDDEGEEDKLFWLEACYKALTWHRKNKHVQEAACWALNNL
LMYQNSLHEKIGDEDGHFPAHREVMLSMLMHSSSKEVFQASANALSTLLEQNVNFRKILLSK
GIHLNVLELMQKHIHSPEVAESGCKMLNHLFEGSNTSLDIMAAVVPKILTVMKRHETSLPVQL
EALRAILHFIVPGMPEESREDTEFHHKLNMVKKQCFKNDIHKLVLAALNRFIGNPGIQKCGLK
VISSIVHFPDALEMLSLEGAMDSVLHTLQMYPDDQEIQCLGLSLIGYLITKKNVFIGTGHLLAKI
LVSSLYRFKDVAEIQTKGFQTILAILKLSASFSKLLVHHSFDLVIFHQMSSNIMEQKDQQFLNLC
CKCFAKVAMDDYLKNVMLERACDQNNSIMVECLLLLGADANQAKEGSSLICQVCEKESSPK
LVELLLNSGSREQDVRKALTISIGKGDSQIISLLLRRLALDVANNSICLGGFCIGKVEPSWLGPL
FPDKTSNLRKQTNIASTLARMVIRYQMKSAVEEGTASGSDGNFSEDVLSKFDEWTFIPDSSMD
SVFAQSDDLDSEGSEGSFLVKKKSNSISVGEFYRDAVLQRCSPNLQRHSNSLGPIFDHEDLLKR
KRKILSSDDSLRSSKLQSHMRHSDSISSLASEREYITSLDLSANELRDIDALSQKCCISVHLEHLE
KLELHQNALTSFPQQLCETLKSLTHLDLHSNKFTSFPSYLLKMSCIANLDVSRNDIGPSVVLDP
TVKCPTLKQFNLSYNQLSFVPENLTDVVEKLEQLILEGNKISGICSPLRLKELKILNLSKNHISSL
SENFLEACPKVESFSARMNFLAAMPFLPPSMTILKLSQNKFSCIPEAILNLPHLRSLDMSSNDIQ
YLPGPAHWKSLNLRELLFSHNQISILDLSEKAYLWSRVEKLHLSHNKLKEIPPEIGCLENLTSLD
VSYNLELRSFPNEMGKLSKIWDLPLDELHLNFDFKHIGCKAKDIIRFLQQRLKKAVPYNRMKL
MIVGNTGSGKTTLLQQLMKTKKSDLGMQSATVGIDVKDWPIQIRDKRKRDLVLNVWDFAGR
EEFYSTHPHFMTQRALYLAVYDLSKGQAEVDAMKPWLFNIKARASSSPVILVGTHLDVSDEK
QRKACMSKITKELLNKRGFPAIRDYHFVNATEESDALAKLRKTIINESLNFKIRDQLVVGQLIP
DCYVELEKIILSERKNVPIEFPVIDRKRLLQLVRENQLQLDENELPHAVHFLNESGVLLHFQDP
ALQLSDLYFVEPKWLCKIMAQILTVKVEGCPKHPKGIISRRDVEKFLSKKRKFPKNYMSQYFK
LLEKFQIALPIGEEYLLVPSSLSDHRPVIELPHCENSEIIIRLYEMPYFPMGFWSRLINRLLEISPY
MLSGRERALRPNRMYWRQGIYLNWSPEAYCLVGSEVLDNHPESFLKITVPSCRKGCILLGQV
VDHIDSLMEEWFPGLLEIDICGEGETLLKKWALYSFNDGEEHQKILLDDLMKKAEEGDLLVNP
DQPRLTIPISQIAPDLILADLPRNIMLNNDELEFEQAPEFLLGDGSFGSVYRAAYEGEEVAVKIF
NKHTSLRLLRQELVVLCHLHHPSLISLLAAGIRPRMLVMELASKGSLDRLLQQDKASLTRTLQ
HRIALHVADGLRYLHSAMIIYRDLKPHNVLLFTLYPNAAIIAKIADYGIAQYCCRMGIKTSEGT
PGFRAPEVARGNVIYNQQADVYSFGLLLYDILTTGGRIVEGLKFPNEFDELEIQGKLPDPVKEY
GCAPWPMVEKLIKQCLKENPQERPTSAQVFDILNSAELVCLTRRILLPKNVIVECMVATHHNS
RNASIWLGCGHTDRGQLSFLDLNTEGYTSEEVADSRILCLALVHLPVEKESWIVSGTQSGTLL
VINTEDGKKRHTLEKMTDSVTCLYCNSFSKQSKQKNFLLVGTADGKLAIFEDKTVKLKGAAP
LKILNIGNVSTPLMCLSESTNSTERNVMWGGCGTKIFSFSNDFTIQKLIETRTSQLFSYAAFSDS
NIITVVVDTALYIAKQNSPVVEVWDKKTEKLCGLIDCVHFLREVMVKENKESKHKMSYSGRV
KTLCLQKNTALWIGTGGGHILLLDLSTRRLIRVIYNFCNSVRVMMTAQLGSLKNVMLVLGYN
RKNTEGTQKQKEIQSCLTVWDINLPHEVQNLEKHIEVRKELAEKMRRTSVE
SEQ ID No 14: >human c-Rel ENSG00000162924.16
MASGAYNPYIEIIEQPRQRGMRFRYKCEGRSAGSIPGEHSTDNNRTYPSIQIMNYYGKGKVRIT
LVTKNDPYKPHPHDLVGKDCRDGYYEAEFGQERRPLFFQNLGIRCVKKKEVKEAIITRIKAGI
NPFNVPEKQLNDIEDCDLNVVRLCFQVFLPDEHGNLTTALPPVVSNPIYDNRAPNTAELRICRV
NKNCGSVRGGDEIFLLCDKVQKDDIEVRFVLNDWEAKGIFSQADVHRQVAIVFKTPPYCKAIT
EPVTVKMQLRRPSDQEVSESMDFRYLPDEKDTYGNKAKKQKTTLLFQKLCQDHVNFPERPRP
GLLGSIGEGRYFKKEPNLFSHDAVVREMPTGVSSQAESYYPSPGPISSGLSHHASMAPLPSSSW
SSVAHPTPRSGNTNPLSSFSTRTLPSNSQGIPPFLRIPVGNDLNASNACIYNNADDIVGMEASSM
PSADLYGISDPNMLSNCSVNMMTTSSDSMGETDNPRLLSMNLENPSCNSVLDPRDLRQLHQM
SSSSMSAGANSNTTVFVSQSDAFEGSDFSCADNSMINESGPSNSTNPNSHGFVQDSQYSGIGSM
QNEQLSDSFPYEFFQV
SEQ ID No 15: >human ATG7 ENSG00000197548.13
MAAATGDPGLSKLQFAPFSSALDVGFWHELTQKKLNEYRLDEAPKDIKGYYYNGDSAGLPA
RLTLEFSAFDMSAPTPARCCPAIGTLYNTNTLESFKTADKKLLLEQAANEIWESIKSGTALENP
VLLNKFLLLTFADLKKYHFYYWFCYPALCLPESLPLIQGPVGLDQRFSLKQIEALECAYDNLC
QTEGVTALPYFLIKYDENMVLVSLLKHYSDFFQGQRTKITIGVYDPCNLAQYPGWPLRNFLVL
AAHRWSSSFQSVEVVCFRDRTMQGARDVAHSIIFEVKLPEMAFSPDCPKAVGWEKNQKGGM
GPRMVNLSECMDPKRLAESSVDLNLKLMCWRLVPTLDLDKVVSVKCLLLGAGTLGCNVART
LMGWGVRHITFVDNAKISYSNPVRQPLYEFEDCLGGGKPKALAAADRLQKIFPGVNARGFNM
SIPMPGHPVNFSSVTLEQARRDVEQLEQLIESHDVVFLLMDTRESRWLPAVIAASKRKLVINAA
LGFDTFVVMRHGLKKPKQQGAGDLCPNHPVASADLLGSSLFANIPGYKLGCYFCNDVVAPGD
STRDRTLDQQCTVSRPGLAVIAGALAVELMVSVLQHPEGGYAIASSSDDRMNEPPTSLGLVPH
QIRGFLSRFDNVLPVSLAFDKCTACSSKVLDQYEREGFNFLAKVFNSSHSFLEDLTGLTLLHQE
TQAAEIWDMSDDETI
SEQ ID No 16: >human VMAT2 ENSG00000165646.14
MALSELALVRWLQESRRSRKLILFIVFLALLLDNMLLTVVVPIIPSYLYSIKHEKNATEIQTARP
VHTASISDSFQSIFSYYDNSTMVTGNATRDLTLHQTATQHMVTNASAVPSDCPSEDKDLLNEN
VQVGLLFASKATVQLITNPFIGLLTNRIGYPIPIFAGFCIMFVSTIMFAFSSSYAFLLIARSLQGIGS
SCSSVAGMGMLASVYTDDEERGNVMGIALGGLAMGVLVGPPFGSVLYEFVGKTAPFLVLAA
LVLLDGAIQLFVLQPSRVQPESQKGTPLTTLLKDPYILIAAGSICFANMGIAMLEPALPIWMME
TMCSRKWQLGVAFLPASISYLIGTNIFGILAHKMGRWLCALLGMIIVGVSILCIPFAKNIYGLIA
PNFGVGFAIGMVDSSMMPIMGYLVDLRHVSVYGSVYAIADVAFCMGYAIGPSAGGAIAKAIG
FPWLMTIIGIIDILFAPLCFFLRSPPAKEEKMAILMDHNCPIKTKMYTQNNIQSYPIGEDEESESD
SEQ ID No 17: >human GBA ENSG00000177628.16
MEFSSPSREECPKPLSRVSIMAGSLTGLLLLQAVSWASGARPCIPKSFGYSSVVCVCNATYCDS
FDPPTFPALGTFSRYESTRSGRRMELSMGPIQANHTGTGLLLTLQPEQKFQKVKGFGGAMTDA
AALNILALSPPAQNLLLKSYFSEEGIGYNIIRVPMASCDFSIRTYTYADTPDDFQLHNFSLPEEDT
KLKIPLIHRALQLAQRPVSLLASPWTSPTWLKTNGAVNGKGSLKGQPGDIYHQTWARYFVKF
LDAYAEHKLQFWAVTAENEPSAGLLSGYPFQCLGFTPEHQRDFIARDLGPTLANSTHHNVRLL
MLDDQRLLLPHWAKVVLTDPEAAKYVHGIAVHWYLDFLAPAKATLGETHRLFPNTMLFASE
ACVGSKFWEQSVRLGSWDRGMQYSHSIITNLLYHVVGWTDWNLALNPEGGPNWVRNFVDS
PIIVDITKDTFYKQPMFYHLGHFSKFIPEGSQRVGLVASQKNDLDAVALMHPDGSAVVVVLNR
SSKDVPLTIKDPAVGFLETISPGYSIHTYLWRRQ
SEQ ID No 18: >human SNCA ENSG00000145335.17
MDVFMKGLSKAKEGVVAAAEKTKQGVAEAAGKTKEGVLYVGSKTKEGVVHGVATVAEKT
KEQVTNVGGAVVTGVTAVAQKTVEGAGSIAAATGFVKKDQLGKNEEGAPQEGILEDMPVDP
DNEAYEMPSEEGYQDYEPEA
SEQ ID No 19: >human MAPT ENSG00000186868.18
MAEPRQEFEVMEDHAGTYGLGDRKDQGGYTMHQDQEGDTDAGLKESPLQTPTEDGSEEPGS
ETSDAKSTPTAEAEEAGIGDTPSLEDEAAGHVTQEELRVPGRQRKAPERPLANEISAHVQPGPC
GEASGVSGPCLGEKEPEAPVPLTASLPQHRPVCPAPPPTGGPQEPSLEWGQKGGDWAEKGPAF
PKPATTAYLHTEPESGKVVQEGFLREPGPPGLSHQLMSGMPGAPLLPEGPREATRQPSGTGPE
DTEGGRHAPELLKHQLLGDLHQEGPPLKGAGGKERPGSKEEVDEDRDVDESSPQDSPPSKASP
AQDGRPPQTAAREATSIPGFPAEGAIPLPVDFLSKVSTEIPASEPDGPSVGRAKGQDAPLEFTFH
VEITPNVQKEQAHSEEHLGRAAFPGAPGEGPEARGPSLGEDTKEADLPEPSEKQPAAAPRGKP
VSRVPQLKARMVSKSKDGTGSDDKKAKTSTRSSAKTLKNRPCLSPKHPTPGSSDPLIQPSSPAV
CPEPPSSPKYVSSVTSRTGSSGAKEMKLKGADGKTKIATPRGAAPPGQKGQANATRIPAKTPP
APKTPPSSGEPPKSGDRSGYSSPGSPGTPGSRSRTPSLPTPPTREPKKVAVVRTPPKSPSSAKSRL
QTAPVPMPDLKNVKSKIGSTENLKHQPGGGKVQIINKKLDLSNVQSKCGSKDNIKHVPGGGSV
QIVYKPVDLSKVTSKCGSLGNIHHKPGGGQVEVKSEKLDFKDRVQSKIGSLDNITHVPGGGNK
KIETHKLTFRENAKAKTDHGAEIVYKSPVVSGDTSPRHLSNVSSTGSIDMVDSPQLATLADEVS
ASLAKQGL
SEQ ID No 20: >human GDNF ENSG00000168621.15
MKLWDVVAVCLVLLHTASAFPLPAGKRPPEAPAEDRSLGRRRAPFALSSDSNMPEDYPDQFD
DVMDFIQATIKRLKRSPDKQMAVLPRRERNRQAAAANPENSRGKGRRGQRGKNRGCVLTAI
HLNVTDLGLGYETKEELIFRYCSGSCDAAETTYDKILKNLSRNRRLVSDKVGQACCRPIAFDD
DLSFLDDNLVYHILRKHSAKRCGCI

### BRIEF DESCRIPTION OF THE FIGURES

FIG. 1 depicts the different neural lineage cells and their markers.

FIG. 2 depicts the research strategy to identify Genomic Safe Harbor (GSH) sites suitable for iPSC gene editing and expression in dopaminergic neurons.

FIG. 3 is an illustration of the stepwise approach to prioritize GSH sites for therapeutic gene insertion.

### DETAILED DESCRIPTION

Unless specifically defined herein, all technical and scientific terms used herein have the same meaning as commonly understood by a skilled artis an in the fields of gene therapy, biochemistry, genetics, and molecular biology.

All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the gene editing disclosed herein will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds. -in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes 1-IV (D. M. Weir and C. C. Blackwell, eds., 1986); and Manipulating the Mouse Embryo, (Cold Spring Harbor Laboratory Press, Cold Spring Harbor, N.Y., 1986).

Generally, disclosed herein is a composition comprising an engineered cell. An engineered cell can be administered to a subject in a therapeutically effective amount. Administration of an engineered cell can produce a therapeutic outcome in a subject, wherein a therapeutic outcome is modulated by the exogenous gene that was added to the cell.

### Definitions

### Amino Acid Residues

Amino acid residues in a polypeptide sequence are designated herein according to the one-letter code, in which, for example, Q means Gln or Glutamine residue, R means Arg or Arginine residue and D means Asp or Aspartic acid residue.

### Amino Acid Substitution

Amino acid substitution means the replacement of one amino acid residue with another, for instance the replacement of an Arginine residue with a Glutamine residue in a peptide sequence is an amino acid substitution.

### Cleavage

The term “cleavage” refers to the breakage of the covalent backbone of a polynucleotide. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. Double stranded DNA, RNA, or DNA RNA hybrid cleavage can result in the production of either blunt ends or staggered ends.

### DA Neuronal Cells

DA neuronal cells can be differentiated in vitro from pluripotent cells through a variety of methods known in the art, as for instance by mono-SMAD or dual-SMAD techniques as discussed herein.

### DNA Target

By “DNA target”, “DNA target sequence”, “target DNA sequence”, “nucleic acid target sequence”, “target sequence”, or “processing site” is intended a polynucleotide sequence that can be targeted and processed by a rare-cutting endonuclease. These terms refer to a specific DNA location, preferably a genomic location in a cell, but also a portion of genetic material that can exist independently to the main body of genetic material such as plasmids, episomes, virus, transposons or in organelles such as mitochondria as non-limiting example. As non-limiting examples of RNA guided target sequences, are those genome sequences that can hybridize the guide RNA which directs the RNA guided endonuclease to a desired locus.

### Dopaminergic Neurons

Dopaminergic neurons are cells that express TH, DAT, FOXA2, GIRK2, Nurr1 and LMX1B.

### Engineered DA Neuronal Cells

“Engineered DA neuronal cells” means a population of DA neuronal cells comprising an exogenous knock-in PARK2, PINK1, DJ-1, LRRK2, c-Rel, ATG7, VMAT2, GBA, SNCA, and/or GDNF gene

### Enhancing the Therapeutic Activity

By “enhancing the therapeutic activity” is meant that the DA neuronal cells, or population of cells, engineered as described herein, become more viable, or have improved engraftment, proliferation, migration, innervation and/or differentiation than non-engineered cells or population of cells.

### Endonuclease

The term “endonuclease” refers to any wild-type or variant enzyme capable of catalyzing the hydrolysis (cleavage) of bonds between nucleic acids within a DNA or RNA molecule, preferably a DNA molecule. Endonucleases do not cleave the DNA or RNA molecule irrespective of its sequence but recognize and cleave the DNA or RNA molecule at specific polynucleotide sequences, further referred to as “target sequences” or “target sites”. Endonucleases can be classified as rare-cutting endonucleases when having typically a polynucleotide recognition site greater than 10 base pairs (bp) in length, more preferably of 14-55 bp. Rare-cutting endonucleases significantly increase homologous recombination by inducing DNA double-strand breaks (DSBs) at a defined locus thereby allowing gene repair or gene insertion therapies (Pingoud, A. and G. H. Silva (2007). PreSNCAion genome surgery. Nat. Biotechnol. 25 (7): 743-4.).

### Exogenous Sequence

“Exogenous sequence” refers to any nucleotide or nucleic acid sequence that was not initially present at the selected locus. This sequence may be homologous to, or a copy of, a genomic sequence, or be a foreign sequence introduced into the cell. By opposition “endogenous sequence” means a cell genomic sequence initially present at a locus. The exogenous sequence preferably codes for a polypeptide which expression confers a therapeutic advantage over sister cells that have not integrated this exogenous sequence at the locus. An endogenous sequence that is gene edited by the insertion of a nucleotide or polynucleotide as per the disclosed methods, in order to express a different polypeptide is broadly referred to as an exogenous coding sequence

The method disclosed herein can be associated with other methods involving physical of genetic transformations, such as a viral transduction or transfection using nanoparticles, and also may be combined with other gene inactivation and/or gene insertions.

### GBA Gene

The term “GBA gene” encompasses a GBA gene and functional fragments and variants thereof.

### GDNF Gene

The term “GDNF gene” encompasses a GDNF gene and functional fragments and variants thereof.

### GABAergic Neurons

GABAergic neurons are cells that express GAT1, GABAB receptor 1, GABAB receptor 2, GAD65 and GAD67.

gRNA Molecule

A gRNA molecule is able to localize to a site comprising, consisting of, or consisting essentially of a nucleic acid sequence fully or partially complementary to a target domain such as a gene or part of a gene. In certain embodiments, a unimolecular, or chimeric, gRNA comprises, preferably from 5′ to 3′: a targeting domain complementary to a target domain in a nucleotide in a cell such as a chromosome; a first complementarity domain; a linking domain; a second complementarity domain (which is complementary to the first complementarity domain); a proximal domain; and optionally, a tail domain.

### Gene Editing System

Gene editing system or Genome editing system is a group of technologies that give scientists the ability to change an organism's DNA. These technologies allow genetic material to be added, removed, or altered at particular locations in the genome.

### Genomic Safe Harbors

Genomic safe harbors (GSHs) are sites in the genome able to accommodate the integration of new genetic material in a manner that ensures that the newly inserted genetic elements: (i) function predictably and (ii) do not cause alterations of the host genome posing a risk to the host cell or organism. Table 1 below is a list of genomic safe harbor loci that can be used as described herein. Table 1 shows genomic coordinates in GRCh38/hg38 human genome assembly.

TABLE 1

| Chromosome | Start | End |
|---|---|---|
| chr1 | 214186905 | 214187961 |
| chr1 | 91164906 | 91165855 |
| chr1 | 88180241 | 88181223 |
| chr1 | 72546731 | 72548018 |
| chr1 | 199684394 | 199685410 |
| chr1 | 104647871 | 104648861 |
| chr10 | 109456554 | 109457498 |
| chr10 | 84613037 | 84614143 |
| chr10 | 128540929 | 128541943 |
| chr10 | 128693340 | 128695044 |
| chr10 | 36778899 | 36780002 |
| chr10 | 36725218 | 36726233 |
| chr11 | 121721801 | 121724194 |
| chr11 | 42524286 | 42525334 |
| chr11 | 81647375 | 81648903 |
| chr11 | 116192498 | 116194401 |
| chr11 | 114774331 | 114775325 |
| chr11 | 128021283 | 128022329 |
| chr11 | 116224105 | 116225916 |
| chr11 | 20309899 | 20311995 |
| chr12 | 92649604 | 92650584 |
| chr12 | 94763797 | 94764936 |
| chr13 | 103882813 | 103883879 |
| chr13 | 52967785 | 52968920 |
| chr13 | 52904228 | 52906942 |
| chr13 | 59317916 | 59319213 |
| chr13 | 70309932 | 70311460 |
| chr13 | 76473185 | 76474229 |
| chr15 | 96947520 | 96948565 |
| chr16 | 66127013 | 66128412 |
| chr17 | 56715590 | 56716621 |
| chr18 | 61088416 | 61089493 |
| chr18 | 40576326 | 40578671 |
| chr18 | 27749740 | 27750778 |
| chr18 | 67199846 | 67201011 |
| chr2 | 133886374 | 133887591 |
| chr2 | 163347709 | 163348850 |
| chr2 | 147082542 | 147083491 |
| chr2 | 180125287 | 180126295 |
| chr2 | 121974721 | 121975718 |
| chr2 | 122660609 | 122661835 |
| chr2 | 75920596 | 75921844 |
| chr2 | 103401408 | 103402401 |
| chr2 | 133843863 | 133844997 |
| chr2 | 180123973 | 180124967 |
| chr2 | 160582472 | 160583418 |
| chr20 | 55667282 | 55669174 |
| chr22 | 49103355 | 49104388 |
| chr3 | 67113422 | 67114525 |
| chr3 | 74614218 | 74615242 |
| chr3 | 5426026 | 5426982 |
| chr3 | 117399281 | 117400399 |
| chr3 | 28915343 | 28917419 |
| chr3 | 117439560 | 117440548 |
| chr3 | 117237013 | 117239704 |
| chr3 | 137162914 | 137164165 |
| chr3 | 106609403 | 106610465 |
| chr3 | 67109328 | 67110455 |
| chr3 | 16048355 | 16049420 |
| chr3 | 117397641 | 117399034 |
| chr3 | 104908816 | 104909868 |
| chr3 | 104596179 | 104597355 |
| chr4 | 180058397 | 180059507 |
| chr4 | 180057145 | 180058232 |
| chr4 | 27684257 | 27685441 |
| chr4 | 116925637 | 116926672 |
| chr4 | 166165437 | 166166501 |
| chr4 | 156168620 | 156169693 |
| chr4 | 18689105 | 18690087 |
| chr4 | 85370123 | 85371213 |
| chr4 | 64537927 | 64540013 |
| chr4 | 154982074 | 154983202 |
| chr4 | 130563237 | 130564339 |
| chr5 | 71937993 | 71940439 |
| chr5 | 113806007 | 113808129 |
| chr5 | 18393819 | 18394795 |
| chr5 | 18465687 | 18466781 |
| chr5 | 123874455 | 123875520 |
| chr5 | 123875580 | 123876580 |
| chr5 | 71932489 | 71933717 |
| chr5 | 34474174 | 34475918 |
| chr5 | 18113286 | 18115121 |
| chr5 | 103808080 | 103809225 |
| chr5 | 144598204 | 144599350 |
| chr5 | 108521605 | 108522526 |
| chr5 | 113692453 | 113694380 |
| chr5 | 101678253 | 101679223 |
| chr5 | 87944237 | 87945265 |
| chr5 | 101389859 | 101390987 |
| chr5 | 113678518 | 113679693 |
| chr5 | 88089287 | 88091255 |
| chr5 | 121797421 | 121798482 |
| chr5 | 87949701 | 87952291 |
| chr5 | 103894512 | 103895553 |
| chr5 | 63401153 | 63402160 |
| chr6 | 85922657 | 85923735 |
| chr6 | 104472033 | 104473578 |
| chr6 | 104525429 | 104526360 |
| chr6 | 47097062 | 47097998 |
| chr6 | 87968250 | 87969278 |
| chr6 | 16964556 | 16965560 |
| chr6 | 137415153 | 137416177 |
| chr6 | 91081186 | 91082896 |
| chr6 | 99746005 | 99747236 |
| chr6 | 99822427 | 99823526 |
| chr6 | 140439792 | 140441984 |
| chr6 | 137313918 | 137314874 |
| chr6 | 48765024 | 48766063 |
| chr6 | 90890535 | 90891762 |
| chr7 | 152925055 | 152926297 |
| chr7 | 12111174 | 12112347 |
| chr7 | 42493270 | 42494306 |
| chr7 | 31192574 | 31193687 |
| chr7 | 22000950 | 22002150 |
| chr7 | 96785947 | 96787328 |
| chr8 | 141715497 | 141716580 |
| chr8 | 26189940 | 26191013 |
| chr8 | 131803831 | 131805003 |
| chr8 | 106856111 | 106857217 |
| chr8 | 75975412 | 75977242 |
| chr8 | 115076228 | 115077322 |
| chr8 | 131815871 | 131817817 |
| chr8 | 137492454 | 137493716 |
| chr9 | 85258346 | 85259505 |
| chr9 | 17904998 | 17907161 |
| chr9 | 78439098 | 78440151 |
| chr9 | 16132791 | 16134459 |
| chr9 | 29513212 | 29515120 |
| chr9 | 75265233 | 75266237 |
| chr9 | 7542343 | 7543943 |
| chr9 | 118169738 | 118170714 |
| chr9 | 71517517 | 71519983 |
| chr9 | 7401713 | 7402906 |
| chr9 | 26325995 | 26327156 |
| chr9 | 1453442 | 1454897 |
| chr9 | 105156511 | 105157922 |
| chr9 | 12425914 | 12426947 |
| chrX | 68894085 | 68895495 |
| chrX | 20530099 | 20531285 |
| chrX | 40996550 | 40997770 |
| chrX | 20527023 | 20528155 |
| chrX | 94058277 | 94059471 |
| chrX | 138127607 | 138128791 |

### Gene Targeting Integration

By “gene targeting integration” is meant any known site-specific methods allowing to insert, replace or correct a genomic sequence into a living cell. In some embodiments, the gene targeted integration involves homologous gene recombination at the locus of the targeted gene to result the insertion or replacement of at least one exogenous nucleotide, preferably a sequence of several nucleotides (i.e., polynucleotide), and more preferably a coding sequence.

### Glutamatergic Neurons

Glutamatergic neurons are cells that express VGLUT1, VGLUT2, NMDAR1, NMDAR2B, Glutaminase, and Glutamine synthetase.

### Identity

“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.

### Immature Neurons

Immature neurons are cells that express Doublecortin, NeuroD1, TBR1, Beta III tubulin, and Stathmin 1. Immature neurons can differentiate into mature neurons.

### Improving Therapeutic Potential

By “improving therapeutic potential” is meant that the engineered DA neuronal cells gain at least one advantageous property for their use in cell therapy by comparison to their sister non-engineered DA neuronal cells. The therapeutic properties sought may be any measurable one as referred to in the relevant scientific literature and include but are not limited to viability, engraftment, proliferation, migration, innervation and/or differentiation of administered engineered DA neuronal cells.

Improved therapeutic potential can be more particularly reflected by a resistance of the DA neuronal cells to a drug, an increase in their persistence in-vitro or in vivo, or a safer/more convenient handling during manufacturing of therapeutic compositions and treatments.

In general, the molecule improving the therapeutic potential is a polypeptide, but it can also be a nucleic acid able to direct or repress expression of other genes, such as interference RNAs or guide-RNAs. The polypeptides may act directly or indirectly, such as signal transducers or transcriptional regulators.

### Locus

As used herein, the term “locus” is the specific physical location of a DNA sequence (e.g., of a gene) into a genome. The term “locus” can refer to the specific physical location of a rare-cutting endonuclease target sequence on a chromosome or on an infection agent's genome sequence. Such a locus can comprise a target sequence that is recognized and/or cleaved by a sequence-specific endonuclease. It is understood that the locus of interest can not only qualify a nucleic acid sequence that exists in the main body of genetic material (i.e., in a chromosome) of a cell but also a portion of genetic material that can exist independently to the main body of genetic material such as plasmids, episomes, virus, transposons or in organelles such as mitochondria as non-limiting examples.

### Mature Neurons

Mature neurons are cells that express NeuN, MAP2, 160 kDa Neurofilament Medium, Neurofilament Heavy, Synaptophysin and PSD95. Mature neurons can differentiate into Neurons.

### Modified DA Neuronal Cell

By “Modified DA neuronal cell” or “engineered DA neuronal cell” is intended a DA neuronal cell that either itself has been genetically modified or is derived from a genetically modified pluripotent cell.

### Modified Pluripotent Cell

By “Modified pluripotent cell” or “engineered pluripotent cell” is intended a pluripotent cell that has been genetically modified.

### Mutation

By “mutation” is intended the substitution, deletion, insertion of up to one, two, three, four, five, six, seven, eight, nine, ten, eleven, twelve, thirteen, fourteen, fifteen, twenty, twenty-five, thirty, forty, fifty, or more nucleotides/amino acids in a polynucleotide (cDNA, gene) or a polypeptide sequence. The mutation can affect the coding sequence of a gene or its regulatory sequence. It may also affect the structure of the genomic sequence or the structure/stability of the encoded mRNA.

### Neurons

Glutamatergic neurons, GABAergic neurons, Dopaminergic neurons, Serotonergic neurons, Cholinergic neurons are collectively referred to as “Neurons.”

### Nucleotides

Nucleotides are designated as follows: one-letter code is used for designating the base of a nucleoside: a is adenine, tis thymine, c is cytosine, and g is guanine. For the degenerated nucleotides, r represents g or a (purine nucleotides), k represents g or t, s represents g or c, w represents a ort, m represents a or c, y represents t or c (pyrimidine nucleotides), d represents g, a ort, v represents g, a or c, b represents g, t or c, h represents a, t or c, and n represents g, a, t or c.

### Nucleic Acid

As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally occurring nucleotides (such as DNA and RNA), or analogs of naturally occurring nucleotides (e.g., enantiomeric forms of naturally occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.

### Nuclease Reagent

By “nuclease reagent” is meant a nucleic acid molecule that contributes to a nuclease catalytic reaction in the target cell, preferably an endonuclease reaction, by itself or as a subunit of a complex such as a guide RNA/Cas protein, preferably leading to the cleavage of a nucleic acid sequence target.

The nuclease reagents used herein are generally “sequence-specific reagents”, meaning that they can induce DNA cleavage in the cells at predetermined loci, referred to as “targeted gene”. The nucleic acid sequence which is recognized by the sequence specific reagents is referred to as the “target sequence”. The target sequence is usually selected to be rare or unique in the cell's genome, and more extensively in the human genome, as can be determined using software and data available from human genome databases, such as http://www.ensembl.org/index.html.

### Neuronal Mature Cell Type

Neuronal mature cell type refers to immature neurons, mature neurons or neurons collectively.

### Patient

The term “subject” or “patient” as used herein includes all members of the animal kingdom including non-human primates and humans.

Promoters for GDNF knock-in include SEQ ID NOS 21-30 (these sequence listings are a “synthetic construct”)

Monoamine Oxidase B (MAOB) SEQ ID NO 21:
ACAGAAGCACTTCACCTGTTTATTTCTTGAAAAGCCATTCTATGTTGCTGCTTCCCA
CTTTTAGTTCTCAGAGGTGGCTTTCCTACTGTCCAGAAGACATAAACTTCTGTTTTC
CCTGGAGTCACCTTAGCTTAAACTCCTATAATCTTGTTGAATCATCCCTGTGTTTGG
CTAAACCAGGCTCCTCATACCCACGTTCCAGGGTCACACAGGTAACTTGGCTTGCT
GCTTCTGGGGGCTCTGAATCCCACAATGCCGATCCACCACCTCTTCCCTGCTGCCAC
TAGTCTCCTGGGGTCTAGCCTCAACAGCTTCCACCAAGGACACACTGCATTGCTTA
AGAGTGTATTTAAGGTGCGGCATTTGTGATGCTAGGGAGGAGCCTTCCTGACTTAA
TCACGTAGGGGGAGTGGGGAAAGAAGTGGGTATTTGGAAGGAGGGTGAGGTGGGC
TGGGAATGCCAAGAAATGGTAGTACTGTGTGGTGAATCAAAGTGGGTCTTGGAATC
TTCCTAGCTTTTCTTTTCTTTTCTTTTCTTTTTTTTTCTTTCTGACTTTTTATCTTATGT
TGTCACTCACCATGTTTTGTTTTCCTCATCTGCAAAATGGATTGTTGTAAGCTTGAA
CAGGAATCATGCATAAAGTACTGAGCACAGTGCCTGGTACCTTGAAATCACTCAAT
GGAAGTTTCTTATTATTTGCCCTACACCCAAGGAGTAATTGGGGCCCTGAAGGAAG
ACACCCCAATTTGCCGTCCTCCCAGCCGCCAAGCCTGATAGTGCACTAGGGAGGCT
GATGGGAAAATACCGTGCAGAAGGCTGAGGAGTGGGGATTCTGGGGCGGGACTCT
TTGAAGTCCTAGGTGACCTCTCCGCCCAGGCACCCGCCCTCCCCGGGCCTGGGCTG
CAGAGCTGCGGGCGGGGGCGGTGGCCCTGCGCTGCACCCGGCCTGCCTGCGCGTA
GGCGGGCGGGCGGGGCTGCGCGTCCGGGCTCCCGGGGCTGGTAATATAGCGGCTC
GCCGAGGCGCTGGTGCACGGGGGCAGCGCGCAGCAGGCCGGCGGGCAGGCGGGC
GGGCTGGCTGGCAGGCAGGACTGGGATCGAGGCCCAGAAAACGGAGCAGCGGGC
ACCAGGGAGGCCTGGAACGGGGCGAGCGCC
GTP cyclohydrolase 1 (GCH1)
SEQ ID NO: 22
CATTATGAGATAATCCCTCAAGTGAGGAAAAAGGTCCATTTATTAATCTCAAAGAA
AACAGTTACAGCAGATGTCACTGGTTAAGAGTTCAGTTGGTGAATAGCATTTCACA
ATTTGTACCAACATCTGGGGAAAGACGCTTTGCATGGAACTGTAAAACAATTGAGC
ACCAAATCTGCACAACTGCGTTTCTAGAAAATGCGATGGGTTTTATAGAGATGAGG
TCTTGCTATGTTTTCCAGGCTGGTCTCGAACTCTTGGCCTCAAGCGATCCTCCCGCC
TCGGTCTCCCCAAGCGCCGGGAGTACAGGCGTGAGCCACCGACGGAAATGGATTTT
AAGTGAAAGTCCTATCTTCGTTTGCAAATCAATTTTTCCAGGATCAAAGTTCTAGCA
ACCATAAATCCTATCCTTCTAGACATAGGGACCCACATAAGGGCCACTTGATGTGA
CACTTGCCCCGACAGCGGTGCCACCTGGCCCCTAGGTGGCAGGTGCGTTTTTAACT
AAACCCAAGAGTCTGAAGTCACATTATTCTCTTGCCTTGAAAGCTTAAGAGTTGGT
TCCTAATCGGTGGCTTAACTCGGTCTCTCTCCAGGCAAGGCCACCTGTTTGCTGATC
TTTCACAGGGCGAGGGGACCGGAGCCCTTCAATGCAGCGCCCTCTTGGCCTGAAGA
GGGGGCGACTGGGCGCCCAGACCGCGTCTTTCTCACTGAGATCCCAGCTCCTGGAC
GACTGCCTCTTTTCGGGTTCGGCTCATTCCGCAATAAGTGGAGGGGTCCCGCCTGA
TCCTAGGGGCCGGCGCCCTTTTCCTTCCCTCCCTGCGCTTGCGAACCCCTCCGGGCG
TCTCCGGAGCCGCGACCCCTGCTGGGGACCCCGGGGGGGGAGGCCGAGGGGCGC
GGCGAGGGCCGTGACGCGAGGCGGGGCCGGCCAATGGGAGCGCTCCGCGGCGCAG
GCTGAGCCGCCGGGCCATAAAAAGGAGGCGCGGCCGGGCTTTCCAGCCTGTGGCC
GCTCCCGGCTCGGAGTGTGATCTAAGCAGGTTGCGTACCTTCCTCAGGTGACTCCG
GCCACAGCCCATTGTCCGCGGCCACCGGCGGAGTTTAGCCGCAGACCTCGAAGCGC
CCCGGGGTCCTTCCCGAACGGCAGCGGCTGCGGCGGGTCC
Nuclear Receptor Subfamily 4 Group A Member 2 (NR4A2)
SEQ ID NO: 23
CCACTTCCTGGTTTTATTTTTTTTCTCGCGCCCGCTAGGGCATCGCCGAGCAGCGGC
GGCGGCGGCAGCAGCAGCGGCAGCCGCAACATCTGGGGGAAACTTAAGGTGGTCA
CGTAGGTTTCCCCGAGGCTGCGGCGCAAAAGAGCCCGCGGCTTCCTGGGGCAGCC
GCAGGGCATCTGATGGCAACGCCTCCCCAGCCGCCGGCGAGCTGGGGGCAGCAGG
GAGTAGGCAAAGGCGGGGCTGCAGATTAGGGTTGAGCAGCCCGGGATCCCGAATA
GTTCCACGGAGCCATCGGCCGGTACTGAGAACACGAGTAGGGGGGCAAGTTGCAA
AAGGGTCCCGGCTGCCTGGAAACGAGCCACACCGAGAAAGGAGGGGCGGCTGGG
ACGCTCTGCGCGAGTAGAAAAGCGGGGGCGGCTCCGAACTCTCCCTCCCAAGGCC
CAGCCGCTGGAAGCCCCGCGGGAGAAGCCCTAGGAAGCCCTCCCAGCTCCGGCCG
GGAGACGGGGAGGGGGCTTGCCCTCCGGAGCTGCGGCGTTTCCTTTGCTAGAAAGC
TCCCTGCGCTCTCGGCGTGGCTAGGGGAAATGACTTGCAAAGCAGAAGCGGCGGC
GACTTGGACGGCCGCGGGGGTGGGACACGCTCCCAGAGCCAGCTCGGGCCCCTCT
CCTGGCTGGGCTGTAGGCTCACACCTTACGCTTTGCGGAGACGCTGCCCCCAATCC
CCACCCGCTTCGCTTTTCTTGTCCAGTCTTTCTGGGCTCTAAGATTGAGGCTGTGAT
AGACCTTGTGTTTCAAACGAAGATCGATTTATTCCCTTAAAACTCGTGGAAGGGGT
ACGAGTTGGTGGGCACAGAGGAGTATCGAAAGTATTAGAGTTTGCAGAAGGTGGG
AATCAGAGTACCTTTCTGCAGAAGGCGCACCTGTTGCCTGCTGCTCGGCTGCATTG
TCTGCGAAAGGAAAAGGGGCGACGCTTCACTCTGACTTTTGGGGTTTCTAAAGAGC
TGGGTCACGGTCACTGTCTACCCTCTCCCCTTCCTCTCCCCCCCCCCCGCAAGCACC
ACGTCCTCCATCGAACGTGGGCACTGCATGGAAATAAGGAAACATAGAAAAATAA
GCCCTACCCCCACTCCCATTCCCTTTCAGATGGGAGTGTGGGGGGTGGGGGTGGGG
TAGAGAGAGTGAGAGAGAGAGATAATTAGAATGAATATATGCCAGAAGAGGAGG
AGGCCTGGGACAGGAAAAGGGAGTAAAAGGGGATGAACCGGGTAGGGAAATCCC
ACCCGAACTGCGTGAGCCCTCTGAGCGTCTCGTGTCATGGGACATCTGTACGCTCT
TCCGCTAAGGGGGTGACGAAGGTGGGTGGGAGAGGTTAGGGCGCTGCAAGGCACA
TCCCATTCCGTGCGGCTCCAGGTGCGTACTACCTGGCCACGCCGGCCTTGCCTTCCG
CCGGTGCTTTTGTTGCACCCTCCCCACATGTGAGCGGCCGAGCCGCTGCGCGGGCG
CAGGGGCTTCGGGGAAAGTGAAGTGTCGCGACGCTGCGGGCTGCGCAGACCTGGG
AGAGGTCACACCTCTTTCGGAAAAAAAAAAAGAAAAGAAAAAAAACACCAAAAA
CCACCCAAGCTGGCTACCAAGGTGAACGCAGAGCGGTTCCCACCTTAAAATCGGCC
CTGCTCGTGACGTCAGGTCGGAAATATACCAAAGCGAGCGCGGGCCAGGAGTCCA
GGGAGCGCGGCAGCGCGGCGATTGGGCGGCGGGCCGCTGACGCGCGCTGACGCGC
GGAGACTTTAGGTGCATGTTGGCAGCGGCAGCGCAAGCCACATAAACAAAGGCAC
ATTGGCGGCCAGGGCCAGTCCGCCCGGCGGCTCGCGCACGGCTCCGCGGTCCCTTT
TGCCTGTCCAGCCGGCCGCCTGTCCCTGCTCCCTCCCTCCGTGAGGTGTCCGGGTTC
CCTTCGCCCAGCTCTCCCACCCCTACCCGACCCCGGCGCCCGGGCTCCCAGAGGGA
ACTGCACTT
Mouse Tyrosine Hydroxylase (TH)
SEQ ID NO: 24
CTGCTAGGGGCTGCTTCCCAGCTACTCCTCTTGGCTCCGTGGCTTGCCTTCCAGCCT
GTGTGCTGTCTGGAGAGCCTTTAAAGCCTCACTTCCACCAACTAGAAGTCTCTCCCC
AACCCTGCCCTGACCTCAAGTGCACCTCTTCAAAGTCAGGTTTAGCAGCTGCAGCT
GGGGGCCCTGAATCCCACCCCTGCTGTCTTCCTTGAAGACAGAAGTGTTGGGAGCT
GAGGATCTGGGCTAGAGACTGGCTGTATGATCCAGAGAAGTAGTGTGCTTCTGGGC
CTCAGATTTCCCTTCTGTAGAACAGGTTTGTCTGAAATGGAGAGGTTGGTGCTCCTC
TGCAGGGCCTAGTGGGAGTCACCATGAGTGGTTAAAAGATCCAGCTTGTCTTTTGG
TGAGCTTTGAGAGGAGGTAACAGGGCTGAGTTCTGGAAGCCTGACCAAGGGCAGA
CTTAAGGGGCCTCTTGGAGTTGTTCTCATCAAATGGGGATGGGACACAGCTAAAGT
GCCCAGGGCTTCTCTGTGCCCACAGATGCTTTAGATCTTGGCACAGTGTGGTCTACC
AGCTGTCTCTCTCTGTGTATATATATGTATTTCATAGACAGTGTACAGTGGCCTGGT
TTGTGCTATCAGGCTGGATATGGACAGAGGCAAGAGTTTGTGGCAGCAGTTATCTC
CCAAGAGAGTCCAAAGACATCATGTTTTCAAGTTTAGGCCAGGTGCTACTTGAGAG
AGCTCAGACACAGACAAAGGTCTGGAGAGCACATGTCCTCCACCCCCACCTAGCTT
CTGTTGCAAGCACCTCCAGCCGAGACAAGAGAACGAATTAAAAAGCAATATTTGT
GTCAGTGTAAGACATTTGCCGAAAGGTTAAATCCACATTCGTGTTGCTGCAGAGCA
GCCCCCTATGCAGGATTTGTTAGATACAGCTCCGTCCTACCCTGTGCCAGCTGAGC
AAACGCCAGGCTGGGTGGGGTGGAACCCAGCCTGGGTTTGCCTCACCCTGCAATCC
CCCCAGCACCCTCTAAAGGAGGACCCTGTGGTGGGCATGCAGACCTAGGGACTGG
GCATAGATAACCTTTGGGTTTGGGCAACAGCCCCCACTCCTCAGGATTGAAGGCTA
AGGTGCAGCCAGCTCTGCCTTCATGGTGGGAATGTCTCCACGTGACCCCTTTCTGG
GCTGTGGAGAACACTCAGAGAAGAGTCCTGGGATGCCAGGCAGGCCAGGGATGTG
CTGGGCATGTTGAGACAGGAGTGGGCTAAGCCAGCAGAGTTGCTGACCCAGGAAG
AGTTCAGAAAGGGGCATGGAACATGGGGAGGGGTCCATAGTGAGAGAGAGCAGG
CAGTGCAGAGTAAATAGTCCCTGAGCTGGGGGTTATGGGATTTGCAGGAGCTTGCT
CAGAGAAGGCAGAGGAGAGATGCTGCGCCAAGCTGGGTATCACAGAGCCTCAGAC
TCCTGGAACAGGAACTGTGGGGGTCAGGTCAGCAGGGGAGGTTAGGGAGTGTTCC
CTTTGTACTGACTTAGCATTTATCCTGCTTCTAGGGGGGAAGGGGGGCCAGTGGGG
GATGCACAGCAAGGCAGTGATGTGGCAGGCAGCCTGCGGGAGCTCCTGGTTCCTG
GTGTGAAAAAGCTGGGAAGGAAGAGGGCTGGGTCTGGTAAGTACAGCAGGCAGTT
GGCTCCTGAGAGTCCAAGCCCTGTCTAGAGGGTGGAGTGAGATTTCAGAGGGAGA
GCTAAACGGGGGGGGGCTGGGGAGTCCAGGCTTCTGGCTCCTGCTAATACTCAGT
GTGCTGGGTCCTCAGAACCTCAGGGTGGCCATTTTCAGGGTGAGAGCTCTGTCCTT
TGGCACTTCTGCAGACTCCAGTATCCAGAGGAATAAAGATGGTACTCTTCCTCAGT
TCCCTTAGTGAGAGGACACCTTTCTCTGAAGGGCTTGGGCAGTTGTCCTGAACCAT
TGCCTGAAGGAAGGACTTGACTCCAGGGACATAGAATGGGCTCAGCATAAGTCCC
CTGTAGTAGAGAAAGGTCCCCTCTCTGGTCTCCTTAGAGATCCTGTTTCCTTGGCTG
AGGAAGCTAGGGTGGATCTTTGTGTAAGTGGGTGTGGATGCTCACTGGAAATCAAA
AGGCCCCTTGGTGTTAGACCTTGGGGTGCCATGGGAGAGTTGATCACTGAGTGCGC
CCTTACATGGGGGCCAGCTGAGAATGGGGCTGCCTCTAGCTCGAGACCATGATGCA
GGGAGTGAGTGGGGGAGTTCAGGATACTCTTAACTAAAGCAGAGGTCTGTCCCCCC
AGGGAGGGGAGGTCAGAAGACCCTAGGGAGATGCCAAAGGCTAGGGTTGGCACC
ATGTTGCAGGCTGTGTCTTCAAGGAGATGATAATCAGAGGAATCGAACCTGCAAA
AGTGGGCCAGTCTTAGATACACTATAGAGGAATAATCTTCTGAAACATTCTGTGTC
TCATAGGACCTGCCTGAGGACCCAGCCCCAGTGCCAGCACATACACTGGGGCAGT
GAGTAGATAGTATACTTTGTTACATGGGCTGGGGGGACATGGCCTGTGCCCTGGAG
GGGACTTGAAGACATCCAAAAAGCTAGTGAGAGGGCTCCTAGATTTATTTGTCTCC
AAGGGCTATATATAGCCTTCCTAACATGAACCCTTGGGTAATCCAGCATGGGCGCT
CCCATATGCCCTGGTTTGATTAGAGAGCTCTAGATGTCTCCTGTCCCAGAACACCA
GCCAGCCCCTGTCTTCATGTCGTGTCTAGGGCGGAGGGTGATTCAGAGGCAGGTGC
CTGCGACAGTGGATGCAATTAGATCTAATGGGACGGAGGCCTCTCTCGTCCGTCGC
CCTCGCTCTGTGCCCACCCCCGCCTCCCTCAGGCACAGCAGGCGTGGAGAGGATGC
GCAGGAGGTAGGAGGTGGGGGACCCAGAGGGGCTTTGACGTCAGCCTGGCCTTTA
AGAGGCCGCCTGCCTGGCAAGGGCCGTGGAGACAGAACTCGGGACCACCAGCTTG
CACT
Solute carrier family 6 member 3 (SLC6A3)
SEQ ID NO: 25
TTGAGGAATTCTGTGCTTCTTCCCTCTTGGTCTTCAGCCAATATGAAAAATAAACAC
ACAACAGGAACGCAACAATTCAGCAGCCAGGGCTAGGGACATACCTGGTGCGGAG
GGCAGAGCCCCGAGGCATTCACGGGCATTCCTCTGGGAAGGGATGGGTGCGTTCTC
AGCGCCTGAGATGGTACAGCTGGGGCACGGGAGCTGGGAAGTGCCAGCCCTCACC
ACAGGGCTGGGGCACTGGCCCATGGAGGCCTCAAGACAGACACTCTGGTTTGCTCC
TCCTTCCCCTGCACCCCTCCCCACTCCTATCAAAGTGCAAGGCTGTGTGAGCTCTAG
CGTGGCTTTCTACTAACAATGCATGGATATCACTTCCACCTTCCCCCACACTTCCAG
AGAGCAATTTTACAATCTTCTAAGAGCATTCAATAATGTCTTCATGACTGTTCATCT
GTATTCTTTTTTCTTTTTTTTTTCTTTTTTAACATGAGGTCTCGCTCTGTCACCCAGG
ATGGAATACAGTTGTGATTACAACTCACTGCCTCAACCTCCTAGGTTCAAGTGATC
CTTGCACCTCAGCCTCCTGAGCAGCTGAGACTACAGGTGTGCACCACAGCACCGGC
TAAGTTATTTTTGTGTGTGTTTTTTTGTTGTTGTTGTTGTTTTTTTGTAGAGACAGGG
TCTGACTATTGTTGCCAAGGCTGGTTTTGAACTCCTGGTCTCAAGCGATCCTCCTGC
CTCAGCCTCCCAAAGTGCTGAGATGACAGGCATGAGCCACCAAGCCTGGGCTTGTA
TTGTTTATACTCAGTAATAAAGAGCAAAATTTATATAAGCAAAATCAGCCACTGTA
AAGAGAGTTTAGACATAACCATCACAACTGCTTCCTCAAAAAGTGTCTGTACAAGC
CACATCCACCTAGGGCAGGTGGCACCACCCCGCGTGAGAGAGCTGGGCGGAGGAT
GGACAGGGCTTCATCGCGGGAACTAGCTCCTGGGCCAGCAGGGTAACCCGGGAAA
TCCTGTCCAAACGGCTACATAAACCCCCCTGCAACGCTGAGCAGGAGAGCAGGAG
CGTCTACACCGCCCCAGCCAGGGCGACTTCTGTCATTTGGATCCAGCAGCAGCGTG
TTTCAGTCGAGCGCACGCACACACAGACACACAGACACACACAGACTCACAAACA
CACATACACACTCACACACACACAGGCGAGCACATACACTTACACACATACACACT
CACACTCACACAGGTGCGCACACACACACAACGACTGAAGCAGGTCGCAGGTGGA
GGCTCTAACAGGCAACTTTCCCTGCATCCAAGTTTGCACAAAACCTCACCCATGAG
CTATCACGAGCTATGCCACGCACCCGGTGACAACCTCAACCGGCACGACCCCTCCG
GTGGGTAAAACACCCGACGAAGGGGTTCCGCGGCGCGAGCAGGAGGCCGCGTTGG
GAGAGGGCGTCGGGTGCGGAGCTCGCGAGTCTCCGGCAAGCCGCCGCCGCCCTGG
GGCCACCTGACCCCGCTCCTGAACGCGGGGCCTCAGAGGCGAGTTTTGGGTCTACA
CAGCAAAATGTGGCCCGTTAGAAAGCGTCCTTCCTCACCGCGCCCAGAACAAGAA
GCGGCGCTGCCTGGCGAGTCCTCGCCCGGCCTCCCCGCCCTGCCCAGAGCACCAGC
GCGCAGCCCGCGCCCGCCACCACCCCCAACACAGACAAAGCCCCCGCGAGATGGA
GCGGCGGTGTACAAAACCCACTCCGATCGGATCTGGGATGCGCCGCACCCCTAGTA
GGGTTAAGTTCCACGAGAAGAAACCAGGACCCCCGAGTCACAGCCATAGACACCC
ACGCGTCCCCTAAGCCGGTGACTGCACCAGGTGCCCACCCGGTTCTGAGCCTGGAG
CGGGGCCAGGGGCAGCCCTGCACCCTCCTCGCGCCTCGGACGGCCTCAGGGTCCCA
TGTGGCGCTATCGGGGGGCGCTCGGCACCCGCGACTGTTGGCGACTTTGGAGACGG
CGCGGTCTGCGGGACGCGGGGACCCCAGACTGTGCCCGGTCCCCGGCCCCCGCCCC
TGCGCCCCACTTCGGGGTCTGCGGAAGGAGCGGGGCGCCCCGATGCCGCGAGCGA
CGCTGGCCTCACCTGGCCGCCCGGGCCTGGGCTTTGCACGCGAGTCCTGGCGCCGA
GAGCGTTCCGCGAAGCCTCCCCTCCCGCTCCGCAGCGCTGGGCGGTCTCAGCCTCG
GCCTCGGGCTCTTATCCAGTAGACAGGGTCCGCCCCGCCCCGCCCTCCCGCCGACG
GGGTCGCCCTCGCGTCCTGGCTCCTCCTCCTCACCTCCCCGGGCCGCGCCCCCGCCC
CCGCCTCCCGGACGCAGATGGCTGGGAGCAAGCGGCGGCCACGGCTCCACTGTCCT
GCGCGTCCCGGAGCCTCGGACACGGGGTGCGGGGGCCTGGGGGTCCGAGACGTCG
CGGATCTAGACGGGCGCCTTCCACGCGGGAACTGTCTGGGCCGCCCTGCGCCCTCG
CCCGCACCCCTGGGGGCTGCCCCGGGCACCCGCTATCTTCGGACGCCCAGGGCTGG
CGGCTCCCAGCTGGACGGGGAGGGAGCCGAGCGGCTGCGCGCGGGGAACTGCTGG
GACCCCGTCGGGCCGACCCTTCCCTCCCTCCCCATCAGGGGCCCCTACGGTGGCCC
CGCGCTCTGGGGACCACGTCCCTCCCACCCCGCCGCTCCACGCTCTGACCAGCGGG
CTCGGGACCCCGCCGAGGACTCTGCACGCTTCCCGGCGTGCGGGCTGCGGAGACGC
GGGTCCTTCCGTGGCCTTGGGGTCTCCGCGACCTCGAGGCGACCGAGGTCCCCTAG
GCCTAGTCCCCGCCTTGCCGGCATCTAGTGCGCCTGCATCGGAACCGAGGGGCCCG
CCTCGCACTCGCCTAAGAAAACCATTTCCCGGGACGGGCCTGGAAAGCCCTGGCGG
TTGGTATCCGGCTCCAGGGCCGAATGGACTCCGAGGCTTTCCTTCCGAGATGCCCG
GGCGGGATTTCTTCCATTCAGCGCGCGGAGGAATGGAGCCCCCAGGCCGCCAAGG
CCCAGGATGTCCAGGTCCTTAGAAGGCACCGAGGTGAGTGGCCGCGCCCAGCTCTG
TGCGTCGCCCCTAGGGTCGCCCGAACCCCGGCGTTCCTCCCCGTGTACCCTCGATG
CTCCCGTGAGCACCCTGCCCTGGGCGTCCAGGCGAGCCCTGGGGCTCTCCAGCCAT
TCCAGCCTGCAGCCCGCGCTCAGCAGTGAGGGAGGCAGGGACCCTTGGTGCCTGA
CTCCGCAGTACCCGCCCGCAGCGCGGTGCCCCACCCCCTGCGCCCGCCCCGCACTC
CCCGCACACCACCACTCCCCTCCCCGCCCCCCGCTCCCCCGCTCCCCGGCTCCGGG
GCTGGCACCCAGGCAGAAGGCACTCAGGGCTGTGACTTCACGCTGAGTTGGAATC
AAGTGGGAGCCTAGTGACCCCCTGGGGCTGCCATGACAAAAAACTCCACAGCCTG
GGGGCTCACACAGAAGCCTTCAGTTCTAACGGTTCCGGTGTCCCGAGGCCAAGGCC
TGGCCGACTCTGTGTCTGGTGAGGGCCGCTTCGGGGTTGCAGACAGTTCTCTCCCT
GTATCTCCCCACGCTAGGGGCTGGGGAGAGCTCTCCGGGGTCTGTTTTATGAGGGC
ACTGGTCCCATTCCTCAGGGTGGAGCCCTCCTGGCCTGACCACCTGAAGGCCTCCT
GACACCACCTCCCATCAGGGGTGGGACTTCATCGCCAGGGAGGTGAGGGGACTTC
AGGAGAGGGGGATGTGCCACCTCGAAGCTGTAGTGTGTCAGTGGCTGCCACAGAA
GGGATCTCTCTACCCCTATTATCTCCATGGCACAGGTTTCAATAAAAAGGAGTGAG
CCAGGGCTTGGCCCCAGAGCAGCAGGGTTGAGGAAACAGTCTCCCAAGGAAAAGA
CTTTGCAGGTGACTAAGGGAGGCTGTTAAATAAAACACACAGAGAGAATGGATGT
TCAGGTATATTAAAAATACACAGGCCAGGAGTGAGGGCTCACGCTTGTAATCCCAG
CACTCTGGGAGGCCGAGGAAGGAGGATCTCTTGAGGCCAGGAGTTTGAGACCAGC
CTGGGCCACATGGCAAAACCCTGACTTTGAAAAAAAAAATTAGCCAGGCATGGTG
GCCCACACCTGTAGTCCCAGCTACTGGGGAGGCAGAGGTGGGAGAATAGCTTGAA
GCTGGGAGGTGGAGGTTGTAGTGAGCTGAGATTGCGCCATGGGCCACAGGATGAG
ACCCTGTCTCGAAACAAAACAACACACAGGCAAACAGCGTTTGGGAAGGGATTGC
AAGCGCTGCAGGAGCGCAGAATTGTGACCCAAAGCTAACAGGGTACAGGCAGGAA
GGTGCAGGGACTAGAGGTCACACAGATCCAGGCAGCCACATCCTTACTGACCAGT
AGCACACAGAGAATGCACCTGCCACACTCAGGAGGGGCCACTGAGTGATCACACG
CATGTAACCTGCACTCTGCCAAGACAAAGAGCATTCCTCCACCCCTGCCTCAACCC
CGACTTCCAACCACATGAATTAGGATAAAGGAGCTTTGGGTGCTCTAAGAACAAAC
TAACTGCGGGAGTTGGGCTCATGGATGACACAGAGAAAGCGCAGCTGCCAGACCC
ATGGGTCTCCATCCTCCCACAACAGGAATGCTCCGTGAACTGTGCAGGTAGAAGCA
GTGTGGTTATGAGCTGATCAGACCTGATCAAACGGCCGACCTTTGCATGCTTGGAA
CTTTGCCCTAAATGCCAAGTCGGCTTTATTATCATTGTGGTTGCTGCTATGGCTGCT
GTGGGCTCAGGGCGTTGGGTGCAGGCAGGTGGAGCCCCCCTGCCTCTGCACTTCCT
GCCCATGCTCGGCACCTCTCCCCACTGGCTGGGACCGCAGCTGCAGAGCCCACAAG
GAGCCCCTGGCCACTGACTGCTGTCTCCAAAGCAACCTTGCCCCACGGGTCCCCCC
CTCCTTGGGTGCTGCCTCCTGGGAGGCCCTCAGTGCTGGATGTTGCGCCGTGTCTCG
CCTGATTTGGAAGACAGCGTGGATGCTCCCCCCATGAGTGCTGTATCTGAGAGGGG
ACAAGGACAGTGGCAGTGAGATGACAGTCATTTACCTGCCTGCGTATTTACTATAT
TGAGGGTGTCCTGTGTGCTTTCAGAGGAAGTCCCAGGGAGCCCAGCTCAGACTTTA
AAGACCCTGACCTCTCACTGGTGCTGGGAAGGCCCTGGGGGTAGGGGGCTTGGGG
AGGGCAAGGGTGAGGGGACCAGTAGGCAGGAGTGGCTTTTGTCCAGGGGTGTGGG
AGGTCAGATCCCAGACCTTGTCTGTCCAGGTTCTGAGAATGATGGAAGGCGCTGGC
TCCAGGCAGGAGGCCCTGCTCACTGCTGCACCCCTGTGCCCTCCCCGGGGGCCACC
TCAGGAGGAAGATAGTTTCCTCTCGCATGGCAGGGACAGCAGTACACTTCCAATGT
CTTCTCTCAGAGCTGTATCATTTTCCGGCTCACTGCTGGTTTTAGTCACAGACATCT
TGATGGTCTTGCCACTGCACAGCATGTCACAGTGGTCTGCCACACTGATGACATCA
GGCTGAATCAACCTGGGGAGCAAAAAATGAAAAATGCCAAGATGCCTTGGTAAGA
CACGTGCTTGCTAAAAATTGAAAGAGAAAATAACACATTTTCAAAACCTGCCACCT
TGGTGAACTTTCTGTGGGTCCATTGGTCTGATTGGTCAAAATATATATCCAAAATG
AAAGAAAACACGCTGTCTCTGGCAAGCAAGACTACCACTAAGAAAGAAGTATCA
Synapsin 1 (SYN1)
SEQ ID NO: 26
CTGCAGAGGGCCCTGCGTATGAGTGCAAGTGGGTTTTAGGACCAGGATGAGGCGG
GGTGGGGGTGCCTACCTGACGACCGACCCCGACCCACTGGACAAGCACCCAACCC
CCATTCCCCAAATTGCGCATCCCCTATCAGAGAGGGGGAGGGGAAACAGGATGCG
GCGAGGCGCGTGCGCACTGCCAGCTTCAGCACCGCGGACAGTGCCTTCGCCCCCGC
CTGGCGGCGCGCGCCACCGCCGCCTCAGCACTGAAGGCGCGCTGACGTCACTCGCC
GGTCCCCCGCAAACTCCCCTTCCCGGCCACCTTGGTCGCGTCCGCGCCGCCGCCGG
CCCAGCCGGACCGCACCACGCGAGGCGCGAGATAGGGGGGCACGGGCGCGACCAT
CTGCGCTGCGGCGCCGGCGACTCAGCGCTGCCTCAGTCTGCGGTGGGCAGCGGAG
GAGTCGTGTCGTGCCTGAGAGCGCAG
Mouse alpha-calcium-calmodulin dependent kinase II (Camk2a)
SEQ ID NO: 27
CCCTTAATTAACATTATGGCCTTAGGTCACTTCATCTCCATGGGGTTCTTCTTCTGA
TTTTCTAGAAAATGAGATGGGGGTGCAGAGAGCTTCCTCAGTGACCTGCCCAGGGT
CACATCAGAAATGTCAGAGCTAGAACTTGAACTCAGATTACTAATCTTAAATTCCA
TGCCTTGGGGGCATGCAAGTACGATATACAGAAGGAGTGAACTCATTAGGGCAGA
TGACCAATGAGTTTAGGAAAGAAGAGTCCAGGGCAGGGTACATCTACACCACCCG
CCCAGCCCTGGGTGAGTCCAGCCACGTTCACCTCATTATAGTTGCCTCTCTCCAGTC
CTACCTTGACGGGAAGCACAAGCAGAAACTGGGACAGGAGCCCCAGGAGACCAAA
TCTTCATGGTCCCTCTGGGAGGATGGGTGGGGAGAGCTGTGGCAGAGGCCTCAGG
AGGGGCCCTGCTGCTCAGTGGTGACAGATAGGGGTGAGAAAGCAGACAGAGTCAT
TCCGTCAGCATTCTGGGTCTGTTTGGTACTTCTTCTCACGCTAAGGTGGCGGTGTGA
TATGCACAATGGCTAAAAAGCAGGGAGAGCTGGAAAGAAACAAGGACAGAGACA
GAGGCCAAGTCAACCAGACCAATTCCCAGAGGAAGCAAAGAAACCATTACAGAGA
CTACAAGGGGGAAGGGAAGGAGAGATGAATTAGCTTCCCCTGTAAACCTTAGAAC
CCAGCTGTTGCCAGGGCAACGGGGCAATACCTGTCTCTTCAGAGGAGATGAAGTTG
CCAGGGTAACTACATCCTGTCTTTCTCAAGGACCATCCCAGAATGTGGCACCCACT
AGCCGTTACCATAGCAACTGCCTCTTTGCCCCACTTAATCCCATCCCGTCTGTTAAA
AGGGCCCTATAGTTGGAGGTGGGGGAGGTAGGAAGAGCGATGATCACTTGTGGAC
TAAGTTTGTTCGCATCCCCTTCTCCAACCCCCTCAGTACATCACCCTGGGGGAACAG
GGTCCACTTGCTCCTGGGCCCACACAGTCCTGCAGTATTGTGTATATAAGGCCAGG
GCAAAGAGGAGCAGGTTTTAAAGTGAAAGGCAGGCAGGTGTTGGGGAGGCAGTTA
CCGGGGCAACGGGAACAGGGCGTTTCGGAGGTGGTTGCCATGGGGACCTGGATGC
TGACGAAGGCTCGCGAGGCTGTGAGCAGCCACAGTGCCCTGCTCAGAAGCCCCAA
GCTCGTCAGTCAAGCCGGTTCTCCGTTTGCACTCAGGAGCACGGGCAGGCGAGTGG
CCCCTAGTTCTGGGGGCAGCTCTAGAGCGGGGGATCCACTAGTTCTAGAGCGGCCG
C
Neurofilament medium chain (NEFM) minipromoter
SEQ ID NO: 28
CTTGGTATGTTCTTTTGGACTGAAAACTGATGGAGTTGGAAGTGACTTTTGTAGTGA
AAATGAGGCTGGAGTAATTAATATTGTTCCAGTGTTCTGGTTATGCGCAGGAGTGA
ATCATGATACCCAAATTACAATGACTCATCTCTTCTGAGAGAATAAATTGTGATTCT
ATGAAAGTTTTGCTTCACTAGGAATTTTTTGAAGGGTTCTGTTTGTTCTAGTTTAGA
AACCTACACAGGCAATATGGGCTTTGCAAAGGTATAAAATTTTCTTAAATAAAACT
AAACAAAGATCTATTTTGATTTTCAGCGTTGGATTTTTTTTTTTTCTTGCCTTTCCTT
TGCTTCTGGGAGAAAGTATCTTCAGACAGGAAGCTTTGAGCTACTCATAAATTCTC
CCTAACACTGCAGAAACATATTTCACTATGAAATGGTGGAGATGACAGTAAGAGC
CTCTTGTTTGAGGGGCAAAGCTGAGACATCCTGAATGCTATGGAAGTTTAATCTTC
CATGCTGACGCAATCACGGGAATATTGTGCTCCTGGGGTTTTCTTTATTGCCAGCTA
CTGTCTAATGCCAGGACAAAAGGAATAATTTGATTACAGCCGAGGAAAGCACAGT
TTGTTCCTGTTACTTTTAAGATGTGCACGCATATTAAGGTTAACAAGATTTGTGGTT
GTGAAGTATAATTCTAAAGCCTCACAAGCATGAAGGGTGTGTGGACGTTTTCCTGA
GTCATCCTAGGGAACAAAAGGGAGTGGGCAGAGCTAGTTACCAGAGTTGGAAAGG
GATGCATCAGTCATTACATTCAGGGGCTGTTAGTTGGTTCCTTTTGCAAATGGGCTG
TGAACTGCAGATGTCCCTGGGAGAGAAAATAGAAAATTACTAGCAAATTCCTAAG
AGAACCTGGCCTCTTTAAAGAAGAGAAGACACATTGGGGCTGGGGATGACATCCC
AAAACTCCCAGCGCCGGGGAGCTCAGGCTCGGGCGGTGCCTGGCTCCCCTCCCCGG
TTCCCGCCGCAGGAGGCTGCTGAGTCACTCCCGCCTGAGCCACGGCCGCCCCCAGC
CAGCCACGCGATTGGCGGGTTAGCCCGGGCGCAGCGCCCTCTTCCCAGCTCCCGCA
CCCCCAGGGCTTCCCGCAGGCAGAGGGTGGGAGCCCCGGCGAGCGACCAGTTGGG
GCTTGGTAGGAACCCCCTAAAAATGCGCCCCACTTGTGCATTCGTGCATGGGCGCA
TTTTGGTTCAGAATTAATTGAGAGTTGTTCACTCTACTATTTATTAACTAGATGATC
CGATAGTTGCTGCAAAGTAACTTGTGGTTAATGCAGGTCAGTAGAGAGCTGATCTC
ACTGCAGGCCGGAGGCAACGCCCAATTCCCACGTCTTCCCCTCCCCCACCCAACAA
GGGCTGGGCAGCTTTAATGCGGAACGCCGGTGCAGCAGCACGGAGCACACTCCCA
GACTACAGTGGCGCCCGCACTCACGCCCTTCAAGTGCCCCACCCCTCCCACACAGT
GGCCAAGGAGCCAAGCCCTATCCCTCTGCGATCTGTCCATTGTGCCAACCCTGCAT
TGCAGGGAAGTCTTAGCCTGTGATTCCAGCTTCGCTCCTGCAAAGATAGTCACTAC
AGGCAGGTTTAAATTGTATTTACTCGGAGAGAAGAGGGGGAAGGGAACCCCCTGC
CCCCTGCCAACCTGACAGGCCAGGCTGGGTGACAAGCCCCCCGGGTGGCTGCACCT
CCCACTCGCCGACCTGGGCGCTGTCCTTGGTGCTGCCGGCGCCATAGCGAGCACAA
GCGCCTTACCTTCTGCCCCTGCGGAAGCTGCGGACGTCCCCCAAACGTTAAAAACG
CCATATCCCACTTTTTATACAAATAACTACCCCGGCCTGTTCTCTTTGTCCCCTTCCC
GAAGCCTTCAGGCAGACCGTTCCGCAAAGTCTTTTTTTTTGTTTTGTTTTGTCTTCTA
GACCCAGCTCCTCGGCGCTCATCTCCAGCTGCTTTAAGACAAGGGGTGGGGGAAGG
GGAGGGAGGCAAGAAAAGATGAGGGTGGGGGAGGGGAAAAGAGGGAATGCAAG
GGGAAGGAGGGAGGAGACGGGGAGAAGGAAAGATTGGAAGAAAAGGATCTCCGA
GGAAGGGGCTGAGAGAAGGGCAGGGTGAACTGGACTAAAGGCCAGAGTAGGAAG
GAGAAGAGGGGCCAAAAAAGAAGGGGATGAAATTAAGCACAGAAGATGGGTAAA
GAAAAAAGTATCAGGGAAAGGGCAAAATAAGAGAAAGCCTTGAGGATAAGAGGG
TAGAAGGCTAAAGAACAAGGGGACCACTGGGTCGGGGAAGCGCTGCCTGAACGGC
GGGACAGTGACAAAGAAAGGGCGCTGGCGATATTCGCACCAAGGGTGCGAAACGC
AATCGGGAGGTGAGAAATGGAAAGAAGGCGAATGCCCGGCTACAAGTAGCCTGGG
ACTGAAAGGGGACCTGGGGGAGGGGCTGGGCCCAGGGCAGAAAAGTCCAGGTTCC
CATGCGGCCTGGGCCCACGTGGAGCGGGCGCTGAATCACCGTTCAGCCGCCCCCCT
CCCCTCCTCCCCGACCGGTGCCCGCAGTCCCCGCCTCCTCGGCCGCCGCCTCCACG
GGGCGGGGCCCTGGCCCGGGACCAGCGCCGCGGCTATAAATGGGCTGCGGCGAGG
CCGGCAGAACGCTGTGACAGCCACACGCCCCAA
Neurofilament light chain (NEFL) minipromoter
SEQ ID NO: 29
CTTGGTATGTTCTTTTGGACTGAAAACTGATGGAGTTGGAAGTGACTTTTGTAGTGA
AAATGAGGCTGGAGTAATTAATATTGTTCCAGTGTTCTGGTTATGCGCAGGAGTGA
ATCATGATACCCAAATTACAATGACTCATCTCTTCTGAGAGAATAAATTGTGATTCT
ATGAAAGTTTTGCTTCACTAGGAATTTTTTGAAGGGTTCTGTTTGTTCTAGTTTAGA
AACCTACACAGGCAATATGGGCTTTGCAAAGGTATAAAATTTTCTTAAATAAAACT
AAACAAAGATCTATTTTGATTTTCAGCGTTGGATTTTTTTTTTTTCTTGCCTTTCCTT
TGCTTCTGGGAGAAAGTATCTTCAGACAGGAAGCTTTGAGCTACTCATAAATTCTC
CCTAACACTGCAGAAACATATTTCACTATGAAATGGTGGAGATGACAGTAAGAGC
CTCTTGTTTGAGGGGCAAAGCTGAGACATCCTGAATGCTATGGAAGTTTAATCTTC
CATGCTGACGCAATCACGGGAATATTGTGCTCCTGGGGTTTTCTTTATTGCCAGCTA
CTGTCTAATGCCAGGACAAAAGGAATAATTTGATTACAGCCGAGGAAAGCACAGT
TTGTTCCTGTTACTTTTAAGATGTGCACGCATATTAAGGTTAACAAGATTTGTGGTT
GTGAAGTATAATTCTAAAGCCTCACAAGCATGAAGGGTGTGTGGACGTTTTCCTGA
GTCATCCTAGGGAACAAAAGGGAGTGGGCAGAGCTAGTTACCAGAGTTGGAAAGG
GATGCATCAGTCATTACATTCAGGGGCTGTTAGTTGGTTCCTTTTGCAAATGGGCTG
TGAACTGCAGATGTCCCTGGGAGAGAAAATAGAAAATTACTAGCAAATTCCTAAG
AGAACCTGGCCTCTTTAAAGAAGAGAAGACACATTGGGGCTGGGGATGACATCCC
AAAACTCCCAGCGCCGGGGAGCTCAGGCTCGGGCGGTGCCTGGCTCCCCTCCCCGG
TTCCCGCCGCAGGAGGCTGCTGAGTCACTCCCGCCTGAGCCACGGCCGCCCCCAGC
CAGCCACGCGATTGGCGGGTTAGCCCGGGCGCAGCGCCCTCTTCCCAGCTCCCGCA
CCCCCAGGGCTTCCCGCAGGCAGAGGGTGGGAGCCCCGGCGAGCGACCAGTTGGG
GCTTGGTAGGAACCCCCTAAAAATGCGCCCCACTTGTGCATTCGTGCATGGGCGCA
TTTTGGTTCAGAATTAATTGAGAGTTGTTCACTCTACTATTTATTAACTAGATGATC
CGATAGTTGCTGCAAAGTAACTTGTGGTTAATGCAGGTCAGTAGAGAGCTGATCTC
ACTGCAGGCCGGAGGCAACGCCCAATTCCCACGTCTTCCCCTCCCCCACCCAACAA
GGGCTGGGCAGCTTTAATGCGGAACGCCGGTGCAGCAGCACGGAGCACACTCCCA
GACTACAGTGGCGCCCGCACTCACGCCCTTCAAGTGCCCCACCCCTCCCACACAGT
GGCCAAGGAGCCAAGCCCTATCCCTTCAGGGCTGGCCGCAGCGGCTTCCCTGAAAA
TCAGCCAACTGCAAGGCTTATCGAAATCATCAGGTCGTGTGCTACACATGTGTAAA
AAGAGGAAAGCGGACTTTAAATGTGCTGCGGTTGGTGGTAGCAAGCAGGAATTTA
GCTTGGTGAGGATCCAGGCAGCTTGAAGCTCCCGGCTGCGGAGCGCGCGGCTGCCT
CAGCAAGTCAGTCTCTGTGTTTCCAACTCTTTCTCTGCCCTCCACCACCCACACACT
GCAACACGTTAAAGCCATCTGCGGCTTCATTCTCAGTTAAGAAATGTGAAAATCTA
GAAACACTAACAGGCGGATTAACTGCTGTAAAGGTTTAAAAATGCTAAACCAATA
CCTGCAGTAGTGCCGCAGTTTCACGAGTGTGTGTGTGTGTGTGTGTGTGTGTGCGC
GCGCGCGCGCGCACTCGCGCGCACATTCCCTATGTGTTAAGCAGCTCATTAAAGAA
AAAGAAAAATAATCAGGAGAAAGGAAGATGAATTGCAGAAAGTGCCAGAAAGCT
AGAAAGAAATTAAAACTCTTCTCCATACATACTGCATACACATAACCTAGCCTATT
TATTTGTATCTAAAATTCCCTAGCCGCACCATCACCGTAAACACCAAGGGAAAAAA
TTAAGGAGGTTCCTGGTGGGAAAAGGGCGAGTTGGGGGGACAGGGTGTCTGCGAG
GTGACGGGATACAGAAAACTAGGGTGTCAAAAGGGAGCAAGAACCTGTTTTGGGG
GCAACTTAAGGATCCAAGTGTCACGGGGTCTGGGCAATGCAGGACGGGAGGGGCT
GCGTGAGTGAGTACAGAAGGGAAATGAGTGAGGGGGCATGGGATCTCAGAGAAA
ATCAGGGCCCTCTGAGCAAAGTGGAAAGGACGACCGCCGCAGCTCCTCGGGCCGT
AGCTCGACCCCGCCTTCCCTTTTGCGCAGAATCCTCGCCTTGGCTGCAGCAGCGCG
CTGCCCCCACTGGCCGGCGTGCCGTGATCGATCGCAGGCTGCGTCAGGAGCCTCCC
GGCGTATAAATAGGGGTGGCAGAACGGCGCCGAGCCGCACACAGCCATCCATCCT
CCCCCTTCCCTCTCTCCCCTGTCCTCTCTCTCCGGGCTCCCACCGCCGCCGCGGGCC
GGGGAGCCACCG
Neurofilament heavy chain (NEFH) promoter
SEQ ID NO: 30
TTGGGAAACTCAGCACACTCTACTTGCCTCTGGGAGATTCACAATGCTCACTGCCA
AGTCCAGGCTTTGGGAAGATAACTCCAAGAACTGCGGGTGGATGGATGGATGGAT
GGATAGATAGATGAATATATCGTTCTACTCACTTCCAAATGGTAAGAATCACAGGG
AGGAAAGTGAAAGCGATTCGAGCCCCAAGGACTCAGGGCAGAACCATTCACAGCC
CCTCCAGACCACAAGTTTGGCTGGAAAACAGACGGAACTTATTCATTCATTGATTC
ATTTACACACCACAGAGTGGTAAGAGCAGCATTAGAGATTAGCACTGGGAACCAT
GGGAACCACCAGGAGGGGACAATTAACTAAATGTGGGGGTGAAGGCGGCCAGGG
ATGGCTTTCTGGAGGTGAGCCTGAAGGGTCCTTTGGGGGAACTGACCTCAGGGCTC
CAGCCCTCATGCCATTTTCTCCAGCCACAAGAGTCATGCTTGGGGCTTCTTGGACTA
CATAGGCAGCTTCAATCTGATGGCTGTGGCCCCTTGGCCTCAACAGAATACATCTT
GGAGCCCCCTTTTTACCCCAAACCCCCATTCCTCCTTGCTGTCAGCTGCTTGTGAGC
CTTCTCACATCCAGAGAATGTATCAGCATTGTGCAGACTGAAAAGACCCAGAGGA
ACAAGGCTCCAATGGCAAAATTCCAAGTAGAATGACAAATAAATGGGGAGCCATC
TGAGAGCAAGGGAGTCCTGCCCAACACCCGCCCCATGCCTTTCTCAGGGACCTCAG
ACCAGCCACTCACCTCCATCCTCCCAGCACCACCTGCAACCAGCCCCTTGCCCTCTG
CAAACTGGAGCACGACTGGATCTTTAGATGGGGGAAAAATGCTTCATCATGTTCTG
CTGCTTCATGCAAAACCAGAAACTCCCTCCCCCTCTTCCCTCCTCCCAGCGCACTCT
CCTTCCAGTAAAAAGTGGTTAAAGGGACAGCGCCATCAATTTCCCAGCTCTGAGGG
TCTGCTTAGAACTAGGGGGCTGGAAGGAGACAGAGGGCAAAGAGAAAGGAACTG
GCAGAGGTCTTTCCTGGGGGATATGTCTGTTCTGTCCTGGGGATCCTGGAGCAGGA
AAACCCGCGTAAAGTAGGGGTGTAGTGGGTGTTGAGATAACTGCCTGGGGGAGGT
TCAGAGTGGAAGTACGAGTCTACAAACTCTCAAGGGCGTCTCAGGGCTCCCAGCAT
CCCCAGGGGTCCTTTCGCAGGGGTCCCTAAGCAGGAGGGGAACAGCCCAGAAAAC
ACGGAACTGGACCCCCGACAGGAAGTCCAGGGAGGGGTCCCTGGCTCACTATGTG
ACCCTGCTGGATCACTTGCCTCCCCTCTCGGGTCCCCTCAGCACAGTGTCCCTCCCT
TCCTTCCCCTAAAGTAAAAGCAGAGGGTTAATCTCTTTCCCCGCCCCACGCCCAAC
AAAGAGCAGGCCCTGTCCCCGGTGCTGAAGCGCCAGCCGCAGCACCACCCCCACT
CCCACAGCATAAAACATGAGCCAAAACCAATAAAGAGCCAAATGTCACAGCCGTT
GCAGGGCCCCCTAAATCCTGGGGACCCCTTCTTCTACCTGACATCCTATTGGGGTG
AGGGACTTTGGTACTCAGAAAGCATCTCATCACTTCCCTGTAAGAGAGAAGGGATG
CCGACTCAGGCGCCTGCTTGTCTGTTACAGGAGTGGGGGAAGAGAGGACAAGTTG
AGGCTGAGAAGATGGGGAGGGGGAGGGAGAAAAGAGGACTTCCTAGTGTTGACA
GAACGGCAAGATGTGGGTTCCCCATCCCCAGTTCAGCCAGAGACCCCTCAAAGTGG
AACTTCCTGGGGCAGTCGGGGGTCAGGAGTTGGAGCTTGTCTCTGGGGCAAGACCC
CTTCGTTGTACAGATGGAAAAACAAGGGTGGGAGGACACAGCTTGTCCAAGGTCA
TTCGACCAGCAAACTGCCTAGCTGACCCCAGTGTGCAGAAGCTGGCTCGGGTGACA
CCCATCATTTCCCCCCACCCCACACAGGGGCCAGCTCTCTCAACTTCATGCCCAAG
CCCTCCTACGGTACCCCCACTGTAGGTTCTCTGCCCCTCAAACTCAGCCCAGCTTTC
TCCTGCCTGTTCAGGGGACCTTCTGCCCGCTTCGCTGAGGGTCCGTCCCCTTTACTG
GGGCTGGCAGCAGGGTCTCCCATCTCCTCTCTCGGGGGCCACTGCAGACTTTTTAG
AGAACGCCTTGCCTCCCCCCAACCCCACCCATCCGGGGTTCCCTCTCTCCATCCTCT
GCAGTGTCTCCCATACCCCCATTCAGGGTAGCCTTGCTATTCTCCCCAACTCCAGGT
CCCCCTTCATCTATTCCGGGGCTGGCCGCGGAGTTTCCTGAGCGCTCTCCAAGTGG
GTCCTCTAGATGTTAGGAGAACACTGTACCTCCCCCGGTCAGGGGTCTCCTGTCTCC
GTTCTATGGAGCGTCCATGCTCCCATTCAGGACTGCCTTGCTCCCTCCTCTGTTCCG
GGGCTGGCTGCACAGTCTCTGCACCCCCTATCCTGAAAGCCTCTCTTAACTATTTGG
AAAGCCTCGTGTCCTGTCTCATACAGGGATCCCCTCATCCTAATGACTGCAATCTTC
CATTGCTCCATCCCGAGGGCATCCTGCCCCTATTCCCATCAGGTTTCTCCTTGTCCT
CTCCCTGTTTCAAGTCCCCTTTCTTATTCCGAACACACTCGCAGGCTCTTCCGACGC
GCACCCGGGGGTCCTCACTGGCCCACTCCGGGAGTCCTCTGCCCGCTTCCCCGACC
TCGAGGGTCTCCTCTGACGCAGCGTCGATTCCCCTTCCCTCCTCGGTCCCCTGCCCC
GCCCCTCTCACTGCGGCGGAGCCGGTCGGCCGGGGGGCCGCAGGGGAGGAGGCGG
AGAGGGCGGGGCCCTCCTCCCCACCCTCTCACTGCCAAGGGGTTGGACCCGGCCGC
GGCGGCTATAAAAGGGCCGGCGCCCTGGTGCTGCCGCAGTGCCTCCCGCCCCGTCC
CGGCCTCGCGCACCTGCTCAGGCC

Promoters for GBA knockin include the following SEQ ID NOS 31-40 and 43 (these sequence listings are a “synthetic construct”):

Mouse Thy-1 cell surface antigen (mTHY1)
SEQ ID NO: 31
GGTTATCCAGGCTTCTGAAGGTTCAAGCAAAGAAAGGGTTACAACCTTAAAAGGA
GAGCGTCCCGGGGTATGGGTAGAAGACTGCTCCACCCCGACCCCCAGGGTCCCTAA
CCGTCTTTTCCCTGGGCGAGTCAGCCCAATCACAGGACTGAGAGTGCCTCTTTAGT
AGCAGCAAGCCACTTCGGACACCCAAATGGAACACCTCCAGTCAGCCCTCGCCGA
CCACCCCACCCCCTCCATCCTTTTCCCTCAGCCTCCGATTGGCTGAATCTAGAGTCC
CTCCCTGCTCCCCCCTCTCTCCCCACCCCTGGTGAAAACTGCGGGCTTCAGCGCTGG
GTGCAGCAACTGGAGGCGTTGGCGCACCAGGAGGAGGCTGCAGCTAGGGGAGTCC
AGGTGAGAGCAGGCCGACGGGAGGGACCCGCACATGCAAGGACCGCCGCAGGGC
GAGGATGCAAGCCTTCCCCAGCTACAGTTTTGGGAAAGGATACCAGGGCGCTCCTA
TATGGGGGCGCGGGAACTGGGGAAAGAAGGTGCTCCCAGGTCGAGGTGGGAGAG
GAAGGCAGTGCGGGGTCACGGGCTTTCTCCCTGCTAACGGACGCTTTCGAAGAGTG
GGTGCCGGAGGAGAACCATGAGGAAGGACATCAAGGACAGCCTTTGGTCCCCAAG
CTCAAATCGCTTTAGTGGTGCGAATAGAGGGAGGAGGTGGGTGGCAAACTGGAGG
GAGTCCCCAGCGGGTGACCTCGTGGCTGGCTGGGTGCGGGGCACCGCAGGTAAGA
AAACCGCAATGTTGCGGGAGGGGACTGGGTGGCAGGCGCGGGGGAGGGGAAAGC
TAGAAAGGATGCGAGGGAGCGGAGGGGGGAGGGAGCGGGAGAATCTCAACTGGT
AGAGGAAGATTAAAATGAGGAAATAGCATCAGGGTGGGGTTAGCCAAGCCGGGCC
TCAGGGAAAGGGCGCAAAGTTTGTCTGGGTGTGGGCTTAGGTGGGCTGGGTATGA
GATTCGGGGCGCCGAAAACACTGCTGCGCCTCTGCCAAATCACGCTACCCCTGTAT
CTAGTTCTGCCAGGCTTCTCCAGCCCCAGCCCCAATTCTTTTCTCTAGTGTTCCCCCT
TCCCTCCCCTGAATCTCAAGCCCACACTCCCTCCTCCATAACCCACTGTTATCAAAT
CTAAGTCATTTGCCACCCAACAACCATCAGGAGGCGGAAGCAGACGGGAGGAGTT
TGAGATCAACTTGGGCTACATCACGAGTTCCAGGCTCACCAAGGCTTCTTAAGGAG
ACCTTGTCTCTAAAATTAATTAATTAATTAATTAATAGTCCCCTTTCTCTGCCACAG
AACCTTGGGATCTGGCTCCTGGTCGCAGCTCCCCCCACCCCAGGCTGACATTCACT
GCCATAGCCCATCCGGAAATCCTAGTCTATTTCCCCATGGATCTTGAACTGCAGAG
AGAATGGCAGAGTGGCCCGCCCTGTGCAAAGGATGTTCCTAGCCTAGGTGGAGCTC
GCGAACTCGCAGACTGTGCCTCTCTTGGGCAAGGACAGGCTAGACAGCCTGCCGGT
GTGTTGAGCTAGGGCACTGTGGGGAAGGCAGAGAACCTGTGCAGGGCAGCAATGA
ACACAGGACCAGAAAACTGCAGCCCTAGGAACACTCAAGAGCTGGCCATTTGCAA
GCATCTCTGGCCTCCGTGCTTCTCACTCATGTCCCATGTCTTATACAGGCCTCTGTG
GCACCTCGCTTGCCTGATCTCATCCCTAGCCGTTAAGCTTTCTGCATGACTTATCAC
TTGGGGCATAATGCTGGATACCTACCATTTTCTTAGACCCCATCAAAATCCTATTTG
AGTGTACGGTTCGGAGAACCTCATTTATCCGGTAAATGTCTTTTACTCTGCTCTCAG
GGAGCTGAGGCAGGACATCCTGAGATACATTGGGAGAGGAGATACAGTTTCAATA
AAATAATAGGTTGGGTGGAGGTACATGCCTATAATGCCACCACTCAGGAAATGGT
GGCAGCTTCGTGAGTTTGAGGCCAACCCAAGAAACATAGTGAAACCCTGTCAGTA
AATAAGTAAGCAAGTATTTGAGTATCTACTATATGCTAGGGCTGACCTGGACATTA
GGGGTCATCTTCTGAACAAACTAGTGCTTGAGGGAGGTATTTGGGGTTTTTGTTTGT
TTAATGGATCTGAATGAGTTCCAGAGACTGGCTACACAGCGATATGACTGAGCTTA
ACACCCCTAAAGCATACAGTCAGACCAATTAGACAATAAAAGGTATGTATAGCTTA
CCAAATAAAAAAATTGTATTTTCAAGAGAGTGTCTGTCTGTGTAGCCCTGGCTGTT
CTTGAACTCACTCTGTAGACCAGGCTGGCCTGGAAATCCATCTGCCTGCCTCTGCCT
CTCTGCCTCTCTGCCTCTCTGCCTCTCTCTCTGCCTCTCTCTGCCTCTCTCTGCCCCTC
TCTGCCCCTCTCTGCCCCTCTCTGCCGCCCTCTGCCTTTTGCCCTCTGCCCTCTGTTC
TCTGGCCTCTGCCCTCTGCCCTCTGGCCTCTGGCCTCTGCCTCTGCCTCTTGAGTGCT
GGAATCAAAGGTGTGAGCTCTGTAGGTCTTAAGTTCCAGAAGAAAGTAATGAAGT
CACCCAGCAGGGAGGTGCTCAGGGACAGCACAGACACACACCCAGGACATAGGCT
CCCACTTCCTTGGCTTTCTCTGAGTGGCAAAGGACCTTAGGCAGTGTCACTCCCTAA
GAGAAGGGGATAAAGAGAGGGGCTGAGGTATTCATCATGTGCTCCGTGGATCTCA
AGCCCTCAAGGTAAATGGGGACCCACCTGTCCTACCAGCTGGCTGACCTGTAGCTT
TCCCCACCACAGAATCCAAGTCGGAACTCTTGGCACC
Rat Neuron specific enolase (rNSE) promoter
SEQ ID NO: 32
GAGCTCCTCCTCTGCTCGCCCAATCCTTCCAACCCCCTATGGTGGTATGGCTGACAC
AGAAAATGTCTGCTCCTGTATGGGACATTTGCCCCTCTTCTCCAAATATAAGACAG
GATGAGGCCTAGCTTTTGCTGCTCCAAAGTTTTAAAAGAACACATTGCACGGCATT
TAGGGACTCTAAAGGGTGGAGGAGGAATGAGGGAATTGCATCATGCCAAGGCTGG
TCCTCATCCATCACTGCTTCCAGGGCCCAGAGTGGCTTCCAGGAGGTATTCTTACA
AAGGAAGCCCGATCTGTAGCTAACACTCAGAGCCCATTTTCCTGCGTTAACCCCTC
CCGACCTCATATACAGGAGTAACATGATCAGTGACCTGGGGGAGCTGGCCAAACT
GCGGGACCTGCCCAAGCTGAGGGCCTTGGTGCTGCTGGACAACCCCTGTGCCGATG
AGACTGACTACCGCCAGGAGGCCCTGGTGCAGATGGCACACCTAGAGCGCCTAGA
CAAAGAGTACTATGAGGACGAGGACCGGGCAGAAGCTGAGGAGATCCGACAGAG
GCTGAAGGAGGAACAGGAGCAAGAACTCGACCCGGACCAAGACATGGAACCGTA
CCTCCCGCCAACTTAGTGGCTCCTCTAGCCTGCAGGGACAGTAAAGGTGATGGCAG
GAAGGCAGCCCCCGGAGGTCAAAGGCTGGGCACGCGGGAGGAGAGGCCAGAGTC
AGAGGCTGCGGGTATCTCAGATATGAAGGAAAGATGAGAGAGGCTCAGGAAGAG
GTAAGAAAAGACACAAGAGACCAGAGAAGGGAGAAGAATTAGAGAGGGAGGCAG
AGGACCGCTGTCTCTACAGACATAGCTGGTAGAGACTGGGAGGAAGGGATGAACC
CTGAGCGCATGAAGGGAAGGAGGTGGCTGGTGGTATATGGAGGATGTAGCTGGGC
CAGGGAAAAGATCCTGCACTAAAAATCTGAAGCTAAAAATAACAGGACACGGGGT
GGAGAGGCGAAAGGAGGGCAGAGTGAGGCAGAGGGACTGAGAGGCCTGGGGATG
TGGGCATTCCGGTAGGGCACACAGTTCACTTGTCTTCTCTTTTTCCAGGAGGCCAAA
GATGCTGACGTCAAGAACTCATAATACCCCAGTGGGGACCACCGCATTCATAGCCC
TGTTACAAGAAGTGGGAGATGTTCCTTTTTGTCCCAGACTGGAAATCCATTACATC
CCGAGGCTCAGGTTCTGTGGTGGTCATCTCTGTGTGGCTTGTTCTGTGGGCCTACCT
AAAGTCCTAAGCACAGCTCTCAAGCAGATCCGAGGCGACTAAGATGCTAGTAGGG
GTTGTCTGGAGAGAAGAGCCGAGGAGGTGGGCTGTGATGGATCAGTTCAGCTTTCA
AATAAAAAGGCGTTTTTATATTCTGTGTCGAGTTCGTGAACCCCTGTGGTGGGCTTC
TCCATCTGTCTGGGTTAGTACCTGCCACTATACTGGAATAAGGGGACGCCTGCTTC
CCTCGAGTTGGCTGGACAAGGTTATGAGCATCCGTGTACTTATGGGGTTGCCAGCT
TGGTCCTGGATCGCCCGGGCCCTTCCCCCACCCGTTCGGTTCCCCACCACCACCCGC
GCTCGTACGTGCGTCTCCGCCTGCAGCTCTTGACTCATCGGGGCCCCCGGGTCACA
TGCGCTCGCTCGGCTCTATAGGCGCCGCCCCCTGCCCACCCCCCGCCCGCGCTGGG
AGCCGCAGCCGCCGCCACTCCTGCTCTCTCTGCGCCGCCGCCGTCACCACCGCCAC
CGCCACCGGCTGAGTCTGCAGTCCTCGAG
Dopa Carboxylase (DDC) promoter
SEQ ID NO: 33
CCATGGTGGGAGGAGAGACTGAGCCCAAACCATGTTTTAGATGCTGATAGGCTTAA
GGGTAACAGCACAGGAGTTTGAGATGCATGCGGCTCAACACCTAATCTACATCTCA
CTTCACTTTCTCATCTGGGGAAGTGGGCTTGGGACCCTGAGCCTCCCGGGTATCAC
AGGGTCCTAATAGTCCCTCACAGAAGGAGCAGACCCAGAGTGAGCACTCCCCAAA
TGCCACGCCGTCCCTTCCTCACCCTTGGAGTGGAGCCTGGTCGTTCTCAAAGTTGCT
GGGAGAGTCCCAGGAGCCCTGGCCCCAAATCTGCATCCTACACAGTGCCTGGGAA
CACAGGGCCCATTTTTTCCTTGGCCTCTCGCCAGTCCCAGCAGGCCCTGATGCTCCT
CTCCATCCTGCTAGGATGGCTCTCTCCCCCTGGGGGCAGAGTGGGGGCAGGAGGTG
GTGGGAGGGTGGAGGGATGCTGCTCAGTAAATAATGCAGAGCCGGCAGCTCTGAT
TGGCTTCGGGGAGGCAGACACTCTGTCTACATAAATGGCAATCACATCTTCTGTGC
CT
catechol-O-methyltransferase (COMT) promoter
SEQ ID NO: 34
ACAGAAGCACTTCACCTGTTTATTTCTTGAAAAGCCATTCTATGTTGCTGCTTCCCAC
TTTTAGTTCTCAGAGGTGGCTTTCCTACTGTCCAGAAGACATAAACTTCTGTTTTCCC
TGGAGTCACCTTAGCTTAAACTCCTATAATCTTGTTGAATCATCCCTGTGTTTGGCTA
AACCAGGCTCCTCATACCCACGTTCCAGGGTCACACAGGTAACTTGGCTTGCTGCTT
CTGGGGGCTCTGAATCCCACAATGCCGATCCACCACCTCTTCCCTGCTGCCACTAGT
CTCCTGGGGTCTAGCCTCAACAGCTTCCACCAAGGACACACTGCATTGCTTAAGAGT
GTATTTAAGGTGCGGCATTTGTGATGCTAGGGAGGAGCCTTCCTGACTTAATCACGT
AGGGGGAGTGGGGAAAGAAGTGGGTATTTGGAAGGAGGGTGAGGTGGGCTGGGA
ATGCCAAGAAATGGTAGTACTGTGTGGTGAATCAAAGTGGGTCTTGGAATCTTCCTA
GCTTTTCTTTTCTTTTCTTTTCTTTTTTTTTCTTTCTGACTTTTTATCTTATGTTGTCACT
CACCATGTTTTGTTTTCCTCATCTGCAAAATGGATTGTTGTAAGCTTGAACAGGAATC
ATGCATAAAGTACTGAGCACAGTGCCTGGTACCTTGAAATCACTCAATGGAAGTTTC
TTATTATTTGCCCTACACCCAAGGAGTAATTGGGGCCCTGAAGGAAGACACCCCAAT
TTGCCGTCCTCCCAGCCGCCAAGCCTGATAGTGCACTAGGGAGGCTGATGGGAAAA
TACCGTGCAGAAGGCTGAGGAGTGGGGATTCTGGGGGGGGACTCTTTGAAGTCCTA
GGTGACCTCTCCGCCCAGGCACCCGCCCTCCCCGGGCCTGGGCTGCAGAGCTGCGG
GCGGGGGCGGTGGCCCTGCGCTGCACCCGGCCTGCCTGCGCGTAGGCGGGGGGC
GGGGCTGCGCGTCCGGGCTCCCGGGGCTGGTAATATAGCGGCTCGCCGAGGCGCTG
GTGCACGGGGGCAGCGCGCAGCAGGCCGGCGGGCAGGCGGGCGGGCTGGCTGGC
AGGCAGGACTGGGATCGAGGCCCAGAAAACGGAGCAGCGGGCACCAGGGAGGCC
TGGAACGGGGCGAGCGCC
Glucosylceramidase beta 1 Promoter 1 (GBA P1)
SEQ ID NO: 35
ATAGAATGTTAAGGTCCCAAGATCAGAAACTTGCTCATTGCAGTGGGTCTAACACT
CAGTAGGTCCTCAACAAACATTCGTTAAGATACTAAAGTGGCAGGGTGGGGCCCTG
TAAACAGCTTCAGGACCCTGTGCTTGTAGGGGCAACGTGGTGCCCTCCAAGGAAGA
CAGGGAGGTGGGAGGAGCACTGCCCAGAGATGGCGTCAGGCTGCAAGACTTCTTG
AATAATTCAGCATCATAACAACCCAGCCTCAGGAAGGGATAGGGCACGGCCAGGA
CGAAACATTAGGAGGCGATGGACAATGGGATTCCCACGGGGCAGCTTCTGCGCAC
TGGACGTTCCCTAACCTGAGGCTCTCTAAAGAGGAAGGTTAGGAATCCTCTGAGCT
TCGGTGGGCTGGACTCACTGTGGGAATTCAATCGCCCCATCCACCAACAGTGTGCT
GGCGGGAAAACGCCGACACGCATGCGTAGTTCTCGCGCCGGCTCCTCTCTCTCT
Glucosylceramidase beta 1 Promoter 2 (GBA P2)
SEQ ID NO: 36
CCTCCCTCCAGTCCTGTCATCCCACTGCCAGTGTCCCACCCTCTTGTGCCCTGCACT
TCACTGGCTAATAACCCCCCTCACTTTTTCCTCTGTGCCGCCATCCTGGATAATTCC
CCACCCACGAATGGTCCCTCCTCATCTCAGAGAGCTCTCCATGCACACCTGTTACC
GTTTCTGTCTTTATCTGTAAATATCTGTGTGTCTGACTTCCATGCCTCACACACCTCT
ATAGGGCAAAGACTGTCTTAAACATCTTGGTAGTGTCAAGTATTTTGCACAGTGAA
GTTTTTTTTTTTAAATTATATCAGCTTTATTTGTACCTTTTTGACATTTCTATCAAAA
AAGAAGTGTGCCTGCTGTGGTTCCCATCCTCTGGGATTTAGGAGCCTCTACCCCATT
CTCCATGCAAATCTGTGTTCTAGGCTCTTCCTAAGTIGTCACCCATACATGCCCTCC
AGAGTTTTATAGGGCATATAATCTGTAACAGATCAGAGGAAGCC
Human eukaryotic translation elongation factor 1 alpha 1 short
form (EFS) promoter
SEQ ID NO: 37
GGCTCCGGTGCCCGTCAGTGGGCAGAGCGCACATCGCCCACAGTCCCCGAGAAGTT
GGGGGGAGGGGTCGGCAATTGATCCGGTGCCTAGAGAAGGTGGCGCGGGGTAAAC
TGGGAAAGTGATGTCGTGTACTGGCTCCGCCTTTTTCCCGAGGGGGGGGAGAACC
GTATATAAGTGCAGTAGTCGCCGTGAACGTTCTTTTTCGCAACGGGTTTGCCGCCA
GAACACAGG
Human eukaryotic translation elongation factor 1 alpha 1 (EEF1A1)
promoter
SEQ ID NO: 38
GGCTCCGGTGCCCGTCAGTGGGCAGAGCGCACATCGCCCACAGTCCCCGAGAAGTT
GGGGGGAGGGGTCGGCAATTGAACCGGTGCCTAGAGAAGGTGGCGCGGGGTAAAC
TGGGAAAGTGATGTCGTGTACTGGCTCCGCCTTTTTCCCGAGGGTGGGGGAGAACC
GTATATAAGTGCAGTAGTCGCCGTGAACGTTCTTTTTCGCAACGGGTTTGCCGCCA
GAACACAGGTAAGTGCCGTGTGTGGTTCCCGCGGGCCTGGCCTCTTTACGGGTTAT
GGCCCTTGCGTGCCTTGAATTACTTCCACCTGGCTGCAGTACGTGATTCTTGATCCC
GAGCTTCGGGTTGGAAGTGGGTGGGAGAGTTCGAGGCCTTGCGCTTAAGGAGCCC
CTTCGCCTCGTGCTTGAGTTGAGGCCTGGCCTGGGCGCTGGGGCCGCCGCGTGCGA
ATCTGGTGGCACCTTCGCGCCTGTCTCGCTGCTTTCGATAAGTCTCTAGCCATTTAA
AATTTTTGATGACCTGCTGCGACGCTTTTTTTCTGGCAAGATAGTCTTGTAAATGCG
GGCCAAGATCTGCACACTGGTATTTCGGTTTTTGGGGCCGCGGGCGGCGACGGGGC
CCGTGCGTCCCAGCGCACATGTTCGGCGAGGCGGGGCCTGCGAGCGCGGCCACCG
AGAATCGGACGGGGGTAGTCTCAAGCTGGCCGGCCTGCTCTGGTGCCTGGTCTCGC
GCCGCCGTGTATCGCCCCGCCCTGGGCGGCAAGGCTGGCCCGGTCGGCACCAGTTG
CGTGAGCGGAAAGATGGCCGCTTCCCGGCCCTGCTGCAGGGAGCTCAAAATGGAG
GACGCGGCGCTCGGGAGAGCGGGCGGGTGAGTCACCCACACAAAGGAAAAGGGC
CTTTCCGTCCTCAGCCGTCGCTTCATGTGACTCCACGGAGTACCGGGCGCCGTCCA
GGCACCTCGATTAGTTCTCGAGCTTTTGGAGTACGTCGTCTTTAGGTTGGGGGGAG
GGGTTTTATGCGATGGAGTTTCCCCACACTGAGTGGGTGGAGACTGAAGTTAGGCC
AGCTTGGCACTTGATGTAATTCTCCTTGGAATTTGCCCTTTTTGAGTTTGGATCTTG
GTTCATTCTCAAGCCTCAGACAGTGGTTCAAAGTTTTTTTCTTCCATTTCAGGTGTC
GTGA
Chicken beta actin (CBA) promoter
SEQ ID NO: 39
CGTTACATAACTTACGGTAAATGGCCCGCCTGGCTGACCGCCCAACGACCCCCGCC
CATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGT
GTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACT
CGAGGCCACGTTCTGCTTCACTCTCCCCATCTCCCCCCCCTCCCCACCCCCAATTTT
GTATTTATTTATTTTTTAATTATTTTGTGCAGCGATGGGGGCGGGGGGGGGGGGGG
GGCGCGCGCCAGGCGGGGCGGGGCGGGGCGAGGGGCGGGGCGGGGCGAGGCGGA
GAGGTGCGGCGGCAGCCAATCAGAGCGGCGCGCTCCGAAAGTTTCCTTTTATGGCG
AGGCGGCGGCGGCGGCGGCCCTATAAAAAGCGAAGCGCGCGGCGGGCGGGAGCG
GGATCAGCCACCGCGGTGGCGGCCTAGAGTCGACGAGGAACTGAAAAACCAGAAA
GTTAACTGGTAAGTTTAGTCTTTTTGTCTTTTATTTCAGGTCCCGGATCCGGTGGTG
GTGCAAATCAAAGAACTGCTCCTCAGTGGATGTTGCCTTTACTTCTAGGCCTGTAC
GGAAGTGTTACTTCTGCTCTAAAAGCTGCGGAATTGTACCCGCGGCCGATCCACCG
GTCCGGAATTCCCGGGATATCGTCGACCCACGCGTCCGGGCCCCACGCTGCGCACC
CGCGGGTTTGCT
Cytomegalovirus (CMV) promoter
SEQ ID NO: 40
TAGTTATTAATAGTAATCAATTACGGGGTCATTAGTTCATAGCCCATATATGGAGTT
CCGCGTTACATAACTTACGGTAAATGGCCCGCCTGGCTGACCGCCCAACGACCCCC
GCCCATTGACGTCAATAATGACGTATGTTCCCATAGTAACGCCAATAGGGACTTTC
CATTGACGTCAATGGGTGGAGTATTTACGGTAAACTGCCCACTTGGCAGTACATCA
AGTGTATCATATGCCAAGTACGCCCCCTATTGACGTCAATGACGGTAAATGGCCCG
CCTGGCATTATGCCCAGTACATGACCTTATGGGACTTTCCTACTTGGCAGTACATCT
ACGTATTAGTCATCGCTATTACCATGGTGATGCGGTTTTGGCAGTACATCAATGGG
CGTGGATAGCGGTTTGACTCACGGGGATTTCCAAGTCTCCACCCCATTGACGTCAA
TGGGAGTTTGTTTTGGCACCAAAATCAACGGGACTTTCCAAAATGTCGTAACAACT
CCGCCCCATTGACGCAAATGGGCGGTAGGCGTGTACGGTGGGAGGTCTATATAAG
CAGAGCTGGTTTAGTGAACCGTCAGATC
Human phosphoglycerate kinase 1 (hPGK) promoter
SEQ ID NO: 43
GGGTTGCGCCTTTTCCAAGGCAGCCCTGGGTTTGCGCAGGGACGCGGCTGCTCTGG
GCGTGGTTCCGGGAAACGCAGCGGCGCCGACCCTGGGTCTCGCACATTCTTCACGT
CCGTTCGCAGCGTCACCCGGATCTTCGCCGCTACCCTTGTGGGCCCCCCGGCGACG
CTTCCTGCTCCGCCCCTAAGTCGGGAAGGTTCCTTGCGGTTCGCGGCGTGCCGGAC
GTGACAAACGGAAGCCGCACGTCTCACTAGTACCCTCGCAGACGGACAGCGCCAG
GGAGCAATGGCAGCGCGCCGACCGCGATGGGCTGTGGCCAATAGCGGCTGCTCAG
CAGGGCGCGCCGAGAGCAGCGGCCGGGAAGGGGCGGTGCGGGAGGCGGGGTGTG
GGGCGGTAGTGTGGGCCCTGTTCCTGCCCGCGCGGTGTTCCGCATTCTGCAAGCCT
CCGGAGCGCACGTCGGCAGTCGGCTCCCTCGTTGACCGAATCACCGACCTCTCTCC
CCAGG

### Pluripotent Cells

pluripotent cell” means a cell capable of differentiating into cells of all three germ layers. Pluripotent cells include stem cells, such as cord blood stem cells, progenitor cells, bone marrow stem cells, embryonic stem cells (ESC) and induced pluripotent stem cells (IPS).

### Rare-Cutting Endonucleases

“Rare-cutting endonucleases” are sequence-specific endonuclease reagents of choice, insofar as their recognition sequences generally range from 10 to 50 successive base pairs, preferably from 12 to 30 bp, and more preferably from 14 to 20 bp.

### Serotonergic Neurons

Serotonergic neurons are cells that express TPH, SERT, and Pet1.

### SNCA-Modified DA Neuronal Cells

The term “SNCA-modified DA neuronal cells” as used herein, refer to DA neuronal cells in which SNCA activity is suppressed by any of a number of strategies alone or in combination. For example, SNCA-modified DA neuronal cells include, but are not limited to, SNCA “knock out” DA neuronal cells in which the SNCA gene has been genetically deleted or modified such as by gene editing, i.e., SNCA^{-/-}. In some embodiments, only a single allele of the SNCA gene is affected by gene editing, i.e., the cell is SNCA+/−; SNCA protein “knock down” DA neuronal cells in which expression of SNCA protein has been reduced by use of a gene silencing strategy (e.g., with siRNA or RNAi) or expression of a dominant-negative SNCA sequence variant, or dominant-negative SNCA fragment; or, alternatively, SNCA-modified DA neuronal cells are DA neuronal cells that have been exposed to a SNCA inhibitor (e.g., a small molecule compound, a peptide, or peptidomimetic agent) that inhibits the activity of SNCA, e.g., by inhibiting its binding to target proteins (e.g., a-Synuclein). Alternatively, the SNCA inhibitor may be a peptide or fragment derived from SNCA that acts in trans to inhibit SNCA activity. In some embodiments the SNCA-modified DA neuronal cells are irreversibly SNCA-inhibited, e.g., by genetic modification. In some embodiments the SNCA-modified DA neuronal cells are reversibly SNCA-inhibited so that over time SNCA inhibition in the cells decreases.

CIS inhibition, as used herein, refers to reducing one or more of net SNCA gene expression, net SNCA protein levels.

### Sequence-Specific Reagent

By “sequence-specific reagent” is meant any active molecule that has the ability to specifically recognize a selected polynucleotide sequence at a genomic locus, preferably of at least 9 bp, more preferably of at least 10 bp and even more preferably of at least 12 pb in length, in view of modifying the genomic locus. In some embodiments, the sequence-specific reagent is preferably a sequence-specific nuclease reagent.

### Target Nucleic Acid

As used herein, the term “target nucleic acid” or “target gene” refers to a nucleic acid which is being targeted for alteration, e.g., generation of a precise deletion, by a Cas system, Talen or ZNF system described herein. In certain embodiments, a target nucleic acid comprises one gene. In certain embodiments, a target nucleic acid comprises a portion of one gene. In certain embodiments, a target nucleic acid may comprise one or more genes, e.g., two genes, three genes, four genes, or five genes. In one embodiment, a target nucleic acid comprises two strands: a first strand and a second strand.

### Target Position

“Target position” as used herein, refers to a site on a target nucleic acid (e.g., the chromosome) that is modified by a nuclease. For example, the target position can be modified by a Cas protein molecule-mediated cleavage of the target nucleic acid and template nucleic acid directed modification, e.g., correction, of the target position. In an embodiment, a target position can be a site between two nucleotides, e.g., adjacent nucleotides, on the nucleic acid into which one or more nucleotides is added. The target position may comprise one or more nucleotides that are altered, e.g., corrected, by a template nucleic acid. In an embodiment, the target position is within a “target sequence” (e.g., the sequence to which the gRNA binds). In an embodiment, a target position is upstream or downstream of a target sequence (e.g., the sequence to which the gRNA binds).

### Target Position Region

“Target position region,” as used herein, is a region that comprises a target position. In certain embodiments, the target position is flanked by sequences of the target position region, i.e., the target position is disposed in the target position region such that there are target position region sequences both 5′ and 3′ to the target position. In certain embodiments, the target position region provides sufficient sequences on each side (i.e., 5′ and 3′) of the target position to allow gene conversion of the target position, wherein the gene conversion uses an endogenous sequence homologous with the target position region as a template.

### Target Sequence

“Target sequence” as used herein refers to a nucleic acid sequence comprising a target position of a target gene.

### Targeting Domain

The “targeting domain” (sometimes referred to alternatively as the guide sequence or complementarity region) comprises, consists of, or consists essentially of a nucleic acid sequence that is complementary or partially complementary to a target nucleic acid sequence.

### Template Nucleic Acid

A “template nucleic acid”, as that term is used herein, refers to a nucleic acid sequence which can be used in conjunction with a nuclease to alter the structure of a target position.

### Therapeutic Potential

“Therapeutic potential” reflects the therapeutic activity, as measured through in-vitro experiments.

### Sequence IDs

SEQ ID No 1: >human PARK2

SEQ ID No 2: >human PINK1

SEQ ID No 3: >human LRRK2

SEQ ID No 4: >human c-Rel

SEQ ID No 5: >human ATG7

SEQ ID No 6: >human VMAT2

SEQ ID No 7: >human GBA

SEQ ID No 8: >human SNCA

SEQ ID No 9: >human MAPT

SEQ ID No 10: >human GDNF

SEQ ID No 11: >human PARK2

SEQ ID No 12: >human PINK1

SEQ ID No 13: >human LRRK2

SEQ ID No 14: >human c-Rel

SEQ ID No 15: >human ATG7

SEQ ID No 16: >human VMAT2

SEQ ID No 17: >human GBA

SEQ ID No 18: >human SNCA

SEQ ID No 19: >human MAPT

SEQ ID No 20: >human GDNF

### GDNF Knockin Promoters

SEQ ID No 21: >Monoamine Oxidase B (MAOB)

SEQ ID No 22: >GTP cyclohydrolase 1 (GCH1)

SEQ ID No 23: >Nuclear Receptor Subfamily 4 Group A Member 2 (NR4A2)

SEQ ID No 24: >Mouse Tyrosine Hydroxylase (TH)

SEQ ID No 25: >Solute carrier family 6 member 3 (SLC6A3)

SEQ ID No 26: >Synapsin 1 (SYN1)

SEQ ID No 27: >Mouse alpha-calcium-calmodulin dependent kinase II (Camk2a)

SEQ ID No 28: >Neurofilament medium chain (NEFM) minipromoter

SEQ ID No 29: >Neurofilament light chain (NEFL) minipromoter

SEQ ID No 30: >Neurofilament heavy chain (NEFH) promoter

### GBA Knockin Promoters

SEQ ID No 31: >Mouse Thy-1 cell surface antigen (mTHY1)

SEQ ID No 32: >Rat Neuron specific enolase (rNSE) promoter

SEQ ID No 33: >Dopa Carboxylase (DDC) promoter

SEQ ID No 34: >Catechol-O-methyltransferase (COMT) promoter

SEQ ID No 35: >Glucosylceramidase beta 1 Promoter 1 (GBA P1)

SEQ ID No 36: >Glucosylceramidase beta 1 Promoter 2 (GBA P2)

SEQ ID No 37: >Human eukaryotic translation elongation factor 1 alpha 1 short form (EFS) promoter

SEQ ID No 38: >Human eukaryotic translation elongation factor 1 alpha 1 (EEF1A1) promoter

SEQ ID No 39: >Chicken beta actin (CBA) promoter

SEQ ID No 40: >Cytomegalovirus (CMV) promoter

### Protospacer Adjacent Motif (PAM)

SEQ ID No 41: NNGRRT

SEQ ID NO 42: NNGRRV

### SNCA Gene

The term “SNCA gene” encompasses a SNCA gene and functional fragments and variants thereof. Hemizygous null SNCA genes may be hemizygous null via genetic engineering of the cell or via cell selection.

### SNCA-Modified DA Neuronal Cells

SNCA-modified DA neuronal cell means a DA neuronal cell which is SNCA+/−.

### Vector

By “vector” is meant a nucleic acid molecule capable of transporting another nucleic acid to which it has been linked. A “vector” includes, but is not limited to, a viral vector, a plasmid, an RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available. Viral vectors include retrovirus, adenovirus, parvovirus (e. g. adenoassociated viruses (AAV), coronavirus, negative strand RNA viruses such as ortho myxovirus (e. g., influenza virus), rhabdovirus (e. g., rabies and vesicular stomatitis virus), paramyxovirus (e. g. measles and Sendai), positive strand RNA viruses such as picor-navirus and alphavirus, and double-stranded DNA viruses including adenovirus, herpesvirus (e. g., Herpes Simplex virus types 1 and 2, Epstein-Barr virus, cytomega lovirus), and poxvirus (e. g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

### Compositions

Disclosed herein are engineered cells comprising at least one exogenous nucleic acid sequence, where the exogenous nucleic acid sequence can be inserted into an endogenous gene. In some embodiments, the endogenous gene is a safe harbor. In some embodiments, the safe harbor loci is selected from Table 1. In some embodiments, the knock-in of the exogenous nucleic acid sequence knocks-out the endogenous gene. In some embodiments, the endogenous gene comprises a promotor. In some embodiments, the endogenous gene comprises a ubiquitous promotor. In some embodiments, the endogenous gene comprises a promotor that is only expressed once the engineered cells are transplanted, i.e., not during differentiation in vitro. In some embodiments, the endogenous gene comprises a promotor that is only expressed once the engineered cells are engrafted, i.e., not during differentiation in vivo.

DA Neuronal Cells with Delayed Expression of GDNF

Disclosed herein are engineered DA neuronal cells which when administered to a patient exhibit delayed expression of the exogenous GDNF gene in the engineered DA neuronal cells, i.e., the exogenous GDNF gene is behind an endogenous promotor that is not expressed upon transplantation. In some embodiments, the exogenous GDNF gene is behind an endogenous promotor that is inactive during pluripotent cell differentiation to DA neuronal cells in vitro. In some embodiments, the exogenous GDNF gene is behind an endogenous promotor that is inactive during DA neuronal cell differentiation to Neuronal Mature Cells in vivo. In some embodiments, the exogenous GDNF gene is behind an GDNF knock-in promoter selected from the group comprising or consisting of SEQ ID NOS: 21-30. In some embodiments, the exogenous GDNF gene is behind an GBA knock-in promoter selected from the group comprising or consisting of SEQ ID NOS: 31-40 or 43. In some embodiments, the delayed expression of the exogenous GDNF gene is delayed until graft function has been established. In some embodiments, the delayed expression of the exogenous GDNF gene is delayed until the transplanted engineered DA neuronal cells are differentiated to a Neuronal Mature Cell Type.

DA Neuronal Cells which Immediately Express GDB and are Hemizygous Null for SNCA

Disclosed herein are engineered DA neuronal cells which when administered to a patient, exhibit expression of the exogenous GBA gene and SNCA+/− (exogenous or endogenous) gene in the engineered DA neuronal cells as soon as the engineered DA neuronal cells are transplanted, i.e., the exogenous GBA gene is behind an endogenous promotor that is expressed upon transplantation. In some embodiments, the expression of the exogenous GBA gene occurs before the engineered DA neuronal cells have graft function. In some embodiments, the expression of the exogenous GBA gene occurs before the transplanted engineered DA neuronal cells are differentiated to a Neuronal Mature Cell Type. In some embodiments, the expression of the exogenous GBA gene occurs before and after the transplanted engineered DA neuronal cells are differentiated to a Neuronal Mature Cell Type. In some embodiments, the GDNF coding sequences are inserted under the transcriptional control of endogenous gene promoters that are not expressed until the administered engineered DA neuronal cells have differentiated to mature cell types in vivo. In some embodiments, the exogenous GBA gene is behind an GDNF knock-in promoter selected from the group comprising or consisting of SEQ ID NOS: 21-30. In some embodiments, the exogenous GBA gene is behind an GBA knock-in promoter selected from the group comprising or consisting of SEQ ID NOS: 31-40 or 43.

In some embodiments, the engineered DA neuronal cells that have been engineered to include an exogenous GBA gene are hemizygous null for SNCA and/or MAPT. In some embodiments, the engineered DA neuronal cells are engineered to be hemizygous null for SNCA and/or MAPT and in some embodiments they are not engineered to be hemizygous null for SNCA and/or MAPT.

In some embodiments, the engineered DA neuronal cells that have been engineered to include an exogenous GBA gene and are hemizygous null for SNCA and/or MAPT have a changed GBA protein expression ratio compared to alpha-synuclein and/or MAPT protein. In some embodiments, the GBA: alpha-synuclein protein expression ratio in the administered engineered DA neuronal cells is higher. In some embodiments, the GBA: alpha-synuclein protein expression ratio in the administered engineered DA neuronal cells is lower.

### Engineered Pluripotent Cell Populations

Disclosed is a population of engineered pluripotent cells, wherein the engineered pluripotent cells comprise an exogenous knock-in PARK2, PINK1, DJ-1, LRRK2, c-Rel, ATG7, VMAT2, GBA, SNCA+/+, SNCA+/−, SNCA−/−, and/or GDNF gene or functional fragment thereof. In some embodiments, the exogenous knock-in gene is selected from the group comprising or consisting of Seq ID Nos: 1-10.

Disclosed is a population of engineered pluripotent cells, wherein the engineered pluripotent cells comprise an exogenous PARK2, PINK1, DJ-1, LRRK2, c-Rel, ATG7, VMAT2, GBA, SNCA+/+, SNCA+/−, SNCA−/−, and/or GDNF gene or functional fragment thereof in a genomic safe harbor. In some embodiments, the exogenous gene is selected from the group comprising or consisting of Seq ID Nos: 1-10. In some embodiments, the genomic safe harbor is selected from the group comprising or consisting of safe harbors in Table 1.

Disclosed is a population of engineered pluripotent cells, wherein the engineered DA neuronal cells comprise an exogenous PARK2, PINK1, DJ-1, LRRK2, c-Rel, ATG7, VMAT2, GBA, SNCA+/+, SNCA+/−, SNCA−/−, and/or GDNF gene or functional fragment thereof under control of an endogenous gene promotor. In some embodiments, the endogenous gene promotor is selected from the group comprising or consisting of SEQ ID NOS: 21-30. In some embodiments, the endogenous gene promotor is selected from the group comprising or consisting of SEQ ID NOS: 31-40 or 43.

Disclosed is a population of engineered pluripotent cells, wherein the engineered pluripotent cells comprise an exogenous GDNF gene or functional fragment thereof. Disclosed is a population of engineered pluripotent cells, wherein the engineered pluripotent cells comprise an exogenous SEQ ID NO: 10 or functional fragment thereof.

Disclosed is a population of engineered pluripotent cells, wherein the engineered pluripotent cells comprise an exogenous PARK2, PINK1, DJ-1, LRRK2, c-Rel, ATG7, VMAT2, GBA, hemizygous SNCA and/or GDNF gene and an endogenous knock-out PARK2, PINK1, DJ-1, LRRK2, c-Rel, ATG7, VMAT2, GBA, SNCA and/or GDNF gene.

Disclosed is a population of engineered pluripotent cells, wherein the engineered pluripotent cells comprise an exogenous GDNF gene, SEQ ID NO: 10, or functional fragment thereof under control of a promotor that is not expressed until graft function in vivo has been established. Disclosed is a population of engineered pluripotent cells, wherein the engineered pluripotent cells comprise an exogenous GDNF gene or functional fragment thereof under control of a promotor that is not expressed until the transplanted population of engineered pluripotent cells differentiate in vivo to a Neuronal Mature Cell Type. Disclosed is a population of engineered pluripotent cells, wherein the engineered pluripotent cells comprise an exogenous GDNF gene or functional fragment thereof under control of one of SEQ ID NOS: 21-40 or 43.

Disclosed is a population of engineered pluripotent cells, wherein the engineered pluripotent cells comprise an exogenous knock-in GBA gene or functional fragment thereof. Disclosed is a population of engineered pluripotent cells, wherein the engineered pluripotent cells comprise an exogenous knock-in SEQ ID NO: 7 or functional fragment thereof.

Disclosed is a population of engineered pluripotent cells, wherein the engineered pluripotent cells comprise an exogenous GBA gene or functional fragment thereof under control of a promotor that is expressed upon cell transplantation in vivo. Disclosed is a population of engineered pluripotent cells, wherein the engineered pluripotent cells comprise an exogenous GBA gene or functional fragment thereof under control of one of SEQ ID NOS: 21-40 or 43.

Disclosed is an engineered pluripotent cell, wherein the engineered pluripotent cell comprises an exogenous GDNF gene or functional fragment thereof under control of a promotor that is not expressed until graft function in vivo has been established and further comprises an exogenous GBA gene or functional fragment thereof under control of a promotor that is expressed upon cell transplantation.

Disclosed is a population of engineered pluripotent cells, wherein the engineered pluripotent cells comprise (i) an exogenous PARK2, PINK1, DJ-1, LRRK2, c-Rel, ATG7, VMAT2, GBA, and/or GDNF gene and (ii) is hemizygous null for the SNCA and/or MAPT gene.

Disclosed is a population of engineered pluripotent cells, wherein the engineered pluripotent cells comprise (i) an exogenous GDNF gene or functional fragment thereof and (ii) is hemizygous null for the SNCA and/or MAPT gene.

Disclosed is a population of engineered pluripotent cells, wherein the engineered pluripotent cells comprise a disrupted SNCA and/or MAPT gene.

Disclosed is a population of engineered pluripotent cells, wherein the engineered pluripotent cells comprise (i) an exogenous GBA gene or functional fragment thereof and (ii) a hemizygous null SNCA and/or MAPT gene.

Disclosed is a population of engineered pluripotent cells, wherein the engineered pluripotent cells comprise (i) an exogenous GBA gene or functional fragment thereof and (ii) a hemizygous null SNCA gene.

In some embodiments, the population of engineered pluripotent cells do not express a detectable level of alpha-synuclein and/or MAPT protein in vivo and/or in vitro.

In some embodiments, the population of engineered pluripotent cells express less alpha-synuclein and/or MAPT protein compared to a wildtype pluripotent cell in vivo and/or in vitro.

In some embodiments, the population of engineered pluripotent cells express less alpha-synuclein and/or MAPT protein compared to an engineered pluripotent cell without an edit to the SNCA and/or MAPT gene in vivo and/or in vitro.

Disclosed is a population of engineered pluripotent cells, wherein the engineered pluripotent cells comprise an exogenous SNCA gene.

Disclosed is a population of engineered pluripotent cells, wherein the engineered pluripotent cells comprise an exogenous SNCA+/− gene.

Disclosed is a population of engineered pluripotent cells, wherein the engineered pluripotent cells comprise an exogenous SNCA gene wherein the SNCA gene is at a genomic safe harbor. In some embodiments, the SNCA gene is SNCA+/+, SNCA+/− or SNCA−/−. In some embodiments, the genomic safe harbor is selected from the group comprising or consisting of safe harbors in Table 1.

In some embodiments, the population of engineered pluripotent cells are human.

Disclosed is a population of engineered pluripotent cells, wherein the engineered pluripotent cells comprise an exogenous hemizygous null SNCA gene. Disclosed is a population of engineered pluripotent cells, wherein the engineered pluripotent cells comprise an exogenous hemizygous null SNCA gene under control of a promotor that is expressed upon cell transplantation. Disclosed is a population of engineered pluripotent cells, wherein the engineered pluripotent cells comprise an exogenous hemizygous null SNCA gene under control of a promotor that is not expressed until graft function in vivo has been established.

In some embodiments, the population of engineered pluripotent cells comprising a hemizygous null SNCA gene comprise a disrupted SNCA gene. In some embodiments, the disrupted SNCA gene comprise the nucleotide sequence of SEQ ID NO: 8.

### Engineered DA Neuronal Cell Populations

Disclosed is a population of engineered DA neuronal cells, wherein the engineered DA neuronal cells comprise an exogenous knock-in PARK2, PINK1, DJ-1, LRRK2, c-Rel, ATG7, VMAT2, GBA, SNCA+/+, SNCA+/−, SNCA−/−, and/or GDNF gene or functional fragment thereof. Disclosed is a population of engineered DA neuronal cells, wherein the engineered DA neuronal cells comprise an exogenous knock-in gene selected from the group comprising or consisting of SEQ ID NO: 1-10 or functional fragment thereof.

Disclosed is a population of engineered DA neuronal cells, wherein the engineered DA neuronal cells comprise an exogenous PARK2, PINK1, DJ-1, LRRK2, c-Rel, ATG7, VMAT2, GBA, SNCA+/+, SNCA+/−, SNCA−/−, and/or GDNF gene or functional fragment thereof in a genomic safe harbor. In some embodiments, the genomic safe harbor is selected from the group comprising or consisting of safe harbors in Table 1.

Disclosed is a population of engineered DA neuronal cells, wherein the engineered DA neuronal cells comprise an exogenous PARK2, PINK1, DJ-1, LRRK2, c-Rel, ATG7, VMAT2, GBA, SNCA+/+, SNCA+/−, SNCA−/−, and/or GDNF gene or functional fragment thereof under control of an endogenous gene promotor. In some embodiments, endogenous gene promotor is one of SEQ ID NOS: 21-40 or 43.

Disclosed is a population of engineered DA neuronal cells, wherein the engineered DA neuronal cells comprise an exogenous GDNF gene or functional fragment thereof.

Disclosed is a population of engineered pluripotent cells, wherein the engineered pluripotent cells comprise an exogenous PARK2, PINK1, DJ-1, LRRK2, c-Rel, ATG7, VMAT2, GBA, hemizygous SNCA and/or GDNF gene and an endogenous knock-out PARK2, PINK1, DJ-1, LRRK2, c-Rel, ATG7, VMAT2, GBA, SNCA and/or GDNF gene.

Disclosed is a population of engineered DA neuronal cells, wherein the engineered DA neuronal cells comprise an exogenous GDNF gene or functional fragment thereof under control of a promotor that is not expressed until a portion of the transplanted population of DA neuronal cells have engrafted in vivo. Disclosed is a population of engineered DA neuronal cells, wherein the engineered DA neuronal cells comprise an exogenous GDNF gene or functional fragment thereof under control of a promotor that is not expressed until the transplanted population of engineered DA neuronal cells differentiate in vivo to a Neuronal Mature Cell Type.

Disclosed is a population of engineered DA neuronal cells, wherein the engineered DA neuronal cells comprise an exogenous GBA gene or functional fragment thereof. Disclosed is a population of engineered DA neuronal cells, wherein the engineered DA neuronal cells comprise an exogenous SEQ ID NO:7 or functional fragment thereof.

Disclosed is a population of engineered DA neuronal cells, wherein the engineered DA neuronal cells comprise an exogenous GBA gene or functional fragment thereof under control of a promotor that is expressed upon cell transplantation. In some embodiments, endogenous gene promotor is one of SEQ ID NOS: 21-40 or 43.

Disclosed is an engineered DA neuronal cell, wherein the engineered DA neuronal cell comprises an exogenous GDNF gene or functional fragment thereof under control of a promotor that is not expressed until graft function in vivo has been established and further comprises an exogenous GBA gene or functional fragment thereof under control of a promotor that is expressed upon cell transplantation.

Disclosed is a population of engineered DA neuronal cells, wherein the engineered DA neuronal cells comprise (i) an exogenous PARK2, PINK1, DJ-1, LRRK2, c-Rel, ATG7, VMAT2, GBA, and/or GDNF gene and (ii) is hemizygous null for the SNCA and/or MAPT gene.

Disclosed is a population of engineered DA neuronal cells, wherein the engineered DA neuronal cells comprise (i) an exogenous GDNF gene or functional fragment thereof and (ii) is hemizygous null SNCA and/or MAPT gene.

Disclosed is a population of engineered DA neuronal cells, wherein the engineered DA neuronal cells comprise a disrupted SNCA and/or MAPT gene.

Disclosed is a population of engineered DA neuronal cells, wherein the engineered DA neuronal cells comprise (i) an exogenous GBA gene or functional fragment thereof and (ii) a hemizygous null SNCA and/or MAPT gene.

Disclosed is a population of engineered DA neuronal cells, wherein the engineered DA neuronal cells comprise (i) an exogenous GBA gene or functional fragment thereof and (ii) a hemizygous null SNCA gene.

In some embodiments, the population of engineered DA neuronal cells do not express a detectable level of alpha-synuclein and/or MAPT protein in vivo and/or in vitro.

In some embodiments, the population of engineered DA neuronal cells express less alpha-synuclein and/or MAPT protein compared to a wildtype DA neuronal cell.

In some embodiments, the population of engineered DA neuronal cells express less alpha-synuclein and/or MAPT protein compared to an engineered DA neuronal cell without an edit to the SNCA and/or MAPT gene in vivo and/or in vitro.

Disclosed is a population of engineered pluripotent cells, wherein the engineered pluripotent cells comprise an exogenous SNCA gene wherein the SNCA gene is at a genomic safe harbor. In some embodiments, the SNCA gene is SNCA+/+, SNCA+/− or SNCA−/−. In some embodiments, the genomic safe harbor is selected from the group comprising or consisting of safe harbors in Table 1.

In some embodiments, the population of engineered DA neuronal cells are human. In some embodiments, the population of engineered DA neuronal cells are derived from pluripotent cells.

Disclosed is a population of engineered DA neuronal cells, wherein the engineered DA neuronal cells comprise an exogenous hemizygous null SNCA gene. Disclosed is a population of engineered DA neuronal cells, wherein the engineered DA neuronal cells comprise an exogenous hemizygous null SNCA gene under control of a promotor that is expressed upon cell transplantation. Disclosed is a population of engineered DA neuronal cells, wherein the engineered DA neuronal cells comprise an exogenous hemizygous null SNCA gene under control of a promotor that is not expressed until graft function in vivo has been established.

In some embodiments, the population of engineered DA neuronal cells comprising a hemizygous null SNCA gene comprise a disrupted SNCA gene. In some embodiments, the disrupted SNCA gene comprises the nucleotide sequence of SEQ ID NO: 8.

In some embodiments, at least 5-95% of the engineered DA neuronal cells do not express a detectable level of alpha-synuclein protein. In some embodiments, at least 20-75% of the engineered DA neuronal cells do not express a detectable level of alpha-synuclein protein. In some embodiments, at least 30% of the engineered DA neuronal cells do not express a detectable level of alpha-synuclein protein. In some embodiments, at least 50% of the engineered DA neuronal cells do not express a detectable level of alpha-synuclein protein. In some embodiments, at least 70% of the engineered DA neuronal cells do not express a detectable level of alpha-synuclein protein. In some embodiments, at least 80% of the engineered DA neuronal cells do not express a detectable level of alpha-synuclein protein. In some embodiments, at least 90% of the engineered DA neuronal cells do not express a detectable level of alpha-synuclein protein.

In some embodiments, the GBA gene is under control of a promotor that is expressed as soon as the engineered cells are transplanted into a patient. In some embodiments, the SNCA gene is under control of a promotor that is expressed as soon as the engineered cells are transplanted into a patient, but the SNCA gene promoter is expressed to a lesser extent compared to the GBA gene promotor.

Disclosed is a DA neuronal cell that promotes expression of GDNF by site directed gene editing. Disclosed is a DA neuronal cell that promotes expression of GBA by site directed gene editing. Disclosed is a DA neuronal cell that promotes expression of GBA and regulates expression of SNCA by site directed gene editing. Disclosed is a DA neuronal cell that regulates expression of SNCA by site directed gene editing. Disclosed is a DA neuronal cell that promotes expression of GBA by site directed gene editing and is hemizygous null for SNCA. Disclosed is a DA neuronal cell that promotes expression of GBA and is hemizygous null for SNCA by site directed gene editing.

Disclosed is a DA neuronal cell that promotes expression of GDNF and GBA and is hemizygous null for SNCA by site directed gene editing. In some embodiments, GDNF expression is delayed until after graft formation has been established. In some embodiments, the GBA gene is expressed as soon as the engineered DA neuronal cell is transplanted. In some embodiments, the GBA gene is expressed and the SNCA gene expression is regulated as soon as the engineered DA neuronal cell is transplanted wherein the SNCA protein expression is less than GBA protein expression. In some embodiments, GDNF expression is promoted after graft formation has been established and The GBA gene is expressed as soon as the engineered DA neuronal cell is transplanted.

In some embodiments, the endogenous promoter is a constitutive promoter. In some embodiments, the endogenous promoter is not a constitutive promoter. In some embodiments, the endogenous promoter is a conditional promoter. In some embodiments, the endogenous promoter is not a conditional promoter. In some embodiments, endogenous gene promotor is one of SEQ ID NOS: 21-40 or 43.

In some embodiments, the exogenous sequence is integrated at an endogenous locus, which is constitutively expressed/constitutively transcribed.

In some embodiments, it can be advantageous to inactivate the endogenous SNCA coding sequence, while having the integrated exogenous sequence encoding the GBA, SNCA+/− or GDNF gene or functional fragment thereof transcribed at this locus.

In some embodiments, the SNCA gene expression in the transplanted engineered DA neuronal cell is prevented or reduced compared to wildtype.

The embodiments disclosed herein can be further understood by the following numbered paragraphs.

Paragraph 1. A DA neuronal cell population having the following features:
an exogenous sequence, which has been integrated under transcriptional control of an endogenous gene promoter.

Paragraph 2. An engineered DA neuronal cell according to paragraph 1, which has a genotype [SNCA]^{neg} [GBA]^{pos}

Paragraph 3. An engineered DA neuronal cell according to paragraph 1, which has a genotype [SNCA]^{neg} [GBA]^{pos} [GDNF]^{pos}

Paragraph 4. An engineered DA neuronal cell according to paragraph 1, which has a genotype [SNCA]^{+/-}[GBA]^{pos} [GDNF]^{pos}

Paragraph 5. A DA neuronal cell population having the following features:
an exogenous GDNF, GBA and/or SNCA sequence, which has been integrated under transcriptional control of an endogenous gene promoter.

Paragraph 6. A therapeutically effective population of DA neuronal cells, comprising at least 30%, preferably at least 50%, more preferably at least 80% of engineered DA Neuronal cells according to any one of paragraphs 1 to 5.

### Types of Exogenous Sequences

In some embodiments, the exogenous sequence added to a cell to be engineered (pluripotent or DA neuronal cell) is a polynucleic acid. In some embodiments, the polynucleic acid is DNA, or RNA. In some embodiments, the RNA can be mRNA. Also disclosed herein are exogenous polynucleic acid sequences comprising at least one exogenous GDNF, GBA or SNCA sequence. The exogenous polynucleic acid sequence can be complementary to a genomic sequence that can be a partial sequence or a full sequence. In some embodiments, the exogenous polynucleic acid sequence comprises a coding sequence. In some embodiments, the exogenous polynucleic acid sequence comprises a non-coding sequence. In some embodiments, the exogenous polynucleic acid sequence comprises one or more genes.

In some embodiments, the polynucleic acid can be a plasmid vector. The plasmid vector can comprise a promoter. In some embodiments, the promotor can be constitutive. In some embodiments, the promoter can be inducible. In some embodiments, the promoter is Synapsin 1, DAT, VMAT, TH, AADC, tamoxifen inducible promoter, RU486 inducible promoter, or other promotor that allows for temporal or small molecule control. In some embodiments, the promoter can be adjacent to the exogenous GDNF, GBA and/or SNCA sequence. In some embodiments, the plasmid vector further comprises a splicing acceptor. In some embodiments, the splicing acceptor can be adjacent to the exogenous GDNF, GBA and/or SNCA sequence.

In some embodiments, the plasmid vector further comprises a translation initiation codon such as an “ATG” sequence. The translation initiation codon sequence can be adjacent to the GDNF, GBA and/or SNCA sequence. In some embodiments, the GDNF, GBA and/or SNCA sequence can be within a multicistronic vector. In some embodiments, the polynucleic acid comprises an exogenous promotor, an endogenous promoter via splicing, and/or an endogenous promoter via in frame translation.

In some embodiments, the plasmid can be modified. The modification can comprise demethylation, addition of CpG methylation, removal of bacterial methylation, and addition of mammalian methylation.

The embodiments can be further understood by the following numbered paragraphs.

Paragraph 1. An engineered DA neuronal cell comprising an exogenous PARK2, PINK1, DJ-1, LRRK2, SNCA, c-Rel, ATG7, VMAT2, GBA, SNCA or GDNF gene or functional fragment thereof.

Paragraph 2. A composition comprising the engineered DA neuronal cell of paragraph 1.

Paragraph 3. A genome editing system comprising:
(a) a gRNA molecule;
(b) a Cas molecule configured to alter PARK2, PINK1, DJ-1, LRRK2, SNCA, c-Rel, ATG7, VMAT2, GBA, SNCA and/or GDNF gene.

Paragraph 4. A method of inhibiting degeneration or death of a dopaminergic neuron comprising:
contacting a neuron with the engineered DA neuronal cell of paragraph 1;
wherein following contact with the engineered DA neuronal cell, the neuron expresses the PARK2, PINK1, DJ-1, LRRK2, c-Rel, ATG7, VMAT2, GDNF, SNCA and/or GBA protein,
optionally wherein the neuron comprises a mutation in a PARK2, PINK1, DJ-1, LRRK2, c-Rel, ATG7, VMAT2, GDNF, SNCA and/or GBA gene.

Paragraph 5. The method of paragraph 4, wherein the neuron expresses a reduced amount of alpha-synuclein following contact with the engineered DA neuronal cell.

Paragraph 6. The method of any of paragraphs 4 to 5, wherein the neuron produces and/or releases an increased amount of dopamine following contact with the engineered DA neuronal cell.

Paragraph 7. The method of any of paragraphs 4 to 6, wherein the neuron expresses a lower amount of alpha-synuclein as compared to an amount of alpha-synuclein expressed in a neuron not contacted with the engineered DA neuronal cell, optionally wherein the lower amount is at least 5%, at least 10%, at least 20%, at least 30%, at least 40%, at least 50%, at least 60%, at least 70%, or at least 80% lower than the amount expressed in the neuron not contacted with the engineered DA neuronal cell.

Paragraph 8. The method of any of paragraphs 4 to 7, wherein the neuron produces and/or releases an increased amount of dopamine as compared to an amount of dopamine produced and/or released by a neuron not contacted with the engineered DA neuronal cell, optionally wherein the increase amount is at least 5%, at least 10%, at least 20%, at least 30%, at least 40%, at least 50%, at least 60%, at least 70%, at least 80%, at least 90%, at least two-fold, at least three-fold, at least four-fold, at least five-fold, or at least 10-fold greater than the amount produced and/or released by the neuron not contacted with the engineered DA neuronal cell.

Paragraph 9. The method of any of paragraphs 4 to 8, wherein the neuron undergoes an increased amount of autophagy as compared to an amount of autophagy undergone by a neuron not contacted with the engineered DA neuronal cell, optionally wherein the increased amount is at least 5%, at least 10%, at least 20%, at least 30%, at least 40%, at least 50%, at least 60%, at least 70%, at least 80%, at least 90%, at least two-fold, at least three-fold, at least four-fold, at least five-fold, or at least 10-fold greater than the amount undergone by the neuron not contacted with the engineered DA neuronal cell.

Paragraph 10. The method of any of paragraphs 4 to 9, wherein the neuron is a primary tyrosine hydroxylase positive neuron.

Paragraph 11. A method of treating or inhibiting onset of a Parkinson's Disease (PD) in a subject suffering from or at risk of the PD, comprising:
administering an engineered DA neuronal cell of paragraph 1 to the subject;
wherein administration of the engineered DA neuronal cell treats or inhibits onset of the Parkinson's Disease in the subject.

Paragraph 12. The method of claim 11, wherein the subject is an adult or child.

Paragraph 13. The method of any of paragraphs 11 to 12, wherein the number of dopaminergic neurons in the subject after the administering step is greater than the number of dopaminergic neurons in the subject before the administering step.

Paragraph 14. The method of any of paragraphs 11 to 13, wherein the level of dopamine in the subject after the administering step is greater than the level of dopamine in the subject before the administering step.

Paragraph 15. The method of any of paragraphs 11 to 14, wherein the number of dopaminergic neurons in a subject treated by the method is increased compared to the number of dopaminergic neurons in a subject not so treated.

Paragraph 16. The method of any of paragraphs 11 to 15, wherein the level of dopamine of a subject treated by the method is increased compared to the level of dopamine in a subject not so treated.

Paragraph 17. The method of any one of paragraphs 11 to 16, wherein the level of dopamine in the substantia nigra of a subject treated by method is increased compared to the level of dopamine in the substantia nigra of a subject not so treated.

Paragraph 18. The method of any of paragraphs 11 to 17, wherein the level of GDNF and/or GBA in the subject's CSF after the administering step is greater than the level of GDNF and/or GBA in the subject's CSF before the administering step.

Paragraph 19. The method of any of paragraphs 11 to 18, wherein the Unified Parkinson's Disease Rating Scale (UPDRS) score of the subject before the administering step is improved compared to the UPDRS score of the subject before the administering step.

Paragraph 20. The method of any of paragraphs 11 to 19, wherein the level of PRKN in the CSF of a subject treated by the method is increased compared to the level of PRKN in the CSF of a subject not so treated.

Paragraph 21. The method of any of paragraphs 11 to 19, wherein the UPDRS score of a subject treated by the method is improved compared to the UPDRS score of a subject not so treated.

Paragraph 22. The method of any of paragraphs 11 to 21, wherein the subject's neurons express a reduced amount of alpha-synuclein and/or comprises a reduced amount of Lewy bodies following contact with the engineered DA neuronal cell.

Paragraph 23. A DA neuronal cell population comprising a recombinant gene vector comprising a polynucleotide encoding a wild-type PINK1, LRRK2, SNCA, c-Rel, ATG7, VMAT2, GDNF, or GBA gene, a Ubiquitin-like modifier-activating enzyme (ATG7) gene, Synaptic vesicular amine transporter (VMAT2) gene, or glucocerebrosidase (GBA) gene, GDNF gene, SNCA+/− gene or a functional variant or fragment thereof; wherein the polynucleotide is operatively linked to a eukaryotically active promoter.

### Types of Cells to be Engineered

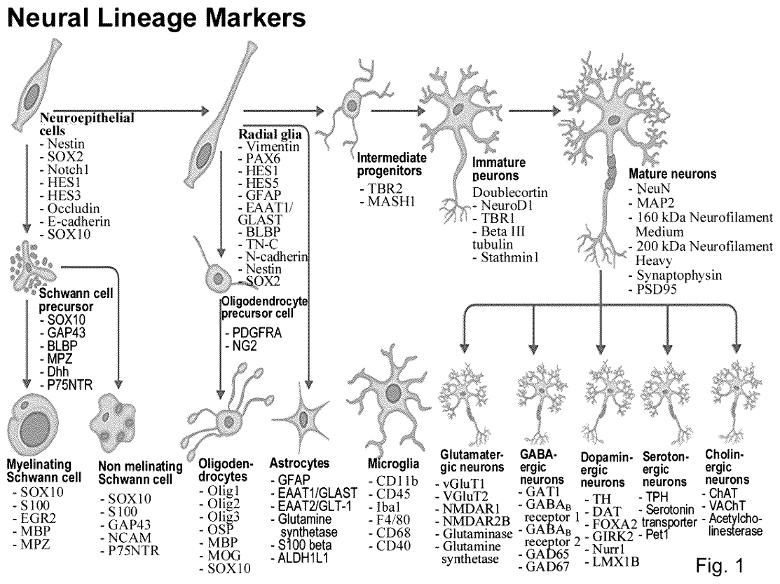

In some embodiments, the cell to be engineered can be a primary cell. The primary cell can be a neural cell, a pluripotent cell, a progenitor cell or combinations thereof. In some embodiments, a progenitor cell is a DA neuronal cell (see markers for different neural lineage cell types in FIG. 1). Examples of pluripotent cells include stem cells, such as cord blood stem cells, progenitor cells, bone marrow stem cells, embryonic stem cells (ESC) and induced pluripotent stem cells (IPS). The engineered cell can be a human cell. The engineered cell can be an animal (non-human cell). The engineered cell can be expanded ex vivo. The engineered cell can be expanded in vitro. The engineered cell can be expanded in vivo. The engineered cell can be autologous to a subject in need thereof. The engineered cell can be non-autologous to a subject in need thereof. The engineered cell can be in vitro. The engineered cell can be in vivo. The engineered cell can be a part of a combination therapy to treat Parkinson's disease and other and secondary Parkinsonian disorders in a subject in need thereof.

### Endogenous Gene is Disrupted by the Exogenous Gene Sequence

In some embodiments, the endogenous gene is disrupted by the exogenous GDNF, GBA and/or SNCA gene sequence. In some embodiments, the endogenous gene is GDNF, GBA and/or SNCA. In some embodiments, the endogenous gene is a wildtype GDNF, GBA and/or SNCA gene. In some embodiments, the endogenous gene is a mutated GDNF, GBA and/or SNCA gene. In some embodiments, the endogenous gene is a mutated PARK2, PINK1, DJ-1, LRRK2, c-Rel, ATG7, VMAT2, GBA, SNCA+/− and/or GDNF gene. In some embodiments, the endogenous gene can be under control of promoter. In some embodiments, the exogenous gene can be under control of promoter selected from the group comprising Synapsin 1, DAT, VMAT, TH, AADC, tamoxifen inducible promoter, RU486 inducible promoter, and/or other promotors that allows for temporal or small molecule control. In some embodiments, the endogenous gene can be under control of promoter. In some embodiments, the exogenous gene can be under control of promoter selected from the group comprising Synapsin 1, DAT, VMAT, TH, AADC, tamoxifen inducible promoter, or a RU486 inducible promoter.

Engineered with More than One Exogenous Gene Sequence

In some embodiments, the engineered cell can comprise a single GDNF, GBA and/or SNCA exogenous sequence. In some embodiments, the engineered cell can comprise multiple GDNF, GBA and/or SNCA exogenous sequences. In some embodiments, the engineered cell can comprise a more than one of GDNF, GBA or SNCA exogenous sequences. The GDNF exogenous sequence can comprise an engineered GDNF exogenous sequence. The GDNF exogenous sequence can produce a functional GDNF protein. The GBA exogenous sequence can comprise an engineered GBA exogenous sequence. The GBA exogenous sequence can produce a functional GBA protein. The SNCA exogenous sequence can comprise an engineered SNCA exogenous sequence. The SNCA exogenous sequence can produce a functional SNCA protein.

### Protospacer Adjacent Motif Sequence

Disclosed herein can be an engineered cell comprising at least one exogenous GDNF. GBA and/or SNCA gene or functional fragment thereof that can be adjacent to a protospacer adjacent motif sequence of genomic DNA. In some embodiments, a protospacer adjacent motif sequence (PAM) can be recognized by a CRISPR endonuclease. An endonuclease can be a Cas protein. A Cas protein can be selected from a list comprising Cas1, Cas1B, Cas2, Cas3, Cas4, Cas5, Cas6, Cas7, Cas8, Cas9 (also known as Csn1 or Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof or modified versions thereof. In some embodiments, a CRISPR endonuclease can be Cas9. A Cas9 disclosed herein can recognize a PAM sequence that may be 5′ NGG 3′.

Disclosed herein can be at least one exogenous GDNF that can disrupt at least one gene. The disrupted gene can be any gene as described in SEQ ID NOS 1-10. Disclosed herein can be at least one exogenous GBA that can disrupt at least one gene. The disrupted gene can be any gene as described in SEQ ID NOS 1-10. Disclosed herein can be at least one exogenous SNCA that can disrupt at least one gene. The disrupted gene can be any gene as described in SEQ ID NOS 1-10. In some embodiments, the disrupted can comprise a protospacer. A protospacer can be disrupted by insertion of an exogenous gene sequence. A GDNF gene sequence can produce a functional GDNF. A GBA gene sequence can produce a functional GBA. A SNCA gene sequence can produce a functional SNCA.

Disclosed herein can be a composition comprising at least one guide RNA that binds to an endogenous GDNF, GBA or SNCA gene and a secondary guide RNA that binds to an endogenous gene selected from the group comprising or consisting of any gene as described in SEQ ID NOS 1-10.

Disclosed herein can be an engineered cell with a disruption in an endogenous PARK2, PINK1, DJ-1, LRRK2, c-Rel, ATG7, VMAT2, GBA, SNCA or GDNF gene sequence and at least one secondary disruption in a second endogenous gene which is not a PARK2, PINK1, DJ-1, LRRK2, c-Rel, ATG7, VMAT2, GBA, SNCA or GDNF.

Disclosed herein is a genetically modified cell derived from a human subject; a polynucleic acid-targeting polynucleic acid, wherein a polynucleic acid-targeting polynucleic acid is engineered to hybridize to a specific region of a target gene in a genome of the cell; a nuclease, wherein a nuclease is capable of associating with a polynucleic acid-targeting polynucleic acid to form a nucleoprotein complex, wherein a nucleoprotein complex can be capable of generating a targeted double-strand break in a target gene in a genome of the cell; and a target polynucleic acid, wherein a target polynucleic acid can be genomic DNA comprising a double-strand break in a target gene, wherein a double-strand break in a target gene results in disruption of a target gene function and wherein a genetically modified cell can be capable of being expanded to generate a clonal population of cell with altered function of a target gene and wherein a clonal population of the modified cell are suitable for administration to a human in need thereof.

### Gene Editing Systems

Genome editing systems disclosed herein include at least two components adapted from naturally occurring CRISPR systems: a guide RNA (gRNA) and an RNA-guided nuclease. These two components form a complex that is capable of associating with a specific nucleic acid sequence in a cell and editing the DNA in or around that nucleic acid sequence, for instance by making one or more of a single-strand break (an SSB or nick), a double-strand break (a DSB) and/or a point mutation. Genome editing systems may comprise, in various embodiments, (a) one or more Cas9/gRNA complexes, and (b) separate Cas9 molecules and gRNAs that are capable of associating in a cell to form one or more Cas9/gRNA complexes. A genome editing system according to the present disclosure may be encoded by one or more nucleotides (e.g. RNA, DNA) comprising coding sequences for Cas9 and/or gRNAs that can associate to form a Cas9/gRNA complex, and the one or more nucleotides encoding the gene editing system may be carried by a vector as described herein.

In certain embodiments, the genome editing system targets neuronal genes selected from the group comprising or consisting of PARK2, PINK1, DJ-1, LRRK2, c-Rel, ATG7, VMAT2, GBA, SNCA or GDNF. In certain embodiments, the genome editing system targets neuronal genes selected from the group comprising or consisting of SEQ ID NOS: 1-10.

The disclosure is directed to a guide RNA (gRNA) comprising a targeting domain comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10 (or portion thereof) or is configured to associate with a target nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 or a complement or RNA equivalent thereof. In certain embodiments, the targeting domain is 3 to 100, 5 to 100, 10 to 100, or 20 to 100 nucleotides in length, and in certain of these embodiments the targeting domain is 3 to 15, 3 to 20, 5 to 20, 10 to 20, 15 to 20, 5 to 50, 10 to 50, or 20 to 50 nucleotides in length. In certain embodiments, the targeting domain is 6, 7, 8, 9, 10, 11, 12, 13, 14, 15, 16, 17, 18, 19, 20, 21, 22, 23, 24, 25, or 26 nucleotides in length. Methods for selecting targeting domains are known in the art (see, e.g., Fu et al. (2014) NAT. BIOTECHNOL. 32 (3): 279-84; Sternberg et al. (2014) NATURE 507 (7490): 62-67, the entire contents of each of which are expressly incorporated by reference herein). Since the targeting domain is part of a gRNA molecule, it comprises the base uracil (U) rather than thymine (T); conversely, any DNA molecule encoding the gRNA molecule will comprise thymine rather than uracil. In a targeting domain/target domain pair, the uracil bases in the targeting domain will pair with the adenine bases in the target domain. In certain embodiments, the degree of complementarity between the targeting domain and target domain is sufficient to allow targeting of a Cas9 molecule to the target nucleic acid. Thus a targeting domain or gRNA described herein includes a portion of Seq ID NOs: 1-10 which is 3 to 100 nucleotides in length and comprises the base uracil (U) rather than thymine (T). In certain embodiments, the targeting domain is fully complementary to the target domain. In certain embodiments, the targeting domain is fully complementary to Seq ID NOs: 1-10 and comprises the base uracil (U) rather than thymine (T). Likewise, where the targeting domain comprises a core domain and/or a secondary domain, in certain embodiments one or both of the core domain and the secondary domain are fully complementary to the corresponding portions of Seq ID NOs: 1-10.

In other embodiments, the targeting domain is partially complementary to the target domain (Seq ID NOs: 1-10). In certain of these embodiments, the nucleic acid sequence of the targeting domain is at least 80, 85, 90, or 95% complementary to the target domain or to the corresponding portion of the target domain. In certain embodiments, the targeting domain include one or more nucleotides that are not complementary with the target domain or a portion thereof, and in certain of these embodiments the targeting domain includes 1, 2, 3, 4, 5, 6, 7, or 8 nucleotides that are not complementary with the target domain. In certain embodiments wherein the targeting domain includes one or more nucleotides that are not complementary with the target domain, one or more of said non-complementary nucleotides are located within five nucleotides of the 5′ or 3′ end of the targeting domain. In certain of these embodiments, the targeting domain includes 1, 2, 3, 4, or 5 nucleotides within five nucleotides of its 5′ end, 3′ end, or both its 5′ and 3′ ends that are not complementary to the target domain. In certain embodiments wherein the targeting domain includes two or more nucleotides that are not complementary to the target domain, two or more of said non-complementary nucleotides are adjacent to one another, and in certain of these embodiments the two or more consecutive non-complementary nucleotides are located within five nucleotides of the 5′ or 3′ end of the targeting domain. In other embodiments, the two or more consecutive non-complementary nucleotides are both located more than five nucleotides from the 5′ and 3′ ends of the targeting domain.

In certain embodiments, the targeting domain consists of, consists essentially of, or comprises 16, 17, 18, 19, 20, 21, 22, 23, 24, 25, or 26 nucleotides (e.g., 16, 17, 18, 19, 20, 21, 22, 23, 24, 25, or 26 consecutive nucleotides) complementary or partially complementary to the target domain or a portion thereof, e.g., the targeting domain is 16, 17, 18, 19, 20, 21, 22, 23, 24, 25, or 26 nucleotides in length. In certain of these embodiments, the targeting domain is complementary to the target domain over the entire length of the targeting domain, the entire length of the target domain, or both.

In certain embodiments, a unimolecular or chimeric gRNA molecule disclosed herein (comprising a targeting domain) comprises the first 20 N's (residues 1-20) of the amino acid sequence set forth in SEQ ID NOS: 1-10. In certain embodiments, a unimolecular or chimeric gRNA molecule disclosed herein (comprising a targeting domain) comprises the second 20 N's (residues 20-40) of the amino acid sequence set forth in SEQ ID NOS: 1-10. In certain embodiments, a unimolecular or chimeric gRNA molecule disclosed herein (comprising a targeting domain) comprises the third 20 N's (residues 40-60) of the amino acid sequence set forth in SEQ ID NOS: 1-10. In certain embodiments, a unimolecular or chimeric gRNA molecule disclosed herein (comprising a targeting domain) comprises the fourth 20 N's (residues 60-80) of the amino acid sequence set forth in SEQ ID NOS: 1-10. In certain embodiments, a unimolecular or chimeric gRNA molecule disclosed herein (comprising a targeting domain) comprises the fifth 20 N's (residues 80-100) of the amino acid sequence set forth in SEQ ID NOS: 1-10. In some embodiments, the targeting domain is listed as 20 Ns (residues 1-20) but may range in length from 16 to 26 nucleotides.

In certain embodiments, a unimolecular or chimeric gRNA molecule disclosed herein (comprising a targeting domain) comprises the last 20 N's of the amino acid sequence set forth in SEQ ID NOS: 1-10. In certain embodiments, a unimolecular or chimeric gRNA molecule disclosed herein (comprising a targeting domain) comprises the second to last 20 N's of the amino acid sequence set forth in SEQ ID NOS: 1-10. In certain embodiments, a unimolecular or chimeric gRNA molecule disclosed herein (comprising a targeting domain) comprises the third to last 20 N's of the amino acid sequence set forth in SEQ ID NOS: 1-10. In certain embodiments, a unimolecular or chimeric gRNA molecule disclosed herein (comprising a targeting domain) comprises the fourth to last 20 N's of the amino acid sequence set forth in SEQ ID NOS: 1-10. In certain embodiments, a unimolecular or chimeric gRNA molecule disclosed herein (comprising a targeting domain) comprises the fifth to last 20 N's of the amino acid sequence set forth in SEQ ID NOS: 1-10.

In certain embodiments, modifications or non-complementary nucleotides to one or more nucleotides in the targeting domain, do not interfere with targeting efficacy, which can be evaluated by testing a candidate modification using a system known in the art. gRNAs having a candidate targeting domain having a selected length, sequence, degree of complementarity, or degree of modification can be evaluated using a system known in the art. The candidate targeting domain can be placed, either alone or with one or more other candidate changes in a gRNA molecule/Cas9 molecule system known to be functional with a selected target, and evaluated.

The disclosure is also directed to a DNA targeting composition comprising a first gRNA and a second gRNA. The first gRNA molecule and the second gRNA molecule comprise a targeting domain comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10 (or portion thereof) or that is configured to associate with a target nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or a complement thereof. In some embodiments, the first gRNA molecule and the second gRNA molecule comprise different targeting domains.

The disclosure is also directed to an isolated polynucleotide comprising the gRNA molecule described above or the DNA targeting composition described above.

The disclosure is directed to a vector comprising the gRNA described above, the DNA targeting composition described above, or the isolated polynucleotide described above.

The disclosure is also directed to a vector comprising the DNA targeting composition described above.

The present invention is also directed to a vector encoding: (a) a first guide RNA (gRNA) molecule, (b) a second gRNA molecule, and (c) at least one Cas9 molecule that recognizes a Protospacer Adjacent Motif (PAM) of either NNGRRT (SEQ ID NO: 41) or NNGRRV (SEQ ID NO: 42). The first gRNA molecule and the second gRNA molecule comprise a targeting domain that is SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10 (or portion thereof) or is configured to associate with a target nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 or a complement thereof. In some embodiments, the first gRNA molecule and the second gRNA molecule comprise different targeting domains.

The disclosure is also directed to a cell comprising the gRNA described above, the DNA targeting composition described above, the isolated polynucleotide described above, and/or the vector of described above.

The disclosure is also directed to a kit comprising the gRNA described above, the DNA targeting system described above, the isolated polynucleotide described above, the vector described above, or the cell described above and optionally instructions for use.

The disclosure is also directed to a method of correcting a mutant PARK2, PINK1, LRRK2, SNCA, c-Rel, ATG7, VMAT2, GDNF, or GBA gene in a cell. The method comprises administering to a cell the gRNA described above, the DNA targeting system described above, the isolated polynucleotide described above, or the vector described above.

The disclosure is also directed to a method of genome editing a mutant PARK2, PINK1, LRRK2, SNCA, c-Rel, ATG7, VMAT2, GDNF, or GBA gene in a subject. The method comprises administering to the subject a genome editing composition comprising the gRNA described above, the DNA targeting system described above, the isolated polynucleotide described above, the vector described above, or the cell described above.

The disclosure is also directed to a method of treating a subject in need thereof having a mutant PARK2, PINK1, LRRK2, SNCA, c-Rel, ATG7, VMAT2, GDNF, or GBA gene. The method comprises administering to the subject the gRNA described above, the DNA targeting system described above, the isolated polynucleotide described above, the vector described above, or the cell described above.

The disclosure is also directed to a modified adeno-associated viral vector for genome editing a mutant PARK2, PINK1, LRRK2, SNCA, c-Rel, ATG7, VMAT2, GDNF, or GBA gene in a subject comprising a first polynucleotide sequence encoding the gRNA described above, and a second polynucleotide sequence encoding a Cas9 molecule that recognizes a Protospacer Adjacent Motif (PAM) of either NNGRRT (SEQ ID NO: 41) or NNGRRV (SEQ ID NO: 42).

The disclosure is also directed to a cell comprising the composition described above.

A vector encoding a guide RNA (gRNA) molecule and a Cas9 molecule, wherein the gRNA molecule comprises a targeting domain comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10 (or fragment thereof) or is configured to associate with a target nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 or a complement thereof.

A genome editing system comprising: (a) a gRNA molecule comprising a targeting domain that is configured to associate with a target nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 or a complement thereof; and (b) a Cas9 molecule.

Non-limiting methods for evaluating chromatin accessibility include micrococcal nuclease (MNase)-assisted isolation of nucleosomes sequencing (MNase-seq), DNase I hypersensitive sites sequencing (DNase-seq), formaldehyde-assisted isolation of regulatory elements sequencing (FAIRE-seq), and assay for transposase-accessible chromatin using sequencing (ATAC-seq). In some embodiments, the chromatin accessibility is determined by an Assay for Transposase-Accessible Chromatin with high-throughput sequencing (ATAC-seq). In some embodiments, the disclosed methods further include selecting a locus as an GSH if the locus is located at a distance of up to about 10 kb, up to about 9 kb, up to about 8 kb, up to about 7 kb, up to about 6 kb, up to about 5 kb, up to about 4 kb, up to about 3 kb, up to about 2 kb, or up to about 1 kb from an ATAC-seq peak or within an ATAC-seq peak.

The Gene Editing Systems disclosed herein can be further understood by the following numbered paragraphs:
Paragraph 1. A gene editing system for a cell comprising:
   a. a Cas protein or a polynucleotide encoding a Cas protein;
   b. a guide-RNA (gRNA); and
   c. a repair template comprising a functional PARK2, PINK1, LRRK2, SNCA, c-Rel, ATG7, VMAT2, GDNF, or GBA gene, or a functional variant or fragment thereof, wherein the gene editing system is capable of repairing an endogenous gene in the cell or inserting a functional gene into the genome of the cell in vivo or in vitro.
Paragraph 2. The gene editing system of paragraph 1, wherein at least one component of the gene editing system is delivered by recombinant AAV.
Paragraph 3. The gene editing system of paragraph 1, wherein the gene editing system is delivered by recombinant AAV.
Paragraph 4. The gene editing system of any of paragraph 1 to 3, wherein the cell is a pluripotent cell, or DA neuronal cell in vitro.
Paragraph 5. The gene editing system of any of paragraph 1 to 3, wherein the cell is a dopaminergic neuron in vivo.

### Recombinant Gene Vector

Disclosed are various embodiments of, and methods related to, a recombinant gene vector comprising a PARK2, PINK1, DJ-1, LRRK2, c-Rel, ATG7, VMAT2, GDNF, and/or GBA or functional fragment or variant thereof. In some embodiments, the vector further comprises an edit to the alpha-synuclein (SNCA) gene to render the cell population the vector is introduced into hemizygous null for SNCA. In some embodiments, the recombinant gene vector renders the cell it is introduced into is homozygous null for SNCA and/or homozygous null for MAPT. In some embodiments, the recombinant gene vector renders the cell it is introduced into hemizygous null for SNCA and/or homozygous null for MAPT. In some embodiments, the recombinant gene vector renders the cell it is introduced into is homozygous null for SNCA and/or hemizygous null for MAPT. In some embodiments, the recombinant gene vector does not include a SNCA gene edit.

Disclosed are various embodiments of, and methods related to, a recombinant gene vector comprising a GDNF gene (SEQ ID NO: 10) or functional fragment or variant thereof. In some embodiments, the vector further comprises an edit to the GBA gene. In some embodiments, the vector further comprises an edit to the SNCA gene.

Disclosed are various embodiments of, and methods related to, a recombinant gene vector comprising a GBA gene (SEQ ID NO: 7) or functional fragment or variant thereof. In some embodiments, the vector further comprises an edit to the SNCA gene.

Various viral or non-viral vectors can be used. In some embodiments, the recombinant gene vector is a recombinant adeno-associated virus (AAV). Any of the known serotypes can be used. In some embodiments, the AAV has serotype AAV1, AAV2, AAV5, AAV8, AAV9, AAVrh10, or AAVrh74. In some embodiments, the recombinant gene vector comprises a self-complementary AAV. In some embodiments, the recombinant gene vector comprises a single-stranded AAV. In some embodiments, the AAV is a wild-type AAV or a modified AAV. In some embodiments, the AAV comprises a capsid protein having at least 95% identity to a wild-type VP1, VP2, or VP3 capsid protein.

The recombinant gene vector may include gene regulatory elements. In some embodiments, the recombinant gene vector comprises a polynucleotide comprising, in the following 5′ to 3′ order, a eukaryotically active promoter sequence and the sequence encoding the wild-type protein, or functional fragment or variant thereof. The sequence encoding the wild-type protein, or functional fragment or variant thereof, is operably linked to the eukaryotically active promoter sequence.

In some embodiments, the vectors and methods disclosed herein, the vector comprises an expression cassette comprising in 5′ to 3′ order:
A promoter (a GDNF knock-in promotor [such as MAOB, GCH1, NR4A2, TH, SLC6A3, SYN1, Camk2a, NEFM, NEFL, or NEFH], a GBA knock-in promotor [such as mTHY1, rNSE, DDC, COMT, GBA P1, GBA P2, EFS, EEF1A1, CBA, CMV, hPGK], Synapsin 1, DAT, VMAT, TH, AADC, tamoxifen inducible promoter, RU486 inducible promoter, or other promotor that allows for temporal or small molecule control), the gene (PARK2, PINK1, DJ-1, LRRK2, c-Rel, ATG7, VMAT2, GBA, SNCA and/or GDNF);
A promoter (a GDNF knock-in promotor [such as MAOB, GCH1, NR4A2, TH, SLC6A3, SYN1, Camk2a, NEFM, NEFL, or NEFH], a GBA knock-in promotor [such as mTHY1, rNSE, DDC, COMT, GBA P1, GBA P2, EFS, EEF1A1, CBA, CMV, hPGK], Synapsin 1, DAT, VMAT, TH, AADC, tamoxifen inducible promoter, RU486 inducible promoter), the gene (PARK2, PINK1, DJ-1, LRRK2, c-Rel, ATG7, VMAT2, GBA, SNCA and/or GDNF);
A promoter (a GDNF knock-in promotor [such as MAOB, GCH1, NR4A2, TH, SLC6A3, SYN1, Camk2a, NEFM, NEFL, or NEFH], a GBA knock-in promotor [such as mTHY1, rNSE, DDC, COMT, GBA P1, GBA P2, EFS, EEF1A1, CBA, CMV, hPGK], Synapsin 1, DAT, VMAT, TH, AADC, tamoxifen inducible promoter, RU486 inducible promoter, or other promotor that allows for temporal or small molecule control), the gene (PARK2, PINK1, DJ-1, LRRK2, c-Rel, ATG7, VMAT2, GBA, SNCA and/or GDNF) and is hemizygous null for the SNCA and/or MAPT gene;
A promoter (a GDNF knock-in promotor [such as MAOB, GCH1, NR4A2, TH, SLC6A3, SYN1, Camk2a, NEFM, NEFL, or NEFH], a GBA knock-in promotor [such as mTHY1, rNSE, DDC, COMT, GBA P1, GBA P2, EFS, EEF1A1, CBA, CMV, hPGK], Synapsin 1, DAT, VMAT, TH, AADC, tamoxifen inducible promoter, RU486 inducible promoter, or other promotor that allows for temporal or small molecule control), a S'enhancer, the gene (PARK2, PINK1, DJ-1, LRRK2, c-Rel, ATG7, VMAT2, GBA, SNCA and/or GDNF);
A promoter (a GDNF knock-in promotor [such as MAOB, GCH1, NR4A2, TH, SLC6A3, SYN1, Camk2a, NEFM, NEFL, or NEFH], a GBA knock-in promotor [such as mTHY1, rNSE, DDC, COMT, GBA P1, GBA P2, EFS, EEF1A1, CBA, CMV, hPGK], Synapsin 1, DAT, VMAT, TH, AADC, tamoxifen inducible promoter, RU486 inducible promoter, or other promotor that allows for temporal or small molecule control), a 5′enhancer, the gene (PARK2, PINK1, DJ-1, LRRK2, c-Rel, ATG7, VMAT2, GBA, SNCA and/or GDNF) and is hemizygous null for the SNCA and/or MAPT gene.

In some embodiments, the recombinant gene vector comprises one or more of a neuron-specific promoter, optionally selected from the group comprising or consisting of hSYN1 (human synapsin), INA (alpha-internexin), NES (nestin), TH (tyrosine hydroxylase), FOXA2 (Forkhead box A2), CaMKII (calmodulin-dependent protein kinase II), and NSE (neuron-specific enolase) promoters.

In some embodiments, the recombinant gene vector comprises a ubiquitous promotor selected from the group comprising or consisting of CMV, CAG, UBC, PGK, EF1-alpha, GAPDH, SV40, HBV, and chicken beta-actin promoters

In some embodiments, the disclosed engineered DA neuronal cell transduced with a recombinant gene vector also comprises creating a mutation in a gene associated with Parkinson's Disease (PD). The mutated gene can be SNCA and/or MAPT. The mutated gene can be one of SEQ ID NOs: 1-10 or combinations thereof.

Disclosed is a vector encoding a guide RNA (gRNA) molecule and one Cas protein molecule, wherein the gRNA molecule comprises a targeting domain that comprises a portion of the nucleotide sequence selected from SEQ ID NOS: 1-10. In some embodiments, the vector is a viral vector. In some embodiments, the viral vector is an adeno-associated viral vector. In some embodiments, the vector is a nucleic acid sequence containing an origin of replication. In some embodiments, the vector may be a viral vector, bacteriophage, bacterial artificial chromosome or yeast artificial chromosome. In some embodiments, the vector is a DNA or RNA vector. In some embodiments, the vector is a self-replicating extrachromosomal vector, and preferably, is a DNA plasmid. For example, the vector may encode a Cas9 protein and at least one gRNA molecule. In some embodiments, the Cas9 protein may be a S. aureus Cas9, such as a SaCas9.

Disclosed is a genome editing system comprising: (a) a gRNA molecule comprising a targeting domain that comprises a portion of the nucleotide sequence selected from SEQ ID NOS: 1-10, (b) a Cas protein molecule.

In some embodiments, the vector or genome editing system is configured to alter a GDNF, GBA or SNCA gene. In some embodiments, the vector or genome editing system is configured to alter a SEQ ID NO: 10, SEQ ID NO: 7, or SEQ ID NO: 8. In some embodiments, the Cas protein molecule is an S. aureus Cas9 molecule. In some embodiments the Cas protein molecule recognizes a Protospacer Adjacent Motif (PAM) of either NNGRRT (SEQ ID NO: 41) or NNGRRV (SEQ ID NO: 42).

The presently disclosed subject matter also provides cells comprising the vector or genome editing system describe herein. In some embodiments, the cells are pluripotent cells or DA neuronal cells or dopaminergic neurons.

The presently disclosed subject matter further provides a method to enhance one or more of viability, engraftment, proliferation, migration, innervation or differentiation, comprising administering to the cell: (a) a vector encoding a gRNA molecule and one Cas protein molecule; or (b) a genome editing system comprising a gRNA molecule and a Cas protein molecule; wherein the gRNA molecule comprises a targeting domain comprising a 20 bae pare portion of the nucleotide sequence selected from a GDNF (Seq ID No: 10), GBA (Seq ID No: 7) and/or SNCA (Seq ID No: 8) gene or functional fragment thereof.

In one aspect, the disclosure relates to a method for treating or altering a cell in a subject (e.g., a human subject or an animal subject) that includes administering to the subject a nucleic acid encoding a Cas protein and first and second guide RNAs (gRNAs) targeted to the GDNF (Seq ID No: 10), GBA (Seq ID No: 7) and/or SNCA (Seq ID No: 8) gene of the subject. In some embodiments, the first and second gRNAs are targeted to one or more target sequences that encompass or are proximal to a 20 nucleic acid target position which may be 5′, 3′ or in the middle of the sequence. The first gRNA may include a targeting domain selected to target the first 20 N's (residues 1-20), while the targeting domain of the second gRNA may be selected to target the second 20 N's (residues 20-40). The first gRNA may include a targeting domain selected to target the second 20 N's (residues 20-40), while the targeting domain of the second gRNA may be selected to target the third 20 N's (residues 40-60). The first gRNA may include a targeting domain selected to target the third 20 N's (residues 40-60), while the targeting domain of the second gRNA may be selected to target the fourth 20 N's (residues 60-80). The first gRNA may include a targeting domain selected to target the second 20 N's (residues 20-40), while the targeting domain of the second gRNA may be selected to target the fifth 20 N's (residues 80-100).

The Cas protein, which may be a modified Cas protein (e.g., a Cas9 engineered to alter PAM specificity, improve fidelity, or to alter or improve another structural or functional aspect of the Cas9), may include one or more of a nuclear localization signal (NLS) and/or a polyadenylation signal. Certain embodiments are characterized by Cas proteins that include both a C-terminal and an N-terminal NLS. The Cas protein is optionally driven by a promoter selected from the group comprising Synapsin 1, DAT, VMAT, TH, AADC, tamoxifen inducible promoter, RU486 inducible promoter, or other promotor that allows for temporal or small molecule control. The nucleic acid also includes, in various cases, first and second inverted terminal repeat sequences (ITRs).

The embodiments disclosed herein can be further understood by the following numbered paragraphs.

Paragraph 1. A vector encoding a guide RNA (gRNA) molecule and a Cas protein molecule, wherein the gRNA molecule comprises a targeting domain configured to alter PARK2, PINK1, DJ-1, LRRK2, SNCA, c-Rel, ATG7, VMAT2, GBA, SNCA or GDNF gene.

Paragraph 2. The vector of paragraph 1, wherein the Cas molecule is an S. aureus Cas9 molecule.

Paragraph 3. The vector of paragraph 1, wherein the Cas molecule recognizes a Protospacer Adjacent Motif (PAM) of either NNGRRT (SEQ ID NO: 41) or NNGRRV (SEQ ID NO: 42).

Paragraph 4. The vector of paragraph 1, wherein the vector is a viral vector.

Paragraph 5. A composition comprising the vector of paragraph 1.

Paragraph 6. A genome editing system comprising:
(a) a gRNA molecule comprising a targeting domain that is configured to alter the nucleotide sequence set forth in SEQ ID NO: 1-10 or comprises the nucleotide sequence set forth in SEQ ID NO: 1-10; and
(b) a Cas molecule.

Paragraph 7. The genome editing system of paragraph 6, wherein the genome editing system is configured to alter a PARK2, PINK1, DJ-1, LRRK2, SNCA, c-Rel, ATG7, VMAT2, GBA, SNCA or GDNF gene.

Paragraph 8. The genome editing system of paragraph 6, wherein the Cas molecule is an S. aureus Cas9 molecule.

Paragraph 9. The genome editing system of paragraph 6, wherein the Cas molecule recognizes a PAM of either NNGRRT (SEQ ID NO: 41) or NNGRRV (SEQ ID NO: 42).

Paragraph 10. An isolated cell comprising the vector of paragraph 1.

Paragraph 11. An isolated cell comprising the genome editing system of paragraph 6.

Paragraph 12. A method of making a gene edited cell, comprising administering to a cell:
(a) a vector encoding a gRNA molecule and a Cas protein molecule; or
(b) a genome editing system comprising a gRNA molecule and a Cas protein molecule;
wherein the gRNA molecule comprises a targeting domain comprising a portion of the nucleotide sequence set forth in SEQ ID NO: 1-10 or is configured to alter the nucleotide sequence set forth in SEQ ID NO: 1-10.

Paragraph 13. The method of paragraph 12, wherein the cell is selected from the group comprising or consisting of a pluripotent cell and a DA neuronal cell.

Paragraph 14. A method of inhibiting degeneration or death of a dopaminergic neuron comprising a mutation in a gene associated with a Parkinson's Disease (PD), comprising: contacting the neuron with the recombinant gene therapy vector of any one of paragraphs 1 to 4; wherein following contact with the recombinant gene therapy vector, the neuron expresses the PARK2, PINK1, DJ-1, LRRK2, c-Rel, ATG7, VMAT2, GDNF, SNCA+/− and/or GBA protein.

Paragraph 15. The method of paragraph 14, wherein the neuron expresses a reduced amount of alpha-synuclein following contact with the recombinant gene therapy vector.

Paragraph 16. The method of any of paragraphs 14 to 15, wherein the neuron produces and/or releases an increased amount of dopamine following contact with the recombinant gene therapy vector.

Paragraph 17. The method of any of paragraphs 14 to 16, wherein the neuron expresses a lower amount of alpha-synuclein as compared to an amount of alpha-synuclein expressed in a neuron not contacted with the recombinant gene therapy vector, optionally wherein the lower amount is at least 5%, at least 10%, at least 20%, at least 30%, at least 40%, at least 50%, at least 60%, at least 70%, or at least 80% lower than the amount expressed in the neuron not contacted with the recombinant gene therapy vector.

Paragraph 18. The method of any of paragraphs 14 to 17, wherein the neuron produces and/or releases an increased amount of dopamine as compared to an amount of dopamine produced and/or released by a neuron not contacted with the recombinant gene therapy vector, optionally wherein the increase amount is at least 5%, at least 10%, at least 20%, at least 30%, at least 40%, at least 50%, at least 60%, at least 70%, at least 80%, at least 90%, at least two-fold, at least three-fold, at least four-fold, at least five-fold, or at least 10-fold greater than the amount produced and/or released by the neuron not contacted with the recombinant gene therapy vector.

Paragraph 19. The method of any of paragraphs 14 to 18, wherein the neuron undergoes an increased amount of autophagy as compared to an amount of autophagy undergone by a neuron not contacted with the recombinant gene therapy vector, optionally wherein the increased amount is at least 5%, at least 10%, at least 20%, at least 30%, at least 40%, at least 50%, at least 60%, at least 70%, at least 80%, at least 90%, at least two-fold, at least three-fold, at least four-fold, at least five-fold, or at least 10-fold greater than the amount undergone by the neuron not contacted with the recombinant gene therapy vector.

Paragraph 20. The method of any of paragraphs 14 to 19, wherein the neuron is a primary tyrosine hydroxylase positive neuron.

Paragraph 21. A method of treating or inhibiting onset of a Parkinson's Disease (PD) in a subject suffering from or at risk of the PD, comprising:
administering a recombinant gene therapy vector of any one of paragraphs 1 to 4 to the subject;
wherein administration of the recombinant gene therapy vector treats or inhibits onset of the Parkinson's Disease in the subject.

Paragraph 22. The method of claim 22, wherein the subject is an adult or child.

Paragraph 23. The method of any of paragraphs 21 to 22, wherein the number of dopaminergic neurons in the subject after the administering step is greater than the number of dopaminergic neurons in the subject before the administering step.

Paragraph 24. The method of any of paragraphs 21 to 23, wherein the level of dopamine in the subject after the administering step is greater than the level of dopamine in the subject before the administering step.

Paragraph 25. The method of any of paragraphs 21 to 24, wherein the number of dopaminergic neurons in a subject treated by the method is increased compared to the number of dopaminergic neurons in a subject not so treated.

Paragraph 26. The method of any of paragraphs 21 to 25, wherein the level of dopamine of a subject treated by the method is increased compared to the level of dopamine in a subject not so treated.

Paragraph 27. The method of any one of paragraphs 21 to 26, wherein the level of dopamine in the substantia nigra of a subject treated by method is increased compared to the level of dopamine in the substantia nigra of a subject not so treated.

Paragraph 28. The method of any of paragraphs 21 to 27, wherein the level of GDNF and/or GBA in the subject's CSF after the administering step is greater than the level of GDNF and/or GBA in the subject's CSF before the administering step.

Paragraph 29. The method of any of paragraphs 21 to 28, wherein the Unified Parkinson's Disease Rating Scale (UPDRS) score of the subject before the administering step is improved compared to the UPDRS score of the subject before the administering step.

Paragraph 30. The method of any of paragraphs 21 to 29, wherein the level of PRKN in the CSF of a subject treated by the method is increased compared to the level of PRKN in the CSF of a subject not so treated.

Paragraph 31. The method of any of paragraphs 21 to 29, wherein the UPDRS score of a subject treated by the method is improved compared to the UPDRS score of a subject not so treated.

Paragraph 32. The method of any of paragraphs 21 to 31, wherein the subject's neurons express a reduced amount of alpha-synuclein and/or comprises a reduced amount of Lewy bodies following contact with the recombinant gene therapy vector.

The embodiments disclosed herein can be further understood by the following numbered paragraphs.

Paragraph 1. An engineered pluripotent cell, which comprises an exogenous sequence encoding a GDNF gene, which has been integrated under transcriptional control of an endogenous gene promoter.

Paragraph 2. An engineered DA neuronal cell, which comprises an exogenous sequence encoding a GDNF gene, which has been integrated under transcriptional control of an endogenous gene promoter.

Paragraph 3. An engineered pluripotent cell according to paragraph 1, which has a genotype SNCA+/−.

Paragraph 4. An engineered DA neuronal cell according to paragraph 2, which has a genotype SNCA+/−

### Methods

### Method for Making an Engineered Cell

In some embodiments, disclosed is a method of making an engineered cell comprising contacting a pluripotent cell with a gene editing system comprising: a Cas protein or a polynucleotide encoding a Cas protein; a guide-RNA (gRNA); and a repair template comprising a functional PARK2, PINK1, DJ-1, LRRK2, c-Rel, ATG7, VMAT2, GBA, hemizygous SNCA, hemizygous MAPT and/or GDNF gene or a functional variant or fragment thereof.

In some embodiments, the pluripotent cell is in vitro. In some embodiments, the engineered pluripotent cell is differentiated in vitro to a DA neuronal cell to form an engineered DA neuronal cell. In some embodiments, the engineered DA neuronal cell is translated into a patient. In some embodiments, the engineered DA neuronal cell is capable of enhancing the viability, engraftment, proliferation, migration, innervation, differentiation, long-term graft integrity, survival of endogenous neurons, or function of the administered engineered DA neuronal cells compared to wildtype neuronal cells. In some embodiments, the administered engineered DA neuronal cells differentiate in vivo to a Neuronal Mature Cell Type and the administered engineered DA neuronal cells enhance the function of the Neuronal Mature Cell Type compared to wildtype Neuronal Mature Cells. In some embodiments, the administered engineered DA neuronal cells enhance the function of the endogenous (non-engineered) Neuronal Mature Cell Types in the patient.

In some embodiments, the pluripotent cell and/or subject to be genetically engineered comprises a mutation in a PARK2 gene, PINK1 gene, LRRK2 gene, c-Rel gene, ATG7 gene, VMAT2, GBA, SNCA, MAPT and/or GDNF gene. In some embodiments, the pluripotent cell and/or subject to be genetically engineered comprises a mutation in one of SEQ ID NOS: 1-10 or combinations thereof.

Disclosed herein is a method for making an engineered cell comprising; introducing into a cell a guiding polynucleotide comprising a spacer region that is complementary to a target nucleic acid in a genomic region of the cell; a nuclease that is guided by the guiding polynucleotide; and a polynucleotide encoding an exogenous GDNF, GBA or SNCA; site-specifically cleaving the target nucleic acid inside the cell by the nuclease guided by the guiding polynucleotide; and inserting the polynucleotide encoding the exogenous GDNF, GBA and/or SNCA into the genomic region of the cell at the cleavage site. The nuclease can be Cas9. In some embodiments, the guiding polynucleotide can be a single guiding polynucleotide. The guiding polynucleotide can be RNA. The target nucleic acid can be DNA. The spacer region can be between 10-30 nucleotides in length. The nuclease can produce a double stranded break in the target nucleic acid. The nuclease can produce a single strand break in the target nucleic acid.

In some embodiments the guiding polynucleotide can be introduced into a cell by electroporation. A guide nucleic acid can be introduced into a cell by nucleofection. A nuclease can also be introduced into a cell by a delivery vector. A polynucleotide encoding an exogenous GDNF, GBA and/or SNCA can further comprise a promoter sequence. The promoter sequence can be selected from one of SEQ ID NOS 21-40 or 43. An exogenous GDNF, GBA and/or SNCA can be inserted by homologous recombination. A guiding polynucleotide and a nuclease can form a nucleoprotein complex.

Cleaving a target nucleic acid can remove a genomic nucleic acid sequence that is replaced with a polynucleotide encoding an exogenous GDNF, GBA and/or SNCA. A polynucleotide encoding an exogenous GDNF, GBA and/or SNCA can further comprise a first recombination arm and a second recombination arm. A first recombination arm can comprise a first sequence that is identical to a first portion of a target nucleic acid and a second recombination arm can comprise a second sequence that is identical to a second portion of a target nucleic acid. In some embodiments, a first recombination arm can comprise a first sequence that is identical to a first portion adjacent to a target nucleic acid and a second recombination arm can comprise a second sequence that is identical to a second portion adjacent to a target nucleic acid. A target nucleic acid can be within a gene. A gene can be selected from GDNF, GBA and/or SNCA.

In some embodiments, insertion of an exogenous GDNF, GBA and/or SNCA sequence at a cleavage site can result in disruption of a gene. A target nucleic acid can be within an intergenic site. An exogenous GDNF, GBA and/or SNCA can be expressed in a cell. An engineered cell can be introduced into an organism. Engineered cells can be expanded ex vivo.

Disclosed herein is a method for efficient target gene disruption in DA neuronal cells comprising contacting a pluripotent cell with a Cas protein nuclease and a guide RNA, wherein a guide RNA contains a region of 17 to 22 nucleotides that is substantially complementary to a region in a target gene; cleaving a target gene, wherein a target gene can be GDNF, GBA and/or SNCA and wherein an exogenous knock out event occurs in at least 30%, at least 40%, at least 50%, at least 60%, at least 70%, or at least 80% of pluripotent cells when a population of pluripotent cells are contacted with a Cas protein nuclease and a guide RNA.

In some embodiments, the method comprises one or more cells, and the one or more cells are pluripotent cells, DA neuronal cells, neurons or any combination thereof. In some embodiments, the method comprises a first nucleic acid, and the first nucleic acid is DNA, RNA or a hybrid thereof. In some embodiments, the method comprises a first nucleic acid, and the first nucleic acid is single stranded or double stranded. In some embodiments, the method comprises a second nucleic acid, and the second nucleic acid is DNA, RNA or a hybrid thereof. In some embodiments, the method comprises a second nucleic acid, and the second nucleic acid is single stranded or double stranded. In some embodiments, the method comprises introducing a first nucleic acid, and introducing the first nucleic acid comprises non-viral transfection, biolistics, chemical transfection, electroporation, nucleofection, heat-shock transfection, lipofection, microinjection, or viral transfection. In some embodiments the method comprises viral transduction, and the viral transduction comprises an adeno-associated virus. In some embodiments, the method comprises introducing a second nucleic acid, and introducing the second nucleic acid comprises non-viral transfection, biolistics, chemical transfection, electroporation, nucleofection, heat-shock transfection, lipofection, microinjection, or viral transfection.

In some embodiments, the method comprises a double strand break, and creating the double strand break comprises CRISPR, TALEN, transposon-based, ZFN, meganuclease, or Mega-TAL. In some embodiments, the method comprises a double strand break, and creating the double strand break comprises CRISPR. In some embodiments, the method comprises a double strand break, and the double strand break is repaired by insertion of a gene encoding an engineered GDNF, GBA and/or SNCA gene. In some embodiments, the method comprises a second nucleic acid, and the second nucleic acid comprises recombination arms, and wherein the second gene encoding an engineered GDNF, GBA and/or SNCA gene is flanked by the recombination arms. In some embodiments, the method comprises recombination arms, and the recombination arms are at least in part complementary to at least a portion of the at least one endogenous GDNF, GBA and/or SNCA gene. In some embodiments of the methods of the present disclosure, an increase in isogenicity between the recombination arms and the at least one endogenous GDNF, GBA and/or SNCA gene corresponds to an increase in efficiency of the insertion of a second gene. In some embodiments, the method comprises insertion of a second gene, and an efficiency of the insertion of the second gene is measured using fluorescence-expressed cell sorting. In some embodiments, the method comprises introducing a second nucleic acid, and introducing the second nucleic acid comprises non-viral transfection, biolistics, chemical transfection, electroporation, nucleofection, heat-shock transfection, lipofection, microinjection, or viral transfection. In some embodiments, the method comprises insertion of a second gene, and the insertion of the second gene encoding an engineered GDNF, GBA and/or SNCA gene comprises homology directed repair (HDR). In some embodiments, the method comprises insertions of a second gene, and the insertion of the second gene is assisted by a homologous recombination (HR) enhancer. In some embodiments, the method comprises an enhancer, and the enhancer is derived from a viral protein. In some embodiments, the method comprises an HR enhancer, and the HR enhancer is selected from the group comprising or consisting of E4orf6, E1b55K, E1b55K-H354, E1b55K-H373A, Scr7, L755507, or any combination thereof. In some embodiments, the method comprises an HR enhancer, and the HR enhancer is a chemical inhibitor. In some embodiments, the methods comprise an HR enhancer, and the HR enhancer inhibits Ligase IV. In some embodiments, the method comprises a reduction in cytotoxicity, and the cytotoxicity comprises at least one of DNA cleavage, cell death, apoptosis, nuclear condensation, cell lysis, necrosis, altered cell motility, altered cell stiffness, altered cytoplasmic protein expression, altered membrane protein expression, swelling, loss of membrane integrity, cessation of metabolic activity, hypoactive metabolism, hyperactive metabolism, increased reactive oxygen species, cytoplasmic shrinkage, or any combination thereof. In some embodiments, the method comprises measuring viability, and the viability is measured using at least one of fluorescence-expressed cell sorting, trypan blue exclusion, CD4+ cell-surface markers, CD8+ cell-surface markers, telomere length, or any combination thereof. In some embodiments, the method comprises a subject, and the subject is a human subject.

### Methods of Making a Therapeutically Effective Composition

In one embodiment, disclosed are methods of making a therapeutically effective composition comprising one or more cells. In some embodiments, the method comprises gene editing, and the gene editing comprises introducing into the one or more cells a first nucleic acid. In some embodiments, the method comprises a first nucleic acid, and the first nucleic acid comprises a first gene encoding a PARK2, PINK1, DJ-1, LRRK2, c-Rel, ATG7, VMAT2, GBA, SNCA or GDNF protein. In some embodiments, the method comprises gene editing, and the gene editing comprises introducing into the one or more cells a second nucleic acid. In some embodiments, the method comprises a second nucleic acid, and the second nucleic acid comprises a second gene encoding a PARK2, PINK1, DJ-1, LRRK2, c-Rel, ATG7, VMAT2, GBA, SNCA or GDNF protein wherein the second gene and first gene are different.

Disclosed is a method of treating or inhibiting onset of a Parkinson's Disease (PD) in a subject suffering from or at risk of the PD, comprising:
administering a DA Neuronal Cell wherein the DA Neuronal Cell comprises a recombinant gene vector comprising a polynucleotide sequence encoding a wild-type PARK2, PINK1, DJ-1, LRRK2, c-Rel, ATG7, VMAT2, GBA, SNCA or GDNF gene, or a functional variant or fragment thereof, to the subject; wherein administration of the DA neuronal cells comprising a recombinant gene vector treats or inhibits onset of the Parkinson's Disease in the subject.

### Methods of Identifying a Safe Harbor Locus

Method of identifying a safe harbor locus is disclosed. The method of identifying a safe harbor locus suitable for editing PSCs and expression in post-mitotic dopaminergic neurons, comprising, consisting of or consisting essentially of: a. generating from a clinically compliant PSC line and/or dopaminergic neuronal progenitor prioritized genomic safe harbor sites using scATAC-seq data; b. comparing the data from step a with published ATAC-seq data from dopaminergic neurons isolated from human healthy and Parkinson's brains; c. defining shared regions of chromatin accessibility between the data in a and the comparison in step b thereby generating a prioritized catalog of GSH sites suitable for editing in PSCs and expression in post-mitotic dopaminergic neurons. In some embodiments, prioritized catalog of GSH sites are further prioritized based on the GSH site being remote from genes, regulatory regions, telomeres, and centromeres to prevent transgene interference with gene expression, division, and function. In some embodiments, the GSH site is selected based on highest efficiency for transgene insertion, durable expression following prolonged culture, and/or absence of oncogenic gene expression. In some embodiments, the GSH site is selected based on peak size, and/or distribution data generated with processed ATAC-seq data.

Disclosed is a method of identifying a safe harbor locus suitable for editing PSCs and expression in post-mitotic dopaminergic neurons, comprising, consisting of or consisting essentially of: a. generating from a clinically compliant PSC line and/or dopaminergic neuronal progenitor prioritized genomic safe harbor sites using scATAC-seq data; b. comparing the data from step a with published ATAC-seq data from dopaminergic neurons isolated from human healthy and Parkinson's brains; c. defining shared regions of chromatin accessibility between the data in a and the comparison in step b; and d. selecting the safe harbor locus based on threshold parameters; wherein the safe harbor locus is selected for insertion of at least one sequence encoding a transgene within a cell. In some embodiments, the threshold parameters are based on peak size, and/or distribution data generated with processed ATAC-seq data. In some embodiments, the threshold parameters are based on peak size data generated with processed ATAC-seq data, distribution data generated with processed ATAC-seq data, highest efficiency for transgene insertion, durable expression following prolonged culture, and/or absence of oncogenic gene expression. In some embodiments, defining chromatin accessibility is based on the (1) PSCs, to optimize editing efficiency in step a, (2) DA neuronal cells, to optimize editing efficiency in step a, and (3) post-mitotic dopaminergic neurons from human brain samples. In some embodiments, defining chromatin accessibility is based on the (1) PSCs, to optimize editing efficiency in step a, (2) DA neuronal cells, to optimize editing efficiency in step a, (3) post-mitotic dopaminergic neurons from human brain samples and (4) Neuronal Mature Cell Type cells.

In some embodiments, the method of targeting a transgene (e.g. GDNF) into a safe harbor locus in PSCs for expression in post-mitotic dopaminergic neurons further comprises selecting an sgRNAs with highest on-target and lowest off-target activities and empirically defining the best cutting sgRNA.

In some embodiments, identifying a safe harbor locus in PSCs for expression in post-mitotic dopaminergic neurons further comprises further defining chromatin accessibility based on ATAC-seq peak data in a first cell type (PSC, DA neuronal, and/or Neuronal Mature Cell Type) compared to ATAC-seq peak data in a second, different cell type (PSC cells, DA neuronal cells, Neuronal Mature Cell Type, and/or HEK cells). In some embodiments, chromatin accessibility is based on ATAC-seq peak data compared to validated transgene insertions, and/or the alignment between the ATAC-seq peak and the sgRNA positions. In some embodiments, chromatin accessibility is based on ATAC-seq peak data compared scATAC-seq data from substantia nigra (SN) cells of healthy control and Parkinson's disease patients. In some embodiments, chromatin accessibility is based on ATAC-seq peak data compared scATAC-seq data from substantia nigra (SN) cells of healthy control and Parkinson's disease patients as compared to published ATAC-seq from human iPSCs and mature dopaminergic neurons. In some embodiments, chromatin accessibility is based on ATAC-seq peak data compared scATAC-seq data from dopaminergic cells of healthy control and Parkinson's disease patients. In some embodiments, chromatin accessibility is based on ATAC-seq peak data compared scATAC-seq data from substantia nigra (SN) cells of healthy control and Parkinson's disease patients as compared to published ATAC-seq from human iPSCs and mature dopaminergic neurons. In some embodiments, chromatin accessibility is based on the candidate GSH sites being classified as ‘open’ in healthy control, but ‘closed’ in Parkinson's patients. In some embodiments, chromatin accessibility is based on the candidate GSH sites being classified as ‘closed’ in healthy control, but ‘open’ in Parkinson's patients. In some embodiments, chromatin accessibility is based on the candidate GSH sites being classified as ‘open’ in healthy control, and ‘open’ in Parkinson's patients. In some embodiments, chromatin accessibility is based on any combination of factors discussed in this paragraph. In some embodiments, transgene insertion (e.g. GDNF) has no effect on differentiation capacity, i.e., the DA neuronal cells can differentiate (in vivo and in vitro) into a Neuronal Mature Cell Type.

Disclosed is a method of identifying a safe harbor locus, comprising, consisting of or consisting essentially of: a. identifying genes or non-coding regions in a chromosome that are above a threshold level for accessibility of chromatin; b. generating a model that correlates the gene or non-coding region from step (a) with published ATAC-seq data from dopaminergic neurons isolated from human healthy and Parkinson's brains on the chromosome; and c. selecting the safe harbor locus based on threshold parameters; wherein the safe harbor locus is selected for insertion of at least one sequence encoding a transgene within a cell. In some embodiments, the threshold parameters are based on peak size data generated with processed ATAC-seq data, distribution data generated with processed ATAC-seq data, highest efficiency for transgene insertion, durable expression following prolonged culture, absence of oncogenic gene expression, stable expression of a transgene, knockout of the gene confers benefit to the function of the cell, no known function within the cell, stable transgene expression in vitro, negligible off-target cleavage as detected by iGuide-Seq or CRISPR-Seq, less off-target cleavage relative to other loci as detected by iGuide-Seq or CRISPR-Seq, negligible transgene-independent cytotoxicity, negligible transgene-independent cytokine expression, negligible transgene-independent chimeric antigen receptor expression, negligible deregulation or silencing of nearby genes wherein the accessibility of chromatin is measured using an assay for transposase-accessible chromatin using sequencing (ATAC-seq). In some embodiments, chromatin accessibility is based on the (1) PSCs, to optimize editing efficiency in step a, and/or (2) DA neuronal cells, to optimize editing efficiency in step a, and (3) post-mitotic dopaminergic neurons from human brain samples. In some embodiments, defining chromatin accessibility is based on the (1) PSCs, to optimize editing efficiency in step a, (2) DA neuronal cells, to optimize editing efficiency in step a, (3) post-mitotic dopaminergic neurons from human brain samples to the extent the engineered cells are able to mature to a Neuronal Mature Cell Type to promote survival of endogenous neurons and (4) Neuronal Mature Cell Type cells.

Disclosed is an ex vivo method of obtaining an engineered cell or population thereof, comprising: a. obtaining a cell; b. genetically modifying the cell by inserting at least one sequence encoding a transgene within a safe harbor locus, wherein the safe harbor locus is selected from any one of the target loci in Table 1. Wherein genetically modifying in step (b) comprises contacting the cell with one or more guide ribonucleic acids (gRNAs), the at least one sequence, and one or more Cas9 endonucleases, wherein the one or more gRNAs and Cas9 endonucleases facilitate the insertion of the at least one sequence into chromosomal DNA within the safe harbor locus. Wherein the at least one sequence comprises an exogenous promoter and the exogenous promoter is operably linked to the transgene. Wherein the transgene comprises, consists of or consists essentially of one or more genes selected from the group functional PARK2, PINK1, DJ-1, LRRK2, c-Rel, ATG7, VMAT2, GBA, hemizygous SNCA, hemizygous MAPT and/or GDNF gene or a functional variant or fragment thereof.

### Gene Targeted Insertion in Pluripotent Stem Cells (PSCs)

Provided is a method of performing gene targeted insertion in PSCs to introduce exogenous coding sequences under the control of endogenous promoters, especially endogenous promoters of genes that are specifically expressed into cells of a particular neuronal lineage or at particular differentiation stage, preferably at a late stage of differentiation. In some embodiments, the PSCs can be transduced with a polynucleotide vector (donor template), such as an AAV vector, during an ex-vivo treatment as discussed herein, whereas a sequence specific nuclease reagent is expressed as to promote the insertion of the coding sequences at the selected locus. In some embodiments, the PSCs can be engineered using CRISPR, TALEN, transposon-based, ZFN, meganuclease, or Mega-TAL to create a double stand break which is repaired by insertion of a gene encoding an engineered GDNF, GBA and/or SNCA gene. The resulting engineered PSCs can be then differentiated into engineered DA neuronal cells and the engineered DA neuronal cells engrafted into a patient in need thereof for a long-term in vivo production of the exogenous coding sequences. Depending on the activity of the selected endogenous promoter, the coding sequences will be selectively expressed in certain lineages or in response to the local environment of the DA neuronal cells in vivo, thereby providing treatment for Parkinson's disease and other and secondary Parkinsonian disorders.

In some embodiments, the exogenous coding sequences are placed under the control of promoters of a gene, which transcriptional activity is specifically induced in cells after graft function has been established, i.e., cells which are a Neuronal Mature Cell Type. In some embodiments, the exogenous coding sequences are placed under the control of promoters of a gene, which transcriptional activity is specifically induced in cells after cell transplantation has occurred. In some embodiments, the exogenous coding sequences are placed under the control of promoters of a gene, which transcriptional activity is specifically not induced in cells differentiating in vitro. In some embodiments, the exogenous coding sequences are placed under the control of promoters of a gene, which transcriptional activity is specifically not induced in cells expanding in vitro. In some embodiments, the exogenous coding sequences are placed under the control of promoters of a gene, which transcriptional activity is specifically not induced in cells until after graft function has been established.

Disclosed is the introduction of an exogenous sequence encoding a GDNF, GBA, SNCA+/− or a component thereof, into PSCs, preferably under the transcriptional control of a promoter of a gene that is not expressed in pluripotent cells or differentiating cells or DA neuronal cells. In some embodiments, the gene is under transcriptional control of a gene that is only expressed upon transplantation in vivo. In some embodiments, the gene is under transcriptional control of a gene that is only expressed in the neural cells produced by the administered engineered ESC and/or DA neuronal cell. In some embodiments, the gene is under transcriptional control of a gene that is only expressed in Neuronal Mature Cell Types. In some embodiments, the gene is under transcriptional control of one of SEQ ID NOS: 21-40 or 43.

In some embodiments the exogenous sequence encoding a GDNF, or a component thereof, is introduced into the ESC under the transcriptional control of a gene that is described as being specifically expressed in Neuronal Mature Cell Types, preferably only in these types of cells. In some embodiments, the GDNF gene is under transcriptional control of one of SEQ ID NOS: 21-40 or 43.

In some embodiments, the PSCs comprise an exogenous coding sequence to be expressed exclusively in selected neural lineage(s). More broadly, disclosed is a method of engineering PSCs by gene targeted insertion of an exogenous coding sequences to be selectively expressed in Neuronal Mature Cells derived from the PSCs. As an embodiment, the Neuronal Mature cells produced by the engineered PSCs express the exogenous coding sequences in response to selected environmental factors or in vivo stimuli to improve their therapeutic potential. More broadly, disclosed is a method of engineering PSCs by gene targeted insertion of an exogenous coding sequences to be selectively expressed in an in vivo environment where Neuronal Mature Cells are. As an embodiment, the engineered cells derived from the engineered PSCs express the exogenous coding sequences in response to selected environmental factors or in vivo stimuli to improve their therapeutic potential on Neuronal Mature Cells.

Combining Targeted Sequence Insertion(s) in DA Neuronal Cells with the Inactivation of Endogenous Genomic Sequences

In some embodiments, gene inactivation in DA neuronal cells at a locus, by integrating exogenous coding sequence at the locus, the expression of which improves the therapeutic potential of the engineered cells.

In some embodiments, the insertion of the coding sequence has the effect of reducing or preventing the expression of genes involved.

### Expansion of DA Neuronal Cells

Whether prior to or after genetic modification, the engineered pluripotent cells and/or DA neuronal cells can be expanded. Pluripotent cells and/or DA neuronal cells can be expanded in vitro or in vivo. In some embodiments, the engineered pluripotent cells and/or DA neuronal cells do not express the exogenous gene during expansion. In some embodiments, the engineered pluripotent cells and/or DA neuronal cells do express the exogenous gene during expansion.

### Gene Editing

In some embodiments, disclosed is a method comprises the steps of:
providing a population of pluripotent cells;
introducing into a proportion of the pluripotent cells:
   i) at least one nucleic acid comprising an exogenous nucleotide or polynucleotide sequence thereby producing an engineered pluripotent cell wherein the exogenous nucleotide or polynucleotide is integrated at a selected endogenous locus to encode at least one molecule for improving the therapeutic potential of a DA neuronal cells population derived from the engineered pluripotent cells;
   ii) At least one sequence-specific reagent that specifically targets the selected endogenous locus, wherein the exogenous nucleotide or polynucleotide sequence is inserted by targeted gene integration into the endogenous locus, so that the exogenous nucleotide or polynucleotide sequence forms an exogenous coding sequence under transcriptional control of an endogenous promoter present at the locus.

In some embodiments of the method, the sequence specific reagent is a nuclease, and the targeted gene integration is operated by homologous recombination or non-homologous end joining (NHEJ) into the pluripotent cells. In some embodiments, NHEJ can be suppressed in a cell. Suppressing NHEJ in a cell can comprise inhibiting Ligase IV. Suppressing NHEJ in a cell can also comprise introducing a homologous recombination (HR) enhancer. An enhancer can be derived from a viral protein. An enhancer can be E1B55K, E4orf6, Scr7, or L755507. Suppressing NHEJ in a cell can facilitate insertion of a polynucleotide encoding an exogenous GDNF, GBA and/or SNCA at a cleavage site by homologous recombination.

In some embodiments, the endogenous promoter is selected to be inactive during in vitro cell differentiation and preferably up regulated after graft function has been established in vivo. In some embodiments, the endogenous promoter is selected to be inactive during in vitro cell differentiation and preferably up regulated after cell transplantation into a subject.

The methods can be further understood by the flowing numbered paragraphs:
Paragraph 1. A method of treating Parkinson's disease and other and secondary Parkinsonian disorders in a human subject, the method comprising: administering to the human subject an effective amount of a pharmaceutical composition that comprises (i) a population of genetically modified human DA neuronal cells that comprise a genomic disruption in an endogenous gene that suppresses or eliminates expression of a protein encoded by said gene; and (ii) a pharmaceutically acceptable carrier or excipient.
Paragraph 2. The method of paragraph 1, wherein said gene is a GDNF, GBA and/or SNCA gene.
Paragraph 3. The method of paragraph 1, wherein said DA neuronal are autologous to said human subject.
Paragraph 4. The method of paragraph 1, wherein said genomic disruption is a nucleotide insertion or deletion in said gene.
Paragraph 5. The method of paragraph 1, wherein said genetically modified human DA neuronal cells comprise an exogenous GDNF, GBA and/or SNCA gene.
Paragraph 7. The method of paragraph 1, wherein said genetically modified DA neuronal cells are derived from genetically modified pluripotent stem cells.

### Gene Editing for Hemizygous Null for SNCA

In one embodiment, provided is a method for adoptive cell therapy or prophylaxis comprising administering SNCA-modified DA neuronal cells to a subject suffering from a α-synuclein accumulation condition or at risk of suffering from a α-synuclein accumulation condition. In some embodiments the SNCA-modified DA neuronal cells are autologous. In some embodiments the SNCA-modified DA neuronal cells are allogeneic.

In some embodiments the SNCA-modified DA neuronal cells are DA neuronal cells genetically modified to have reduced expression of SNCA. In some embodiments the SNCA-modified DA neuronal cells are DA neuronal cells genetically modified to have reduced expression of SNCA and increased expression of GBA.

In some embodiments the SNCA-modified DA neuronal cells are derived from pluripotent cells genetically modified to have reduced expression of SNCA such as by gene editing with programmable nucleases or by gene silencing (RNA interference). In some embodiments the genetically modified SNCA-modified DA neuronal cells are SNCA^{-/-}. In some embodiments the genetically modified SNCA-modified DA neuronal cells are SNCA^{+/-}. In some embodiments the genetically modified SNCA-modified DA neuronal cells are SNCA^{+/+}.

SNCA-modified DA neuronal cells can be further understood by the following numbered paragraphs:
Paragraph 1. A method of obtaining human DA neuronal cells, comprising differentiating human pluripotent stem cells into human DA neuronal cells, wherein the human pluripotent cells are genetically modified cells in which one or both SNCA alleles have been unexpressed by a genetic modification.
Paragraph 2. The method of paragraph 1, wherein both SNCA alleles have been unexpressed by a genetic modification.
Paragraph 3. The method of paragraph 1, wherein human pluripotent stem cells are induced pluripotent stem cells.
Paragraph 4. The method of paragraph 3, wherein both SNCA alleles have been unexpressed by a genetic modification.
Paragraph 5. The method of paragraph 1, comprising expanding the human DA neuronal cells.
Paragraph 6. The method of paragraph 1, wherein the human pluripotent cells are genetically modified using CRISPR/Cas protein gene editing.
Paragraph 7. A population of human cells comprising human DA neuronal cells wherein the human DA neuronal cells are genetically modified human DA neuronal cells in which one or both SNCA alleles have been unexpressed by a genetic modification.

### Cellular Toxicity

Disclosed herein, can further comprise introducing into a cell a modifier to reduce cellular toxicity. A modifier can be Pan Caspase Inhibitor Z-VADFMK and/or BX795. A cell can be a pluripotent cell, DA neuronal cell or neural cell. A cell can be a mammalian cell. A cell can be a human cell.

### CIS and IRES

In some embodiments, disclosed is a method comprising gene editing of pluripotent cells (or in some embodiments DA neuronal cells) to have integrated gene transcription under the control of an endogenous promoter while maintaining the expression of the native gene through the use of CIS-regulatory elements (e.g., 2A CIS-acting hydrolase elements) or of internal ribosome entry site (IRES) in the donor template.

### GDNF Upregulated Upon Completion of DA Neuronal Cell Differentiation

In some embodiments, disclosed is a method comprising generating a double-strand break at a locus highly transcribed once graft function has been established by expressing sequence-specific nuclease reagents, such as TALEN, ZFN or RNA-guided endonucleases as non-limiting examples, in the presence of a DNA repair matrix preferably set into an AAVSI based vector. This DNA donor template generally includes two homology arms embedding unique or multiple open reading frames and regulatory genetic elements (stop codon and polyA sequences).

Disclosed is a method of expressing GDNF at selected gene loci that are upregulated upon completion of DA neuronal cell's differentiation, engraftment, proliferation, migration, and innervation in vivo, i.e., once graft function has been established or once the administered engineered DA neuronal cells have differentiated to a Neuronal Mature Cell Type. The exogenous sequence(s) encoding the GDNF gene or functional fragment thereof and the endogenous gene coding sequence(s) may be co-transcribed, for instance by being separated by CIS-regulatory elements (e.g., 2A CIS-acting hydrolase elements) or by an internal ribosome entry site (IRES). For instance, the exogenous sequences encoding a GDNF gene or functional fragment thereof can be placed under transcriptional control of the promoter of endogenous genes that are expressed by the graft microenvironment.

### GBA Upregulated Upon Completion of DA Neuronal Cell Transplantation

In some embodiments, disclosed is a method comprising generating a double-strand break at a locus highly transcribed as soon as engineered cells are transplanted by expressing sequence-specific nuclease reagents, such as TALEN, ZFN or RNA-guided endonucleases as non-limiting examples, in the presence of a DNA repair matrix preferably set into an AAVS1 based vector. This DNA donor template generally includes two homology arms embedding unique or multiple Open Reading Frames and regulatory genetic elements (stop codon and polyA sequences).

Disclosed is a method of expressing GBA at selected gene loci that are upregulated not upon completion of DA neuronal cell's engraftment but upon DA neuronal cell transplantation, i.e., before graft function has been established or before the administered DA neuronal cells have differentiated into a Neuronal Mature Cell Type. The exogenous sequence(s) encoding the GBA gene or functional fragment thereof and the endogenous gene coding sequence(s) may be co-transcribed, for instance by being separated by CIS-regulatory elements (e.g., 2A CIS-acting hydrolase elements) or by an internal ribosome entry site (IRES). For instance, the exogenous sequences encoding a GBA gene or functional fragment thereof can be placed under transcriptional control of the promoter of endogenous genes that are expressed by the graft transplantation, or they may be ubiquitous promoters.

A novel framework for identification of candidate genomic safe harbors (“GSH”) is disclosed in Aznauryan et al., Cell Reports Methods, 2,100154 (2022). Candidate GSHs will be selected based on ATAC-seq data showing favorable chromatin accessibility profiles in DA neurons. Fullard et al., Genome Research, 28:1243-1252 (2021) shows candidate GSH locus with evidence of accessible chromatin in human striatum (nucleus accumbens and putamen), and Corces et al., Nature Genetics, 52:1158-1168 (2020).

Contacting Neural Cells In Vivo with Engineered DA Neuronal Cells

In some embodiments, disclosed is a method of promoting viability, engraftment, proliferation, migration, innervation, differentiation, long-term graft integrity, survival of endogenous neurons, or function of the administered engineered DA neuronal cells of administered engineered DA neuronal cells, comprising contacting a neuron with a gene editing system comprising: Cas protein or a polynucleotide encoding a Cas protein; a guide-RNA (gRNA); and a repair template comprising a functional PARK2, PINK1, DJ-1, LRRK2, c-Rel, ATG7, VMAT2, GBA, SNCA+/− and/or GDNF gene or a functional variant or fragment thereof; wherein the gene editing system is capable of enhancing the viability, engraftment, proliferation, migration, innervation, differentiation, long-term graft integrity, survival of endogenous neurons, or function of the administered engineered DA neuronal cells of neuron compared to wildtype neuronal cells. In some embodiments, the neuron is in the patient's body when it is contacted with the engineered DA neuronal cells.

Disclosed is a method of engrafting, proliferating, or promoting viability, migration, innervation, differentiation or function of administered engineered DA neuronal cells comprising a recombinant gene vector comprising a polynucleotide encoding a wild-type PARK2, PINK1, DJ-1, LRRK2, c-Rel, ATG7, VMAT2, GBA, SNCA+/− and/or GDNF gene or a functional variant or fragment thereof; wherein the polynucleotide is operatively linked to a eukaryotically active promoter; and wherein a DA neuronal cell transduced with the recombinant gene expresses the wild-type protein, or functional variant or fragment thereof upon implantation into the host/patient. In some embodiments, a DA neuronal cell transduced with the recombinant gene expresses the wild-type protein, or functional variant or fragment thereof upon maturation in the host/patient.

Non-Viral Introduction of Exogenous Genes into a Cell

Also disclosed herein are methods for making an engineered cell comprising a) non-virally introducing into a cell one or more polynucleic acids comprising at least one exogenous GDNF, GBA and/or SNCA sequence; and b) contacting the at least one exogenous GDNF, GBA and/or SNCA exogenous sequence with a double stranded break region that comprises at least one gene. The at least one gene can be GDNF, GBA and/or SNCA. The double strand break region can be repaired by insertion of the at least one exogenous GDNF GBA and/or SNCA sequence. The insertion of the at least one exogenous GDNF, GBA and/or SNCA sequence can comprise disruption of the at least one gene.

In some embodiments, gene introduction can comprise non-viral transfection, biolistics, chemical transfection, electroporation, nucleofection, heat-shock transfection, lipofection, microinjection, or viral transfection. A polynucleic acid can be co-delivered with at least one modifier that alters cellular response to a polynucleic acid. At least one modifier can reduce cellular toxicity. A modifier can comprise abPan Caspase Inhibitor Z-VAD-FMK or BX795.

Introduction of Exogenous Gene into a Cell Via a Double Strand Break Region

Also disclosed herein are methods for making an engineered cell comprising introducing not a cell a double strand break region. In some embodiments, the double strand break region can be created by CRISPR, TALEN, transposon-based, ZEN, meganuclease, or Mega-TAL. In some embodiments, the double strand break region can be created by CRISPR. In some embodiments, CRISPR can be multiplexed. In some embodiments, multiplexing can be performed by adding at least 2 guide RNAs. The GDNF, GBA and/or SNCA exogenous sequence can be inserted near the double strand break region.

Introduction of Exogenous Gene into a Cell Via Reverse Transcriptase.

In some embodiments, the cell to be engineered can be contacted with reverse transcriptase (RT). In some embodiments, the cell can be contacted with primers that are complementary to the polynucleic acid. In some embodiments, the RT transcribes the mRNA into a first ssDNA template. In some embodiments, the RT transcribes the first ssDNA template into a second dsDNA template. In some embodiments, transcribing can be performed in situ. The ssDNA or dsDNA can comprise the at least one exogenous GDNF sequence. In some embodiments, primer sequences can be used to determine the presence of an RT. A Reverse Transcriptase (RT) reporter forward primer can be determined by known methods by one having ordinary skill in the art. A Reverse Transcriptase (RT) reporter forward primer can be determined by being able to (selectively) bind to a portion of one of SEQ ID NOS: 1-10. In some embodiments, the forward primer is 10 nucleic acids long and can bind to the first 10 N's (residues 1-10), second 10 N's (residues 10-20), third 10 N's (residues 20-30), fourth 10 N's (residues 30-50), or fifth 10 N's (residues 50-60) of the amino acid sequence set forth in SEQ ID NOS: 1-10.

### Homology Directed Repair

Disclosed herein are also methods for facilitating homology directed repair (HDR) comprising a) non-virally introducing into a cell an mRNA, reverse transcriptase (RT), enhancer, and primer; b) reverse transcribing the mRNA into one or more copies of a polynucleic acid; and c) facilitating HDR between the genome of the cell and of the polynucleic acid. In some embodiments, the method can comprise generating a double stranded break. In some embodiments, the double strand break can be performed by CRISPR, TALEN, transposon-based, ZFN, meganuclease, and Mega-TAL. In some embodiments, the double strand break can be performed by CRISPR. In some embodiments, the HDR of c) repairs the double strand break. In some embodiments, the CRISPR can be multiplexed with at least two (2) guide RNAs. In some embodiments, the polynucleic acid can be DNA. In some embodiments, the polynucleic acid can be cDNA. In some embodiments, the polynucleic acid can be single stranded.

In some embodiments, the RT transcribes the mRNA into a first ssDNA template. In some embodiments, the polynucleic acid can be double stranded. In some embodiments, the RT transcribes the mRNA into a second dsDNA template in situ. The mRNA or polynucleic acid can comprises at least one GDNF sequence. In some embodiments, the GDNF sequence comprises at least two flanking recombination arms having a sequence complementary to a genomic region. In some embodiments, the GDNF sequence can be used in HDR of c). In some embodiments, the GDNF sequence can be used in HDR of c) and further comprises binding of the recombination arms to a complementary portion of the genome of the cell. In some embodiments, the GDNF sequence can be used in HDR of c) and further comprises binding of the recombination arms to a complementary portion of the genome of the cell and further comprises insertion of the GDNF sequence. In some embodiments, HDR between the genome of the cell and of the polynucleic acid disrupts one or more genes. One or more genes can comprise XX. In some embodiments, one or more genes comprise a GDNF. In some embodiments, HDR between the genome of the cell and of the polynucleic acid can be assisted by one or more homologous recombination (HR) enhancers.

### Targeted Homologous Recombination

In some embodiments, the exogenous sequence is introduced into the endogenous chromosomal DNA by targeted homologous recombination. Accordingly, the exogenous nucleic acid introduced into the pluripotent cell comprises at least one coding sequence(s), along with sequences that can hybridize endogenous chromosomal sequences under physiological conditions. In general, such homologous sequences show at least 70%, preferably 80% and more preferably 90% sequence identity with the endogenous gene sequences located at the insertion locus. These homologous sequences may flank the coding sequence to improve the precision of recombination as already taught for instance in U.S. Pat. No. 6,528,313 (which is incorporated in its entirety by reference).

Using available software and on-line genome databases, it is possible to design vectors that include the coding sequence(s), in such a way that the sequence(s) is (are) introduced at a precise locus, under transcriptional control of at least one endogenous promoter, which is a promoter of an endogenous gene. The exogenous coding sequence(s) is (are) then preferably inserted “in frame” with the endogenous gene. The sequences resulting from the integration of the exogenous polynucleotide sequence(s) can encode many different types of proteins, including fusion proteins, tagged protein or mutated proteins. Fusion proteins allow adding new functional domains to the proteins expressed in the cell, such as a dimerization domain that can be used to switch-on or switch-off the activity of the protein, such as caspase-9 switch. Tagged proteins can be advantageous for the detection of the engineered DA neuronal cells and the follow-up of the patients treated with the cells. Introducing mutation into proteins can confer resistance to drugs as further described below.

### Rare-Cutting Endonuclease

The sequence specific reagent used in this method is preferably a rare-cutting endonuclease as known by one skilled in the art. Targeted gene integration is generally operated by homologous recombination or NHEJ into the pluripotent cells. The specific endonuclease reagent is preferably selected from an RNA or DNA-guided endonuclease, such as Cas9 or Cpf1, an RNA or DNA guide, a TAL-endonuclease, a zing finger nuclease, a homing endonuclease or any combination thereof.

### TALE-Nucleases

In some embodiments, the endonuclease reagent is a nucleic acid encoding an “engineered” or “programmable” rare-cutting endonuclease, such as a homing endonuclease as described for instance by Arnould S., et al. (WO2004067736), a zing finger nuclease (ZFN) as described, for instance, by Urnov F., et al. (Highly efficient endogenous human gene correction using designed zinc-finger nucleases (2005) Nature 435:646-651), a TALE-Nuclease as described, for instance, by Mussolino et al. (A novel TALE nuclease scaffold enables high genome editing activity in combination with low toxicity (2011) Nucl. Acids Res. 39 (21): 9283-9293), or a MegaTAL nuclease as described, for instance by Boissel et al. (MegaTALs: a rare-cleaving nuclease architecture for therapeutic genome engineering (2013) Nucleic Acids Research 42 (4): 2591-2601).

In some embodiments, the endonuclease reagent is an RNA-guide to be used in conjunction with a RNA guided endonuclease, such as Cas9 or Cpf1, as per, inter alia, the teaching by Doudna, J., and Chapentier, E., (The new frontier of genome engineering with CRISPR-Cas9 (2014) Science 346 (6213): 1077), which is incorporated herein by reference.

In some embodiments, the endonuclease reagent is transiently expressed into the cells, meaning that the reagent is not supposed to integrate into the genome or persist over a long period of time, such as be the case of RNA, more particularly mRNA, proteins or complexes mixing proteins and nucleic acids (e.g.: Ribonucleoproteins).

In general, 80% the endonuclease reagent is degraded by 30 hours, preferably by 24, more preferably by 20 hours after transfection.

An endonuclease under mRNA form is preferably synthetized with a cap to enhance its stability according to techniques well known in the art, as described, for instance, by Kore A. L., et al. (Locked nucleic acid (LNA)-modified dinucleotide mRNA cap analogue: synthesis, enzymatic incorporation, and utilization (2009) J Am Chem Soc. 131 (18): 6364-5).

In general, electroporation steps that are used to transfect pluripotent cells are typically performed in closed chambers comprising parallel plate electrodes producing a pulse electric field between the parallel plate electrodes greater than 100 volts/cm and less than 5,000 volts/cm, substantially uniform throughout the treatment volume such as described in WO/2004/083379, which is incorporated by reference. One such electroporation chamber preferably has a geometric factor (cm¹) defined by the quotient of the electrode gap squared (cm2) divided by the chamber volume (cm³), wherein the geometric factor is less than or equal to 0.1 cm¹, wherein the suspension of the cells and the sequence-specific reagent is in a medium which is adjusted such that the medium has conductivity in a range spanning 0.01 to 1.0 milliSiemens. In general, the suspension of cells undergoes one or more pulsed electric fields. With the method, the treatment volume of the suspension is scalable, and the time of treatment of the cells in the chamber is substantially uniform.

Due to their higher specificity, TALE-nuclease have proven to be particularly appropriate sequence specific nuclease reagents for therapeutic applications, especially under heterodimeric forms—i.e., working by pairs with a “right” monomer (also referred to as “5” or “forward”) and left” monomer (also referred to as “3” or “reverse”) as reported for instance by Mussolino et al. (TALEN® facilitate targeted genome editing in human cells with high specificity and low cytotoxicity (2014) Nucl. Acids Res. 42 (10): 6762-6773).

As previously stated, the sequence specific reagent is preferably under the form of nucleic acids, such as under DNA or RNA form encoding a rare cutting endonuclease a subunit thereof, but they can also be part of conjugates involving polynucleotide(s) and polypeptide(s) such as so-called “ribonucleoproteins”. Such conjugates can be formed with reagents as Cas9 or Cpf1 (RNA-guided endonucleases) or Argonaute (DNA-guided endonucleases) as recently respectively described by Zetsche, B. et al. (Cpf1 Is a Single RNA-Guided Endonuclease of a Class 2 CRISPR-Cas System (2015) Cell 163 (3): 759-771) and by Gao F. et al. (DNA-guided genome editing using the Natronobacterium gregoryi Argonaute (2016) Nature Biotech), which involve RNA or DNA guides that can be complexed with their respective nucleases.

### AAV Vectors

Improving the efficiency of gene targeted insertion in DA neuronal cells using AAV vectors

The donor templates are generally polynucleotide sequences which can be included into a variety of vectors described in the art to deliver the donor templates into the nucleus at the time the endonuclease reagents get active to obtain their site directed insertion into the genome generally by NHEJ or homologous recombination.

Disclosed is a method of inserting an exogenous nucleic acid sequence into an endogenous polynucleotide sequence in a cell, comprising at least the steps of transducing into the cell an AAV vector comprising the exogenous nucleic acid sequence and sequences homologous to the targeted endogenous DNA sequence, and

Inducing the expression of a sequence specific endonuclease reagent to cleave the endogenous sequence at the locus of insertion.

The obtained insertion of the exogenous nucleic acid sequence may result in the introduction of genetic material, correction or replacement of the endogenous sequence, more preferably “in frame” with respect to the endogenous gene sequences at that locus.

In some embodiments, from 10⁵ to 10⁷ preferably from 10⁶ to 10⁷, more preferably about 5.10⁶ viral genomes are transduced per cell. In some embodiments, the cells can be treated with proteasome inhibitors, such as Bortezomib to further help homologous recombination. In some embodiments, the AAV vector used in the method can comprise a promoter-less exogenous coding sequence in order to be placed under control of an endogenous promoter at one locus selected among those listed in the present specification. In some embodiments, the AAV vector used in the method can comprise a 2A peptide cleavage site followed by the cDNA (minus the start codon) forming the exogenous coding sequence. Disclosed is any AAV vectors designed to perform the method herein described, especially vectors comprising a sequence homologous to a locus of insertion. Many other vectors known in the art, such as plasmids, episomal vectors, linear DNA matrices, etc. can also be used following the teachings herein.

As stated before, the DNA vector comprises: (1) the exogenous nucleic acid comprising the exogenous coding sequence to be inserted by homologous recombination, and (2) a sequence encoding the sequence specific endonuclease reagent that promotes the insertion. In some embodiments, the exogenous nucleic acid under (1) does not comprise any promoter sequence, whereas the sequence under (2) has its own promoter. In some embodiments, the nucleic acid under (1) comprises an Internal Ribosome Entry Site (IRES) or “self-cleaving” 2A peptides, such as T2A, P2A, E2A or F2A, so that the endogenous gene where the exogenous coding sequence is inserted becomes multi-cistronic. The IRES of 2A Peptide can precede or follow the exogenous coding sequence.

### Transplantation

In some embodiments, the GDNF engineered DA neuronal cells are transplanted into patients with rapidly progressing and mild Parkinson's disease. In some embodiments, the GBA/SNCA engineered DA neuronal cells are transplanted into patients in order to improve cognitive symptoms in Parkinson's disease.

In some embodiments, an engineered cell can be administered to a subject in need thereof as a monotherapy. The engineered DA neuronal cells, recombinant gene vector or gene editing system can be administered in various ways. In some embodiments, the administering step comprises systemic, parenteral, intravenous, cerebral, cerebrospinal, intrathecal, intracisternal, intraputaminal, intrahippocampal, intra-striatal, or intra-cerebroventricular administration. In some embodiments, the administering step comprises intravenous, cerebral, cerebrospinal, intrathecal, intracisternal, intraputaminal, intrahippocampal, intra-striatal, or intra-cerebroventricular injection. In some embodiments, the administering step comprises intrathecal injection with Threndelenburg tilting. In some embodiments, the administering step comprises direct injection into the pars compacta of the substantia nigra of the brain. In some embodiments, the administering step comprises introducing the engineered DA neuronal cells, recombinant gene vector or gene editing system into the subject's brain or cerebrospinal fluid (CSF).

The administration of the cells or population of cells can consist of the administration of 10⁴-10⁹ cells per kg body weight, preferably 10⁵ to 10⁶ cells/kg body weight including all integer values of cell numbers within those ranges. The administration of the cells or population of cells can consist of more than 10, generally more than 50, more generally more than 100 and usually more than 1000 doses comprising between 10⁶ to 10⁸ gene edited cells originating from a single donor's or patient's sampling.

In some embodiments, 1×10⁹-1×10¹⁴ recombinant gene vector genomes per kilogram body mass of the subject (vg/kg) of the gene therapy vector are administered to the subject. In some embodiments, 1×10⁹-1×10¹⁴ recombinant gene vector genomes per kilogram body mass of the subject (vg/kg) of the gene therapy vector are administered to the subject's brain. In some embodiments, 1×10⁹-1×10¹⁴ recombinant gene vector genomes per kilogram body mass of the subject (vg/kg) of the gene therapy vector are administered to the subject's CSF. In some embodiments, 1×10⁷-1×10⁹ recombinant gene vector genomes per kilogram body mass of the subject (vg/kg) of the gene therapy vector are administered to the subject.

In some embodiments, a subject in need thereof receives treatment comprising administering to the subject a therapeutically effective amount of a pharmaceutical composition comprising an engineered cell. In some embodiments, a subject in need thereof receives treatment comprising administering to the subject a therapeutically effective amount of a pharmaceutical composition comprising an engineered cell comprising an exogenous GDNF, GBA or SNCA gene or functional fragment thereof. A pharmaceutical composition can be administered intravenously. A pharmaceutical composition can be administered locally. In some embodiments, a method can further comprise administering one more or more additional therapies. The one or more additional therapies can comprise transplantation. The one or more additional therapies can comprise immunotherapy. In some embodiments, the engineered cell can be autologous to the subject. In some embodiments, the engineered cell can be allogenic to the subject.

Engineered DA neuronal cells can be administered to an individual, in any amount or number that results in a detectable therapeutic or prophylactic benefit to the individual, e.g., an effective amount. In some embodiments the dose of engineered DA neuronal cells to be administered is simply an absolute numbers of cells, e.g., said individual can be administered about 1×10⁵ cells, 5×10⁵ cells, 1×10⁶ cells, 7×10⁶ cells, 1×10⁷ cells, 6×10⁷ cells, 2×10⁸ cells, 5×10⁸ cells, 1×10⁹ cells, 6×10⁹ cells, 2×10¹⁰ cells, 5×10¹⁰ cells, or 1×10¹¹ cells.

In some embodiments, engineered DA neuronal cells are administered to a subject by a numbers of cells relative to the weight of the subject to be treated, e.g., at about, 1×10⁵ cells, 5×10⁵ cells, 1×10⁶ cells, 7×10⁶ cells, 1×10⁷ cells, 6×10⁷ cells, 2×10⁸ cells, 5×10⁸ cells, 1×10⁹ cells, or 6×10⁹ cells per kilogram of the subject to be treated.

The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration.

The disclosure also encompasses means for detecting the engineered cells comprising the desired genetic insertions, especially by carrying out steps of using PCR methods for detecting insertions of exogenous coding sequences at the endogenous loci. In some embodiments, allogeneic tags will be used to monitor graft integrity and functionality.

The above written description provides a manner and process of making and using it such that any person skilled in this art is enabled to make and use the same, this enablement being provided in particular for the subject matter of the appended paragraphs, which make up a part of the original description.

### Combination Treatments

Modified DA neuronal cell compositions can also be used in combination with other agents of therapeutic value in the treatment of PD symptoms. According to an embodiment, the treatment can be administrated into patients undergoing an immunosuppressive treatment. In general, other agents do not necessarily have to be administered in the same pharmaceutical composition, and may, because of different physical and chemical characteristics, preferably be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

In some embodiments, treatment of the modified DA neuronal cell composition can be used in conjunction with a medication that is designed to effect the cholinergic system in the brain. Examples of such medications can be, but are not limited to the group comprising or consisting of the following: donepezil (Aricept), rivastigmine (Exelon), and galantamine (Razadyne).

It is known to those of skill in the art that therapeutically effective dosages can vary when the drugs are used in treatment combinations. Methods for experimentally determining therapeutically effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

For combination therapies, dosages of co-administered therapeutic agents will of course vary depending on the type of co-agents employed, on the specific Modified DA neuronal cell, and Parkinson's disease and other secondary Parkinsonian disorder symptom to be treated.

It is understood that the dosage regimen to treat, prevent, or ameliorate the condition(s) for which relief is sought, can be modified in accordance with a variety of factors. These factors include the condition from which the subject suffers, as well as the age, weight, sex, diet, and general medical condition of the subject. Thus, the dosage regimen actually employed can vary widely and therefore can deviate from the dosage regimens set forth herein.

The Modified DA neuronal cell and additional therapeutic agent which make up a combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of various physiological parameters may also be evaluated to determine the optimal dose interval.

Initial administration can be via any route practical, such as, for example, an intravenous injection, a bolus injection, infusion over 5 minutes to about 5 hours, a pill, a capsule, inhaler, injection, transdermal patch, buccal delivery, and the like, or combination thereof. A compound should be administered as soon as is practicable after the onset of a disease or condition is detected or suspected, and for a length of time necessary for the treatment or prevention of the PD condition.

### Dosage Forms

Useful compositions can be formulated for administration to a subject via any conventional means including, but not limited to, oral, parenteral (e.g., intravenous, subcutaneous, or intramuscular), buccal, inhalation, intranasal, rectal or transdermal administration routes.

The pharmaceutical compositions which include a modified DA neuronal cell alone or in combination with one or more other therapeutic agents, can be formulated into any suitable dosage form, including but not limited to, aqueous oral dispersions, liquids, mists, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use can be obtained by mixing one or more solid excipient with one or more of the compounds, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as:
polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

In another aspect, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Microencapsulated formulations of a DA neuronal cell population may be formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

The pharmaceutical solid oral dosage forms including formulations can be further formulated to provide a controlled release of the DA neuronal cell population. Controlled release refers to the release of one or more active agents from a dosage form in which they are incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms can be formulated as enteric coated delayed release oral dosage forms, i.e., as an oral dosage form of a pharmaceutical composition which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5 but does dissolve at pH about 5 and above. It is expected that any anionic polymer exhibiting a pH-dependent solubility profile can be used as an enteric coating in the methods and compositions to achieve delivery to the lower gastrointestinal tract. In some embodiments the polymers are anionic carboxylic polymers.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Liquid formulation dosage forms for oral administration can be aqueous suspensions selected from the group including, but not limited to, pharmaceutically acceptable aqueous oral dispersions, emulsions, solutions, elixirs, gels, and syrups.

The aqueous suspensions and dispersions can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

### Injectable Formulations

Formulations suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, compounds may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

Parenteral injections may involve bolus injection or continuous infusion. Formulations for injection may be presented in unit dosage form, e.g., in ampoules or in multi-dose containers, with an added preservative. The pharmaceutical composition may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

The pharmaceutical compositions may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

### Treatment Outcomes

In some embodiments, the number of Neuronal Mature Cells in the subject after the administering step (administering engineered DA neuronal cells, recombinant gene vector or gene editing system) is greater than the number of Neuronal Mature Cells in the subject before the administering step. In some embodiments, the endogenous Neuronal Mature Cells in the subject after the administering step is greater than the number of endogenous Neuronal Mature Cells in the subject before the administering step.

In some embodiments, the number of dopaminergic neurons in the subject after the administering step (administering engineered DA neuronal cells, recombinant gene vector or gene editing system) is greater than the number of dopaminergic neurons in the subject before the administering step. In some embodiments, the level of dopamine in the subject after the administering step is greater than the level of dopamine in the subject before the administering step. In some embodiments, the number of dopaminergic neurons in a subject treated by the method is increased compared to the number of dopaminergic neurons in a subject not so treated. In some embodiments, the level of dopamine of a subject treated by the methods disclosed herein (administering engineered DA neuronal cells, recombinant gene vector or gene editing system) is increased compared to the level of dopamine in a subject not so treated. In some embodiments, the level of dopamine in the substantia nigra of a subject treated by the methods disclosed herein is increased compared to the level of dopamine in the substantia nigra of a subject not so treated. In some embodiments, the level of GDNF in the subject's Cerebrospinal fluid (CSF) after the administering (administering engineered DA neuronal cells, recombinant gene vector or gene editing system) step is greater than the level of GDNF in the subject's CSF before the administering step. In some embodiments, the level of GBA in the subject's CSF after the administering (administering engineered DA neuronal cells, recombinant gene vector or gene editing system) step is greater than the level of GBA in the subject's CSF before the administering step. In some embodiments, the ratio of GBA to SNCA in the subject after the administering (administering engineered DA neuronal cells, recombinant gene vector or gene editing system) step is less than the ratio of GBA to SNCA in the subject before the administering step. In some embodiments, the ratio of GBA to SNCA in the subject after the administering (administering engineered DA neuronal cells, recombinant gene vector or gene editing system) step is more than the ratio of GBA to SNCA in the subject before the administering step.

In some embodiments, the ratio of GBA protein to alpha-synuclein protein in the subject after the administering (administering engineered DA neuronal cells, recombinant gene vector or gene editing system) step is less than the ratio of GBA protein to alpha-synuclein in the subject before the administering step. In some embodiments, the ratio of GBA protein to alpha-synuclein protein in the subject after the administering (administering engineered DA neuronal cells, recombinant gene vector or gene editing system) step is more than the ratio of GBA protein to alpha-synuclein protein in the subject before the administering step.

In some embodiments, the Unified Parkinson's Disease Rating Scale (UPDRS) score of the subject before the administering step (administering engineered DA neuronal cells, recombinant gene vector or gene editing system) is improved compared to the UPDRS score of the subject before the administering step. In some embodiments, the subject's neurons express a reduced amount of alpha-synuclein and/or comprises a reduced amount of Lewy bodies following the administering step.

In some embodiments, the cognitive symptoms of the subject before the administering step (administering engineered DA neuronal cells, recombinant gene vector or gene editing system) is improved compared to the cognitive symptoms of the subject before the administering step.

In some embodiments, the long term graft integrity of the engineered cells administered to the subject is improved compared to long term graft integrity of non-engineered cells administered to a subject.

### Sequences

In some embodiments, the PARK2, PINK1, LRRK2, c-Rel, ATG7, VMAT2, GBA, SNCA, MAPT or GDNF gene (or vector) comprises the nucleic acid sequence set forth in SEQ ID NOs: 1-10, respectively.

In some embodiments, the PARK2, PINK1, LRRK2, c-Rel, ATG7, VMAT2, GBA, SNCA, MAPT or GDNF protein comprises the amino acid sequence set forth in SEQ ID NOs: 11-20, respectively.

In some embodiments, the gene is the GDNF gene, and the wild-type GDNF protein comprises the amino acid sequence set forth in any of SEQ ID NOs: 20.

In some embodiments, the gene is the GBA gene, and the wild-type GBA protein comprises the amino acid sequence set forth in any of SEQ ID NOs: 17.

In some embodiments, the polynucleotide comprises a sequence having at least 70%, 75%, 80%, 85%, 95%, or 99% identity to a PARK2, PINK1, LRRK2, c-Rel, ATG7, VMAT2, GBA, or GDNF polynucleotide sequence set forth in SEQ ID NOs: 1-10, respectively. In some embodiments, the polynucleotide is codon optimized. In some embodiments, the polynucleotide comprises less than 40, less than 30, less than 20, or 10 or fewer CpG islands. In some embodiments, the polynucleotide comprises at least 1, at least 2, at least 3, at least 4, at least 5, at least 6, or at least 10 CpG islands. In some embodiments, it comprises between 5 and 20 CpG islands.

Polynucleotide sequences of the vectors, donor templates comprising the exogenous coding sequences and/or sequences homologous to the endogenous loci, the sequences pertaining to the resulting engineered cells, as well as those permitting the detection of the engineered cells are all part of the present disclosure.

In some embodiments, the transgene encodes the GDNF gene, and the transgene polynucleotide sequence shares at least 95% identity to one of SEQ ID NO: 10 and is behind a promotor selected from SEQ ID NOs: 21-30.

In some embodiments, the transgene encodes the GBA gene, and the transgene polynucleotide sequence shares at least 95% identity to one of SEQ ID NO: 7 and is behind a promotor selected from SEQ ID NOs: 31-40 or 43.

In some embodiments, the transgene encodes the SNCA gene, and the transgene polynucleotide sequence shares at least 95% identity to one of SEQ ID NOs: 8 and is behind a promotor selected from SEQ ID NOs: 21-40 or 43.

In some embodiments, the first transgene encodes the GBA, and second transgene encodes the SNCA, and the first transgene is behind a promotor selected from SEQ ID NOs: 31-40 or 43 and the second transgene is behind a promotor selected from SEQ ID NOs: 21-40 or 43.

The Genetic Sequences Encoding GDNF, GBA and/or SNCA

The GDNF, GBA and/or SNCA sequence(s) can be introduced at selected loci, more particularly under control of endogenous promoters by targeted gene recombination. In some embodiments, the exogenous GDNF is expressed preferably only after the DA neuronal cell has differentiated and graft function has been established and the exogenous GBA and SNCA+/− is expressed preferably as soon as the engineered cell is transplanted into the patient.

According to an embodiment the exogenous sequence encodes a polypeptide displaying at least 80% amino acid sequence identity with PARK2, PINK1, LRRK2, c-Rel, ATG7, VMAT2, GBA, SNCA+/− or GDNF or a functional variant thereof. These exogenous sequences can be introduced into the genome by deleting or modifying the endogenous coding sequence(s) present at the locus (knock-out by knock-in), so that a gene inactivation can be combined with genesis.

In some embodiments, the exogenous sequences can be introduced into the genome without deleting or modifying the endogenous coding sequence(s) present at the locus. This occurs whether the endogenous coding sequence(s) is in an in vitro pluripotent cell or in vivo.

Depending on the locus targeted and its involvement in DA neuronal cell activity, the targeted endogenous gene may be unexpressed or maintained in its original function (whether the targeted gene is in an in vitro cell or in vivo). Should the targeted gene be essential for DA neuronal cell activity or differentiation, this insertion procedure can generate a single (Kl) without gene inactivation. Alternatively, if the targeted gene is deemed involved in DA neuronal cell differentiation, the insertion procedure is designed to prevent expression of the endogenous gene, preferably by knocking-out the endogenous sequence, while enabling expression of the introduced exogenous coding sequence(s).

In some embodiments, the method relies on up-regulating, with various kinetics, the target gene expression upon activation of the GDNF/GBA signaling pathway(s) and/or regulation of the SNCA signaling pathway by targeted integration (with or without the native gene disruption) at the specific loci such as, as non-limiting example, SEQ ID NOS: 21-40 or 43.

In some embodiments, disclosed are engineered DA neuronal cells, and preferably DA neuronal cells for infusion into patients, comprising exogenous sequences encoding PARK2, PINK1, LRRK2, c-Rel, ATG7, VMAT2, GBA, SNCA+/− or GDNF polypeptide(s), which are integrated at the PARK2, PINK1, LRRK2, c-Rel, ATG7, VMAT2, GBA, SNCA+/− or GDNF endogenous locus for their expression under the control of the endogenous promoters present at these loci. In some embodiments, the endogenous promoters comprise or consist of SEQ ID NOS: 21-40 or 43.

The engineered DA neuronal cells can be SNCA^{+/+}, SNCA^{-/-}, or SNCA^{+/-}, depending on the therapeutic indications and recipient patients. In some embodiments, the engineered DA neuronal cells are further made T-cell receptor^{negative} for allogeneic transplantation.

The gene editing step of integrating an exogenous sequence encoding PARK2, PINK1, LRRK2, c-Rel, ATG7, VMAT2, GBA, SNCA+/− or GDNF can be combined with any other step contributing to enhance the potency or the safety of the engineered DA neuronal cells.

According to an embodiment, the method relies on introducing the sequence specific endonuclease reagent and/or the donor template containing the gene of interest and sequences homologous to the target gene by transfecting ssDNA (oligonucleotides as non-limiting example), dsDNA (plasmid DNA as non-limiting example), and more particularly adeno-associated virus (AAV) as non-limiting example.

### Kit

Disclosed are kits for pluripotent and/or DA neuronal cells transfection comprising polynucleotides encoding the sequence-specific endonuclease reagents and the donor sequences designed to integrate the exogenous sequence at the locus targeted by the reagents. Examples of such kits are a kit comprising mRNA encoding rare-cutting endonuclease targeting Synapsin 1, DAT, VMAT, TH, AADC, tamoxifen inducible promoter, RU486 inducible promoter, or other promotor that allows for temporal or small molecule control locus and an AAV vector comprising an exogenous sequence encoding GDNF, a kit comprising mRNA encoding rare-cutting endonuclease targeting Synapsin 1, DAT, VMAT, TH, AADC, tamoxifen inducible promoter, RU486 inducible promoter, or other promotor that allows for temporal or small molecule control locus and an AAV vector comprising an exogenous sequence encoding GBA.

Disclosed are kits for pluripotent cell and/or DA neuronal cells transfection comprising polynucleotides encoding sequence-specific endonuclease reagents and an exogenous polynucleotide sequence, in some embodiments comprised into a AAV vector, the exogenous sequence comprising a sequence encoding GBA, GBA and/or SNCA^{+/-} or functional fragments or variants thereof.

### EXAMPLES

### Example 1

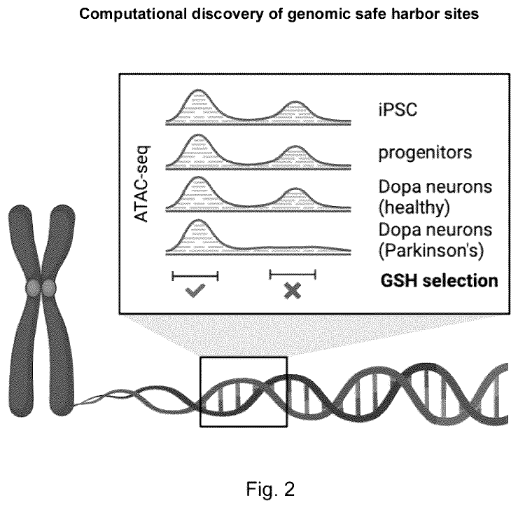

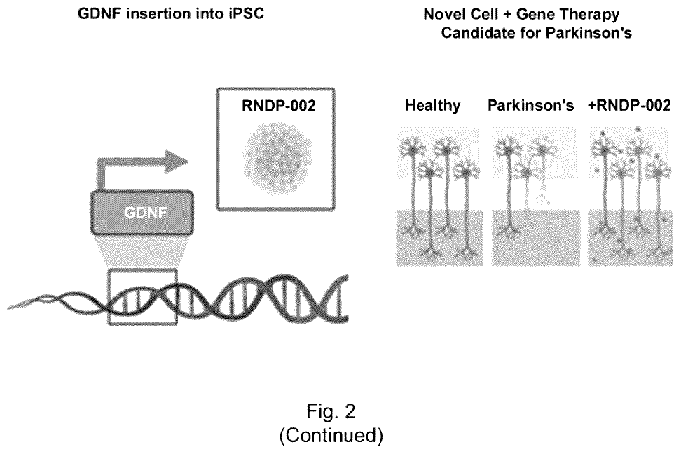

Applicants identified Genomic Safe Harbor (GSH) sites suitable for iPSC gene editing and expression in dopaminergic neurons to enable development of a novel GNDF-secreting stem cell replacement therapy for Parkinson's disease. See FIG. 2 regarding identification of GSH sites based on a computational study and additional predictive and verification modeling. Based on these results, GDNF can be inserted into a GSH in iPSC and a novel cell and gene therapy can be identified for the treatment of Parkinson's.

scATAC-seq data was generated from a clinically compliant iPSC line and resultant dopaminergic neuronal progenitor drug product RNDP-001. This data was compared with published ATAC-seq data from dopaminergic neurons isolated from human healthy and Parkinson's brains to define shared regions of chromatin accessibility based on catalog data of GSH site suitability. First, applicant defined 1,875 novel GSH candidates, identified based on being remote from genes, regulatory regions, telomeres, and centromeres to prevent transgene interference with gene expression, division, and function. A major challenge for defining precisely where a transgene should be targeted is that these 1,875 putative GSH regions span >142 million nucleotides of genomic space, necessitating implementation of additional criterial for prioritization.

The top predicted sgRNAs will be selected for editing using published tools to predict sgRNAs with highest on-target and lowest off-target activities and empirically define the best cutting sgRNA. Leading sgRNA can then be inserted into hGDNF into the identified genomic safe harbor (GSH) using a DNA donor template. Single cell clones can also be isolated and tested for safety and GSH accessibility.

Selected clones can then be differentiated into dopaminergic progenitors and post-mitotic neurons to measure GDNF secretion. Existing release criteria involving a panel of flow cytometry and ddPCR assays will be used to establish whether the edited iPSCs have the capacity for correct dopaminergic progenitor fate specification. RNAseq profiling can also be performed to assess the impact of GDNF insertion on differentiation capacity.

Genomic accessibility may be used as a condition for narrowing down GSH loci optimal for GDNF insertion into iPSCs with the end goal of expression in postmitotic dopaminergic neurons.

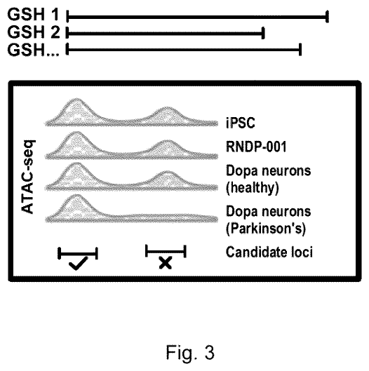

FIG. 3 is illustrative as a starting point for GSH selection, which can be further selected based on publicly available criteria including subregion selectivity.

The disclosure can better be understood by the following numbered paragraphs:
1. An engineered cell, comprising at least one sequence encoding a transgene, wherein the at least one sequence is inserted within a safe harbor locus, the safe harbor locus is at any one or more of loci provided in Table 1.
2. An engineered cell, comprising at least one sequence encoding a transgene, wherein the at least one sequence is inserted within a safe harbor locus, the safe harbor locus is at any one or more loci provided in Table 1; and wherein expression of the at least one sequence encoding the transgene is operatively linked to an endogenous promoter or an exogenous promoter.
3. An engineered cell, comprising at least one sequence encoding a transgene, wherein the at least one sequence is inserted within a safe harbor locus, wherein the safe harbor locus is at any one target loci in Table 1; and wherein expression of the at least one sequence encoding the transgene is operatively linked to an endogenous promoter or an exogenous promoter, and wherein the engineered cell is undifferentiated.
4. The engineered cell of paragraph 1 or 2 or 3, wherein the target locus is selected from: chr1: 214186905-214187961; chr1: 91164906-91165855; chr1: 88180241-88181223; chr1: 72546731-72548018; chr1: 199684394-199685410; chr1: 104647871-104648861; chr10: 109456554-109457498; chr10: 84613037-84614143; chr10: 128540929-128541943; chr10: 128693340-128695044; chr10: 36778899-36780002; chr10: 36725218-36726233; chr11: 121721801-121724194; chr11: 42524286-42525334; chr11: 81647375-81648903; chr11: 116192498-116194401; chr11: 114774331-114775325; chr11: 128021283-128022329; chr11: 116224105-116225916; chr11: 20309899-20311995; chr12: 92649604-92650584; chr12: 94763797-94764936; chr13: 103882813-103883879; chr13: 52967785-52968920; chr13: 52904228-52906942; chr13: 59317916-59319213; chr13: 70309932-70311460; chr13: 76473185-76474229; chr15: 96947520-96948565; chr16: 66127013-66128412; chr17: 56715590-56716621; chr18: 61088416-61089493; chr18: 40576326-40578671; chr18: 27749740-27750778; chr18: 67199846-67201011; chr2: 133886374-133887591; chr2: 163347709-163348850; chr2: 147082542-147083491; chr2: 180125287-180126295; chr2: 121974721-121975718; chr2: 122660609-122661835; chr2: 75920596-75921844; chr2: 103401408-103402401; chr2: 133843863-133844997; chr2: 180123973-180124967; chr2: 160582472-160583418; chr20: 55667282-55669174; chr22: 49103355-49104388; chr3: 67113422-67114525; chr3: 74614218-74615242; chr3: 5426026-5426982; chr3: 117399281-117400399; chr3: 28915343-28917419; chr3: 117439560-117440548; chr3: 117237013-117239704; chr3: 137162914-137164165; chr3: 106609403-106610465; chr3: 67109328-67110455; chr3: 16048355-16049420; chr3: 117397641-117399034; chr3: 104908816-104909868; chr3: 104596179-104597355; chr4: 180058397-180059507; chr4: 180057145-180058232; chr4: 27684257-27685441; chr4: 116925637-116926672; chr4: 166165437-166166501; chr4: 156168620-156169693; chr4: 18689105-18690087; chr4: 85370123-85371213; chr4: 64537927-64540013; chr4: 154982074-154983202; chr4: 130563237-130564339; chr5: 71937993-71940439; chr5: 113806007-113808129; chr5: 18393819-18394795; chr5: 18465687-18466781; chr5: 123874455-123875520; chr5: 123875580-123876580; chr5: 71932489-71933717; chr5: 34474174-34475918; chr5: 18113286-18115121; chr5: 103808080-103809225; chr5: 144598204-144599350; chr5: 108521605-108522526; chr5: 113692453-113694380; chr5: 101678253-101679223; chr5: 87944237-87945265; chr5: 101389859-101390987; chr5: 113678518-113679693; chr5: 88089287-88091255; chr5: 121797421-121798482; chr5: 87949701-87952291; chr5: 103894512-103895553; chr5: 63401153-63402160; chr6: 85922657-85923735; chr6: 104472033-104473578; chr6: 104525429-104526360; chr6: 47097062-47097998; chr6: 87968250-87969278; chr6: 16964556-16965560; chr6: 137415153-137416177; chr6: 91081186-91082896; chr6: 99746005-99747236; chr6: 99822427-99823526; chr6: 140439792-140441984; chr6: 137313918-137314874; chr6: 48765024-48766063; chr6: 90890535-90891762; chr7: 152925055-152926297; chr7: 12111174-12112347; chr7: 42493270-42494306; chr7: 31192574-31193687; chr7: 22000950-22002150; chr7: 96785947-96787328; chr8: 141715497-141716580; chr8: 26189940-26191013; chr8: 131803831-131805003; chr8: 106856111-106857217; chr8: 75975412-75977242; chr8: 115076228-115077322; chr8: 131815871-131817817; chr8: 137492454-137493716; chr9: 85258346-85259505; chr9: 17904998-17907161; chr9: 78439098-78440151; chr9: 16132791-16134459; chr9: 29513212-29515120; chr9: 75265233-75266237; chr9: 7542343-7543943; chr9: 118169738-118170714; chr9: 71517517-71519983; chr9: 7401713-7402906; chr9: 26325995-26327156; chr9: 1453442-1454897; chr9: 105156511-105157922; chr9: 12425914-12426947; chrX: 68894085-68895495; chrX: 20530099-20531285; chrX: 40996550-40997770; chrX: 20527023-20528155; chrX: 94058277-94059471; or chrX: 138127607-138128791.
5. The engineered cell of paragraphs 1-4, wherein the endogenous promoter is any one of SEQ ID NOS 21-40 or 43.
6. The engineered cell of any one of paragraphs 2-5, wherein the endogenous promoter is expressed in Neuronal Mature Cell Types.
7. The engineered cell of any one of paragraphs 1-6, wherein the engineered cell is a stem cell, pluripotent cell, iPSC, a human cell, a primary cell, a DA neuronal cell, a DA neuronal cell progenitor or combinations thereof.
8. The engineered cell of any one of paragraphs 1-7, wherein the engineered cell is undifferentiated.
9. The engineered cell of any one of paragraphs 1-8, wherein the engineered cell is capable of differentiating into a DA neuronal cell.
10. The engineered cell of any one of paragraphs 1-9, wherein the transgene encodes a gene selected from the group comprising, consisting of, or consisting essentially of GDNF, GBA PARK2, PINK1, DJ-1, LRRK2, c-Rel, ATG7, VMAT2 or SNCA and combinations thereof.
11. A composition comprising the engineered cell of any one of paragraphs 1-10 and a pharmaceutical excipient.
12. A guide ribonucleic acids (gRNA) for editing a cell at a safe harbor locus located at a. loci in Table 1.
13. A method of editing a cell having chromosomal DNA, comprising inserting at least one sequence encoding a transgene within a safe harbor locus in the chromosomal DNA of the cell, wherein the safe harbor locus is any one or more of the target loci provided in Table 1.
14. A method of editing a pluripotent cell, comprising contacting a pluripotent cell with one or more guide ribonucleic acids (gRNAs), at least one sequence encoding a transgene, and one or more Cas9 endonucleases, wherein the one or more gRNAs and Cas9 endonucleases facilitate the insertion of the at least one sequence into chromosomal DNA within a safe harbor locus, wherein the safe harbor locus is selected from any one target loci in Table 1.
15. The method of paragraph 13 or 14, wherein the target locus is selected from: chr1: 214186905-214187961; chr1: 91164906-91165855; chr1: 88180241-88181223; chr1: 72546731-72548018; chr1: 199684394-199685410; chr1: 104647871-104648861; chr10: 109456554-109457498; chr10: 84613037-84614143; chr10: 128540929-128541943; chr10: 128693340-128695044; chr10: 36778899-36780002; chr10: 36725218-36726233; chr11: 121721801-121724194; chr11: 42524286-42525334; chr11: 81647375-81648903; chr11: 116192498-116194401; chr11: 114774331-114775325; chr11: 128021283-128022329; chr11: 116224105-116225916; chr11: 20309899-20311995; chr12: 92649604-92650584; chr12: 94763797-94764936; chr13: 103882813-103883879; chr13: 52967785-52968920; chr13: 52904228-52906942; chr13: 59317916-59319213; chr13: 70309932-70311460; chr13: 76473185-76474229; chr15: 96947520-96948565; chr16: 66127013-66128412; chr17: 56715590-56716621; chr18: 61088416-61089493; chr18: 40576326-40578671; chr18: 27749740-27750778; chr18: 67199846-67201011; chr2: 133886374-133887591; chr2: 163347709-163348850; chr2: 147082542-147083491; chr2: 180125287-180126295; chr2: 121974721-121975718; chr2: 122660609-122661835; chr2: 75920596-75921844; chr2: 103401408-103402401; chr2: 133843863-133844997; chr2: 180123973-180124967; chr2: 160582472-160583418; chr20: 55667282-55669174; chr22: 49103355-49104388; chr3: 67113422-67114525; chr3: 74614218-74615242; chr3: 5426026-5426982; chr3: 117399281-117400399; chr3: 28915343-28917419; chr3: 117439560-117440548; chr3: 117237013-117239704; chr3: 137162914-137164165; chr3: 106609403-106610465; chr3: 67109328-67110455; chr3: 16048355-16049420; chr3: 117397641-117399034; chr3: 104908816-104909868; chr3: 104596179-104597355; chr4: 180058397-180059507; chr4: 180057145-180058232; chr4: 27684257-27685441; chr4: 116925637-116926672; chr4: 166165437-166166501; chr4: 156168620-156169693; chr4: 18689105-18690087; chr4: 85370123-85371213; chr4: 64537927-64540013; chr4: 154982074-154983202; chr4: 130563237-130564339; chr5: 71937993-71940439; chr5: 113806007-113808129; chr5: 18393819-18394795; chr5: 18465687-18466781; chr5: 123874455-123875520; chr5: 123875580-123876580; chr5: 71932489-71933717; chr5: 34474174-34475918; chr5: 18113286-18115121; chr5: 103808080-103809225; chr5: 144598204-144599350; chr5: 108521605-108522526; chr5: 113692453-113694380; chr5: 101678253-101679223; chr5: 87944237-87945265; chr5: 101389859-101390987; chr5: 113678518-113679693; chr5: 88089287-88091255; chr5: 121797421-121798482; chr5: 87949701-87952291; chr5: 103894512-103895553; chr5: 63401153-63402160; chr6: 85922657-85923735; chr6: 104472033-104473578; chr6: 104525429-104526360; chr6: 47097062-47097998; chr6: 87968250-87969278; chr6: 16964556-16965560; chr6: 137415153-137416177; chr6: 91081186-91082896; chr6: 99746005-99747236; chr6: 99822427-99823526; chr6: 140439792-140441984; chr6: 137313918-137314874; chr6: 48765024-48766063; chr6: 90890535-90891762; chr7: 152925055-152926297; chr7: 12111174-12112347; chr7: 42493270-42494306; chr7: 31192574-31193687; chr7: 22000950-22002150; chr7: 96785947-96787328; chr8: 141715497-141716580; chr8: 26189940-26191013; chr8: 131803831-131805003; chr8: 106856111-106857217; chr8: 75975412-75977242; chr8: 115076228-115077322; chr8: 131815871-131817817; chr8: 137492454-137493716; chr9: 85258346-85259505; chr9: 17904998-17907161; chr9: 78439098-78440151; chr9: 16132791-16134459; chr9: 29513212-29515120; chr9: 75265233-75266237; chr9: 7542343-7543943; chr9: 118169738-118170714; chr9: 71517517-71519983; chr9: 7401713-7402906; chr9: 26325995-26327156; chr9: 1453442-1454897; chr9: 105156511-105157922; chr9: 12425914-12426947; chrX: 68894085-68895495; chrX: 20530099-20531285; chrX: 40996550-40997770; chrX: 20527023-20528155; chrX: 94058277-94059471; or chrX: 138127607-138128791.
16. The method of any one of paragraphs 13-15, wherein the at least one sequence comprises an exogenous promoter is any one of Seq ID nos. 21-40 or 43.
17. The method of any one of paragraphs 13-16, wherein the cell is a stem cell, pluripotent cell, iPSC, a human cell, a primary cell, a DA neuronal cell, a DA neuronal cell progenitor or combinations thereof.
18. The method of any one of paragraphs 13-17, wherein the engineered cell is undifferentiated.
19. The method of any one of paragraphs 13-18, wherein the at least one sequence is inserted using a homology-directed repair.
20. The method of any one of paragraphs 13-19, wherein the at least one sequence is inserted using a homology independent targeted insertion.
21. The method of any one of paragraphs 13-20, wherein the at least one sequence is inserted using one or more guide ribonucleic acids (gRNAs) and one or more Cas9 endonucleases.
22. The method of any one of paragraphs 13-21, wherein the transgene is selected from the group comprising GDNF, GBA PARK2, PINK1, DJ-1, LRRK2, c-Rel, ATG7, VMAT2 or SNCA and combinations thereof.
23. An ex vivo method of obtaining an engineered cell or population thereof, comprising: a. obtaining a cell; b. genetically modifying the cell by inserting at least one sequence encoding a transgene within a safe harbor locus, wherein the safe harbor locus is selected from any one target loci in Table 1.
24. The method of paragraph 23, wherein obtaining the cell comprises: (i) collecting a tissue sample from a subject, (ii) isolating the cells from the tissue samples, and (iii) culturing the cells in vitro.
25. The method of paragraph 23, wherein the tissue sample is a blood sample.
26. The method of any one of paragraphs 23-25, wherein the cell is a stem cell, a human cell, a primary cell, a hematopoietic cell, an adaptive immune cell, an innate immune cell, a T cell, or T cell progenitor or combinations thereof.
27. The method of any one of paragraphs 23-26, wherein the engineered cell is undifferentiated.
28. The method of any one of paragraphs 23-27, wherein the at least one sequence is selected from the group comprising GDNF, GBA PARK2, PINK1, DJ-1, LRRK2, c-Rel, ATG7, VMAT2 or SNCA and combinations thereof.
29. The method of any one of paragraphs 23-28, wherein the at least one sequence is inserted using a homology-directed repair.
30. The method of any one of paragraphs 23-29, wherein the at least one sequence is inserted using a homology independent targeted insertion.
31. The method of any one of paragraphs 23-30, wherein the genetically modifying in step (b) comprises contacting the cell with one or more guide ribonucleic acids (gRNAs), the at least one sequence, and one or more Cas9 endonucleases, wherein the one or more gRNAs and Cas9 endonucleases facilitate the insertion of the at least one sequence into chromosomal DNA within the safe harbor locus.
32. The method of any one of paragraphs 23-31, wherein the transgene encodes a gene selected from the group comprising GDNF, GBA PARK2, PINK1, DJ-1, LRRK2, c-Rel, ATG7, VMAT2 or SNCA and combinations thereof.
33. The method of any one of paragraph 23-32, wherein the at least one sequence comprises an exogenous promoter and the exogenous promoter is operably linked to the transgene.
34. The method of any one of paragraph 23-33, wherein the exogenous promoter is any one of SEQ ID NOS 21-40 or 43.
35. A method of treating a subject having or at risk of having a disease, comprising administering to the subject an effective amount of the cell of any one of paragraphs 13-24, a population thereof, or the composition of paragraphs 1-12.
36. A method of treating a subject having or at risk of having a disease, comprising: a. conducting the method of any one of paragraphs 13-35; and b. administering to the subject an effective amount of a composition comprising the cell or a population thereof.
37. The method of paragraph 35 or 36, wherein the composition is administered to the subject by infusion.
38. The method of paragraph 35-37, wherein the disease is Parkinson's.
39. The method of any one of paragraphs 35-38 wherein the disease is a disorder associated with central nervous system degeneration.
40. A method of treating a subject having or at risk of having a disease, comprising administering to the subject an effective amount of the cell of any one of paragraphs 1-11, a population thereof, or the composition of paragraph 12.
41. A method of treating a subject having or at risk of having a disease, comprising: c. conducting the method of any one of paragraphs 13-34; and d. administering to the subject an effective amount of a composition comprising the cell or a population thereof.
42. The method of paragraph 40-41, wherein the composition is administered to the subject by infusion.
43. The method of paragraph 40-42, wherein the disease is Parkinson's.
44. A method of identifying a safe harbor locus, comprising: a. identifying genes or non-coding regions in a chromosome that are above a threshold level for accessibility of chromatin; b. generating a model that correlates the gene or non-coding region from step (a) with published ATAC-seq data from dopaminergic neurons isolated from human healthy and Parkinson's brains on the chromosome; and c. selecting the safe harbor locus based on threshold parameters; wherein the safe harbor locus is selected for insertion of at least one sequence encoding a transgene within a cell.
45. The method of paragraph 44, wherein the threshold parameters include one or more of: stable expression of a transgene, knockout of the gene confers benefit to the function of the cell, no known function within the cell, stable transgene expression in vitro, negligible off-target cleavage as detected by iGuide-Seq or CRISPR-Seq, less off-target cleavage relative to other loci as detected by iGuide-Seq or CRISPR-Seq, negligible transgene-independent cytotoxicity, negligible transgene-independent cytokine expression, negligible transgene-independent chimeric antigen receptor expression, negligible deregulation or silencing of nearby genes, peak size data generated with processed ATAC-seq data, distribution data generated with processed ATAC-seq data, highest efficiency for transgene insertion, durable expression following prolonged culture, and absence of oncogenic gene expression.
46. The method of any one of paragraphs 44-45, wherein the accessibility of chromatin is measured using an assay for transposase-accessible chromatin using sequencing (ATAC-seq).
47. The method of any one of paragraphs 44-46, wherein the engineered cell is a stem cell, pluripotent cell, iPSC, a human cell, a primary cell, a DA neuronal cell, a DA neuronal cell progenitor or combinations thereof.
48. The engineered cell, composition, or method of any one of the preceding paragraphs, wherein insertion within the safe harbor locus increase GDNF secreted proteins the GDNF secreted proteins are made and taken up by the endogenous cells after the administered engineered cells have matured to a Neuronal Mature Cell Type.
49. The engineered cell, composition, or method of any one of the preceding paragraphs wherein knock-in efficiency at any one of the safe harbor locus in Table 1 is increased relative to other locations along the chromosome.

1. A population of engineered cells, wherein the engineered cells comprise at least one exogenous GBA gene or functional fragment thereof.

2. The population of cells of claim 1, wherein the engineered cells are human DA neuronal cells.

3. The population of cells of claim 2, wherein the human DA neuronal cells are derived from pluripotent cells.

4. The population of cells of claim 1, wherein the engineered cells are engineered to be hemizygous null for an SNCA gene.

5. The population of cells of claim 1, wherein the exogenous GBA gene or functional fragment thereof is within or near a safe harbor locus.

6. The population of cells of claim 5, wherein the safe harbor locus is chr1: 214186905-214187961.

7. The population of cells of claim 5, wherein the safe harbor locus is selected from chr1: 91164906-91165855; chr1: 88180241-88181223; chr1: 72546731-72548018; chr1: 199684394-199685410; chr1: 104647871-104648861; chr10: 109456554-109457498; chr10: 84613037-84614143; chr10: 128540929-128541943; chr10: 128693340-128695044; chr10: 36778899-36780002; chr10: 36725218-36726233; chr11: 121721801-121724194; chr11: 42524286-42525334; chr11: 81647375-81648903; chr11: 116192498-116194401; chr11: 114774331-114775325; chr11: 128021283-128022329; chr11: 116224105-116225916; chr11: 20309899-20311995; chr12: 92649604-92650584; chr12: 94763797-94764936; chr13: 103882813-103883879; chr13: 52967785-52968920; chr13: 52904228-52906942; chr13: 59317916-59319213; chr13: 70309932-70311460; chr13: 76473185-76474229; chr15: 96947520-96948565; chr16: 66127013-66128412; chr17: 56715590-56716621; chr18: 61088416-61089493; chr18: 40576326-40578671; chr18: 27749740-27750778; chr18: 67199846-67201011; chr2: 133886374-133887591; chr2: 163347709-163348850; chr2: 147082542-147083491; chr2: 180125287-180126295; chr2: 121974721-121975718; chr2: 122660609-122661835; chr2: 75920596-75921844; chr2: 103401408-103402401; chr2: 133843863-133844997; chr2: 180123973-180124967; chr2: 160582472-160583418; chr20: 55667282-55669174; chr22: 49103355-49104388; chr3: 67113422-67114525; chr3: 74614218-74615242; chr3: 5426026-5426982; chr3: 117399281-117400399; chr3: 28915343-28917419; chr3: 117439560-117440548; chr3: 117237013-117239704; chr3: 137162914-137164165; chr3: 106609403-106610465; chr3: 67109328-67110455; chr3: 16048355-16049420; chr3: 117397641-117399034; chr3: 104908816-104909868; chr3: 104596179-104597355; chr4: 180058397-180059507; chr4: 180057145-180058232; chr4: 27684257-27685441; chr4: 116925637-116926672; chr4: 166165437-166166501; chr4: 156168620-156169693; chr4: 18689105-18690087; chr4: 85370123-85371213; chr4: 64537927-64540013; chr4: 154982074-154983202; chr4: 130563237-130564339; chr5: 71937993-71940439; chr5: 113806007-113808129; chr5: 18393819-18394795; chr5: 18465687-18466781; chr5: 123874455-123875520; chr5: 123875580-123876580; chr5: 71932489-71933717; chr5: 34474174-34475918; chr5: 18113286-18115121; chr5: 103808080-103809225; chr5: 144598204-144599350; chr5: 108521605-108522526; chr5: 113692453-113694380; chr5: 101678253-101679223; chr5: 87944237-87945265; chr5: 101389859-101390987; chr5: 113678518-113679693; chr5: 88089287-88091255; chr5: 121797421-121798482; chr5: 87949701-87952291; chr5: 103894512-103895553; chr5: 63401153-63402160; chr6: 85922657-85923735; chr6: 104472033-104473578; chr6: 104525429-104526360; chr6: 47097062-47097998; chr6: 87968250-87969278; chr6: 16964556-16965560; chr6: 137415153-137416177; chr6: 91081186-91082896; chr6: 99746005-99747236; chr6: 99822427-99823526; chr6: 140439792-140441984; chr6: 137313918-137314874; chr6: 48765024-48766063; chr6: 90890535-90891762; chr7: 152925055-152926297; chr7: 12111174-12112347; chr7: 42493270-42494306; chr7: 31192574-31193687; chr7: 22000950-22002150; chr7: 96785947-96787328; chr8: 141715497-141716580; chr8: 26189940-26191013; chr8: 131803831-131805003; chr8: 106856111-106857217; chr8: 75975412-75977242; chr8: 115076228-115077322; chr8: 131815871-131817817; chr8: 137492454-137493716; chr9: 85258346-85259505; chr9: 17904998-17907161; chr9: 78439098-78440151; chr9: 16132791-16134459; chr9: 29513212-29515120; chr9: 75265233-75266237; chr9: 7542343-7543943; chr9: 118169738-118170714; chr9: 71517517-71519983; chr9: 7401713-7402906; chr9: 26325995-26327156; chr9: 1453442-1454897; chr9: 105156511-105157922; chr9: 12425914-12426947; chrX: 68894085-68895495; chrX: 20530099-20531285; chrX: 40996550-40997770; chrX: 20527023-20528155; chrX: 94058277-94059471; or chrX: 138127607-138128791.

8. A population of engineered cells, wherein the engineered cells comprise: at least one exogenous GDNF gene or functional fragment thereof.

9. A method for preparing the engineered cells of claim 8, the method comprising:
providing a population of cells comprising pluripotent cells;
introducing into a portion of the pluripotent cells: i) at least one nucleic acid comprising an exogenous polynucleotide sequence to be integrated at a selected endogenous locus to encode at least one GDNF gene;
ii) at least one sequence-specific reagent that specifically targets the selected endogenous locus, wherein the exogenous polynucleotide sequence is inserted by targeted gene integration into the endogenous locus thereby forming engineered pluripotent cells.

10. A method for preparing engineered pluripotent cells, the method comprising:
providing a population of cells comprising pluripotent cells;
introducing into a portion of the pluripotent cells:
i) at least one nucleic acid comprising an exogenous polynucleotide sequence to be integrated at a selected endogenous locus wherein the exogenous polynucleotide sequence encodes at least one GBA gene wherein the engineered pluripotent cells are engineered to be hemizygous null for a SNCA gene;
ii) at least one sequence-specific reagent that specifically targets the selected endogenous locus, wherein the exogenous polynucleotide sequence is inserted by targeted gene integration into the endogenous locus thereby forming engineered pluripotent cells.

11. The method according to claim 10, wherein the engineered pluripotent cells are differentiated to DA neuronal cells to form engineered DA neuronal cells.

12. The method according to claim 11, wherein the engineered DA neuronal cells are transplanted into a subject.

13. The method according to claim 12, wherein the transplanted engineered DA neuronal cells express GBA and SNCA upon transplantation.

14. The method according to claim 12, wherein the transplanted engineered DA neuronal cells do not exhibit α-synuclein accumulation.

15. The method according to claim 12, wherein the transplanted engineered DA neuronal cells have increased cell viability, engraftment, proliferation, migration, innervation, differentiation, long-term graft integrity, survival of endogenous neurons, or function compared to wildtype DA neuronal cells.

16. The method according to claim 12, wherein the transplanted engineered DA neuronal cells treat or inhibit onset of Parkinson's Disease and secondary Parkinsonian disorders in the subject.

17. The population of cells of claim 1, wherein the exogenous GBA gene or functional fragment thereof is under the control of a GBA promoter selected from the group comprising SEQ ID NOS: 31-40 or 43.

18. The population of cells of claim 1, wherein the exogenous GBA gene is behind a GDNF knock-in promoter selected from the group comprising SEQ ID NOS: 21-30.

19. The population of cells of claim 1, wherein the exogenous GBA gene or functional fragment thereof comprises the nucleic acid sequence set forth in SEQ ID NO: 7.

20. The population of cells of claim 2, wherein after transplantation of the human DA neuronal cells into a subject, the exogenous GBA gene or functional fragment thereof is expressed before the transplanted human DA neuronal cells have graft function.
